(19) United States
(12) Patent Application Publication
(10) Pub. No.: US 2010/0256145 A1
(43) Pub. Date: Oct. 7, 2010

(54) USE OF KCNQ POTASSIUM CHANNEL OPENERS FOR REDUCING SYMPTOMS OF OR TREATING DISORDERS OR CONDITIONS WHEREIN THE DOPAMINERGIC SYSTEM IS DISRUPTED

(75) Inventors: Henriette Husum Bak-Jensen, Kobenhavn S (DK); Klaus Peter Hertel, Jystrup (DK)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. LUNDBECK A/S, Valby (DK)

(21) Appl. No.: 12/671,505

(22) PCT Filed: Jul. 31, 2008
(86) PCT No.: PCT/DK08/50191

§ 371 Date: May 20, 2010

Related U.S. Application Data
(60) Provisional application No. 60/953,308, filed on Aug. 1, 2007.

(30) Foreign Application Priority Data
Aug. 1, 2007 (DK) PA 2007 01110

(51) Int. Cl. A61K 31/5355; A61K 31/44; A61K 31/245; A61P 25/00

(52) U.S. Cl. 514/235.5; 514/352; 514/538

(57) ABSTRACT

The invention relates, inter alia, to a method for reducing symptoms of, or for treating, one or more disorders or conditions wherein the dopaminergic system is disrupted, the method comprising administering to a host in need thereof an effective amount of a compound that is able to increase the ion flow through KCNQ potassium channels.

### FIELD OF THE INVENTION

The present invention relates, inter alia, to a novel method for reducing symptoms of, or for treating, one or more disorders or conditions wherein the dopaminergic system is disrupted, such as one or more disorders or conditions independently selected from the group consisting of: schizophrenia and other psychotic states; mood disorders ADHD, aggression; movement disorders; and substance use and/or abuse; the method comprising administering to a host in need thereof an effective amount of a compound able to increase the ion flow through KCNQ potassium channels.

Furthermore, the present invention relates to the use of KCNQ potassium channel openers for the preparation of a pharmaceutical composition for reducing symptoms of, or for treating, disorders wherein the dopaminergic system is disrupted.

Furthermore, the present invention relates to a method of screening for a compound which is a KCNQ potassium channel opener and which is capable of having a potential for reducing symptoms of or for treating, one or more disorders wherein the dopaminergic system is disrupted.

### BACKGROUND OF THE INVENTION

The dopaminergic system is known to be disrupted in schizophrenia and other psychotic states (Meltzer and Stahl Schizophrenia Bulletin, 1976, 2, 19-76) and the compounds currently available for the treatment of schizophrenia all modulate the dopaminergic system. These compound do so by inhibiting the signalling properties of a number of brain-expressed receptors, most notably the dopamine D2 receptor. However, a number of other receptors are also involved in the activity of many antipsychotic drugs, including serotonergic, noradrenergic, histaminergic and muscarinic receptors (Scolnick, Schizophrenia Bulletin, 2004, 72, 75-77).

The known antipsychotic compounds all produce a range of side-effects in addition to their effect of reducing the symptoms of schizophrenia and other psychotic states. The nature of the side-effects depend upon the exact pharmacology of the compound in question. All clinically used antipsychotics interact with the dopamine D2 receptor to some degree or other (Seeman et al., Nature 261, 717-719). Those compounds that require a high degree of dopamine D2 receptor blockade, for example haloperidol, cause extrapyramidal side-effects and elevations in prolactin levels. Extrapyramidal side-effects are also experienced by patients treated with partial dopamine D2 receptor agonists such as aripiprazole (Kasper et al, Int J Neuropsychopharmacol, 2003, 6, 325-337). Extrapyramidal side-effects include Parkinsonism, rigidity, akinesia and after prolonged treatment tardive dyskinesia may develop (Pierre, Drug Safety, 2005, 28, 191-208). Prolactin elevation can cause a number of endocrine disturbances, such as gynaecomastia, galactorrhoea, sexual disfunction, infertility, oligomenorrhoea and amenorrhoea (Haddad and Wieck Drugs, 2004, 64, 2291-2314).

The known antipsychotics, particularly the newer class of atypical antipsychotics, such as olanzapine, quetiapine and risperidone, are also associated with insulin resistance', disturbances in glucose and lipid metabolism, diabetes and excessive weight gain (Melkersson and Dahl, Drugs 2004, 64, 701-723).

In addition, the known antipsychotics may cause “slowness of thinking”, which contributes to the cognitive symptoms of schizophrenia. Furthermore, anhedonia, the decrease in mood, may also occur with some antipsychotics and may appear to worsen the negative symptoms of schizophrenia (Heinz et al, Schizophrenia Research, 1998, 31, 19-26). Furthermore, the known antipsychotics may also cause an array of other disturbing side-effects such as hypotension and dizziness, tachycardia, sedation, agamalocytoses, leukopenia, hypersalivation, hepatotoxicity and blurred vision Stanniland and Taylor, Drug Safety, 2000, 22, 195-214).

The known antipsychotics also inadequately treat the symptoms of schizophrenia. The symptoms of schizophrenia fall into four broad categories: positive, negative, cognitive and affective symptoms, such as depressive symptoms. The positive symptoms are those which represent an ‘excess’ of normal behaviour, such as one or more of hallucinations, delusions, thought disorders, distortions or exaggerations in language and communication, disorganized speech, disorganized behaviour or agitation. The negative symptoms are those where the patients show a lack of normal behaviour, such as one or more of blunted affect, aphasia, asociality, anhedonia, avolition, emotional withdrawal, difficulty in abstract thinking, lack of spontaneity, stereotyped thinking, alogia and attentional impairment. The cognitive symptoms, relate to the cognitive deficits in schizophrenia, such as one or more of lack of sustained attention, deficits in executive function and memory. Affective symptoms of schizophrenia may include depressive symptoms such as depressed mood in general, anhedonic symptoms, sleep disturbances, psychomotor agitation or retardation, sexual dysfunction, weight loss, concentration difficulties, delusional ideas, loss of energy, feelings of worthlessness, recurrent thoughts of death or suicidal ideation. Depressive symptoms in schizophrenia appear to be associated with a generally poor treatment outcome and are relatively frequently with an estimated prevalence of 25-60% (Montgomery and van Zwieten-Boot, Eur Neuropychopharmacol., 2007, 17, 70-77).

Schizophrenia can be subdivided based on the clinical picture. The paranoide subtype of schizophrenia is characterized by the presence of prominent delusions or auditory hallucinations in the context of a relative preservation of cognitive functioning and affect whereas disorganized speech and behaviour, flat or inappropriate affect are essential features for the disorganized subtype of schizophrenia. The essential feature of the catatonic subtype of schizophrenia is a marked psychomotor disturbance that may involve both motoric immobility as well as excessive motor activity. Finally, the residual subtype of schizophrenia is characterized by a lack of prominent positive symptoms.

The known antipsychotics largely treat the positive symptoms of schizophrenia and have limited impact on primary negative, cognitive or depressive symptoms (Mishara and Goldberg, Biological Psychiatry, 2004, 55, 1013-1022; Conley et al., Schizophrenia Res., 2007, 90, 186-197). In addition, the clinical benefit derived from antipsychotics takes several weeks of treatment to develop. In a recent large comparative study (the CATIE study) approximately 30-40% of patients discontinued treatment (switched to another drug) because of lack of efficacy (Lieberman et al New England Journal Of Medicine, 2005, 353, 1209-1223).

Besides schizophrenia, known antipsychotics with their mentioned shortcomings and disadvantages are also used in other psychotic disorders such as schizophreniform disorder, schizoaffective disorder, delusional disorder and brief psychotic disorder. Psychotic symptoms can also be induced by substances (such as central stimulants) or appear in other general medical conditions such as Alzheimer's disease, dementia or bipolar disorder (Tamminga and Davis, Schizophrenia Bull., published on-line Jun. 11, 2007).

Also, psychosis in Parkinson's disease is rather common; approximately 20-30% of Parkinson's patients manifest psychotic symptoms (Chou et al, Expert Opin. Pharmacother., 2007, 8, 935-943). The psychotic symptoms in this disorder could be induced by standard treatments (such as L-DOPA) but may also be consequence of the underlying pathophysiology of Parkinson's disease.

It is also well established that psychotic symptoms may be present in depression, so called psychotic depression. Psychotic depression is not uncommon and epidimeological studies suggests that around 15% of patients diagnosed with major depression had a history of episodes with psychotic symptoms (Wijkstra et al., Cochrane Database Sys Rev., 2005, 19, 1-43). Psychotic depression is typically treated with antidepressant or antipsychotic drugs alone or in combination. Like the current antipsychotics previously described, the currently available antidepressant drugs also have an array of disturbing side-effects including nausea, diarrhoea, dizziness, insomnia, tremor, reduced appetite, blurred vision, sexual dysfunctions, decreased libido, etc.

Mood disorders include disorders that have a disturbance in mood as the predominat feature. Thus, both depressive disorders like major depressive disorder, dysthymic disorder, depressive disorder not otherwised specified, minor depression and brief recurrent depression mood disorders as well as bipolar spectrum disorders like bipolar I disorder, bipolar II disorder and cyclothymic disorder are classified as mood disorders. Major depressive disorder is a chronic recurring disease with considerable morbidity in the general population. The hallmark of the disease is a depressed mood. The clinical picture may be further characterised by anhedonic symptoms, sleep disturbances, psychomotor agitation or retardation, sexual dysfunction, weight loss, concentration difficulties and delusional ideas. However, the most serious complication of a depressive episode is that of suicidal ideation leading to suicide attempts (DSM IV, American Psychiatric Association, Washington D.C. 1994). Besides major depressive disorder, other disorders are characterized by depressed mood, such as dysthymic disorder, depressive disorder not otherwised specified, minor depression and recurrent brief depressive disorder (DSM IV, American Psychiatric Association, Washington D.C. 1994). Dysthymic disorder is differentiated from major depressive disorder based on severity, chronicity and persistence. Dysthymic disorder is characterized by chronic, less severe depressive symptoms that have been present for many years. The depressive disorder not otherwised specified category includes disorders with depressive features, like minor depressive disorder and recurrent brief depressive disorder, that do not meet the criteria for other depressive disorders like major depressive disorder or dysthymic disorder. The essential feature of minor depression is one or more periods of depressive symptoms that are identical to those expressed in major depressive disorder in duration but which involves fewer symptoms and less impairment. Brief recurrent depression is characterised by recurrent brief episodes of depressive symptoms that are identical to those expressed in major depressive disorder in number and severity but with shorter duration. Consequently, it is the goal of treatment of the depression that the symptoms are effectively alleviated, the treatment is safe and highly tolerable and the treatment has an early on set of effect.

Bipolar spectrum disorders, previously referred to as manic-depressive illness, are mood disorders where depressive symptoms are combined with at least one manic, hypomanic or mixed episode. A manic episode is characterised by a distinct period of abnormally and persistently elevated, expansive or irritable mood. A mixed episode is characterized by a period lasting at least one week in which both the criteria for a manic and major depressive episode are met. In similarity to a manic episode, a hypomanic episode is characterized by a distinct period during which there is an abnormally and persistently elevated, expansive or irritable mood. However, in contrast to a manic episode, a hypomanic episode is not severe enough to cause marked impairment in social or occupational functioning or to require hospitalisation and there are no psychotic features. The symptoms of a bipolar depressive episode are not different from those characterizing a major depressive episode. This is also the reason why many bipolar patients are initially diagnosed as suffering from major depression. As mentioned, it is the occurrence of manic, or mixed or hypomanic episodes that give rise to a bipolar diagnosis, which is distinct from a major depression diagnosis.

Bipolar spectrum disorders may be subdivided into bipolar I disorder, bipolar II disorder, cyclothymic disorder and bipolar disorder not otherwise specified. Bipolar I disorder is characterized by the occurrence of one or more manic or mixed episodes and often individuals have also had one or more major depressive episodes. Bipolar II disorder is characterized by the occurrence of one or more major depressive episodes accompanied by at least one hypomanic episode. Due to the progressive nature of bipolar I and II disorder, the patients experience an increasing risk of recurrence of symptoms with every new episode, as well as a growing risk of increasing duration and severity of subsequent episodes, if untreated. For this reason, both bipolar I or bipolar II disorder patients may eventually be classified as rapid cycling patients where the patient experiences at least four episodes per year. Cyclothymic disorder is a sub-group of bipolar spectrum disorders, where the mood disturbances are characterized by chronic, fluctuating mood disturbances involving numerous periods of hypomania and periods of depressive symptoms. Bipolar disorder not otherwise specified refers to a category of disorders with bipolar features that do not meet the criteria for any specified bipolar disorder mentioned above. Bipolar spectrum disorders are life-threatening conditions since patients diagnosed with a bipolar disorder have an estimated suicide risk 15 times higher than in the general population (Harris and Barraclough, 1997, British Journal of Psychiatry, 170:205-228). At present bipolar spectrum disorders are treated by maintaining the bipolar patients on mood-stabilisers (mainly lithium or antiepileptics) and adding antimanic agents (lithium or antipsychotics) or antidepressants (tricyclic antidepressants or selective serotonin re-uptake inhibitors) when the patients relapse into a manic or depressive episode, respectively (Liebermann and Goodwin, Curr. Psychiatry Rep. 2004, 6:459-65). Thus, there is a desire to develop novel therapeutic treatments for bipolar spectrum disorders in order to meet the need of effectively treating all three crucial elements in these disorders with only one therapeutic agent: such novel agents should alleviate manic symptoms with a fast onset of action (antimanic activity), alleviate depression symptoms with a fast onset of action (antidepressant activity), prevent the recurrence of mania as well as depression symptoms (mood stabilising activity).

Attention-deficit/hyperactivity disorder (ADHD) refers clinically to a relatively common syndrome, as epidemiologic studies have suggested that the prevalence of ADHD among the general population is between 2-10%. ADHD begins in childhood and typically but not necessarily remits by adulthood (Szatmari Child Adolesc. Psychiat. Clin. North Am. 1982, 1, 361-371). ADHD is clinically characterised by inattention (e.g. failure to give close attention, difficulties in sustaining attention, difficulties in organising tasks and activities and easily distracted by extraneous stimuli), hyperactivity (e.g. difficulties in remaining seated, excessive motor activity in inappropriate situations, the patient acts as if “driven by a motor”) and impulsivity (e.g. difficulties in awaiting turn, answer questions before they have been completed and often interrupts or intrudes ongoing conversation; American Psychiatric Association, Diagnostic and Statistical Manual of Mental Disorders (DSM-IV), 10, 1994). Twin studies of ADHD have indicated that around 80% of the etiology of ADHD is attributed to genetic factors (Gjone et al. J. Am. Acad. Child Adolesc. Psychiat. 1996, 35, 588-596). Although, the strong genetic component of the disease, the patophysiology of ADHD, is currently not known. Psychostimulants, particularly methylphenidate and dextroamphetamine, have been and continue to be the drugs of choice in treating patients with ADHD (Faraone and Biederman, In: Neurobiology of Mental Illness, eds: Charney, Nestler and Bunney, Oxford University Press, 1999, 60, 788-801). Although psychostimulants appear effective, there are a number of problems associated with their use in the treatment of ADHD patients. For example, some patients do not respond at all or only partially to treatment. Furthermore, adverse effects such as insomnia, decreased appetite, irritability, tics and depressive symptoms after long-term treatment are relatively frequent in ADHD patients treated with psychostimulants. Consequently, there is still a large unmet need for efficient and better tolerated drugs for the treatment of this condition.

The use and/or abuse of substances such as nicotine, CNS depressants such as alcohol, opioids such as heroin and morphine, psychostimulants such as amphetamine and cocaine, and cannabis represents a heavy burden on the society as a whole and causes significant personal suffering. The precise biological mechanisms underlying substance dependence and substance abuse are not known but several lines of evidence suggest that the mesocorticolimbic dopamine system play a major role in this context. For example, several, if not all, of the substances with an addictive property increases the amount of dopamine in limbic regions of the brain such as the nucleus accumbens (Di Chiara and Bassareo, Curr Opin Pharmacol, 2007, 7, 69-76). Despite the societal burden and personal suffering related to drug abuse/addiction, efficacious pharmacotherapeutic strategies are lacking (Heidbreder, Eur J Pharmacol, 2005, 526, 101-112). Therefore, it is a great unment need for more successful pharmacological treatments for nicotine, alcohol, opiates, central stimulants and cannabis addiction and/or abuse.

Aggression is traditionally defined as overt behaviour that has the intention of inflicting harm. Aggression is seen as an important issue with a relatively high incidence. For example, in studies in the mental health care system, an incidence of 9.3 incidencts per bed year has been reported (Geodhard, et al, J Clin Psychiatry, 2006, 67, 1013-1024). Aggression is a symptom that may be present in several other clinical conditions such as in impulse-control disorders (intermittent explosive disorder), schizophrenia, bipolar spectrum disorders, Alzheimer's disease, dementia, Parkinson's disease, etc. The standard treatment of aggression includes sedative drugs like benzodiazepines, antipsychotic agents, beta-adrenergic blockers, anticonvulsants and antidepressant drugs (Geodhard, et al, J Clin Psychiatry, 2006, 67, 1013-1024). Although several treatment options have been employed, there remains no consensus on the optimal treatment of aggression. In general, only weak evidence for efficacy against aggression of the currently used medications has been reported (Geodhard, et al, J Clin Psychiatry, 2006, 67, 1013-1024) and several of these drugs have sedative effects and do not appear to selectively target aggression independently. Thus, novel treatment of aggression with better tolerability and efficacy is clearly indicated.

Like Parkinson's disease, Tourette's syndrome as well as Huntington's disease, belong to the group of movement disorders. Tourette's syndrome is an inherited neurological disorder with onset in childhood, characterized by the presence of multiple physical (motor) tics and at least one vocal (phonic) tic. Tourette's syndrome is defined as part of a spectrum of tic disorders, which includes transient and chronic tics. Pharmacological treatment of Tourette's syndrome is indicated in severe cases when the symptoms interfere with daily functioning. Treatment with known antipsychotics is considered as the standard pharmacological treatment of Tourette's syndrome (Sandor, J Psychosomatic Res, 2003, 55, 41-48). However, as already described, these drugs are associated with a variety of disturbing side-effects such as extrapyramidal motor side-effects and metabolic disturbances. Antihypertensive agents such as clonidine and guanfacine are also used to treat tics but studies have showed variable efficacy of such treatments (Sandor, J Psychosomatic Res, 2003, 55, 41-48). Given these shortcomings of the current treatment options for Tourette's syndrome, novel pharmacological treatments with better tolerability and efficacy are indeed needed.

Given the limitations of the known antipsychotics as previously described, there is a great need to develop new compounds with better treatment outcome in disorders wherein the dopaminergic system is disrupted such as one or more disorders independently selected from the group consisting of schizophrenia and other psychotic states; mood disorders; ADHD; aggression; movement disorders; and substance use and/or abuse. The shortcomings of the known antipsychotics are illustrated by a recent study showing that almost 75% of schizophrenic patients discontinued their antipsychotic medication before 18 months (Lieberman et al., N Engl J Med, 2005, 353, 1209-1223). A majority of patients in this study discontinued their medication due to inefficacy or intolerable side-effects. It is well known that side-effects do have a greate negative impact on the treatment compliance. For example, extrapyramidal side-effects, caused by central dopamine D2 receptor blockade, have been identified as a major risk factor for non-compliance in schizophrenic patients (Robinson et al., Schizophrenia Res, 2002, 57, 209-219). In turn, non-compliance to treatment play a major role in the overall outcome of a patient. For example, studies in schizophrenic patients have shown that non-compliance to treatment plays a major role in increasing the risk of psychotic relapses and re-hospitalizations (Kane, J Clin Psychiat, 2006, 67, Suppl. 5, 9-14).

Thus, there is a great need to develop new compounds for the treatment of disorders wherein the dopaminergic system is disrupted such as one or more disorders independently selected from the group consisting of schizophrenia and other psychotic states; mood disorders; ADHD; aggression; movement disorders; and substance use and/or abuse with improved profile in respect of tolerability and/or side-effects, and as a consequence, better compliance and treatment outcome.

Thus there is a great desire for compounds that lack D2 antagonism related side-effects and that are effective in the treatment of disorders wherein the dopaminergic system is disrupted such as one or more disorders independently selected from the group consisting of schizophrenia and other psychotic states; mood disorders; ADHD; aggression; movement disorders; and substance use and/or abuse, but have reduced propensity to cause or are devoid of the above-mentioned side-effects of known antipsychotics.

Additionally a great desire exists for compounds with a fast onset of action that are effective in the treatment of disorders wherein the dopaminergic system is disrupted such as one or more disorders independently selected from the group consisting of schizophrenia and other psychotic states; mood disorders; ADHD; aggression; movement disorders; and substance use and/or abuse.

Moreover a great desire exists for compounds that exhibit a significantly greater efficacy in treating the disorders wherein the dopaminergic system is disrupted such as one or more disorders independently selected from the group consisting of schizophrenia and other psychotic states; mood disorders; ADHD; aggression; movement disorders; and substance use and/or abuse and may treat a greater percentage of patients than currently benefit from existing antipsychotics drugs.

It has now for the first time surprisingly been found that compounds that activate KCNQ channels are able to modulate the dopaminergic system in vivo and display efficacy in the commonly used animal models of conditions, disorders and/or diseases wherein the dopaminergic system is disrupted such as one or more conditions, disorders and/or diseases independently selected from the group consisting of schizophrenia and other psychotic states; mood disorders; ADHD; aggression; movement disorders; and substance use and/or abuse.

Ion channels are cellular proteins that regulate the flow of ions, including potassium, calcium, chloride and sodium into and out of cells. Such channels are present in all animal and human cells and affect a variety of processes including neuronal transmission, muscle contraction, and cellular secretion.

Humans have over 70 genes encoding potassium channel subtypes (Jentsch Nature Reviews Neuroscience 2000, 1, 21-30) with a great diversity with regard to both structure and function. Neuronal potassium channels, which are found in the brain, are primarily responsible for maintaining a negative resting membrane potential, as well as controlling membrane repolarisation following an action potential.

One subset of potassium channel genes is the KCNQ family. Mutations in four out of five KCNQ genes have been shown to underlie diseases including cardiac arrhythmias, deafness and epilepsy (Jentsch Nature Reviews Neuroscience 2000, 1, 21-30).

KCNQ1 (KvLQT1) is co-assembled with the product of the KCNE1 (minimal K(+)-channel protein) gene in the heart to form a cardiac-delayed rectifier-like K(+) current. Mutations in this channel can cause one form of inherited long QT syndrome type 1 (LQT1), as well as being associated with a form of deafness (Robbins Pharmacol Ther 2001, 90, 1-19).

The genes KCNQ2 and KCNQ3 were discovered in 1998 and appear to be mutated in an inherited form of epilepsy known as benign familial neonatal convulsions (Rogawski Trends in Neurosciences 2000, 23, 393-398). The proteins encoded by the KCNQ2 and KCNQ3 genes are localised in the pyramidal neurons of the human cortex and hippocampus, regions of the brain associated with seizure generation and propagation (Cooper et al. Proceedings National Academy of Science USA 2000, 97, 4914-4919).

KCNQ2 and KCNQ3 are two potassium channel subunits that form “M-currents” when expressed in vitro. KCNQ5 has also been shown to contribute to the M-current in cultured hippocampal neurons (Shah et al., Journal of Physiology 2002, 544, 29-37). KCNQ4 potassium channels have been shown to possess M-current-like properties when expressed in cell lines (Sørgaard et al., American Journal of Physiology and Cellular Physiology, 2001, 280, C859-C866). The M-current is a non-inactivating potassium current found in many neuronal cell types. In each cell type, it is dominant in controlling membrane excitability by being the only sustained current in the range of action potential initiation (Marrion Annual Review Physiology 1997, 59, 483-504). Modulation of the M-current has dramatic effects on neuronal excitability, for example activation of the current will reduce neuronal excitability. Openers of these KCNQ channels or activators of the M-current, will reduce neuronal activity and may thus be of use in the treatment of seizures and other diseases and disorders characterised by excessive neuronal activity, such as neuronal hyperexcitability including convulsive disorders, epilepsy, neuropathic pain, anxiety, ADHD, mania, migraine and schizophrenia

The KCNQ4 gene is thought to encode the molecular correlate of potassium channels found in outer hair cells of the cochlea and in Type I hair cells of the vestibular apparatus, in which mutations can lead to a form of inherited deafness.

KCNQ2 and KCNQ4 are also expressed in the substantia nigra and ventral tegmental area (Kharkovets et al, 2000 Proceedings National Academy of Science USA, 97, 4333-4338), which contain the cell bodies of two of the major dopaminergic systems in the brain the nigrostriatal and mesolimbic systems respectively. There is functional coupling between dopamine D2 receptors and KCNQ4 channels when expressed in oocytes or SH-SY5Y cells (Ljungstrom et al., European Journal of Physiology, 2003, 446, 684-694), which suggests similar coupling in vivo when the D2 receptor and KCNQ4 channels are expressed in the same cells.

Retigabine (D-23129; N-(2-amino-4-(4-fluorobenzylamino)-phenyl) carbamic acid ethyl ester) and analogues thereof are disclosed in EP554543. Retigabine is an anti-convulsive compound with a broad spectrum and potent anticonvulsant properties, both in vitro and in vivo. It is active after oral and intraperitoneal administration in rats and mice in a range of anticonvulsant tests (Rostock et al. Epilepsy Research 1996, 23, 211-223). In clinical trials, retigabine has recently shown effectiveness in reducing the incidence of seizures in epileptic patients (Bialer et al. Epilepsy Research 2002, 51, 31-71).

Retigabine has been shown to activate a K(+) current in neuronal cells and the pharmacology of this induced current displays concordance with the published pharmacology of the M-channel. Retigabine has also been shown to bind to KCNQ channels (Wuttke et al, Molecular Pharmacology, 2005, 67, 1009-1017). These data suggest that activation of KCNQ channels is responsible for at least some of the anticonvulsant activity of this agent (Wickenden et al. Molecular Pharmacology 2000, 58, 591-600)—and that other agents working by the same mechanism may have similar uses.

Retigabine has been shown to suppress the firing of dopaminergic neurons in the ventral tegmental area ex vivo (Hansen et al., Society for Neuroscience Abstracts, 2005, 153.11). However, it is not known whether this effect of retigabine translates into an in vivo inhibition of dopaminergic neurons in the ventral tegmental area, or whether this effect is associated with anti-psychotic-like behaviour in animals.

Besides affinity for KCNQ potassium channels, Retigabine has also a modulatory effect on GABA_{A} receptors.

Side-effects such as sedation (Rudolph and Möhler, Curr Opin Pharmacol, 2006, 6, 18-23), cognitive impairments, most prominently anterograde amnesia (Stewart, J Clin Psychiatry, 2005, 66, supl 2, 9-13) and motor impairments (Verster et al, Sleep Med Rev, 2004, 8, 309-325) are associated with modulatory effect on GABA_{A} receptors. Furthermore, compounds with modulatory action on GABA_{A} receptors are also associated with abuse potential (Griffiths and Weerts, Psychopharmacology, 1997, 134, 1-37) and withdrawal symptoms upon discontinuation (Woods et al, Pharmacol Rev, 1992, 44, 151-347).

The provision of compounds for use in a method for reducing symptoms of or for treating one or more disorders wherein the dopaminergic system is disrupted such as one or more disorders independently selected from the group consisting of schizophrenia and other psychotic states; mood disorders; ADHD; aggression; movement disorders; and substance use and/or abuse being KCNQ openers which lack D2 antagonism related side-effects and which furthermore lack GABA_{A} related side-effects are therefore highly desired.

Besides affinity for KCNQ potassium channels and GABA_{A} receptors, Retigabine has now surprisingly been found also to have affinity for noradrenergic α1_{A} receptors.

Side-effects such as orthostatic hypotension and sedation (Casey, J Clin Psychiat, 1997, 10, supl 58, 55-62) are associated with modulation of the noradrenergic α1_{A} receptors.

The provision of compounds that may be used in a method for reducing symptoms of, or for treating, one or more disorders wherein the dopaminergic system is disrupted, such as one or more disorders independently selected from the group consisting of: schizophrenia and other psychotic states; mood disorders; ADHD; aggression; movement disorders; and substance use and/or abuse; and which are KCNQ openers that lack D2 antagonism related side-effects and furthermore lack noradrenergic α1_{A} related side-effects is therefore highly desired.

### Definitions

The term “KCNQ potassium channel” refers to homotetrameric or heterotetrameric potassium channel complexes that are composed of one or more types of subunits selected from the group of KCNQ2, KCNQ3, KCNQ4 and KCNQ5.

A potential of a compound for fast-onset of therapeutic efficacy is defined as the potential for a compound to exert a fast onset of clinical therapeutic efficacy i.e. a faster onset than seen with clinically used compounds within a given indication area, is supported by in vivo electrophysiological assessments of the spontaneous firing rate of dopamine cells in the ventral tegmental area, showing acute inhibitory effects of compound (as opposed to inhibitory effects only after chronic dosing).

The term “modulate” or “modulation” in respect of a channel or a receptor refers to an antagonistic or agonist effect on said channel or receptor.

The term “host” refers to any mammal. The host, such as a human, to be treated with a compound according to the invention may in fact be any subject of the human population, male or female, which may be divided into children, adults, or elderly. Any one of these patient groups relates to an embodiment of the invention.

The term “treatment” as used herein in connection with a disorder or condition includes also prevention, inhibition, amelioration and prevention of recurrence and/or relapse as the case may be.

The term “treating” as used herein in connection with a condition or disorder includes also preventing, inhibiting, ameliorating and prevention of recurrence and/or relapse as the case may be.

The term “acute treatment” refers to the introduction or reintroduction of a compound according to the invention to alleviate (or at least palliate) an exacerbation of psychosis.

The term “long-term treatment” refers to maintenance or life-long treatment.

The term “disorder” as used herein includes also condition or disease as the case may be.

The term “condition” as used herein in connection with a medical and/or clinical condition includes also disease or disorder as the case may be.

The term “effective amount” refers to the amount/dose of a compound or pharmaceutical composition that is sufficient to produce an effective response (i.e., a biological or medical response of a tissue, system, animal or human sought by a researcher, veterinarian, medical doctor or other clinician) upon administration to a subject. The “effective amount” will vary depending on inter alia the disease and its severity, and the age, weight, physical condition and responsiveness of the subject to be treated.

Lack of D2 receptor antagonism related side-effects is defined as avoidance of D2 receptor-related side-effects given the lack of direct involvement of D2 receptors in the mechanism of action of the mentioned compounds.

Lack of GABA_{A} receptor related side-effects is defined as avoidance of GABA_{A} receptor-related side-effects given the lack of direct involvement of GABA_{A} receptors in the mechanism of action of the mentioned compounds.

Lack of noradrenergic α1_{A} receptor related side-effects is defined as avoidance of α1_{A} receptor-related side-effects given the lack of direct involvement of α1_{A} receptors in the mechanism of action of the mentioned compounds.

The term ‘Antipsychotic potential’ in relation to a compound refers to the potential of a compound as an antipsychotic drug for reducing symptoms of or for treating one or more disorders wherein the dopaminergic system is disrupted such as one or more disorders independently selected from the group consisting of schizophrenia and other psychotic states; mood disorders; ADHD; aggression; movement disorders; and substance use and/or abuse.

‘Movement disorder(s)’ refers to one or more disorders that are characterized by the presence of abnormal movements of the body that have a neurological basis. These abnormal movements may also involve the presence of movements that are not voluntary.

The term ‘known antipsychotic compound’ or ‘known antipsychotic(s)’ refer to compounds (more generally compounds other than compounds of formula 1, 2, 3, 4, 5, 6, 7, 8, 9 or 10 as employed in the context of the present invention) that are known to have an antipsychotic potential, i.e. that are able to, or have the potential to, reduce psychotic symptoms. These include, for example, Asenapine, Blonanserin, Iloperidone, Paliperidone, Lurasidone, Ocaperidone, Talnetant, Eplivanserin, Farampator, Sabcomeline, Vabicaserin, ACP 103, ACP 104, AL 108, SLV 310, ACR 16, YKP 1358, GW 773812, RGH 188, SLV 314, SLV 313, TGOF02N, Y-931, BL 1020, AVE 1625, GSK-773812, ABT 089, CX 516, DAR 0100, Chlorpromazine, Levomepromazine, Promazine, Acepromazine, Triflupromazine, Cyamemazine, Chlorproethazine, Dixyrazine, Fluphenazine, Perphenazine, Prochlorperazine, Thiopropazate, Trifluoperazine, Acetophenazine, Thioproperazine, Butaperazine, Perazine, Periciazine, Thioridazine, Mesoridazine, Pipotiazine, Haloperidol, Trifluperidol, Melperone, Moperone, Pipamperone, Bromperidol, Benperidol, Droperidol, Fluanisone, Oxypertine, Molindone, Ziprasidone, Flupentixol, Clopenthixol, Chlorprothixene, Tiotixene, Zuclopenthixol, Fluspirilene, Pimozide, Penfluridol, Loxapine, Clozapine, Olanzapine, Quetiapine, Sulpiride, Sultopride, Tiapride, Remoxipride, Amisulpride, Veralipride, Levosulpiride, Prothipendyl, Risperidone, Clotiapine, Mosapramine, Zotepitie and Aripiprazole.

Other known antipsychotics of relevance include Sertindole.

Potential to treat schizophrenia is defined as a potential to treat or to reduce one or more symptoms of schizophrenia.

Potential to treat psychotic states other than schizophrenia is defined as a potential to treat or to reduce symptom(s) of one or more psychotic states other than schizophrenia.

Potential to treat mood disorders is defined as a potential to treat or to reduce symptom(s) of one or more mood disorders.

Potential to treat bipolar spectrum disorders is defined as a potential to treat or to reduce symptom(s) of one or more bipolar spectrum disorders.

Potential to treat ADHD is defined as a potential to treat or to reduce one or more symptoms of ADHD.

Potential to treat movement disorder is defined as a potential to treat or to reduce symptom(s) of one or more movement disorders.

Potential to treat substance use and/or abuse is defined as a potential to treat or to reduce symptom(s) of substance use and/or abuse or to prevent and/or reduce intake of substance(s) of use and/or abuse as well as prevent relapse of substance(s) use and/or abuse.

### SUMMARY OF THE INVENTION

The invention relates to a method for reducing symptoms of or for treating one or more disorders wherein the dopaminergic system is disrupted, such as schizophrenia and other psychotic states; mood disorders; ADHD; aggression; movement disorders; and substance use and/or abuse; the method comprising administering to a host in need thereof an effective amount of a compound able to increase the ion flow through KCNQ potassium channels. Such a pharmacological profile of compounds employed in the context of the invention is highly novel compared with known antipsychotics, and it may be anticipated that they would be substantially devoid of the side-effects induced by these latter types of drugs.

Furthermore, the pharmacological profile of compounds among those according to formulae 1, 2, 3, 4, 5, 6, 7 or 8 is highly novel as compared with other compounds known to be able to increase the ion flow through KCNQ potassium channels. Thus, compounds among those according to formulae 1, 2, 3, 4, 5, 6, 7 or 8 appear to be devoid of or have only insignificant affinity towards the GABA_{A} receptor and noradrenergic α1_{A} receptors. The compounds in question are thereby more selective than other compounds known to be able to increase the ion flow through KCNQ potassium channels such as retigabine, and are therefore expected to be substantially devoid of potential side-effects related to affinity for GABA_{A} receptors or noradrenergic α1_{A} receptors induced by such drugs.

Compounds that activate KCNQ channels may have a fast onset of action. Furthermore, the distinct and novel mechanism of action may have significantly greater efficacy in reducing symptoms of or for treating one or more disorders wherein the dopaminergic system is disrupted such as one or more disorders independently selected from the group consisting of schizophrenia and other psychotic states; mood disorders; ADHD; aggression; movement disorders; and substance use and/or abuse and may treat a greater percentage of patients than currently benefit from known antipsychotics. Additionally compliance may be improved. They would, therefore, offer a significant advantage in reducing symptoms of or for treating one or more disorders wherein the dopaminergic system is disrupted such as one or more disorders independently selected from the group consisting of schizophrenia and other psychotic states; mood disorders; ADHD; aggression; movement disorders; and substance use and/or abuse. The compounds of formula 1, 2, 3, 4, 5, 6, 7, and 8 have a larger efficacy and/or stronger potency than Retigabine and will therefore exert a therapeutic efficacy that is superior to Retigabine.

Administering a compound able to increase the ion flow through KCNQ potassium channels together with one or more known antipsychotics will provide a double acting therapeutic treatment since two independent mechanisms, which both converge on the same down-stream target, namely extracellular DA levels, are combined. A synergistic effect leading to a superior symptomatic relief is thus expected.

It is furthermore believed that a fast-onset of action will be obtained because compounds which increase the ion flow through the KCNQ potassium channels lead to a faster normalization of DA activity, than conventional antipsychotics do when administered alone.

It is also believed that a broader symptomatic relief will be obtained because a compound able to increase the ion flow through KCNQ potassium channels is able to treat e.g. symptoms of which known antipsychotics provides no or insignificant treatment of, such as negative symptoms of schizophrenia.

Schizophrenic patients have a high rate of comorbid substance abuse. Since compounds able to increase the ion flow through KCNQ potassium channels are believed to be useful in the treatment of substance abuse, such comorbidity is believed to be prevented and the incidence thereof to be significantly reduced.

The above-mentioned D2 antagonism related side-effects seen by use of known antipsychotics are severe and a direct cause for poor compliance with such compounds and it is thus believed that an improved compliance will be obtained by administering a compound able to increase the ion flow through KCNQ potassium channels together with one or more known antipsychotics, as the dose of the latter is expected to be reduced in a combination therapy setting.

### DESCRIPTION OF THE INVENTION

The invention relates to a method for reducing symptoms of or for treating one or more disorders wherein the dopaminergic system is disrupted, said method comprising administering to a host in need thereof an effective amount of a compound able to increase the ion flow through KCNQ potassium channels. Schizophrenia and other psychotic states; mood disorders; ADHD; aggression; movement disorders; and substance use and/or abuse are examples of such disorder(s) wherein the dopaminergic system is disrupted.

A potential of a compound to treat psychotic disorders, where one such compound is able to control agitated psychotic behaviour, alleviate acute psychotic states, reduce psychotic symptoms, and exert a quieting effect, is supported by in vivo behavioural tests reflective of antipsychotic-like behaviour such as inhibition of stimulant-induced hyperactivity (Example 1, part B), inhibition of a sensitised response (hyperactivity) to amphetamine (Example 1, part D), and inhibition of conditioned avoidance responses (Example 1, part C).

An embodiment relates to a method for reducing symptoms of one or more disorders wherein the dopaminergic system is disrupted.

An embodiment relates to a method for treating one or more disorders wherein the dopaminergic system is disrupted.

An embodiment of the invention relates to a method for reducing symptoms of or for treating schizophrenia and one or more other psychotic states.

An embodiment of the invention relates to a method for reducing symptoms of or for treating schizophrenia, such as schizophrenia with one or more of positive, negative, cognitive and affective symptoms.

An embodiment concerns such method wherein said disorder is schizophrenia with affective symptoms and with one or more of positive, negative and cognitive symptoms.

An embodiment concerns such method wherein said disorder is schizophrenia with affective symptoms, such as depressive symptoms.

Affective symptoms of schizophrenia may be depressive symptoms such as one or more of depressed mood in general, anhedonic symptoms, sleep disturbances, psychomotor agitation or retardation, sexual dysfunction, weight loss, concentration difficulties, delusional ideas, loss of energy, feeling of worthlessness, recurrent thoughts of death and suicidal ideation.

Positive symptoms of schizophrenia cover a pattern of psychotic features such as one or more of but not limited to delusions, thought disorders, distortions or exaggerations in language and communication, disorganized speech, disorganized behaviour, catatonic behaviour, agitation and hallucinations such as typically auditory.

Negative symptoms of schizophrenia are typically characterised by one or more of, but not limited to, blunted affect, aphasia, asociality, anhedonia (lack of pleasure), avolition (restrictions in the initiation of goal-directed behaviour), emotional withdrawal, difficulty in abstract thinking, lack of spontaneity, stereotyped thinking, alogia (restrictions in the fluency and productivity of thought and speech) and attentional impairment.

Cognitive symptoms of schizophrenia are characterised by dysfunction across many cognition domains including attention, memory and executive function.

An embodiment of the invention relates to a method for reducing symptoms of or for treating schizophrenia, such as the catatonic-subtype, the paranoid-subtype, the disorganized-subtype and the residual-subtype of schizophrenia. An embodiment concerns a method for reducing symptoms of or for treating the catatonic-subtype of schizophrenia. An embodiment concerns a method for reducing symptoms of or for treating the paranoid-subtype of schizophrenia. An embodiment concerns a method for reducing symptoms of or for treating the disorganized-subtype of schizophrenia. An embodiment concerns a method for reducing symptoms of or for treating the residual-subtype of schizophrenia.

A potential of a compound to treat the positive symptoms of schizophrenia, is supported by in vivo behavioural tests reflective of antipsychotic-like behaviour such as inhibition of stimulant-induced hyperactivity (Example 1, part B), inhibition of a sensitised response (hyperactivity) to amphetamine (Example 1, part D), conditioned avoidance response (Example 1, part C).

A potential of a compound to treat the negative symptoms of schizophrenia is supported by positive effects in the forced swim test, or the chronic mild stress test, or the social interaction test (Example 3).

Positive effects in the chronic mild stress paradigm, the forced swim test (Example 3) or the social interaction test (Example 4) are considered to be indicative of a potential of a compound to treat the negative symptoms of schizophrenia.

Positive effects in the chronic mild stress paradigm, the forced swim test (Example 3) or the social interaction test (Example 4) are considered to be indicative of a potential of a compound to treat affective symptoms of schizophrenia.

Positive effects in the 5-choice serial reaction time task, the morns water maze model, the attentional set-shifting model (Example 8) are considered to be indicative of a potential of a compound to treat cognitive symptoms of schizophrenia.

An embodiment of the invention relates to a method for reducing symptoms of or for treating one or more psychotic states other than schizophrenia, such as psychosis in Parkinson's disease, psychotic depression and other psychotic disorders as well as psychotic symptoms induced by one or more substances and psychotic symptoms appearing in general medical conditions.

An embodiment concerns such method wherein said psychotic state other than schizophrenia is psychosis in Parkinson's disease. In an embodiment said psychotic symptoms are induced by the standard treatments used in Parkinson's disease, such as L-DOPA. In another embodiment said psychotic symptoms are a consequence of the underlying pathophysiology of Parkinson's disease.

An embodiment concerns such method wherein said psychotic state other than schizophrenia is psychotic depression.

An embodiment concerns such method wherein said psychotic state(s) other than schizophrenia is/are psychotic symptoms induced by one or more substances such as nicotine; cannabis; CNS depressants such as alcohol; opioids such as one or more of heroin and morphine; and psychostimulants such as one or more of amphetamine and cocaine. In an embodiment said psychotic state is induced by nicotine. In an embodiment said psychotic state is induced by cannabis. In an embodiment said psychotic state is induced by CNS depressants such as alcohol. In an embodiment said psychotic state is induced by opioids such as one or more of heroin and morphine. In an embodiment said psychotic state is induced by psychostimulants such as one or more of amphetamine and cocaine.

An embodiment concerns such method wherein said psychotic state(s) other than schizophrenia is/are general medical conditions such as one or more of Alzheimer's disease, dementia or bipolar spectrum disorders. In an embodiment said psychotic state is Alzheimer's disease. In an embodiment said psychotic state is dementia. In an embodiment said psychotic state is bipolar spectrum disorders.

An embodiment concerns such method wherein said psychotic state(s) other than schizophrenia is/are one or more psychotic disorders selected from schizophreniform disorder, schizoaffective disorder, delusional disorder and brief psychotic disorder. In an embodiment said psychotic state is schizophreniform disorder. In an embodiment said psychotic state is schizoaffective disorder. In an embodiment said psychotic state is delusional disorder. In an embodiment said psychotic state is brief psychotic disorder

A potential of a compound to treat psychotic symptoms is supported by positive effects in the conditioned avoidance model (Example 1, part C), stimulant induced hyperactivity model (Example 1, part B) and the sensitised response to amphetamine model (Example 1, part D).

Positive effects in these models are believed to be indicative of a compounds potential to treat psychotic symptoms in patients suffering from Parkinson's disease, psychotic symptoms in patients suffering from psychotic depression, and other psychotic disorders as well as psychotic symptoms induced by one or more substances and psychotic symptoms appearing in general medical conditions.

An embodiment of the invention relates to a method for reducing symptoms of or for treating one or more mood disorders, such as one or more depressive disorders and/or bipolar spectrum disorder(s).

In an embodiment said mood disorder(s) is one or more depressive disorders. In an embodiment said depressive disorder(s) is/are selected from the group consisting of major depressive disorder, dysthymic disorder, depressive disorder not otherwised specified, minor depression and brief recurrent depression mood disorders. In an embodiment said depressive disorder is major depressive disorder. In an embodiment said depressive disorders are major depressive disorder and one or more other depressive disorders. In an embodiment said depressive disorder(s) is/are one or more depressive disorders other than major depressive disorder. In an embodiment said depressive disorder(s) other than major depressive disorder is/are selected from the group consisting of a depressed mood such as in dysthymic disorder; depressive disorder not otherwised specified; minor depression; and brief recurrent depression mood disorders. In an embodiment said depressive disorder is dysthymic disorder. In an embodiment said depressive disorder is depressive disorder not otherwised specified. In an embodiment said depressive disorder is minor depression. In an embodiment said depressive disorder is brief recurrent depression mood disorders.

A potential of a compound to treat depressive disorders is supported by positive effects in the forced swim test and in the chronic mild stress test (Example 3).

An embodiment of the invention relates to a method for reducing symptoms of or for treating one or more bipolar spectrum disorders. In an embodiment said bipolar spectrum disorder(s) is/are are selected from the group consisting of bipolar I disorder, bipolar II disorder, cyclothymic disorder and bipolar disorder not otherwise specified. In an embodiment said bipolar spectrum disorder is bipolar I disorder. In an embodiment said bipolar spectrum disorder is bipolar II disorder. In an embodiment said bipolar spectrum disorder is cyclothymic disorder. In an embodiment said bipolar spectrum disorder is bipolar disorder not otherwise specified.

In an embodiment, said method is reducing symptoms of or treating one or more episodes selected from the group consisting of manic episode(s), mixed episode(s), major depressive episode(s) and/or hypomanic episode(s). In an embodiment said episode(s) is/are manic episode(s). In an embodiment said episode(s) is/are mixed episode(s). In an embodiment said episode(s) is/are hypomanic episode(s). In an embodiment said episode(s) is/are major depressive episode(s).

In an embodiment said method provides one or more of antimanic, antidepressant and/or mood stabilising activity. In an embodiment said method provides anitmanic activity. In an embodiment said method provides antidepressant activity. In an embodiment said method provides mood stabilising activity.

A potential of a compound to treat bipolar spectrum disorders is supported by positive effects in the amphetamine+chlordiazepoxide induced hyperactivity rodent model of mania (Example 10) as well as positive effects in the lithium-sensitive stimulant-induced hyperactivity models (Example 1, part B) and sensitized amphetamine response model (Example 1, part D).

An embodiment of the invention relates to a method for reducing symptoms of or for treating attention deficit disorder (ADHD). In an embodiment the symptom(s) of said ADHD is/are one or more of inattention such as failure to give close attention, difficulties in sustaining attention, difficulties in organising tasks and activities and/or easy distraction by extraneous stimuli; hyperactivity such as difficulties in remaining seated, excessive motor activity in inappropriate situations and/or acting as if “driven by a motor”; and impulsivity such as difficulties in awaiting turn, answer questions before they have been completed and/or interrupts or intrudes ongoing conversation. In an embodiment said symptom(s) is/are symptom(s) of inattention such as failure to give close attention, difficulties in sustaining attention, difficulties in organising tasks and activities and/or easy distraction by extraneous stimuli. In an embodiment said symptom(s) is/are symptom(s) of hyperactivity such as difficulties in remaining seated, excessive motor activity in inappropriate situations and/or acting as if “driven by a motor”. In an embodiment said symptom(s) is/are symptom(s) of impulsivity such as difficulties in awaiting turn, answer questions before they have been completed and/or interrupts or intrudes ongoing conversation

A potential of a compound to treat ADHD is supported by positive effects in the preclinical models mentioned in example 9.

An embodiment of the invention relates to a method for reducing symptoms of or for treating aggression. In an embodiment said aggression is present in one or more other clinical conditions such as in impulse-control disorders such as intermittent explosive disorder; schizophrenia; bipolar disorder; Alzheimer's disease; dementia and Parkinson's disease. In an embodiment said aggression is present in impulse-control disorders such as intermittent explosive disorder. In an embodiment said aggression is present in schizophrenia. In an embodiment said aggression is present in bipolar spectrum disorders. In an embodiment said aggression is present in Alzheimer's disease. In an embodiment said aggression is present in dementia. In an embodiment said aggression is present in Parkinson's disease

A potential of a compound to treat aggression is supported by positive effects in the preclinical models mentioned in example 11.

An embodiment of the invention relates to a method for reducing symptoms of or for treating one or more movement disorder(s).

In an embodiment said movement disorder(s) is/are one or more tics disorders such as physical tics such as motor tics and/or vocal tics such as phonic tics which may be transient or chronic. In an embodiment said tics disorder is a physical tics disorder such as motor tics. In an embodiment said tics disorder is a vocal tics disorder such as phonic tics. In an embodiment said tics disorder is a transient tics disorder. In an embodiment said tics disorder is a chronic tics disorder.

In an embodiment said movement disorder(s) is/are selected from Parkinson's disease, Huntingtons disease and/or Tourette's syndrome. In an embodiment said movement disorder is Parkinson's disease. In an embodiment said movement disorder is Huntingtons disease. In an embodiment said movement disorder is Tourette's syndrome.

A potential of a compound to treat movement disorder(s) is supported by positive effects in the preclinical models mentioned in example 7.

An embodiment of the invention relates to a method for reducing symptoms of or for treating the use and/or abuse of one or more substances. In an embodiment said use and/or abuse is characterized by dependency on and/or addiction to said substance(s). In an embodiment said substance(s) is/are one or more substances selected from nicotine; cannabis; the group of CNS depressants such as alcohol; the group of opioids such as heroin and morphine; and the group of psychostimulants such as amphetamine and cocaine. In an embodiment said substance is nicotine. In an embodiment said substance is cannabis. In an embodiment said substance is selected from the group of CNS depressants such as alcohol. In an embodiment said substance is alcohol. In an embodiment said substance is selected from the group of opioids such as heroin and morphine. In an embodiment said substance is heroin. In an embodiment said substance is morphine. In an embodiment said substance is selected from the group of psychostimulants such as amphetamine and cocaine. In an embodiment said substance is amphetamine. In an embodiment said substance is cocaine.

A potential of a compound to treat an individual for his/her addiction to, or abuse of, a substance of abuse is supported by positive effects in the preclinical models mentioned in Example 2.

Another aspect of the present invention relates to a method for reducing symptoms of or for treating one or more disorders wherein the dopaminergic system is disrupted, said method comprising administering to a host in need thereof an effective amount of a compound able to increase the ion flow through KCNQ potassium channels wherein said compound able to increase the ion flow through KCNQ potassium channels is effective in one or more models predictive for an antipsychotic potential of said compound. In an embodiment said compound is effective in one or more models predictive for an antipsychotic potential of said compound, such as the schizophrenia potential, potential to treat psychotic state(s) other than schizophrenia, potential to treat mood disorder(s), potential to treat bipolar spectrum disorder(s), potential to treat ADHD, potential to treat movement disorder(s) and/or potential to treat substance use and/or abuse of said compound. In an embodiment said model(s) is/are predictive for schizophrenia potential, potential to treat psychotic state(s) other than schizophrenia, potential to treat mood disorder(s), potential to treat bipolar spectrum disorder(s), potential to treat ADHD, potential to treat movement disorder(s) and/or potential to treat substance use and/or abuse of said compound.

Another aspect of the present invention relates to a method for reducing symptoms of or for treating one or more disorders wherein the dopaminergic system is disrupted, said method comprising administering to a host in need thereof an effective amount of a compound able to increase the ion flow through KCNQ potassium channels wherein said compound has a fast-onset of action.

An embodiment relates to a method wherein the symptoms are reduced faster than they are by use of known antipsychotics for reducing said symptom(s). An embodiment relates to a method wherein the symptoms are reduced such as after two weeks, preferably after one week, even more preferred within one week, even more preferred after two days, even more preferred within two days, even more preferred after one day and most preferred within one day.

An embodiment relates to a method wherein onset of clinical therapeutical efficacy is faster than for known antipsychotics used. An embodiment relates to a method wherein said clinical therapeutical efficacy is obtained after two weeks, preferably after one week, even more preferred within one week, even more preferred after two days, even more preferred within two days, even more preferred after one day and most preferred within one day

Another aspect of the present invention relates to a method for reducing symptoms of or for treating one or more disorders wherein the dopaminergic system is disrupted, said method comprising administering to a host in need thereof an effective amount of a compound able to increase the ion flow through KCNQ potassium channels wherein said disorder is a sexual dysfunction such as lack of sexual motivation and/or loss of libido. In an embodiment said disorder is lack of sexual motivation. In an embodiment said disorder is loss of libido. Such a potential is supported positive effects in the preclinical models mentioned in example 12.

Another aspect of the present invention relates to a method for reducing symptoms of or for treating one or more disorders wherein the dopaminergic system is disrupted, said method comprising administering to a host in need thereof an effective amount of a compound able to increase the ion flow through KCNQ potassium channels wherein said compound is efficacious in mood stabilisation and antimanic treatment.

In another embodiment the invention relates to long-term treatment.

In another embodiment, the invention relates to acute treatment.

Another aspect of the present invention relates to a method for reducing symptoms of or for treating one or more disorders wherein the dopaminergic system is disrupted, said method comprising administering to a host in need thereof an effective amount of a compound able to increase the ion flow through KCNQ potassium channels wherein said compound does not to any significant extent manifest any side-effects associated with the mechanism of action of known antipsychotics. In an embodiment, said side-effects associated with known antipsychotics are mediated directly through dopamine D2 receptor modulation.

In another embodiment the invention relates to a method wherein said compound does not to any significant extent manifest any side-effects associated with either noradrenergic α1_{A} or GABA_{A} receptor modulation. In an embodiment, said side-effects are associated with noradrenergic α1_{A} receptor modulation. In an embodiment, said side-effects are associated with GABA_{A} receptor modulation.

An embodiment relates to a method wherein said compound is able to increase the ion flow through KCNQ potassium channels, wherein said compound does not to any significant extent manifest any side-effects associated with neither dopamine D2, noradrenergic α1_{A} nor GABA_{A} receptor modulation.

Another embodiment of the invention relates to a method of screening for a compound, which is a KCNQ channel opener and which is capable of having an anti-psychotic potential comprising the steps of:
screening for a KCNQ opener;
contra-screening against other channels and/or receptors, and
testing the compound in a model predictive for an anti-psychotic potential.

Another aspect of the present invention relates to a method for reducing symptoms of or for treating one or more disorders wherein the dopaminergic system is disrupted, said method comprising administering to a host in need thereof an effective amount of a compound able to increase the ion flow through KCNQ potassium channels wherein said compounds have an EC₅₀ of less than 20000 nM. In an embodiment said compounds have an EC₅₀ of less than 2000 nM. In an embodiment said compounds have an EC₅₀ of less than 200 nM. The procedure for the determination of the EC₅₀ value for a KCNQ potassium channel is outline in Example 5 herein.

Another aspect of the invention relates to a method for reducing symptoms of or for treating one or more disorders wherein the dopaminergic system is disrupted, said method comprising administering to a host in need thereof an effective amount of a compound able to increase the ion flow through KCNQ potassium channels, wherein said compound is optionally given together with one or more known antipsychotic(s). In an embodiment, said compound is given as the only compound having an antipsychotic potential. In an embodiment said compound is given as part of adjunctive therapy, ie together with one or more other therapeutic agents. In an embodiment, said compound is given together with one other compound having an antipsychotic potential. In an embodiment, said compound is given together with two or more other compounds having an antipsychotic potential.

An aspect of the present invention relates to a method for reducing symptoms of or for treating one or more disorders wherein the dopaminergic system is disrupted, said method comprising administering to a host in need thereof an effective amount of a compound able to increase the ion flow through KCNQ potassium channels wherein said compound is a compound according to any one of formulae 1, 2, 3, 4, 5, 6, 7, 8, 9 or 10.

An embodiment of the invention relates to a method wherein said compound is a compound according to formula 1:

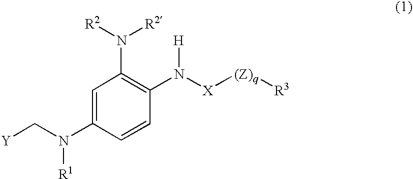

wherein:
R¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl and hydroxy-C₃₋₈-cycloalk(en)yl;
R² and R^{2′} are independently selected from the group consisting of hydrogen, C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, acyl, hydroxy-C₁₋₆-alk(en/yn)yl and hydroxy-C₃₋₈-cycloalk(en)yl;
R³ is selected from the group consisting of hydrogen, C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, hydroxy-C₁₋₆-alk(en/yn)yl, aryl-C₃₋₈-cycloalk(en)yl, NR¹⁰R^{10′}—C₁₋₆-alk(en/yn)yl, NR¹⁰R^{10′}—C₃₋₈-cycloalk(en)yl and hydroxy-C₃₋₈-cycloalk(en)yl; wherein:
   R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or
   R¹⁰ and R^{10′} together with the nitrogen atom to which they are attached form a 4-8 membered saturated or unsaturated ring which optionally contains 1, 2 or 3 further heteroatoms;
X is CO or SO₂;
Z is O or NR⁴, wherein:
   R⁴ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl and hydroxy-C₃₋₈-cycloalk(en)yl; or
   R³ and R⁴ together with the nitrogen atom to which they are attached form a 4-8 membered saturated or unsaturated ring which optionally contains 1, 2 or 3 further heteroatoms, the ring formed by R³ and R⁴ and the nitrogen atom is optionally substituted with one or more substituents independently selected from C₁₋₆-alk(en/yn)yl, aryl and aryl-C₁₋₆-alk(en/yn)yl;
q is 0 or 1; and
Y represents a heteroaryl of formula II or III

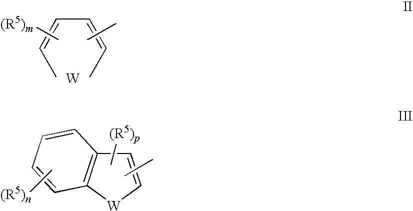

wherein
W is O or S;
m is 0, 1, 2 or 3;
n is 0, 1, 2, 3 or 4;
p is 0 or 1; and
each R⁵ is independently selected from the group consisting of C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, acyl, halogen, halo-C₁₋₆-alk(en/yn)yl, C₁₋₆-alk(en/yn)yloxy, —CO—NR⁶R^{6′}, cyano, nitro, —NR⁷R^{7′}, —S—R⁸, —SO₂R⁸, SO₂OR⁸; wherein:
   R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and aryl;
   R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, aryl and acyl; and
   R⁸ is selected from the group consisting of C₁₋₆-alk(en/yn)yl, C₃₋₈-cycloalk(en)yl, C₃₋₈-cycloalk(en)yl-C₁₋₆-alk(en/yn)yl, aryl and —NR⁹R^{9′}; wherein
      a. R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl;
         or pharmaceutically acceptable salts thereof.

In an embodiment of formula 1:
R¹ is selected from the group consisting of hydrogen and C₁₋₆-alk(en/yn)yl;
at least one of the substituents R² and R^{2′} is a hydrogen atom;
X is CO;
R³ is selected from the group consisting of C₁₋₆-alk(en/yn)yl and aryl-C₁₋₆-alk(en/yn)yl;
Y is of formula II or III; and
each R⁵ is independently selected from the group consisting of C₁₋₆-alk(en/yn)yl, aryl, halogen, C₁₋₆-alk(en/yn)yloxy, —NR⁷R^{7′}, —SO₂R⁸.

In an embodiment of the present invention, the following definitions are applied for formula I:
The term heteroatom refers to a nitrogen, oxygen or sulphur atom.
Halogen means fluoro, chloro, bromo or iodo.
The expression C₁₋₆-alk(en/yn)yl means a C₁₋₆-alkyl, C₂₋₆-alkenyl or a C₂₋₆-alkynyl group.
The term C₁₋₆-alkyl refers to a branched or unbranched alkyl group having from one to six carbon atoms inclusive, including but not limited to methyl, ethyl, 1-propyl, 2-propyl, 1-butyl, 2-butyl, 2-methyl-2-propyl, 2,2-dimethyl-1-propyl and 2-methyl-1-propyl.
Similarly, C₂₋₆-alkenyl and C₂₋₆-alkynyl, respectively, designate such groups having from two to six carbon atoms, including one double bond and one triple bond respectively, including but not limited to ethenyl, propenyl, butenyl, ethynyl, propynyl and butynyl.
The expression C₃₋₈-cycloalk(en)yl means a C₃₋₈-cycloalkyl- or cycloalkenyl group.
The term C₃₋₈-cycloalkyl designates a monocyclic or bicyclic carbocycle having three to eight C-atoms, including, but not limited to, cyclopropyl, cyclopentyl, cyclohexyl, etc.
The term C₃₋₈-cycloalkenyl designates a monocyclic or bicyclic carbocycle having three to eight C-atoms and including one double bond.
When two substituents together with the nitrogen atom to which they are attached form a 4-8 membered saturated or unsaturated ring which optionally contains 1, 2 or 3 further heteroatoms, then a monocyclic ring system is formed by 4 to 8 atoms selected from the nitrogen atom, 1-7 carbon atoms and 0-3 heteroatoms selected from N, S or O. Examples of such ring systems are azetidine, beta-lactame, pyrrolidine, piperidine, piperazine, morpholine, pyrrole, oxazolidine, thiazolidine, imidazolidine, tetrazole and pyrazole.
The term aryl refers to aromatic systems such as pyridine, thiazole, oxazole, phenyl, naphtyl, thiophene and furan, which are optionally substituted with one or more substituents independently being hydroxy, halogen, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, C₁₋₆-alkoxy, C₃₋₈-alkoxy, acyl, nitro or cyano, —CO—NH—C₁₋₆-alk(en/yn)yl, —CO—N(C₁₋₆-alk(en/yn)yl)₂, —NH—C₁₋₆-alk(en/yn)yl, —N(C₁₋₆-alk(en/yn)yl)₂, —S—C₁₋₆-alk(en/yn)yl, —SO₂—C₁₋₆-alk(en/yn)yl, —SO₂O—C₁₋₆-alk(en/yn)yl, —NH₂, —SO₂N(C₁₋₆-alk(en/yn)yl)₂ or —SO₂NH—C₁₋₆-alk(en/yn)yl; or two adjacent substituents may together with the aromatic group to which they are attached form a 4-8 membered ring, which optionally contains one or two heteroatoms.
When two adjacent substituents together with the aromatic group to which they are attached form a 4-8 membered ring, which optionally contains one or two heteroatoms, then a ring system is formed by 4-8 atoms selected from 3-8 carbonatoms and 0-2 heteroatoms selected from N, S, or O. Such two adjacent substituents may together form: —(CH₂)_{n″}—CH₂—, —CH═CH—(CH₂)_{m″}—, —CH₂—CH═CH—(CH₂)_{p″}, —CH═CH—CH═CH—, —(CH₂)_{n″}—O—, —O—(CH₂)_{m″}—O—, —CH₂—O—(CH₂)_{p″}—O—, —CH₂—O—CH₂—(CH₂)_{n″}—S—, —S—(CH₂)_{m″}—S—, —CH₂—S—(CH₂)_{p″}—S—, —CH₂—S—CH₂—S—CH₂—, —(CH₂)_{n″}—NH—, —NH—(CH₂)_{m″}—NH—, —CH₂—NH—(CH₂)_{p″}—NH—, —CH═CH—NH—, —O—(CH₂)_{m″}—NH—, —CH₂—O—(CH₂)_{p″}—NH— or —O—(CH₂)_{p″}—NH—CH₂—, —S—(CH₂)_{m″}—NH—, —N═CH—NH—, —N═CH—O— or —N═CH—S—, wherein m″ is 1, 2 or 3, n″ is 2, 3 or 4 and p″ is 1 or 2.
As used herein, the term acyl refers to formyl, C₁₋₆-alk(en/yn)ylcarbonyl, C₃₋₈-cycloalk(en)ylcarbonyl, arylcarbonyl, aryl-C₁₋₆-alk(en/yn)ylcarbonyl or a C₃₋₈-cycloalk(en)yl-C₁₋₆-alk(en/yn)yl-carbonyl group, wherein C₁₋₆-alk(en/yn)yl, C₃₋₈-cycloalk(en)yl and aryl are as defined above.
The term halo-C₁₋₆-alk(en/yn)yl designates C₁₋₆-alk(en/yn)yl being substituted with one or more halogen atoms, including but not limited to trifluormethyl. Similarly, halo-C₃₋₈-cycloalk(en)yl designates C₃₋₈-cycloalk(en)yl being substituted with one, or more halogen atoms and halo-C₃₋₈-cycloalk(en)yl-C₃₋₈-alk(en/yn)yl, designates C₃₋₈-cycloalk(en)yl-C₁₋₆-alk(en/yn)yl being substituted with one or more halogen atoms.
In the term C₃₋₈-cycloalk(en)yl-C₁₋₆-alk(en/yn)yl, C₃₋₈-cycloalk(en)yl and C₁₋₆-alk(en/yn)yl are as defined above.
Furthermore, terms such as hydroxy-C₁₋₆-alk(en/yn)yl, hydroxy-C₃₋₈-cycloalk(en)yl, aryl-C₁₋₆-alk(en/yn)yl, C₁₋₆-alk(en/yn)yloxy, C₃₋₈-cycloalk(en)yloxy, C₁₋₆-alk(en/yn)ylcarbonyl, C₃₋₈-cycloalk(en)ylcarbonyl, arylcarbonyl, aryl-C₁₋₆-alk(en/yn)ylcarbonyl, C₃₋₈-cycloalk(en)yl-C₁₋₆-alk(en/yn)ylcarbonyl, aryl-C₃₋₈-cycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, cyano-C₁₋₆-alk(en/yn)yl, cyano-C₃₋₈-cycloalk(en)yl, cyano-C₃₋₈-cycloalk(en)yl-C₁₋₆-alk(en/yn)yl, NR¹⁰R^{10′}—C₁₋₆-alk(en/yn)yl and NR¹⁰R^{10′}—C₃₋₈-cycloalk(en)yl etc. designate groups in which the C₁₋₆-alk(en/yn)yl, C₃₋₈-cycloalk(en)yl and aryl are as defined above.

In an embodiment of formula 1 said compound is selected from the group consisting of: {2-Amino-4-[(5-chloro-thiophen-2-ylmethyl)-methyl-amino]-phenyl}-carbamic acid ethyl ester; {2-Amino-4-[(5-chloro-thiophen-2-ylmethyl)-amino]-phenyl}carbamic acid ethyl ester; {2-Amino-4-[(5-methyl-thiophen-2-ylmethyl)-methyl-amino]-phenyl}-carbamic acid ethyl ester; {2-Amino-4-[(5-bromo-thiophen-2-ylmethyl)-amino]-phenyl}-carbamic acid ethyl ester; {2-Amino-4-[(6-chloro-3-methoxy-benzo[b]thiophen-2-ylmethyl)-amino]-phenyl}-carbamic acid ethyl ester; {2-Amino-4-[(benzo[b]thiophen-2-ylmethyl)-amino]-phenyl}-carbamic acid ethyl ester; {2-Amino-4-[(5-methyl-thiophen-2-ylmethyl)-amino]-phenyl}-carbamic acid ethyl ester; {2-Amino-4-[(4-bromo-3-methoxy-thiophen-2-ylmethyl)-amino]-phenyl}-carbamic acid ethyl ester; {2-Amino-4-[(5-phenyl-thiophen-2-ylmethyl)-amino]-phenyl}-carbamic acid ethyl ester; {2-Amino-4-[(3-chloro-thiophen-2-ylmethyl)-amino]-phenyl}-carbamic acid ethyl ester; (2-Amino-4-{[4-(4-chloro-benzenesulfonyl)-3-methyl-thiophen-2-ylmethyl]-amino}-phenyl)-carbamic acid ethyl ester; {2-Amino-4-[(3-methyl-thiophen-2-ylmethyl)-amino]-phenyl}-carbamic acid ethyl ester; {2-Amino-4-[(5-fluoro-benzofuran-3-ylmethyl)-amino]-phenyl}-carbamic acid ethyl ester; {2-Amino-4-[(thiophen-2-ylmethyl)-amino]-phenyl}-carbamic acid ethyl ester; {2-Amino-4-[(4-bromo-thiophen-2-ylmethyl)-amino]-phenyl}-carbamic acid ethyl ester; {2-Amino-4-[(5-ethyl-thiophen-2-ylmethyl)-amino]-phenyl}-carbamic acid ethyl ester; {2-Amino-4-[(thiophen-3-ylmethyl)-amino]-phenyl}-carbamic acid ethyl ester; {2-Amino-4-[(5-chloro-thiophen-2-ylmethyl)-ethyl-amino]-phenyl}-carbamic acid ethyl ester; {2-Amino-4-[(benzo[b]thiophen-3-ylmethyl)-amino]-phenyl}-carbamic acid ethyl ester; {2-Amino-4-[(5-dimethyl-amino-benzo[b]thiophen-3-ylmethyl)-amino]-phenyl}-carbamic acid ethyl ester; {2-Amino-4-[(5-dimethyl-lamino-3-methyl-benzo[b]thiophen-2-ylmethyl)-amino}-phenyl]-carbamic acid ethyl ester; {2-Amino-4-[(5-fluoro-thiophen-2-ylmethyl)-amino]-phenyl}-carbamic acid ethyl ester; {2-Amino-4-[(benzo[b]thiophen-2-ylmethyl)-amino]-phenyl}-carbamic acid propyl ester; {2-Amino-4-[(benzo[b]thiophen-3-ylmethyl)-amino]-phenyl}-carbamic acid propyl ester; N-{2-Amino-4-[(5-chloro-thiophen-2-ylmethyl)-amino]phenyl}-2-(4-fluoro-phenyl)-acetamide; and N-{2-Amino-4-[(5-chloro-thiophen-2-ylmethyl)amino]phenyl}-3,3-dimethyl-butyramide or a pharmaceutically acceptable salt thereof.

Compounds according to formula 1 can be prepared as described in WO2004/058739.

An embodiment of the invention relates to a method wherein said compound is a compound according to formula 2

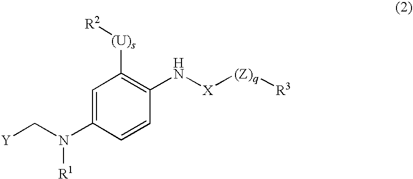

wherein:
s is 0 or 1;
U is O, S, SO₂, SO₂NR¹¹, CO—O or CONR¹¹; wherein
   R¹¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl; or R² and R¹¹ together with the nitrogen atom form a 5-8 membered saturated or unsaturated ring which optionally contains 1, 2 or 3 further heteroatoms;
q is 0 or 1;
X is CO or SO₂; with the proviso that q is 0 when X is SO₂;
Z is O or S;
R¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl;
R² is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, NR¹⁰R^{10′}—C₁₋₆-alk(en/yn)yl, NR¹⁰R^{10′}—C₃₋₈-cycloalk(en)yl and NR¹⁰R^{10′}—C₃₋₈-cycloalk(en)yl-C₁₋₆-alk(en/yn)yl; wherein
   R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or
   R¹⁰ and R^{10′} together with the nitrogen atom form a 5-8 membered saturated or unsaturated ring which optionally contains 1, 2 or 3 further heteroatoms;

provided that when R² is halogen or cyano then s is 0; and

provided that U is O or S when s is 1 and R² is a hydrogen atom or acyl;
R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, heterocycloalk(en)yl-C₁₋₆-alk(en/yn)yl, Ar, Ar—C₁₋₆-alk(en/yn)yl, Ar—C₃₋₈-cycloalk(en)yl, Ar-heterocycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar—C₁₋₆-alk(en/yn)yl-heterocycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar—C₁₋₆-alk(en/yn)yloxy-C₁₋₆-alk(en/yn)yl, C₁₋₆-alk(en/yn)yloxy-carbonyl-C₁₋₆-alk(en/yn)yl, C₃₋₈-cycloalk(en)yl oxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yk(en)yl, acyl-C₁₋₆-alk(en/yn)yl-heterocycloalk(en)yl, NR¹²R^{12′}, optionally substituted NR¹²NR^{12′}—C₁₋₆-alk(en/yn)yl, optionally substituted NR¹²R^{12′}—C₃₋₈cycloalk(en)yl, optionally substituted NR¹²R^{12′}—C₃₋₈-cycloalk(en)yl-C₁₋₆-alk(en/yn)yl; wherein
   R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, Ar-heterocycloalk(en)yl, Ar-oxy-C₁₋₆-alk(en/yn)yl, Ar-oxy-C₃₋₈-cycloalk(en)yl, Ar-oxy-C₃₋₈-cycloalk(en)yl-C₁₋₆-alk(en/yn)yl, Ar-oxy-heterocycloalk(en)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or
   R¹² and R^{12′} together with the nitrogen atom form a 5-8 membered saturated or unsaturated ring which optionally contains 1, 2 or 3 further heteroatoms;
   with the proviso that when R³ is NR¹²R^{12′} then q is 0; and
Y represents a group of formula XXIV, XXV, XXVI, XXVII, XXVIII, XXXXI or XXXXII:

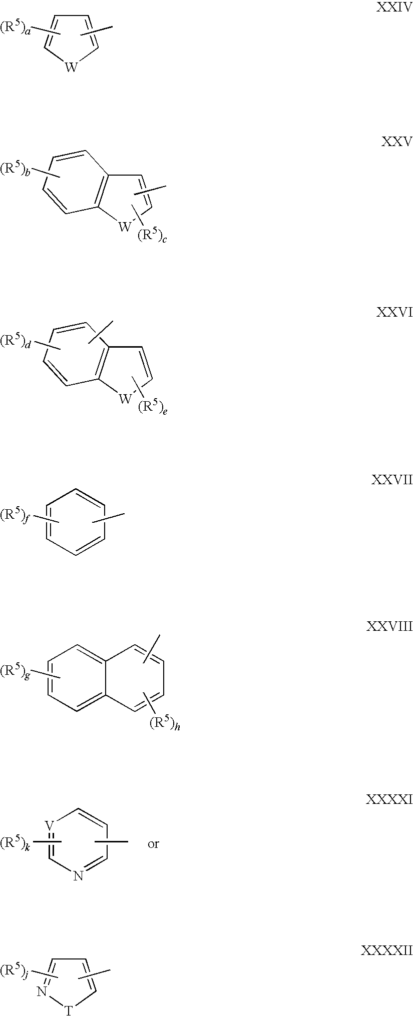

wherein
the line represents a bond attaching the group represented by Y to the carbon atom;
W is O or S;
V is N, C or CH,
T is N, NH or O;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3;
j is 0, 1 or 2;
k is 0, 1, 2 or 3; and
each R⁵ is independently selected from the group consisting of a C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, Ar-oxy, Ar-oxy-C₁₋₆-alk(en/yn)yl, Ar-oxy-C₃₋₈-cycloalk(en)yl, C₁₋₆-alk(en/yn)yl-heterocycloalk(en)yl, Ar-oxy-C₃₋₈-cycloalk(en)yl-C₁₋₆-alk(en/yn)yl, acyl, C₁₋₆-alk(en/yn)yloxy, C₃₋₈-cycloalk(en)yloxy, C₃₋₈-cycloalk(en)yl-C₁₋₆-alk(en/yn)yloxy, C₁₋₆-alk(en/yn)yloxy-carbonyl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —CO—NR⁶R^{6′}, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, NR⁷R^{7′}, S—R⁸ and SO₂R⁸, or two adjacent R⁵ together with the aromatic group form a 5-8 membered ring which optionally contains one or two heteroatoms;
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, heterocycloalk(en)yl-C₁₋₆-alk(en/yn)yl, heterocycloalk(en)yl-C₃₋₈-cycloalk(en)yl, heterocycloalk(en)yl-C₃₋₈-cycloalk(en)yl-C₁₋₆-alk(en/yn)yl, heterocycloalk(en)yl-Ar and acyl; or R⁷ and R^{7′} together with the nitrogen atom form a 5-8 membered saturated or unsaturated ring which optionally contains 1, 2 or 3 further heteroatoms; and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′};
wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl;

or salts thereof.

In an embodiment of formula 2:
R¹ is C₁₋₆-alk(en/yn)yl or a hydrogen atom.
U is an oxygen atom.
R² is selected from the group consisting of hydrogen, C₁₋₆-alk(en/yn)yl, C₃₋₈-cycloalk(en)yl, Ar, Ar—C₁₋₆-alk(en/yn)yl, halogen, halo-C₁₋₆-alk(en/yn)yl and cyano;
provided that when R² is halogen or cyano then s is 0; and
provided that U is O or S when s is 1 and R² is a hydrogen atom.
X is CO.
R³ is C₁₋₆-alk(en/yn)yl, C₃₋₈-cycloalk(en)yl, C₃₋₈-cycloalk(en)yl-C₁₋₆-alk(en/yn)yl, heterocycloalk(en)yl-C₁₋₆-alk(en/yn)yl, heterocycloalk(en)yl, Ar, Ar—C₁₋₆-alk(en/yn)yl, Ar-oxy-C₁₋₆-alk(en/yn)yl, Ar—C₁₋₆-alk(en/yn)yloxy-C₁₋₆-alk(en/yn)yl, C₁₋₆-alk(en/yn)yloxy-carbonyl-C₁₋₆-alk(en/yn)yl, halo-C₁₋₆-alk(en/yn)yl, NR¹²R^{12′}, optionally substituted NR¹²R^{12′}—C₁₋₆-alk(en/yn)yl, and optionally substituted NR¹²R^{12′}—C₃₋₈-cycloalk(en)yl.
R¹² and R^{12′} are independently selected from the group consisting of hydrogen, C₁₋₆-alk(en/yn)yl and Ar.
Y is of formula XXIV or XXV, XXVII or XXXXI.
V is a nitrogen atom or CH.
T is a nitrogen atom or an oxygen atom.
each R⁵ is independently selected from the group consisting of a C₁₋₆-alk(en/yn)yl, C₁₋₆-alk(en/yn)yl-heterocycloalk(en)yl, Ar, C₁₋₆-alk(en/yn)yloxy, Ar-oxy, C₁₋₆-alk(en/yn)yloxy-carbonyl, halogen, halo-C₁₋₆-alk(en/yn)yl, NR⁷R^{7′}, S—R⁸ and SO₂R⁸, or two adjacent R⁵ together with the aromatic group form a 5-8 membered ring, which optionally contains one or two heteroatoms.
both R⁷ and R^{7′} are C₁₋₆-alk(en/yn)yl.
R⁸ is selected from the group consisting of C₁₋₆-alk(en/yn)yl and Ar.

In an embodiment of the present invention, the following definitions are applied for formula 2:
The term heteroatom refers to a nitrogen, oxygen or sulphur atom.
Halogen means fluoro, chloro, bromo or iodo.
The expressions C₁₋₆-alk(en/yn)yl and C₁₋₆-alk(an/en/yn)yl mean a C₁₋₆-alkyl, C₂₋₆-alkenyl or a C₂₋₆-alkynyl group.
The term C₁₋₆-alkyl refers to a branched or un-branched alkyl group having from one to six carbon atoms inclusive, including but not limited to methyl, ethyl, 1-propyl, 2-propyl, 1-butyl, 2-butyl, 2-methyl-2-propyl and 2-methyl-1-propyl.
Similarly, C₂₋₆-alkenyl and C₂₋₆-alkynyl, respectively, designate such groups having from two to six carbon atoms, including one double bond and one triple bond respectively, including but not limited to ethenyl, propenyl, butenyl, ethynyl, propynyl and butynyl.
The expression C₁₋₃-alk(en/yn)yl means a C₁₋₃-alkyl, C₂₋₃-alkenyl or a C₂₋₃-alkynyl group.
The term C₁₋₃-alkyl refers to a branched or un-branched alkyl group having from one to three carbon atoms inclusive, including but not limited to methyl, ethyl, 1-propyl and 2-propyl.
Similarly, C₂₋₃-alkenyl and C₂₋₃-alkynyl, respectively, designate such groups having from two to three carbon atoms, including one double bond and one triple bond respectively, including but not limited to ethenyl, propenyl, ethynyl and propynyl.
The expressions C₃₋₈-cycloalk(en)yl and C₃₋₈-cycloalk(an/en)yl mean a C₃₋₈-cycloalkyl- or cycloalkenyl group.
The term C₃₋₈-cycloalkyl designates a monocyclic or bicyclic carbocycle having three to eight C-atoms, including but not limited to cyclopropyl, cyclopentyl, cyclohexyl, etc.
The expressions C₃₋₆-cycloalk(en)yl and C₃₋₆-cycloalk(an/en)yl mean a C₃₋₆-cycloalkyl- or cycloalkenyl group.
The term C₃₋₆-cycloalkyl designates a monocyclic or bicyclic carbocycle having three to six C-atoms, including but not limited to cyclopropyl, cyclopentyl, cyclohexyl, etc.
The term C₃₋₈-cycloalkenyl designates a monocyclic or bicyclic carbocycle having three to eight C-atoms and including one double bond.
The term heterocycloalk(en)yl designates monocyclic or bicyclic ring systems wherein the ring is formed by 5 to 8 atoms being selected from the group consisting of carbonatoms and heteroatoms; with the proviso that one or two of the ring forming atoms are independently selected heteroatoms. The term heterocycloalk(en)yl may thus designate a monocyclic or bicyclic ring system wherein the ring is formed by 5 to 8 atoms selected from 3-7 carbonatoms and 1 or 2 heteroatoms selected from N, S, or O. Examples of such ring systems are morpholine, pyrrolidine, piperidine and piperazine.
The term halo-C₁₋₆-alk(en/yn)yl designates C₁₋₆-alk(en/yn)yl being substituted with one or more halogen atoms, including but not limited to trifluoromethyl. Similarly, halo-C₃₋₈-cycloalk(en)yl designates C₃₋₈-cycloalk(en)yl being substituted with one or more halogen atoms and halo-heterocycloalk(en)yl designates heterocycloalk(en)yl being substituted with one or more halogen atoms.
The term NR¹⁰R^{10′}—C₁₋₆-alk(en/yn)yl designates C₁₋₆-alk(en/yn)yl being substituted with NR¹⁰R^{10′}; NR¹²R^{12′}—C₁₋₆-alk(en/yn)yl designates C₁₋₆-alk(en/yn)yl being substituted with NR¹²R^{12′}; and NR⁷R^{7′}—C₁₋₆-alk(en/yn)yl designates C₁₋₆-alk(en/yn)yl being substituted with NR⁷R^{7″}. 2-amino-4-methyl-pentane is an example of such group, the example is not intended to be construed as limiting.
The term NR¹⁰R^{10′}—C₃₋₈-cycloalk(en)yl designates C₃₋₈-cycloalk(en)yl being substituted with NR¹⁰R^{10′}; NR¹²R^{12′}—C₃₋₈-cycloalk(en)yl designates C₃₋₈-cycloalk(en)yl being substituted with NR¹²R^{12′}; and NR⁷R^{7′}—C₃₋₈-cycloalk(en)yl designates C₃₋₈-cycloalk(en)yl being substituted with NR⁷R^{7′}. 1-amino-cyclopropane is an example of such group, the example is not intended to be construed as limiting.
The term NR¹⁰R^{10′}—C₃₋₈-cycloalk(en)yl-C₁₋₆-alk(en/yn)yl designates C₃₋₈-cycloalk(en)yl-C₁₋₆-alk(en/yn)yl being substituted with NR¹⁰R^{10′}; NR¹²R^{12′}—C₃₋₈-cycloalk(en)yl-C₁₋₆-alk(en/yn)yl designates C₃₋₈-cycloalk(en)yl-C₁₋₆-alk(en/yn)yl being substituted with NR¹²R^{12′}; and NR⁷R^{7′}—C₃₋₈-cycloalk(en)yl-C₁₋₆-alk(en/yn)yl designates C₃₋₈-cycloalk(en)yl-C₁₋₆-alk(en/yn)yl being substituted with NR⁷R^{7′}.
When any of NR¹²R^{12′}C₁₋₆-alk(en/yn)yl, NR¹²R^{12′}—C₃₋₈-cycloalk(en)yl, NR¹²R^{12′}—C₃₋₈-cycloalk(en)yl-C₁₋₆-alk(en/yn)yl is optionally substituted, then any of C₁₋₆-alk(en/yn)yl, C₃₋₈-cycloalk(en)yl, C₃₋₈-cycloalk(en)yl-C₁₋₆-alk(en/yn)yl is optionally substituted with one or more substituents independently being C₁₋₆-alk(en/yn)yl, C₃₋₈-cycloalk(en)yl, C₃₋₈-cycloalk(en)yl-C₁₋₆-alk(en/yn)yl or Ar.
As used herein, the term acyl refers to formyl, C₁₋₆-alk(en/yn)ylcarbonyl, C₃₋₈-cycloalk(en)ylcarbonyl, Ar-carbonyl, Ar—C₁₋₆-alk(en/yn)ylcarbonyl or a C₃₋₈cycloalk(en)yl-C₁₋₆-alk(en/yn)yl-carbonyl group, wherein C₁₋₆-alk(en/yn)yl, cycloalk(en)yl and Ar are as defined above.
When two substituents together with a nitrogen atom form a 5-8 membered saturated or unsaturated ring which optionally contains one further heteroatom, then a monocyclic ring system is formed by 5 to 8 atoms, one or two of said atoms are heteroatoms selected from N, S, or O. Examples of such ring systems are pyrrolidine, piperidine, piperazine, morpholine, pyrrole, oxazolidine, thiazolidine, imidazolidine, azetidine, beta-lactame, tetrazole and pyrazole.
When two adjacent substituents together with an aromatic group to which they are attached form a 5-8 membered ring, which optionally contains one or two heteroatoms, then a ring is formed by 5-8 atoms selected from 3-8 carbonatoms and 0-2 heteroatoms selected from N, S, or O and. Such two adjacent substituents may together form: —(CH₂)_{n″}—CH₂—, —CH═CH—(CH₂)_{m″}—, —CH₂—CH═CH—(CH₂)_{p″}, —CH═CH—CH═CH—, —(CH₂)_{n″}—O—, —O—(CH₂)_{m″}—O—, —CH₂—O—(CH₂)_{p″}—O—, —CH₂—O—CH₂—(CH₂)_{n″}—S—, —S—(CH₂)_{m″}—S—, —CH₂—S—(CH₂)_{p″}—S—, —CH₂—S—CH₂—S—CH₂—, —(CH₂)_{n″}—NH—, —NH—(CH₂)_{m″}—NH—, —CH₂—NH—(CH₂)_{p″}—NH—, —CH═CH—NH—, —O—(CH₂)_{m″}—NH—, —CH₂—O—(CH₂)_{p″}—NH— or —O—(CH₂)_{p″}—NH—CH₂—, —S—(CH₂)_{m″}—NH—, —N═CH—NH—, —N═CH—O— or —N═CH—S—, wherein m″ is 1, 2 or 3, n″ is 2, 3 or 4 and p″ is 1 or 2.
The term Ar refers to optionally substituted aromatic systems of 5-10 carbon atoms, wherein 0, 1, 2, 3 or 4 carbon atoms may be replaced by heteroatoms independently selected from N, S, or O. Examples of such Ar groups are optionally substituted phenyl, optionally substituted naphtyl, optionally substituted quinoline, optionally substituted indol, optionally substituted pyridine, optionally substituted pyrimidine, optionally substituted thiophene, optionally substituted furan, optionally substituted thiazole and optionally substituted oxazole. Such optionally substituted Ar groups may be substituted with one or more substituents independently being hydroxy, halogen, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, C₁₋₆-alk(en/yn)yloxy, C₃₋₈-alk(en/yn)yloxy, acyl, nitro, cyano, —CO—NH—C₁₋₆-alk(en/yn)yl, —CO—N(C₁₋₆-alk(en/yn)yl)₂, —NH₂, —NH—C₁₋₆-alk(en/yn)yl, —N(C₁₋₆-alk(en/yn)yl)₂, S—C₁₋₆-alk(en/yn)yl, —SO₂N(C₁₋₆-alk(en/yn)yl)₂ and —SO₂NH—C₁₋₆-alk(en/yn)yl, SO₂—C₁₋₆-alk(en/yn)yl and SO₂O—C₁₋₆-alk(en/yn)yl; or two adjacent substituents may together with the aromatic group form a 5-8 membered ring, which optionally contains one or two heteroatoms and which may be saturated or unsaturated.
The terms C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, Ar, Ar—C₁₋₆-alk(en/yn)yl, Ar—C₃₋₈-cycloalk(en)yl, Ar-heterocycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar—C₁₋₆-alk(en/yn)yl-heterocycloalk(en)yl, C₁₋₆-alk(en/yn)yloxy, C₂₋₆-alkenyloxy, C₂₋₆-alkynyloxy, C₃₋₈-cycloalk(en)yl oxy, C₁₋₆alk(en/yn)yl oxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, 6-alk(en/yn)yloxy-heterocycloalk(en)yl, Ar-oxy-C₁₋₆-alk(en/yn)yl, Ar—C₁₋₆-alk(en/yn)yloxy-C₁₋₆-alk(en/yn)yl, C₁₋₆-alk(en/yn)ylcarbonyl, C₃₋₈alk(en/yn)ylcarbonyl, Ar-carbonyl, Ar—C₁₋₆-alk(en/yn)ylcarbonyl, C₃₋₈-cycloalk(en)yl-C₁₋₆-alk(en/yn)ylcarbonyl, —CO—C₁₋₆-alk(en/yn)yl, S—C₁₋₆-alk(en/yn)yl, SO₂—C₁₋₆-alk(en/yn)yl and SO₂O—C₁₋₆-alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, acyl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, halo-heterocycloalk(en)yl-Ar, cyano-C₁₋₆-alk(en/yn)yl, cyano-C₁₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl etc. designate such groups in which the C₁₋₆-alk(en/yn)yl, C₂₋₆-alkenyl, C₂₋₆-alkynyl, C₃₋₈-cycloalk(en)yl, heterocycloalk(en)yl, Ar, cyano, halo-C₁₋₄-alk(en/yn)yl, halo-C₃₋₈-cycloalk(en)yl, halo-heterocycloalk(en)yl and acyl are as defined above.

In an embodiment of formula 2 said compound is selected from the following list:
{4-[(Benzofuran-2-ylmethyl)-amino]-2-methylphenyl}-carbamic acid propyl ester;
(4-[(5-Chloro-thiophen-2-ylmethyl)-amino]-2-methylphenyl)-carbamic acid ethyl ester;
{4-[(Benzo[b]thiophen-2-ylmethyl)-amino]-2-methylphenyl}-carbamic acid ethyl ester;
{2-Methyl-4-[(5-phenyl-thiophen-2-ylmethyl)-amino]-phenyl}-carbamic acid ethyl ester;
[4-(4-Isopropyl-benzylamino)-2-methylphenyl]-carbamic acid ethyl ester;
[4-(4-Fluoro-benzylamino)-2-methylphenyl]-carbamic acid propyl ester;
(4-{[4-(4-Chloro-benzenesulfonyl)-3-methyl-thiophen-2-ylmethyl]-amino}-2-methylphenyl)-carbamic acid propyl ester;
4-[(5-Methyl-thiophen-2-ylmethyl)-amino]-2-methylphenyl)-carbamic acid propyl ester;
{4-[(5-Bromo-thiophen-2-ylmethyl)-amino]-2-methylphenyl}-carbamic acid propyl ester;
{4-[(5-Chloro-thiophen-2-ylmethyl)-amino]-2-methylphenyl}-carbamic acid propyl ester;
{4-[(Benzo[b]thiophen-2-ylmethyl)-amino]-2-methylphenyl}-carbamic acid propyl ester;
{2-Methyl-4-[(5-phenyl-thiophen-2-ylmethyl)-amino]-phenyl}-carbamic acid propyl ester;
[4-(4-Isopropyl-benzylamino)-2-methylphenyl]-carbamic acid propyl ester,
{4-[(5-Bromo-thiophen-2-ylmethyl)-amino]-2-chlorophenyl}-carbamic acid ethyl ester;
{4-[(5-Chloro-thiophen-2-ylmethyl)-amino]-2-chlorophenyl}-carbamic acid ethyl ester;
{4-[(Benzo[b]thiophen-2-ylmethyl)-amino]-2-chlorophenyl}-carbamic acid ethyl ester;
[2-Chloro-4-(4-isopropyl-benzylamino)-phenyl]-carbamic acid ethyl ester;
[2-Chloro-4-(4-fluoro-benzylamino)-phenyl]-carbamic acid propyl ester;
2-Chloro-4-{[4-(4-chloro-benzenesulfonyl)-3-methyl-thiophen-2-ylmethyl]-amino}-phenyl)-carbamic acid propyl ester;
{4-[(5-Methyl-thiophen-2-ylmethyl)-amino]-2-chlorophenyl}-carbamic acid propyl ester;
{4-[(5-Bromo-thiophen-2-ylmethyl)-amino]-2-chlorophenyl}-carbamic acid propyl ester;
{2-Chloro-4-[(5-chloro-thiophen-2-ylmethyl)-amino]-phenyl}-carbamic acid propyl ester;
{4-[(Benzo[b]thiophen-2-ylmethyl)-amino]-2-chlorophenyl}-carbamic acid propyl ester;
{4-[(Benzofuran-2-ylmethyl)-amino]-2-chlorophenyl}-carbamic acid propyl ester;
{4-[(5-Chloro-thiophen-2-ylmethyl)-amino]-2-cyanophenyl}-carbamic acid ethyl ester;
{4-[(Benzo[b]thiophen-2-ylmethyl)-amino]-2-methoxyphenyl}-carbamic acid methyl ester;
{4-[(5-Bromo-thiophen-2-ylmethyl)-amino]-2-methoxyphenyl}-carbamic acid isopropyl ester;
{4-[(4-Fluoro-benzyl)-(methyl)amino]-2-methoxyphenyl}-carbamic acid propyl ester;
[4-(Benzo[b]thiophen-2-ylmethyl-(methyl)amino)-2-methoxy-phenyl]-carbamic acid propyl ester;
{4-[(5-Chloro-thiophen-2-ylmethyl)-(methyl)amino]-2-methoxy-phenyl}-carbamic acid propyl ester;
{4-[(5-Bromo-thiophen-2-ylmethyl)-(methyl)amino]-2-methoxy-phenyl}-carbamic acid propyl ester;
{2-Methoxy-4-[methyl-(5-methyl-thiophen-2-ylmethyl)-amino]-phenyl}-carbamic acid propyl ester;
{4-[(4-Fluorobenzyl)-(methyl)-amino]-2-isopropoxyphenyl}-carbamic acid ethyl ester;
[4-(3-Fluorobenzylamino)-2-methoxyphenyl]-carbamic acid ethyl ester;
[4-(4-Isopropylbenzylamino)-2-methoxyphenyl]-carbamic acid ethyl ester;
{2-Methoxy-4-[(3-methylthiophen-2-ylmethyl)-amino]-phenyl}-carbamic acid ethyl ester;
[4-(2,4-Difluorobenzylamino)-2-methoxyphenyl]-carbamic acid ethyl ester;
[2-Cyclopentyloxy-4-(4-methoxybenzylamino)-phenyl]-carbamic acid ethyl ester;
[2-Cyclopentyloxy-4-(3-fluoro-2-methylbenzylamino)-phenyl]-carbamic acid ethyl ester;
[4-(3-Fluoro-2-methylbenzylamino)-2-phenethyloxyphenyl]-carbamic acid ethyl ester;
[2-Benzyloxy-4-(3-fluoro-2-methylbenzylamino)-phenyl]-carbamic acid ethyl ester;
[2-Benzyloxy-4-(4-methylsulfanylbenzylamino)-phenyl]-carbamic acid ethyl ester;
{4-[(Benzo[b]thiophen-3-ylmethyl)-amino]-2-cyclopentyloxyphenyl}-carbamic acid ethyl ester;
{4-(3-Fluoro-2-methylbenzylamino)-2-isopropoxyphenyl}-carbamic acid ethyl ester;
[2-Benzyloxy-4-(3-methoxybenzylamino)-phenyl]-carbamic acid ethyl ester;
{4-[(Benzo[1,3]dioxol-5-ylmethyl)-amino]-2-isopropoxyphenyl}-carbamic acid ethyl ester;
{4-[(5-Bromo-thiophen-2-ylmethyl)-amino]-phenyl}-carbamic acid propyl ester;
{4-[(5-Chloro-thiophen-2-ylmethyl)-amino]-phenyl}-carbamic acid propyl ester;
[2-Cyano-4-(4-isopropylbenzylamino)-phenyl]-carbamic acid ethyl ester;
{4-[(5-Bromo-thiophen-2-ylmethyl)-(methyl)amino]-2-methyl phenyl}-carbamic acid propyl ester;
{4-[(4-Isopropylbenzyl)-(methyl)amino]-2-methylphenyl}-carbamic acid propyl ester;
{2-Methyl-4-[methyl-(4-trifluoromethyl-benzyl)-amino]-phenyl}-carbamic acid propyl ester;
{2-Methyl-4-[methyl-(4-methylsulfanyl-benzyl)-amino]-phenyl}-carbamic acid propyl ester;
{4-[(4-tert-Butyl-benzyl)-(methyl)amino]-2-chlorophenyl}-carbamic acid ethyl ester;
{2-Chloro-4-[methyl-(4-trifluoromethyl-benzyl)-amino]-phenyl}-carbamic acid ethyl ester;
{2-Chloro-4-[methyl-(4-methylsulfanyl-benzyl)-amino]-phenyl}-carbamic acid ethyl ester;
{4-[(5-Bromo-thiophen-2-ylmethyl)-(methyl)amino]-2-chlorophenyl}-carbamic acid propyl ester;
{2-Chloro-4-[(5-chloro-thiophen-2-ylmethyl)-(methyl)amino]-phenyl}-carbamic acid propyl ester;
{4-[(4-tert-Butyl-benzyl)-(methyl)amino]-2-chlorophenyl}-carbamic acid propyl ester;
{2-Chloro-4-[methyl-(4-trifluoromethyl-benzyl)-amino]-phenyl}-carbamic acid propyl ester;
{4-[(5-Bromo-thiophen-2-ylmethyl)-(methyl)amino]-2-trifluoromethyl-phenyl}-carbamic acid ethyl ester;
{4-[(5-Chloro-thiophen-2-ylmethyl)-(methyl)amino]-2-trifluoromethyl-phenyl}-carbamic acid ethyl ester;
{4-[(4-Isopropyl-benzyl)-(methyl)amino]-2-trifluoromethyl-phenyl}-carbamic acid ethyl ester;
{4-[(4-tert-Butyl-benzyl)-(methyl)amino]-2-trifluoromethyl-phenyl}-carbamic acid ethyl ester;
{4-[Methyl-(4-trifluoromethyl-benzyl)-amino]-2-trifluoromethyl-phenyl}-carbamic acid ethyl ester;
{4-[Methyl-(4-methylsulfanyl-benzyl)-amino]-2-trifluoromethyl-phenyl}-carbamic acid ethyl ester;
{4-[(5-Bromo-thiophen-2-ylmethyl)-(methyl)amino]-2-trifluoromethyl-phenyl}-carbamic acid propyl ester;
{4-[(5-Chloro-thiophen-2-ylmethyl)-(methyl)amino]-2-trifluoromethyl-phenyl}-carbamic acid propyl ester;
{4-[(4-Isopropyl-benzyl)-(methyl)amino]-2-trifluoromethyl-phenyl}-carbamic acid propyl ester;
{4-[(4-tert-Butyl-benzyl)-(methyl)amino]-2-trifluoromethyl-phenyl}-carbamic acid propyl ester;
{4-[Methyl-(4-trifluoromethyl-benzyl)-amino]-2-trifluoromethyl-phenyl}-carbamic acid propyl ester;
{4-[Methyl-(4-methylsulfanyl-benzyl)-amino]-2-trifluoromethyl-phenyl}-carbamic acid propyl ester;
{4-[(5-Bromo-thiophen-2-ylmethyl)-(methyl)amino]-2-cyanophenyl}-carbamic acid propyl ester;
{4-[(4-tert-Butyl-benzyl)-(methyl)amino]-2-cyanophenyl}-carbamic acid propyl ester;
{2-Cyano-4-[methyl-(4-trifluoromethyl-benzyl)-amino]-phenyl}-carbamic acid propyl ester;
{2-Bromo-4-[(5-bromo-thiophen-2-ylmethyl)-(methyl)amino]-phenyl}-carbamic acid propyl ester;
{2-Bromo-4-[(5-chloro-thiophen-2-ylmethyl)-(methyl)amino]-phenyl}-carbamic acid propyl ester;
{2-Bromo-4-[(4-isopropylbenzyl)-(methyl)amino]-phenyl}-carbamic acid propyl ester;
{2-Bromo-4-[(4-tert-butyl-benzyl)-(methyl)amino]-phenyl}-carbamic acid propyl ester;
{2-Bromo-4-[methyl-(4-trifluoromethyl-benzyl)-amino]-phenyl}-carbamic acid propyl ester;
[2-Iodo-4-(4-isopropyl-benzylamino)-phenyl]-carbamic acid propyl ester;
[4-(4-tert-Butyl-benzylamino)-2-iodophenyl]-carbamic acid propyl ester;
[2-Iodo-4-(4-trifluoromethyl-benzylamino)-phenyl]-carbamic acid propyl ester;
[2-Iodo-4-(4-methylsulfanyl-benzylamino)-phenyl]-carbamic acid propyl ester;
{2-Iodo-4-[4-(4-methylpiperazin-1-yl)-benzylamino]-phenyl}-carbamic acid propyl ester;
{4-[(5-Bromo-thiophen-2-ylmethyl)-amino]-2-trifluoromethyl-phenyl}-carbamic acid ethyl ester;
{4-[(5-Chloro-thiophen-2-ylmethyl)-amino]-2-trifluoromethyl-phenyl}-carbamic acid ethyl ester;
[4-(4-tert-Butyl-benzylamino)-2-trifluoromethyl-phenyl]-carbamic acid ethyl ester;
[4-(4-Methylsulfanyl-benzylamino)-2-trifluoromethyl-phenyl]-carbamic acid ethyl ester;
{4-[(5-Bromo-thiophen-2-ylmethyl)-amino]-2-trifluoromethyl-phenyl}-carbamic acid propyl ester;
[4-(4-Isopropylbenzylamino)-2-trifluoromethyl-phenyl]-carbamic acid propyl ester;
[4-(4-tert-Butyl-benzylamino)-2-trifluoromethyl-phenyl]-carbamic acid propyl ester;
[2-Trifluoromethyl-4-(4-trifluoromethyl-benzylamino)-phenyl]-carbamic acid propyl ester;
[4-(4-Dimethylamino-benzylamino)-2-trifluoromethyl-phenyl]-carbamic acid propyl ester;
[4-(4-Methylsulfanyl-benzylamino)-2-trifluoromethyl-phenyl]-carbamic acid propyl ester;
{4-[(5-Bromo-thiophen-2-ylmethyl)-amino]-2-cyanophenyl}-carbamic acid propyl ester;
{4-[(5-Chloro-thiophen-2-ylmethyl)-amino]-2-cyanophenyl}-carbamic acid propyl ester;
[2-Cyano-4-(4-trifluoromethyl-benzylamino)-phenyl]-carbamic acid propyl ester;
{2-Bromo-4-[(5-bromo-thiophen-2-ylmethyl)-amino]phenyl}-carbamic acid propyl ester;
{2-Bromo-4-[(5-chloro-thiophen-2-ylmethyl)-amino]-phenyl}-carbamic acid propyl ester;
[2-Bromo-4-(4-isopropylbenzylamino)-phenyl]-carbamic acid propyl ester;
[2-Bromo-4-(4-tert-butyl-benzylamino)-phenyl]-carbamic acid propyl ester;
[2-Bromo-4-(4-trifluoromethyl-benzylamino)-phenyl]-carbamic acid propyl ester;
[2-Bromo-4-(4-methylsulfanyl-benzylamino)-phenyl]-carbamic acid propyl ester;
N-{4-[(5-Bromo-thiophen-2-ylmethyl)-amino]-2-methoxyphenyl}-butyramide;
N-{4-[(5-Chloro-thiophen-2-ylmethyl)-amino]-2-methoxyphenyl}-butyramide;
N-[4-(4-Isopropylbenzylamino)-2-methoxyphenyl]-butyramide;
N-[4-(4-tert-Butyl-benzylamino)-2-methoxyphenyl]-butyramide;
N-[2-Methoxy-4-(4-trifluoromethyl-benzylamino)-phenyl]-butyramide;
{4-[(5-Chloro-thiophen-2-ylmethyl)-amino]-2-furan-2-yl-phenyl}-carbamic acid propyl ester;
[2-Furan-2-yl-4-(4-isopropylbenzylamino)-phenyl]-carbamic acid propyl ester;
[5-(4-Fluorobenzylamino)-biphenyl-2-yl]-carbamic acid propyl ester;
{5-[(5-Chloro-thiophen-2-ylmethyl)-amino]-biphenyl-2-yl}-carbamic acid propyl ester;
[5-(4-Isopropylbenzylamino)-biphenyl-2-yl]-carbamic acid propyl ester;
N-{2-Chloro-4-[(5-chloro-thiophen-2-ylmethyl)-(methyl)amino]-phenyl}-2-phenylacetamide;
N-{2-Chloro-4-[(5-chloro-thiophen-2-ylmethyl)-(methyl)amino]-phenyl}-3,3-dimethyl butyramide;
N-{2-Chloro-4-[(5-chloro-thiophen-2-ylmethyl)-(methyl)amino]-phenyl}-3-phenylpropionamide;
N-{2-Chloro-4-[(5-chloro-thiophen-2-ylmethyl)-(methyl)amino]phenyl}-butyramide;
Pentanoic acid {2-chloro-4-[(5-chloro-thiophen-2-ylmethyl)-(methyl)amino]-phenyl}-amide;
Cyclopropanecarboxylic acid {2-chloro-4-[(5-chloro-thiophen-2-ylmethyl)-(methyl)amino]-phenyl}-amide;
Cyclobutanecarboxylic acid {2-chloro-4-[(5-chloro-thiophen-2-ylmethyl)-(methyl)amino]-phenyl}-amide;
Cyclopentanecarboxylic acid {2-chloro-4-[(5-chloro-thiophen-2-ylmethyl)-(methyl)amino]-phenyl}-amide;
Cyclohexanecarboxylic acid {2-chloro-4-[(5-chloro-thiophen-2-ylmethyl)-(methyl)amino]-phenyl}-amide;
N-{2-Chloro-4-[(5-chloro-thiophen-2-ylmethyl)-(methyl)amino]-phenyl}-2-thiophen-2-yl-acetamide;
N-{2-Chloro-4-[(5-chloro-thiophen-2-ylmethyl)-(methyl)amino]-phenyl-2-(3-methoxy-phenyl}-acetamide;
N-{2-Chloro-4-[(5-chloro-thiophen-2-ylmethyl)-(methyl)amino]-phenyl}-2-(4-chloro-phenyl)-acetamide;
N-{2-Chloro-4-[(5-chloro-thiophen-2-ylmethyl)-(methyl)amino]-phenyl}-2-(4-methoxy-phenyl)-acetamide;
N-{2-Chloro-4-[(5-chloro-thiophen-2-ylmethyl)-(methyl)amino]-phenyl}-2-(4-fluoro-phenyl)-acetamide;
N-{2-Chloro-4-[(5-chloro-thiophen-2-ylmethyl)-(methyl)amino]-phenyl}-3-cyclohexylpropionamide;
N-{2-Chloro-4-[(5-chloro-thiophen-2-ylmethyl)-amino]-phenyl}-2,2-dimethylpropionamide;
N-{2-Chloro-4-[(5-chloro-thiophen-2-ylmethyl)-amino]-phenyl}-2-phenoxyacetamide;
N-{2-Chloro-4-[(5-chloro-thiophen-2-ylmethyl)-amino]-phenyl}-2-phenyl acetamide;
N-{2-Chloro-4-[(5-chloro-thiophen-2-ylmethyl)-amino]-phenyl}-3,3-dimethylbutyramide;
N-{2-Chloro-4-[(5-chloro-thiophen-2-ylmethyl)-amino]-phenyl}-butyramide;
Pentanoic acid {2-chloro-4-[(5-chloro-thiophen-2-ylmethyl)-amino]-phenyl}-amide;
Cyclopropanecarboxylic acid {2-chloro-4-[(5-chloro-thiophen-2-ylmethyl)-amino]-phenyl}-amide;
Cyclobutanecarboxylic acid {2-chloro-4-[(5-chloro-thiophen-2-ylmethyl)-amino]-phenyl}-amide;
Cyclopentanecarboxylic acid {2-chloro-4-[(5-chloro-thiophen-2-ylmethyl)-amino]-phenyl}-amide;
Cyclohexanecarboxylic acid {2-chloro-4-[(5-chloro-thiophen-2-ylmethyl)-amino]-phenyl}-amide;
N-{2-Chloro-4-[(5-chloro-thiophen-2-ylmethyl)-amino]-phenyl}-2-thiophen-2-yl-acetamide;
N-{2-Chloro-4-[(5-chloro-thiophen-2-ylmethyl)-amino]-phenyl}-2-(3-methoxyphenyl)-acetamide;
N-{2-Chloro-4-[(5-chloro-thiophen-2-ylmethyl)-amino]-phenyl}-2-(4-chlorophenyl)-acetamide;
N-{2-Chloro-4-[(5-chloro-thiophen-2-ylmethyl)-amino]-phenyl}-2-(4-methoxyphenyl)-acetamide;
N-{2-Chloro-4-[(5-chloro-thiophen-2-ylmethyl)-amino]-phenyl}-2-(4-fluorophenyl)-acetamide;
2,3-Dihydro-benzo[1,4]dioxine-6-carboxylic acid {2-chloro-4-[(5-chloro-thiophen-2-ylmethyl)-amino]-phenyl}-amide;
2,3-Dihydro-benzofuran-5-carboxylic acid {2-chloro-4-[(5-chloro-thiophen-2-ylmethyl)-amino]-phenyl}-amide;
N-{2-Chloro-4-[(5-chloro-thiophen-2-ylmethyl)-amino]-phenyl}-3-cyclohexylpropionamide;
N-{4-[(5-Chloro-thiophen-2-ylmethyl)-(methyl)amino]-2-methyl-phenyl}-2,2-dimethylpropionamide;
N-{4-[(5-Chloro-thiophen-2-ylmethyl)-(methyl)amino]-2-methyl-phenyl}-2-phenylacetamide;
N-{4-[(5-Chloro-thiophen-2-ylmethyl)-(methyl)amino]-2-methyl-phenyl}-3,3-dimethylbutyramide;
N-{4-[(5-Chloro-thiophen-2-ylmethyl)-(methyl)amino]-2-methyl-phenyl}-3-phenylpropionamide;
N-{4-[(5-Chloro-thiophen-2-ylmethyl)-(methyl)amino]-2-methyl-phenyl}-butyramide;
2,2,2-Trichloro-N-{4-[(5-chloro-thiophen-2-ylmethyl)-(methyl)amino]-2-methyl-phenyl}-acetamide;
Cyclopropanecarboxylic acid {4-[(5-chloro-thiophen-2-ylmethyl)-(methyl)amino]-2-methyl-phenyl}-amide;
Cyclobutanecarboxylic acid {4-[(5-chloro-thiophen-2-ylmethyl)-(methyl)amino]-2-methylphenyl}-amide;
Cyclopentanecarboxylic acid {4-[(5-chloro-thiophen-2-ylmethyl)-(methyl)amino]-2-methylphenyl}-amide;
Cyclohexanecarboxylic acid {4-[(5-chloro-thiophen-2-ylmethyl)-(methyl)amino]-2-methylphenyl}-amide;
N-{4-[(5-Chloro-thiophen-2-ylmethyl)-(methyl)amino]-2-methylphenyl}-2-thiophen-2-yl-acetamide;
N-{4-[(5-Chloro-thiophen-2-ylmethyl)-(methyl)amino]-2-methylphenyl}-2-(3-methoxyphenyl)-acetamide;
N-{4-[(5-Chloro-thiophen-2-ylmethyl)-(methyl)amino]-2-methylphenyl}-malonamic acid methyl ester;
2-(4-Chlorophenyl)-N-{4-[(5-chloro-thiophen-2-ylmethyl)-(methyl)amino]-2-methylphenyl}-acetamide;
N-{4-[(5-Chloro-thiophen-2-ylmethyl)-(methyl)amino]-2-methylphenyl}-2-(4-methoxyphenyl)-acetamide;
N-{4-[(5-Chloro-thiophen-2-ylmethyl)-(methyl)amino]-2-methylphenyl}-2-(4-fluorophenyl)-acetamide;
N-{4-[(5-Chloro-thiophen-2-ylmethyl)-(methyl)amino]-2-methylphenyl}-3-cyclohexylpropionamide;
{2-Chloro-4-[(5-chloro-thiophen-2-ylmethyl)-(methyl)amino]-phenyl}-carbamic acid phenyl ester;
{2-Chloro-4-[(5-chloro-thiophen-2-ylmethyl)-(methyl)amino]-phenyl}-carbamic acid benzyl ester;
{2-Chloro-4-[(5-chloro-thiophen-2-ylmethyl)-(methyl)amino]-phenyl}-carbamic acid isobutyl ester;
{2-Chloro-4-[(5-chloro-thiophen-2-ylmethyl)-(methyl)amino]-phenyl}-carbamic acid butyl ester;
{2-Chloro-4-[(5-chloro-thiophen-2-ylmethyl)-(methyl)amino]-phenyl}-carbamic acid hexyl ester;
{2-Chloro-4-[(5-chloro-thiophen-2-ylmethyl)-(methyl)amino]-phenyl}-carbamic acid
{2-Chloro-4-[(5-chloro-thiophen-2-ylmethyl)-(methyl)amino]-phenyl}-carbamic acid 4-nitrobenzyl ester;
{2-Chloro-4-[(5-chloro-thiophen-2-ylmethyl)-(methyl)amino]-phenyl}-carbamic acid but-3-enyl ester;
{2-Chloro-4-[(5-chloro-thiophen-2-ylmethyl)-(methyl)amino]-phenyl}-carbamic acid but-2-ynyl ester;
{2-Chloro-4-[(5-chloro-thiophen-2-ylmethyl)-(methyl)amino]-phenyl}-carbamic acid 2,2-dimethylpropyl ester;
{2-Chloro-4-[(5-chloro-thiophen-2-ylmethyl)-(methyl)amino]-phenyl}-carbamic acid 2-chlorobenzyl ester;
{2-Chloro-4-[(5-chloro-thiophen-2-ylmethyl)-(methyl)amino]-phenyl}-carbamic acid 3-chloropropyl ester;
{2-Chloro-4-[(5-chloro-thiophen-2-ylmethyl)-(methyl)amino]-phenyl}-carbamic acid 2-benzyloxyethyl ester;
3-{2-Chloro-4-[(5-chloro-thiophen-2-ylmethyl)-(methyl)amino]-phenyl}-1-methyl-1-propyl-urea;
1-{2-Chloro-4-[(5-chloro-thiophen-2-ylmethyl)-(methyl)amino]-phenyl}-3-(2-fluorophenyl)-urea;
N-{2-Chloro-4-[(5-chloro-thiophen-2-ylmethyl)-(methyl)amino]-phenyl}-2,2,2-trifluoroacetamide;
N-{2-Chloro-4-[(5-chloro-thiophen-2-ylmethyl)-amino]-phenyl}-2,2,2-trifluoroacetamide;
N-{5-[(5-Chloro-thiophen-2-ylmethyl)-amino]-4′-dimethylamino-biphenyl-2-yl}-2-(4-fluorophenyl)-acetamide;
N-{2-Chloro-4-[(5-chloro-thiophen-2-ylmethyl)-(methyl)amino]-phenyl}-2-(4-chlorophenyl)-acetamide;
[4-(3-Fluoro-4-trifluoromethyl-benzylamino)-2-methylphenyl]-carbamic acid ethyl ester;
2-(4-Fluorophenyl)-N-{2-methyl-4-[(6-p-tolyloxypyridin-3-ylmethyl)-amino]-phenyl}-acetamide;
N-[2-Methyl-4-(4-trifluoromethyl-benzylamino)-phenyl]-butyramide;
2-(4-Fluorophenyl)-N-{2-methyl-4-[(6-trifluoromethylpyridin-3-ylmethyl)-amino]-phenyl}-acetamide;
Pentanoic acid {4-[(5-chloro-thiophen-2-ylmethyl)-(methyl)amino]-2-methylphenyl}-amide;
3,3-Dimethyl-N-{2-methyl-4-[(6-p-tolyloxypyridin-3-ylmethyl)-amino]-phenyl}-butyramide;
[2-Methyl-4-(4-trifluoromethyl-benzylamino)-phenyl]-carbamic acid ethyl ester;
N-{2-Chloro-4-[(5-chloro-thiophen-2-ylmethyl)-(methyl)amino]-phenyl}-2-(4-chlorophenyl)-propionamide;
[4-(4-Chloro-benzylamino)-2-methylphenyl]-carbamic acid ethyl ester;
{4-[(6-Methoxy-benzo[b]thiophen-2-ylmethyl)-amino]-2-methylphenyl}-carbamic acid propyl ester;
{4-[(5-Chloro-thiophen-2-ylmethyl)-amino]-2-quinolin-3-yl-phenyl}-carbamic acid ethyl ester;
{4-[(5-Dimethylamino-3-methyl-benzo[b]thiophen-2-ylmethyl)-amino]-2-methylphenyl}-carb amic acid propyl ester;
3,3-Dimethyl-N-{2-methyl-4-[(6-trifluoromethylpyridin-3-ylmethyl)-amino]-phenyl}-butyramide;
N-(4-{[6-(4-Cyanophenoxy)-pyridin-3-ylmethyl]-amino}-2-methylphenyl)-2-(4-fluorophenyl)-acetamide;
{2-Benzyloxy-4-[(4-fluorobenzyl)-(methyl)amino]-phenyl}-thiocarbamic acid S-ethyl ester;
{2-Cyclopentyloxy-4-[(4-fluorobenzyl)-(methyl)amino]-phenyl}-thiocarbamic acid S-ethyl ester;
N-{4-[(6-Chloropyridin-3-ylmethyl)-amino]-2-methyl phenyl}-2-(4-fluorophenyl)-acetamide;
{4-[(7-Dimethylamino-benzo[b]thiophen-2-ylmethyl)-amino]-2-methylphenyl}-carbamic acid propyl ester;
1-{2-Cyclopentyl oxy-4-[(4-fluorobenzyl)-(methyl)amino]-phenyl}-3-ethyl-urea;
2-Amino-4-methyl-pentanoic acid [2-methyl-4-(4-trifluoromethyl-benzylamino)-phenyl]-amide;
{4-[(6-Methoxy-benzo[b]thiophen-2-ylmethyl)-amino]-2-methyl phenyl}-carbamic acid ethyl ester;
2-Amino-4-methyl-pentanoic acid [2-Methyl-4-(4-trifluoromethyl-benzylamino)-phenyl]-amide;
2-(4-Fluorophenyl)-N-{2-methyl-4-[(4-methyl-2-phenylpyrimidin-5-ylmethyl)-amino]-phenyl}-acetamide;
3,3-Dimethyl-N-{2-methyl-4-[(2-phenylpyrimidin-5-ylmethyl)-amino]-phenyl}-butyramide;
{4-[(5-Chloro-thiophen-2-ylmethyl)-amino]-2-pyridin-3-yl-phenyl}-carbamic acid ethyl ester;
1-Amino-cyclopropanecarboxylic acid [2-methyl-4-(4-trifluoromethyl-benzylamino)-phenyl]-amide;
{4-[(5-Chloro-thiophen-2-ylmethyl)-amino]-2-pyridin-4-yl-phenyl}-carbamic acid ethyl ester;
N-[2-Methyl-4-(4-trifluoromethyl-benzylamino)-phenyl]-2-piperidin-1-yl-acetamide;
N-(4-{[5-(4-Chlorophenoxy)-1,3-dimethyl-1H-pyrazol-4-ylmethyl]-amino}-2-methylphenyl)-2,2-dimethylpropionamide;
2,2-Dimethyl-N-{2-methyl-4-[(6-phenoxypyridin-3-ylmethyl)-amino]-phenyl}-propionamide;
N-[2-Methyl-4-(4-trifluoromethyl-benzylamino)-phenyl]-2-pyrrolidin-1-yl-acetamide;
[4-[(5-Chloro-thiophen-2-ylmethyl)-amino]-2-(6-methoxypyridin-3-yl)-phenyl]-carbamic acid ethyl ester;
4-[(3-Methyl-4-propoxycarbonylamino-phenylamino)-methyl]-benzoic acid methyl ester;
N-[2-Methyl-4-(4-trifluoromethyl-benzylamino)-phenyl]-2-morpholin-4-yl-acetamide;
2,2-Dimethyl-N-{2-methyl-4-[(3-methyl-5-phenylisoxazol-4-ylmethyl)-amino]-phenyl}-propionamide;
{4-[(5-Chloro-thiophen-2-ylmethyl)-amino]-2-iodophenyl}-carbamic acid ethyl ester;
N-{4-[(5-Chloro-thiophen-2-ylmethyl)-amino]-2-iodophenyl}-2-(4-fluorophenyl)-acetamide; and
{4-[(5-Chloro-thiophen-2-ylmethyl)-amino]-2-quinolin-5-yl-phenyl}-carbamic acid ethyl ester.
   or salts thereof.

Compounds according to formula 2 can be prepared as described in WO2004/082677.

An embodiment of the invention relates to a method wherein said compound is a compound according to formula 3:

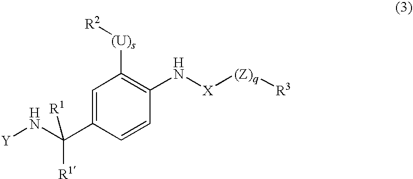

wherein:
U is O, S or NR²;
s is 0 or 1;
X is CO or SO₂;
Z is O, S or NR⁴, wherein R⁴ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl and hydroxy-C₃₋₈-cycloalk(en)yl;
q is 0 or 1;
R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl;
R² is selected from the group consisting of hydrogen, halogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl, halo-C₃₋₈-cycloalk(en)yl and cyano; provided that when R² is halogen or cyano, then s is 0;
when s is 1 and U is NR^{2′} then R^{2′} is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; or R² and R^{2′} together form a 5-8 membered saturated or unsaturated ring which optionally contains one further heteroatom;
R³ is selected from the group consisting of C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; and
Y represents a group of formulae VI, VII, VIII, IX or XXX:

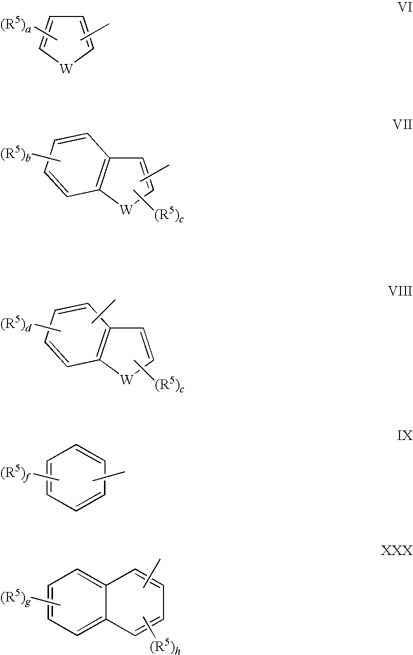

wherein
the line represents a bond attaching the group represented by Y to the nitrogen atom;
W is O or S;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4,
h is 0, 1, 2 or 3; and
each R⁵ is independently selected from the group consisting of a C₁₋₆-alk(en/yn)yl, C₃₋₈-cycloalk(en)yl, Ar, C₃₋₈-cycloalk(en)yl-C₁₋₆-alk(en/yn)yl, Ar—C₁₋₆-alk(en/yn)yl, acyl, C₁₋₆-alk(an/en/yn)yloxy, halogen, halo-C₁₋₆-alk(en/yn)yl, —CO—NR⁶R^{6′}, cyano, nitro, —NR⁷R^{7′}, —S—R⁸, —SO₂R⁸ and SO₂OR⁸, or two substituents together form a 5-8 membered saturated or unsaturated ring which optionally contains one or two heteroatoms;
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl; and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; with the provisos that when R⁵ is SO₂OR⁸ then R⁸ is not —NR⁹R^{9′} and when R⁵ is SO₂R⁸, then R⁸ is not a hydrogen atom;
   or salts thereof;

In an embodiment of formula 3:
R¹ and R^{1′} are independently selected from the group consisting of hydrogen and C₁₋₆-alk(en/yn)yl.
at least one of R¹ and R^{1′} is a hydrogen atom.
R² is selected from the group consisting of hydrogen, C₁₋₆-alk(en/yn)yl, Ar and halogen, provided that when R² is halogen, then s is 0.
X is CO.
Z is an oxygen atom.
R³ is C₁₋₆-alk(en/yn)yl.
Y represents a group of formulae IX or XXX.
each R⁵ is independently selected from the group consisting of a C₁₋₆-alk(en/yn)yl, C₃₋₈-cycloalk(en)yl, Ar, cyano, halogen, halo-C₁₋₆-alk(en/yn)yl and C₁₋₆-alk(an/en/yn)yloxy or two adjacent substituents together form a 5-8 membered saturated or unsaturated ring which optionally contains one or two heteroatoms.

In an embodiment of the present invention, the following definitions are applied for formula 3:
The term heteroatom refers to a nitrogen, oxygen or sulphur atom.
Halogen means fluoro, chloro, bromo or iodo.
The expressions C₁₋₆-alk(en/yn)yl and C₁₋₆-alk(an/en/yn)yl mean a C₁₋₆-alkyl,
C₂₋₆-alkenyl or a C₂₋₆-alkynyl group.
The term C₁₋₆-alkyl refers to a branched or unbranched alkyl group having from one to six carbon atoms inclusive, including but not limited to methyl, ethyl, 1-propyl, 2-propyl, 1-butyl, 2-butyl, 2-Methyl-2-propyl, 2-2-dimethyl-1-propyl and 2-methyl-1-propyl. Similarly, C₂₋₆-alkenyl and C₂₋₆-alkynyl, respectively, designate such groups having from two to six carbon atoms, including one double bond and one triple bond respectively, including but not limited to ethenyl, propenyl, butenyl, ethynyl, propynyl and butynyl.
The expressions C₁₋₄-alkyl and C₁₋₄-alkanyl refer to a branched or unbranched alkyl group having from one to four carbon atoms inclusive, including but not limited to methyl, ethyl, 1-propyl, 2-propyl, 1-butyl, 2-butyl, 2-methyl-2-propyl and 2-methyl-1-propyl.
The expression C₁₋₃-alk(en/yn)yl means a C₁₋₃-alkyl, C₂₋₃-alkenyl or a C₂₋₃-alkynyl group.
The term C₁₋₃-alkyl refers to a branched or unbranched alkyl group having from one to three carbon atoms inclusive, including but not limited to methyl, ethyl, 1-propyl and 2-propyl.
Similarly, C₂₋₃-alkenyl and C₂₋₃-alkynyl, respectively, designate such groups having from two to three carbon atoms, including one double bond and one triple bond respectively, including but not limited to ethenyl, propenyl, ethynyl and propynyl.
The expressions C₃₋₈-cycloalk(en)yl and C₃₋₈-cycloalk(an/en)yl mean a C₃₋₈-cycloalkyl- or cycloalkenyl group.
The term C₃₋₈-cycloalkyl designates a monocyclic or bicyclic carbocycle having three to eight C-atoms, including but not limited to cyclopropyl, cyclopentyl, cyclohexyl, etc.
The expressions C₃₋₆-cycloalk(en)yl and C₃₋₆-cycloalk(an/en)yl mean a C₃₋₆-cycloalkyl- or cycloalkenyl group.
The term C₃₋₆-cycloalkyl designates a monocyclic or bicyclic carbocycle having three to six C-atoms, including but not limited to cyclopropyl, cyclopentyl, cyclohexyl, etc.
The term C₃₋₈-cycloalkenyl designates a monocyclic or bicyclic carbocycle having three to eight C-atoms and including one double bond.
The expression C₅₋₈-cycloalk(en)yl means a C₅₋₈-cycloalkyl- or cycloalkenyl group.
The term C₅₋₈-cycloalkyl designates a monocyclic or bicyclic carbocycle having five to eight C-atoms, including but not limited to cyclopentyl, cyclohexyl, etc.
The term C₅₋₈-cycloalkenyl designates a monocyclic or bicyclic carbocycle having five to eight C-atoms and including one or two double bonds.
In the term C₃₋₈-cycloalk(en)yl-C₁₋₆-alk(en/yn)yl, C₃₋₈-cycloalk(en)yl and
C₁₋₆-alk(en/yn)yl are as defined above.
The term Ar refers to optionally substituted aromatic systems of 5-10 carbon atoms, wherein 0, 1, 2, 3 or 4 carbon atoms may be replaced with independently selected heteroatoms. Examples of such Ar groups are optionally substituted phenyl, optionally substituted naphtyl, optionally substituted thiophene, optionally substituted furan, optionally substituted thiazole, optionally substituted pyridine, optionally substituted pyrimidine, optionally substituted pyrrole and optionally substituted oxazole. Ar may be substituted with one or more substituents independently being hydroxy, halogen, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, C₁₋₆-alk(an/en/yn)yloxy, C₃₋₈-alk(an/en/yn)yloxy, acyl, cyano, —CO—NH—C₁₋₆-alk(en/yn)yl, —CO—N(C₁₋₆-alk(en/yn)yl)₂, —NH—C₁₋₆-alk(en/yn)yl, —N(C₁₋₆-alk(en/yn)yl)₂, —NH₂, —S—C₁₋₆-alk(en/yn)yl, —SO₂—C₁₋₆-alk(en/yn)yl, —SO₂N(C₁₋₆-alk(en/yn)yl)₂, —SO₂NH—C₁₋₆-alk(en/yn)yl and —SO₂O—C₁₋₆-alk(en/yn)yl; or two substituents may together form a 5-8 membered saturated or unsaturated ring which optionally contains one or two heteroatoms. Such two ring forming substituents may be adjacent and may together form: —(CH₂)ₙ**—CH₂—, —CH═CH—(CH₂)ₘ**—, —CH₂—CH═CH—(CH₂)ₚ**, —(CH₂)ₙ**—O—, —O—(CH₂)ₘ**—O—, —CH₂—O—(CH₂))ₚ**—O—, —CH₂—O—CH₂—O—CH₂—, —(CH₂)ₙ**—S—, —S—(CH₂)ₘ**—S—, —CH₂—S—(CH₂)ₚ**—S—, —CH₂—S—CH₂—S—CH₂, —(CH₂)ₙ**—NH—, —NH—(CH₂)ₘ**—NH—, —CH₂NH—(CH₂)ₚ**—NH—, —CH═CH—NH—, —O—(CH₂)ₘ**—NH—, —CH₂—O—(CH₂)ₚ**—NH— or —O—(CH₂)ₚ**—NH—CH₂—, —S—(CH₂)ₘ**—NH, —N═CH—NH—, —N═CH—O— or —N═CH—S—, wherein m** is 1, 2 or 3, n** is 2, 3 or 4 and p** is 1 or 2.
As used herein, the term acyl refers to formyl, C₁₋₆-alk(en/yn)ylcarbonyl, C₃₋₈-cycloalk(en)ylcarbonyl, Ar-carbonyl, Ar—C₁₋₆-alk(en/yn)ylcarbonyl or a C₃₋₈-cycloalk(en)yl-C₁₋₆-alk(en/yn)yl-carbonyl group, wherein C₁₋₆-alk(en/yn)yl, C₃₋₈-cycloalk(en)yl and Ar are as defined above.
The term halo-C₁₋₆-alk(en/yn)yl designates C₁₋₆-alk(en/yn)yl being substituted with one or more halogen atoms, including but not limited to trifluoromethyl. Similarly, halo-C₃₋₈-cycloalk(en)yl designates C₃₋₈-cycloalk(en)yl being substituted with one or more halogen atoms and halo-C₃₋₈-cycloalk(en)yl-C₁₋₆-alk(en/yn)yl, designates C₃₋₈-cycloalk(en)yl-C₁₋₆-alk(en/yn)yl being substituted with one or more halogen atoms.
The terms hydroxy-C₁₋₆-alk(en/yn)yl, hydroxy-C₃₋₈-alk(en/yn)yl, hydroxy-C₃₋₈-cycloalk(en)yl, Ar—C₁₋₆-alk(en/yn)yl, Ar—C₃₋₈-cycloalk(en)yl, C₁₋₆-alk(an/en/yn)yloxy, C₁₋₄-alkanyloxy, C₂₋₆-alkenyloxy, C₂₋₆-alkynyloxy, C₃₋₈-alk(an/en/yn)yloxy, C₁₋₆-alk(en/yn)ylcarbonyl, C₃₋₈-alk(en/yn)ylcarbonyl, Ar-carbonyl, Ar—C₁₋₆-alk(en/yn)ylcarbonyl, C₃₋₈-cycloalk(en)yl-C₁₋₆-alk(en/yn)ylcarbonyl etc. designate such groups in which the C₁₋₆-alk(en/yn)yl, C₂₋₆-alkenyl, C₂₋₆-alkynyl, C₃₋₈-cycloalk(en)yl and Ar are as defined above.
The term “two substituents together form a 5-8 membered saturated or unsaturated ring, which optionally contains one or two heteroatoms,” refers to aliphatic or aromatic carbocyclic or heterocyclic systems wherein the ring is formed by 5 to 8 atoms which may be substituted by one or more substituents independently being C₁₋₆-alk(en/yn)yl, C₃₋₈-alk(en/yn)yl, C₃₋₈-cycloalk(en)yl-C₁₋₆-alk(en/yn)yl, halogen, halo-C₁₋₆-alk(en/yn)yl, halo-C₃₋₈-alk(en/yn)yl or C₃₋₈-cycloalk(en)yl-C₁₋₆-alk(en/yn)yl. The ring forming atoms are selected from 3-8 carbon atoms and 0-2 heteroatoms selected from N, S, or O. When the two ring forming substituents are attached to the same nitrogen atom, then said nitrogen atom becomes one of the atoms forming the ring. When two ring forming substituents are attached to an aliphatic or aromatic carbocyclic or heterocyclic group, then the two ring forming substituents are conveniently attached adjacent to each other and the ring formed by the two substituents is fused to the aliphatic or aromatic carbocyclic or heterocyclic group. Two ring forming substituents may together be represented by: —(CH₂)_{n″}—CH₂—, —CH═CH—(CH₂)_{m″}—, —CH₂—CH═CH—(CH₂)_{p″}, —CH═CH—CH═CH—, —(CH₂)_{n″}—O—, —O—(CH₂)_{m″}—O—, —CH₂—O—(CH₂)_{p″}—O—, —CH₂—O—CH₂—O—CH₂—, —(CH₂)_{n″}—S—, —S—(CH₂)_{m″}—S—, —CH₂—S—(CH₂)_{p″}—S—, —CH₂—S—CH₂—S—CH₂—, —(CH₂)_{n″}—NH—, —NH—(CH₂)_{m″}—NH—, —CH₂—NH—(CH₂)_{p″}—NH—, —CH═CH—NH—, —O—(CH₂)_{m″}—NH—, —CH₂—O—(CH₂)_{p″}—NH— or —O—(CH₂)_{p″}—NH—CH₂—, —S—(CH₂)_{m″}—NH—, —N═CH—NH—, —N═CH—O— or —N═CH—S—, wherein m″ is 1, 2 or 3, n″ is 2, 3 or 4 and p″ is 1 or 2.

In an embodiment of formula 3 said compound is selected from the group consisting of:
{2-Amino-4-[(4-tert-butylphenylamino)-methyl]-phenyl}-carbamic acid ethyl ester;
(2-Amino-4-phenylaminomethyl-phenyl)-carbamic acid ethyl ester;
[2-Amino-4-(naphthalen-2-ylaminomethyl)-phenyl]-carbamic acid ethyl ester;
[2-Amino-4-(p-tolylamino-methyl)-phenyl]-carbamic acid ethyl ester;
{2-Amino-4-[(4-trifluoromethylphenylamino)-methyl]-phenyl}-carbamic acid ethyl ester;
{2-Amino-4-[(4-chlorophenylamino)-methyl]-phenyl}-carbamic acid ethyl ester;
{2-Amino-4-[(3-fluorophenylamino)-methyl]-phenyl}-carbamic acid ethyl ester;
{2-Amino-4-[(4-fluorophenylamino)-methyl]-phenyl}-carbamic acid ethyl ester;
{2-Amino-4-[(2-fluorophenylamino)-methyl]-phenyl}-carbamic acid ethyl ester;
[2-Amino-4-(biphenyl-4-ylaminomethyl)-phenyl]-carbamic acid ethyl ester;
{2-Amino-4-[(2,4-difluorophenylamino)-methyl]-phenyl}-carbamic acid ethyl ester;
{2-Amino-4-[(4-methoxyphenylamino)-methyl]-phenyl}-carbamic acid ethyl ester;
{2-Amino-4-[(4-cyclohexylphenylamino)-methyl]-phenyl}-carbamic acid ethyl ester;
[2-Amino-4-(indan-5-ylaminomethyl)-phenyl]-carbamic acid ethyl ester;
{2-Amino-4-[(4-isopropylphenylamino)-methyl]-phenyl}-carbamic acid ethyl ester;
{2-Amino-4-[(4-butylphenylamino)-methyl]-phenyl}-carbamic acid ethyl ester;
{2-Amino-4-[(4-chloro-3-fluorophenylamino)methyl]phenyl}carbamic acid ethyl ester;
{2-Amino-4-[(2,4-dichlorophenylamino)methyl]phenyl}carbamic acid ethyl ester;
{2-Amino-4-[(2,3-dichlorophenylamino)methyl]phenyl}carbamic acid ethyl ester;
{2-Amino-4-[(3,5-dichlorophenylamino)methyl]phenyl}carbamic acid ethyl ester;
{2-Amino-4-[(3,4-dichlorophenylamino)methyl]phenyl}carbamic acid ethyl ester;
{2-Amino-4-[(3-trifluoromethylphenylamino)methyl]phenyl}carbamic acid ethyl ester;
{2-Amino-4-[(3-fluoro-4-trifluoromethylphenylamino)methyl]phenyl}carbamic acid ethyl ester;
{2-Amino-4-[(3,4-difluorophenylamino)methyl]phenyl}carbamic acid ethyl ester;
{2-Amino-4-[(4-cyanophenylamino)methyl]phenyl}carbamic acid ethyl ester;
{2-Amino-4-[(4-fluoro-3-trifluoromethylphenylamino)methyl]phenyl}carbamic acid ethyl ester;
{2-Amino-4-[(3-chloro-4-methylphenylamino)methyl]phenyl}carbamic acid ethyl ester;
{12-Amino-4-[(3-chlorophenylamino)methyl]phenyl}carbamic acid ethyl ester;
[2-Amino-4-(m-tolylaminomethyl)phenyl]carbamic acid ethyl ester;
{2-Amino-4-[1-(4-chlorophenylamino)ethyl]phenyl}carbamic acid ethyl ester;
{2-Amino-4-[1-(4-trifluoromethylphenylamino)ethyl}phenyl]carbamic acid ethyl ester;
N-{2-Amino-4-[(3-fluorophenylamino)methyl]phenyl}-2,2-dimethylpropionamide;
{4-[(4-Chlorophenylamino)methyl]phenyl}carbamic acid ethyl ester;
{4-[(4-Trifluoromethylphenylamino)methyl]phenyl}carbamic acid ethyl ester;
{4-[1-(4-Chlorophenylamino)ethyl]phenyl}carbamic acid ethyl ester;
{4-[(4-Fluorophenylamino)methyl]-2-methylphenyl}carbamic acid ethyl ester;
{4-[(4-Chlorophenylamino)methyl]-2-methylphenyl}carbamic acid ethyl ester;
{2-Methyl-4-[(4-trifluoromethylphenylamino)methyl]phenyl}carbamic acid ethyl ester;
{4-[(3,4-Difluorophenylamino)methyl]-2-methylphenyl}carbamic acid ethyl ester;
{4-[(3-Fluorophenylamino)methyl]-2-methylphenyl}carbamic acid ethyl ester;
{2-Chloro-4-[(4-chlorophenylamino)methyl]phenyl}carbamic acid ethyl ester;
{2-Chloro-4-[(4-trifluoromethyl-phenylamino)-methyl]-phenyl}-carbamic acid ethyl ester;
{2-Chloro-4-[(4-fluorophenylamino)methyl]phenyl}carbamic acid ethyl ester;
{2-Chloro-4-[(3-fluorophenylamino)methyl]phenyl}carbamic acid ethyl ester;
{2-Chloro-4-[(3,4-dichlorophenylamino)methyl]phenyl}carbamic acid ethyl ester;
{2-Chloro-4-[(4-chloro-3-fluorophenylamino)methyl]phenyl}carbamic acid ethyl ester;
{4-[(4-Chlorophenylamino)methyl]-2-fluorophenyl}carbamic acid ethyl ester;
{4-[(4-Chloro-3-fluorophenylamino)methyl]-2-fluorophenyl}carbamic acid ethyl ester;
{2-Fluoro-4-[(4-trifluoromethylphenylamino)methyl]phenyl}carbamic acid ethyl ester;
{4′-Dimethylamino-5-[(3-fluorophenylamino)methyl]biphenyl-2-yl}carbamic acid ethyl ester;
{4′-Dimethylamino-5-[(4-trifluoromethylphenylamino)methyl]biphenyl-2-yl}carbamic acid ethyl ester;
{4′-Chloro-5-[(3-fluorophenylamino)methyl]biphenyl-2-yl}carbamic acid ethyl ester;
{4′-Chloro-5-[(4-trifluoromethyl phenylamino)methyl]biphenyl-2-yl}carbamic acid ethyl ester;
N-{4-[(4-chlorophenylamino)methyl]phenyl}butyramide;
N-{4-[(3,4-dichlorophenylamino)methyl]phenyl}butyramide;
N-{4-[(4-chloro-3-fluorophenylamino)methyl]phenyl}butyramide;
N-{4-[(4-fluoro-phenylamino)methyl]-2-methylphenyl}butyramide;
N-{4-[(3-fluorophenylamino)methyl]-2-methyl phenyl}butyramide;
N-{4-[(4-chlorophenylamino)methyl]-2-methyl phenyl}butyramide;
N-{4-[(3,4-dichlorophenylamino)methyl]-2-methyl phenyl}butyramide;
N-{4-[(4-chloro-3-fluorophenylamino)methyl]-2-methylphenyl}butyramide,
N-{2-chloro-4-[(4-trifluoromethylphenyl amino)methyl]phenyl}butyramide;
N-{2-chloro-4-[(4-fluorophenylamino)methyl]phenyl}butyramide;
N-{2-chloro-4-[(3-fluorophenylamino)methyl]phenyl}butyramide;
N-{2-chloro-4-[(4-chlorophenylamino)methyl]phenyl}butyramide;
N-{2-chloro-4-[(3,4-dichlorophenylamino)methyl]phenyl}butyramide;
N-{2-chloro-4-[(4-chloro-3-fluorophenylamino)methyl]phenyl}butyramide;
N-{2-fluoro-4-[(3-fluorophenyl amino)methyl]phenyl}butyramide;
N-{4-[(4-chlorophenylamino)methyl]-2-fluorophenyl}butyramide;
N-{2-fluoro-4-[(4-trifluoromethyl-phenylamino)methyl]phenyl}butyramide;
N-{4-[(3,4-di chlorophenylamino)methyl]-2-fluorophenyl}butyramide; and
N-{4-[(4-chloro-3-fluorophenylamino)methyl]-2-fluorophenyl}butyramide.
   or a salt thereof.

Compounds according to formula 3 can be prepared as described in WO2004/080950.

An embodiment of the invention relates to a method wherein said compound is a compound according to formula 4:

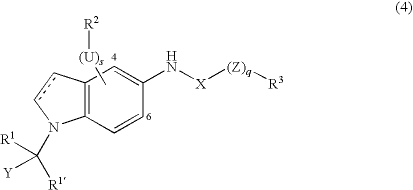

wherein

the dotted line represents an optional bond;
R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or R¹ and R^{1′} together with the carbon atom to which they are attached form a 3-8 membered saturated or unsaturated ring which optionally contains 1 or 2 heteroatoms;
s is 0 or 1;
U is O, NR¹¹, S, SO₂, SO₂NR¹¹, CO—O or CO—NR¹¹; wherein R¹¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl; or R² and R¹¹ together with the nitrogen atom to which they are attached form a 4-8 membered saturated or unsaturated ring which optionally contains 1, 2 or 3 further heteroatoms;
R² is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NO₂, NR¹⁰R^{10′}—C₁₋₆-alk(en/yn)yl, NR¹⁰R^{10′}—C₃₋₈-cycloalk(en)yl and NR¹⁰R^{10′}—C₃₋₈-cycloalk(en)yl-C₁₋₆-alk(en/yn)yl; wherein
R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or R¹⁰ and R^{10′} together with the nitrogen atom to which they are attached form a 4-8 membered saturated or unsaturated ring which optionally contains 1, 2 or 3 further heteroatoms;
with the proviso that when R² is NO₂, halogen or cyano then s is 0; and with the proviso that when R² is a hydrogen atom or acyl and s is 1 then U is NR¹¹, O or S;
wherein the group —(U)ₛ—R² is linked to position 4 or 6 of the indole or indoline;
q is 0 or 1;
Z is O or S;
X is CO or SO₂; with the proviso that q is 0 when X is SO₂;
R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, Ar, Ar—C₁₋₆-alk(en/yn)yl, Ar—C₃₋₈-cycloalk(en)yl, Ar-heterocycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar—C₁₋₆-alk(en/yn)yl-heterocycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar—C₁₋₆-alk(en/yn)yloxy-C₁₋₆-alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl and —NR¹²R^{12′}, optionally substituted NR¹²R^{12′}—C₁₋₆-alk(en/yn)yl, optionally substituted NR¹²R^{12′}—C₃₋₈-cycloalk(en)yl, optionally substituted NR¹²R^{12′}—C₃₋₈-cycloalk(en)yl-C₁₋₆-alk(en/yn)yl; wherein
R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or R¹²R^{12′} and R^{12′} together with the nitrogen atom to which they are attached form a 4-8 membered saturated or unsaturated ring which optionally contains 1, 2 or 3 further heteroatoms;
with the proviso that when R³ is NR¹²R^{12′} then q is 0; and
Y represents a group of formula II, III, IV, V, VI, XXX and XXXI:

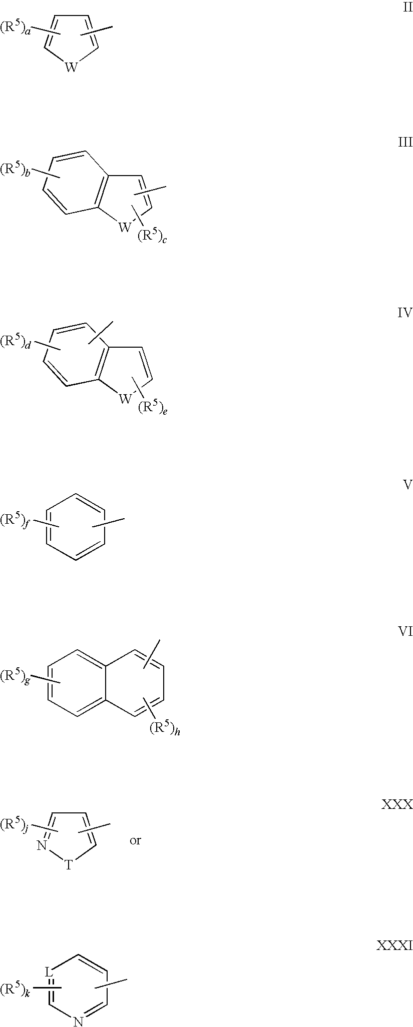

wherein
the line represents a bond attaching the group represented by Y to the carbon atom;
W is O or S;
T is N, NH or O;
L is N, C or CH;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3;
j is 0, 1, 2 or 3; with the proviso that when T is a nitrogen atom then j is 0, 1, 2 or 3; and when T is NH or an oxygen atom then j is 0, 1 or 2;
k is 0, 1, 2, 3 or 4, and
each R⁵ is independently selected from the group consisting of a C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar-thio, Ar-oxy, acyl, C₁₋₆-alk(en/yn)yloxy, C₃₋₈-cycloalk(en)yloxy, C₃₋₈-cycloalk(en)yl-C₁₋₆-alk(en/yn)yloxy, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —CO—NR⁶R^{6′}—, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NR⁷R^{7′}, —S—R⁸ and —SO₂R⁸, or two adjacent R⁵ together with the aromatic group to which they are attached form a 4-8 membered ring which optionally contains one or two heteroatoms;
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl; and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; provided that when R⁸ is —NR⁹R^{9′} then R⁵ is not —S—R⁸;
   or salts thereof.

In an embodiment of formula 4:
at least one of R¹ or R^{1′} is a hydrogen atom.
R² is a hydrogen atom, NO₂ or a halogen atom.
U is NR¹¹
R¹¹ is a hydrogen atom.
Z is an oxygen atom.

R³ is selected from the group consisting of C₁₋₆-alk(en/yn)yl, C₃₋₈-cycloalk(en)yl, Ar, Ar—C₁₋₆-alk(en/yn)yl, Ar-oxy-C₁₋₆-alk(en/yn)yl, Ar—C₁₋₆-alk(en/yn)yloxy-C₁₋₆-alk(en/yn)yl and —NR¹²R^{12′}, with the proviso that when R³ is NR¹²R^{12′} then q is 0.
R¹² and R^{12′} are independently selected from the group consisting of hydrogen, C₁₋₆-alk(en/yn)yl, Ar and Ar—C₁₋₆-alk(en/yn)yl, or R¹² and R^{12′} together with the nitrogen atom to which they are attached form a 4-8 membered saturated or unsaturated ring which optionally contains 1, 2 or 3 further heteroatoms.
Y is of formula II or III and W is a sulphur atom or Y is of formula XXX and T is a nitrogen atom or an oxygen atom or Y is of formula XXXI and L is C or CH
each R⁵ is independently selected from the group consisting of C₁₋₆-alk(en/yn)yl, Ar, Ar-thio, Ar-oxy, halogen and halo-C₁₋₆-alk(en/yn)yl or two adjacent R⁵ together with the aromatic group to which they are attached form a 4-8 membered ring which optionally contains one or two heteroatoms

In an embodiment of the present invention, the following definitions are applied for formula 4:
The term heteroatom refers to a nitrogen, oxygen or sulphur atom.
Halogen means fluoro, chloro, bromo or iodo.
The expressions C₁₋₆-alk(en/yn)yl and C₁₋₆-alk(an/en/yn)yl mean a C₁₋₆-alkyl, C₂₋₆-alkenyl or a C₂₋₆-alkynyl group. The term C₁₋₆-alkyl refers to a branched or un-branched alkyl group having from one to six carbon atoms inclusive, including but not limited to methyl, ethyl, 1-propyl, 2-propyl, 1-butyl, 2-butyl, 2-methyl-2-propyl and 2-methyl-1-propyl. Similarly, C₂₋₆-alkenyl and C₂₋₆-alkynyl, respectively, designate such groups having from two to six carbon atoms, including one double bond and one triple bond respectively, including but not limited to ethenyl, propenyl, butenyl, ethynyl, propynyl and butynyl.
The expression C₁₋₃-alk(en/yn)yl means a C₁₋₃-alkyl, C₂₋₃-alkenyl or a C₂₋₃-alkynyl group. The term C₁₋₃-alkyl refers to a branched or un-branched alkyl group having from one to three carbon atoms inclusive, including but not limited to methyl, ethyl, 1-propyl and 2-propyl. Similarly, C₂₋₃-alkenyl and C₂₋₃-alkynyl, respectively, designate such groups having from two to three carbon atoms, including one double bond and one triple bond respectively, including but not limited to ethenyl, 1-propenyl, 2-propenyl, 3-propenyl, ethynyl, 1-propynyl and 3-propynyl.
   The expressions C₃₋₈-cycloalk(en)yl and C₃₋₈-cycloalk(an/en)yl mean a C₃₋₈-cycloalkyl- or cycloalkenyl group. The term C₃₋₈-cycloalkyl designates a monocyclic or bicyclic carbocycle having three to eight C-atoms, including but not limited to cyclopropyl, cyclobutyl, cyclopentyl, cyclohexyl, etc. The term C₃₋₈-cycloalkenyl designates a monocyclic or bicyclic carbocycle having three to eight C-atoms and including one double bond.
   The expressions C₃₋₆-cycloalk(en)yl and C₃₋₆-cycloalk(an/en)yl mean a C₃₋₆-cycloalkyl- or cycloalkenyl group. The term C₃₋₆-cycloalkyl designates a monocyclic or bicyclic carbocycle having three to six C-atoms, including but not limited to cyclopropyl, cyclobutyl, cyclopentyl, cyclohexyl, etc.
   The term heterocycloalk(en)yl designates a monocyclic or bicyclic ring system wherein the ring is formed by 4 to 8 atoms selected from 2-7 carbonatoms and 1 or 2 heteroatoms selected from N, S, or O.
   When two substituents together with a carbon atom to which they are attached form a 3-8 membered saturated or unsaturated ring which optionally contains 1 or 2 heteroatoms, then a monocyclic ring system is formed by 3 to 8 atoms selected from 1-8 carbonatoms and 0-2 heteroatoms selected from N, S, or O. Examples of such ring systems are cyclopropyl, cyclobutyl, cyclopentyl, cyclohexyl.
   The term halo-C₁₋₆-alk(en/yn)yl designates C₁₋₆-alk(en/yn)yl being substituted with one or more halogen atoms, including but not limited to trifluoromethyl. Similarly, halo-C₃₋₈-cycloalk(en)yl designates C₃₋₈-cycloalk(en)yl being substituted with one or more halogen atoms and halo-heterocycloalk(en)yl designates heterocycloalk(en)yl being substituted with one or more halogen atoms.
The term NR¹²R^{12′}—C₁₋₆-alk(en/yn)yl designates C₁₋₆-alk(en/yn)yl being substituted with NR¹²R^{12′}. The term NR¹²R^{12′}—C₃₋₈-cycloalk(en)yl designates C₃₋₈-cycloalk(en)yl being substituted with NR¹²R^{12′}. The term NR¹²R^{12′}—C₃₋₈-cycloalk(en)yl-C₁₋₆-alk(en/yn)yl designates C₃₋₈-cycloalk(en)yl-C₁₋₆-alk(en/yn)yl being substituted with NR¹²R^{12′}. When any of NR¹²R^{12′}—C₁₋₆-alk(en/yn)yl, NR¹²R^{12′}—C₃₋₈-cycloalk(en)yl and NR¹²R^{12′}—C₃₋₈-cycloalk(en)yl-C₁₋₆-alk(en/yn)yl is optionally substituted, then any of C₁₋₆-alk(en/yn)yl, C₃₋₈-cycloalk(en)yl, C₃₋₈-cycloalk(en)yl-C₁₋₆-alk(en/yn)yl is optionally substituted with one or more substituents independently being C₁₋₆-alk(en/yn)yl, C₃₋₈-cycloalk(en)yl, C₃₋₈-cycloalk(en)yl-C₁₋₆-alk(en/yn)yl or Ar.
As used herein, the term acyl refers to formyl, C₁₋₆-alk(en/yn)ylcarbonyl, C₃₋₈-cycloalk(en)ylcarbonyl, Ar-carbonyl, Ar—C₁₋₆-alk(en/yn)ylcarbonyl or a C₃₋₈-cycloalk(en)yl-C₁₋₆-alk(en/yn)yl-carbonyl group, wherein C₁₋₆-alk(en/yn)yl, C₃₋₈-cycloalk(en)yl and Ar are as defined above.
When two substituents together with a nitrogen atom to which they are attached form a 4-8 membered saturated or unsaturated ring which optionally contains 1, 2 or 3 further heteroatoms, then a monocyclic ring system is formed by 4 to 8 atoms selected from the nitrogen atom, 1-7 carbonatoms and 0-3 further heteroatoms selected from N, S, or O. Examples of such ring systems are azetidine, beta-lactame, pyrrolidine, piperidine, piperazine, morpholine, pyrrole, oxazolidine, thiazolidine, imidazolidine, azetidine, beta-lactame, tetrazole and pyrazole.
When two adjacent substituents together with the aromatic group to which they are attached form a 4-8 membered ring, which optionally contains one or two heteroatoms, then a ring system is formed by 4-8 atoms selected from 3-8 carbonatoms and 0-2 heteroatoms selected from N, S, or O, Such two adjacent substituents may together form: —(CH₂)_{n″}—CH₂—, —CH═CH—(CH₂)_{m″}—, —CH₂—CH═CH—(CH₂)_{p″}—, —CH═CH—CH═CH—, —(CH₂)_{n″}—O—, —O—(CH₂)_{m″}—O—, —CH₂—O—(CH₂)_{p″}—O—, —CH₂—O—CH₂—O—(CH₂)_{n″}—S—, —S—(CH₂)_{m″}—S—, —CH₂—S—(CH₂)_{p″}—S—, —CH₂—S—CH₂—S—CH₂—, —(CH₂)_{n″}—NH—, —NH—(CH₂)_{m″}—NH—, —CH₂—NH—(CH₂)_{p″}—NH—, —CH═CH—NH—, —O—(CH₂)_{m″}—NH—, —CH₂—O—(CH₂)_{p″}—NH— or —O—(CH₂)_{p″}—NH—CH₂—, —S—(CH₂)_{m″}—NH—, —N═CH—NH—, —N═CH—O— or —N═CH—S—, wherein m″ is 1, 2 or 3, n″ is 2, 3 or 4 and p″ is 1 or 2.
The term Ar refers to optionally substituted aromatic systems of 5-10 carbon atoms, wherein 0, 1, 2, 3 or 4 carbon atoms may be replaced by heteroatoms independently selected from N, S, or O. Examples of such Ar groups are optionally substituted phenyl, optionally substituted naphtyl, optionally substituted pyridine, optionally substituted pyrrole, optionally substituted pyrimidine, optionally substituted quinoline, optionally substituted indole, optionally substituted thiophene, optionally substituted furan, optionally substituted thiazole and optionally substituted oxazole. Ar may be substituted with one or more substituents independently being hydroxy, halogen, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, C₁₋₆-alk(en/yn)yloxy, C₃₋₈-alk(en/yn)yloxy, acyl, nitro or cyano, —CO—NH—C₁₋₆-alk(en/yn)yl, —CO—N(C₁₋₆-alk(en/yn)yl)₂, —NH₂, —NH—C₁₋₆-alk(en/yn)yl, —N(C₁₋₆-alk(en/yn)yl)₂, —S—C₁₋₆-alk(en/yn)yl, —SO₂—C₁₋₆-alk(en/yn)yl, —SO₂N(C₁₋₆-alk(en/yn)yl)₂ and —SO₂NH—C₁₋₆-alk(en/yn)yl; or two adjacent substituents may together with the aromatic group to which they are attached form a 4-8 membered ring, which optionally contains one or two heteroatoms and which may be saturated or unsaturated.
The terms C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, Ar, Ar—C₁₋₆-alk(en/yn)yl, Ar—C₃₋₈-cycloalk(en)yl, Ar-heterocycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar—C₁₋₆-alk(en/yn)yl-heterocycloalk(en)yl, C₁₋₆-alk(en/yn)yloxy, C₂₋₆-alkenyloxy, C₂₋₆-alkynyloxy, C₃₋₈-cycloalk(en)yloxy, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar—C₁₋₆-alk(en/yn)yloxy-C₁₋₆-alk(en/yn)yl, C₁₋₆-alk(en/yn)ylcarbonyl, C₃₋₈-alk(en/yn)ylcarbonyl, Ar-carbonyl, Ar—C₁₋₆-alk(en/yn)ylcarbonyl, C₃₋₈-cycloalk(en)yl-C₁₋₆-alk(en/yn)ylcarbonyl, —CO—C₁₋₆-alk(en/yn)yl, —S—C₁₋₆-alk(en/yn)yl, —SO₂—C₁₋₆-alk(en/yn)yl and —SO₂O—C₁₋₆-alk(en/yn)yl, alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, acyl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, halo-heterocycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl etc. designate such groups in which the C₁₋₆-alk(en/yn)yl, C₂₋₆-alkenyl, C₂₋₆-alkynyl, C₃₋₈-cycloalk(en)yl, heterocycloalk(en)yl, Ar, cyano, halo-C₁₋₆-alk(en/yn)yl, halo-C₃₋₈-cycloalk(en)yl, halo-heterocycloalk(en)yl and acyl are as defined above.

In an embodiment of formula 4 said compound is selected from the group consisting of:
N-[4-Chloro-1-(4-trifluoromethylbenzyl)-2,3-dihydro-1H-indol-5-yl]-3,3-dimethylbutyramide;
N-[4-Chloro-1-(5-chlorothiophen-2-ylmethyl)-2,3-dihydro-1H-indol-5-yl]-3,3-dimethylbutyramide;
[1-(4-Fluorobenzyl)-2,3-dihydro-1H-indol-5-yl]-carbamic acid propyl ester;
N-[1-(4-Fluorobenzyl)-2,3-dihydro-1H-indol-5-yl]-C-phenyl-methanesulfonamide;
4-Fluoro-N-[1-(4-fluorobenzyl)-2,3-dihydro-1H-indol-5-yl]-benzamide;
N-[1-(4-Fluorobenzyl)-2,3-dihydro-1H-indol-5-yl]-3,3-dimethylbutyramide;
N-[1-(4-Fluorobenzyl)-2,3-dihydro-1H-indol-5-yl]-2-thiophen-2-ylacetamide;
N-[1-(4-Fluorobenzyl)-2,3-dihydro-M-indol-5-yl]-2-(4-fluorophenyl)-acetamide;
3-[1-(5-Chlorothiophen-2-ylmethyl)-2,3-dihydro-1H-indol-5-yl]-1,1-diisopropylurea;
Morpholine-4-carboxylic acid [1-(5-chlorothiophen-2-ylmethyl)-2,3-dihydro-1H-indol-5-yl]-amide;
Pyrrolidine-1-carboxylic acid [1-(5-chlorothiophen-2-ylmethyl)-2,3-dihydro-1H-indol-5-yl]-amide;
[1-(5-Chlorothiophen-2-ylmethyl)-2,3-dihydro-1H-indol-5-yl]carbamic acid 2-benzyloxyethyl ester;
3-[1-(5-Chlorothiophen-2-ylmethyl)-2,3-dihydro-1H-indol-5-yl]-1-methyl-1-propylurea;
[1-(5-Chlorothiophen-2-ylmethyl)-2,3-dihydro-1H-indol-5-yl]-carbamic acid tent-butyl ester;
N-[1-(5-Chlorothiophen-2-ylmethyl)-2,3-dihydro-1H-indol-5-yl]-C-phenyl-methanesulfonamide;
Butane-1-sulfonic acid [1-(5-chlorothiophen-2-ylmethyl)-2,3-dihydro-1H-indol-5-yl]amide;
N-[1-(5-Chlorothiophen-2-ylmethyl)-2,3-dihydro-1H-indol-5-yl]-4-fluorobenzamide;
N-[1-(5-Chlorothiophen-2-ylmethyl)-2,3-dihydro-1H-indol-5-yl]-2,2-dimethylpropionamide;
N-[1-(5-Chlorothiophen-2-ylmethyl)-2,3-dihydro-1H-indol-5-yl]-2-phenoxyacetamide;
N-[1-(5-Chlorothiophen-2-ylmethyl)-2,3-dihydro-1H-indol-5-yl]-3,3-dimethylbutyramide;
N-[1-(5-Chlorothiophen-2-ylmethyl)-2,3-dihydro-1H-indol-5-yl]-butyramide;
Cyclopentanecarboxylic acid [1-(5-chlorothiophen-2-ylmethyl)-2,3-dihydro-1H-indol-5-yl]-amide;
N-[1-(5-Chlorothiophen-2-ylmethyl)-2,3-dihydro-1H-indol-5-yl]-2-thiophen-2-ylacetamide;
N-[1-(5-Chlorothiophen-2-ylmethyl)-2,3-dihydro-1H-indol-5-yl]-isonicotinamide;
N-[1-(5-Chlorothiophen-2-ylmethyl)-2,3-dihydro-1H-indol-5-yl]-dimethylaminobenzamide;
N-[1-(5-Chlorothiophen-2-ylmethyl)-2,3-dihydro-1H-indol-5-yl]-2-(4-fluorophenyl)-acetamide;
N-[1-(5-Chlorothiophen-2-ylmethyl)-2,3H-dihydro-1H-indol-5-yl]-6-trifluoromethylnicotinamide;
1-tert-Butyl-3-[1-(5-chlorothiophen-2-ylmethyl)-2,3-dihydro-1H-indol-5-yl]-urea;
1-[1-(5-Chlorothiophen-2-ylmethyl)-2,3-dihydro-1H-indol-5-yl]-3-ethylurea;
1-Benzyl-3-[1-(5-chlorothiophen-2-ylmethyl)-2,3-dihydro-1H-indol-5-yl]-urea;
1-[1-(5-Chlorothiophen-2-ylmethyl)-2,3-dihydro-1H-indol-5-yl]-3-phenethylurea;
1-[1-(5-Chlorothiophen-2-ylmethyl)-2,3-dihydro-1H-indol-5-yl]-3-thiophen-2-ylurea;
1-[1-(5-Chlorothiophen-2-ylmethyl)-2,3-dihydro-1H-indol-5-yl]-3-thiophen-3-ylurea;
[1-(5-Chlorothiophen-2-ylmethyl)-2,3-dihydro-1H-indol-5-yl]-carbamic acid propyl ester;
2,2-Dimethyl-N-[6-nitro-1-(4-trifluoromethylbenzyl)-2,3-dihydro-1H-indol-5-yl]-propionamide;
N-[1-(5-Chlorothiophen-2-ylmethyl)-6-nitro-2,3-dihydro-1H-indol-5-yl]-2,2-dimethylpropionamide;
2-(4-Fluorophenyl)-N-[6-nitro-1-(4-trifluoromethylbenzyl)-2,3-dihydro-1H-indol-5-yl]-acetamide;
N-[1-(5-Chlorothiophen-2-ylmethyl)-6-nitro-2,3-dihydro-1H-indol-5-yl]-2-(4-fluorophenyl)-acetamide;
N-[1-(5-Chlorothiophen-2-ylmethyl)-6-nitro-2,3-dihydro-1H-indol-5-yl]-3,3-dimethylbutyramide;
N-[6-Amino-1-(5-chlorothiophen-2-ylmethyl)-2,3-dihydro-1H-indol-5-yl]-3,3-dimethylbutyramide;
N-[6-Amino-1-(4-trifluoromethylbenzyl)-2,3-dihydro-1H-indol-5-yl]-2,2-dimethylpropionamide;
N-[6-Amino-1-(5-chlorothiophen-2-ylmethyl)-2,3-dihydro-1H-indol-5-yl]-2,2-dimethylpropionamide;
N-[6-Amino-1-(4-trifluoromethylbenzyl)-2,3-dihydro-1H-indol-5-yl]-2-(4-fluorophenyl)-acetamide;
N-[6-Amino-1-(4-trifluoromethylbenzyl)-2,3-dihydro-1H-indol-5-yl]-3,3-dimethylbutyramide;
N-[6-Amino-1-(4-fluorobenzyl)-2,3-dihydro-1H-indol-5-yl]-3,3-dimethylbutyramide;
N-[6-Amino-1-(3-fluoro-4-trifluoromethylbenzyl)-2,3-dihydro-1H-indol-5-yl]-3,3-dimethylbutyramide;
N-[1-(5-Chlorothiophen-2-ylmethyl)-1H-indol-5-yl]-3,3-dimethylbutyramide;
N-[6-Bromo-1-(4-trifluoromethylbenzyl)-2,3-dihydro-1H-indol-5-yl]-3,3-dimethylbutyramide;
N-[6-Bromo-1-(5-chlorothiophen-2-ylmethyl)-2,3-dihydro-1H-indol-5-yl]-3,3-dimethylbutyramide;
N-[1-(4-Chlorobenzyl)-2,3-dihydro-1H-indol-5-yl]-3,3-dimethylbutyramide;
N-[1-(4-Isopropylbenzyl)-2,3-dihydro-1H-indol-5-yl]-3,3-dimethylbutyramide;
N-[1-(3-Fluoro-4-trifluoromethylbenzyl)-2,3-dihydro-1H-indol-5-yl]-3,3-dimethylbutyramide;
N-[1-(6-Chlorobenzo[1,3]dioxol-5-ylmethyl)-2,3-dihydro-1H-indol-5-yl]-3,3-dimethylbutyramide;
N-[1-(3,5-Dimethyl-1-phenyl-1H-pyrazol-4-ylmethyl)-2,3-dihydro-1H-indol-5-yl]-3,3-dimethylbutyramide;
N-[1-(2-Chloro-5-trifluoromethylbenzyl)-2,3-dihydro-1H-indol-5-yl]-3,3-dimethylbutyramide;
N-{1-[5-(4-Chlorophenoxy)-1,3-dimethyl-1H-pyrazol-4-ylmethyl]-2,3-dihydro-1H-indol-5-yl}-3,3-dimethylbutyramide;
3,3-Dimethyl-N-[1-(6-p-tolyloxy-pyridin-3-ylmethyl)-2,3-dihydro-1H-indol-5-yl]-butyramide;
N-{1-[6-(4-Chlorophenylsulfanyl)-pyridin-3-ylmethyl]-2,3-dihydro-1H-indol-5-yl}-3,3-dimethylbutyramide;
N-{1-[6-(4-Cyanophenoxy)-pyridin-3-ylmethyl]-2,3-dihydro-1H-indol-5-yl}-3,3-dimethylbutyramide;
3,3-Dimethyl-N-[1-(6-trifluoromethylpyridin-3-ylmethyl)-2,3-dihydro-1H-indol-5-yl]-butylamide;
3,3-Dimethyl-N-[1-(3-methyl-benzo[b]thiophen-2-ylmethyl)-2,3-dihydro-1H-indol-5-yl]-butyramide;
N-[1-(6-Fluoro-4H-benzo[1,3]dioxin-8-ylmethyl)-2,3-dihydro-1H-indol-5-yl]-3,3-dimethylbutyramide;
3,3-Dimethyl-N-[1-(6-phenoxypyridin-3-ylmethyl)-2,3-dihydro-1H-indol-5-yl]-butyramide;
3,3-Dimethyl-N-[1-(3-methyl-5-phenyl-isoxazol-4-ylmethyl)-2,3-dihydro-1H-indol-5-yl]-butyramide;
N-(1-Benzo[b]thiophen-2-ylmethyl-2,3-dihydro-1H-indol-5-yl)-3,3-dimethylbutyramide:
N-{1-[1-(4-Fluorophenyl)-5-methyl-1H-pyrazol-4-ylmethyl]-2,3-dihydro-1H-indol-5-yl}-3,3-dimethylbutyramide;
3,3-Dimethyl-N-[1-(5-methylthiophen-2-ylmethyl)-2,3-dihydro-1H-indol-5-yl]-butyramide;
3,3-Dimethyl-N-[1-(4-pyrrol-1-yl-benzyl)-2,3-dihydro-1H-indol-5-yl]-butyramide;
N-[1-(4-Chlorobenzyl)-2,3-dihydro-1H-indol-5-yl]-2-(4-fluorophenyl)-acetamide;
2-(4-Fluorophenyl)-N-[1-(4-trifluoromethylbenzyl)-2,3-dihydro-1H-indol-5-yl]-acetamide;
2-(4-Fluorophenyl)-N-[1-(4-isopropylbenzyl)-2,3-dihydro-1H-indol-5-yl]-acetamide;
2-(4-Fluorophenyl)-N-[1-(3-fluoro-4-trifluoromethylbenzyl)-2,3-dihydro-1H-indol-5-yl]-acetamide;
N-[1-(6-Chlorobenzo[1,3]dioxol-5-ylmethyl)-2,3-dihydro-1H-indol-5-yl]-2-(4-fluorophenyl)-acetamide;
N-[1-(3,5-Dimethyl-1-phenyl-1H-pyrazol-4-ylmethyl)-2,3-dihydro-1H-indol-5-yl]-2-(4-fluorophenyl)-acetamide;
N-[1-(2-Chloro-5-trifluoromethylbenzyl)-2,3-dihydro-1H-indol-5-yl]-2-(4-fluorphenyl)-acetamide;
N-{1-[5-(4-Chlorophenoxy)-1,3-dimethyl-1H-pyrazol-4-ylmethyl]-2,3-dihydro-1H-indol-5-yl}-2-(4-fluorphenyl)-acetamide:
N-{1-[6-(4-Cyanophenoxy)-pyridin-3-ylmethyl]-2,3-dihydro-1H-indol-5-yl}-2-(4-fluorophenyl)-acetamide;
2-(4-Fluorophenyl)-N-[1-(3-methyl-benzo[b]thiophen-2-ylmethyl)-2,3-dihydro-H-indol-5-yl]-acetamide;
N-[1-(6-Fluoro-4H-benzo[1,3]dioxin-8-ylmethyl)-2,3-dihydro-1H-indol-5-yl]-2-(4-fluorophenyl)-acetamide;
2-(4-Fluorophenyl)-N-[1-(6-phenoxypyridin-3-ylmethyl)-2,3-dihydro-1H-indol-5-yl]-acetamide;
N-(1-Benzo[b]thiophen-2-ylmethyl-2,3-dihydro-1H-indol-5-yl-2-(4-fluorophenyl)-acetamide;
2-(4-Fluorophenyl)-N-{1-[1-(4-fluorophenyl)-5-methyl-1H-pyrazol-4-ylmethyl]-2,3-dihydro-1H-indol-5-yl}-acetamide;
2-(4-Fluorophenyl)-N-[1-(5-methylthiophen-2-ylmethyl)-2,3-dihydro-1H-indol-5-yl]-acetamide; and
2-(4-Fluorophenyl)-N-[1-(4-pyrrol-1-yl-benzyl)-2,3-dihydro-1H-indol-5-yl]-acetamide, or
   a pharmaceutically acceptable salt thereof.

Compounds according to formula 4 can be prepared as described in WO2004/096767.

An embodiment of the invention relates to a method wherein said compound is a compound according to formula 5:

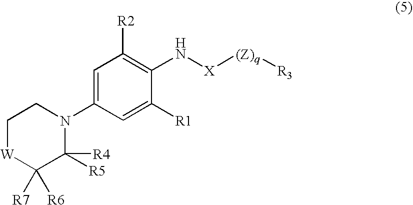

wherein
q is 0 or 1;
W is O or S;
X is CO;
Z is 0;
R1 is selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy;
R2 is selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy, C₃₋₈-cycloalk(en)yl-C₁₋₆-alk(en/yn)yloxy, optionally substituted phenyl and optionally substituted pyridyl; wherein phenyl and pyridyl are optionally substituted with one or more substituents independently being halogen, C₁₋₆-alk(en/yn)yl, C₃₋₈-cycloalk(en)yl or C₃₋₈-cycloalk(en)yl-C₁₋₆-alk(en/yn)yl;
R3 is selected from the group consisting of C₁₋₁₀-alk(en/yn)yl, C₃₋₈-cycloalk(en)yl, C₃₋₈-cycloalk(en)yl-C₁₋₆-alk(en/yn)yl, Ar—C₁₋₆-alk(en/yn)yl, Ar—C₃₋₈-cycloalk(en)yl, Ar—C₃₋₈-cycloalk(en)yl-C₁₋₆-alk(en/yn)yl and Ar; and
each of R4, R5, R6 and R7 is independently selected from the group consisting of hydrogen and Ar;
   as the free base or salts thereof.

In an embodiment of formula 5
R1 is selected from the group consisting of halogen, cyano, C₁₋₆-alk(en/yn)yl, halo-C₁₋₆-alk(en/yn)yl and C₁₋₆-alk(en/yn)yloxy.
R2 is selected from the group consisting of halogen, cyano, C₁₋₆-alk(en/yn)yl, halo-C₁₋₆-alk(en/yn)yl, C₁₋₆-alk(en/yn)yloxy, optionally substituted phenyl and optionally substituted pyridyl.
optionally substituted phenyl and optionally substituted pyridyl may be substituted with one or more substituents independently being halogen or C₁₋₆-alk(en/yn)yl.
R3 is selected from the group consisting of C₁₋₁₀-alk(en/yn)yl, C₃₋₈-cycloalk(en)yl-C₁₋₆-alk(en/yn)yl, Ar—C₁₋₆-alk(en/yn)yl and Ar.
any Ar may be substituted with one or more substituents independently being halogen, C₁₋₆-alk(en/yn)yl, halo-C₁₋₆-alk(en/yn)yl or C₁₋₆-alk(en/yn)yloxy.

In an embodiment of formula 5:
q is 0 or 1;
W is O or S;
X is CO;
Z is O;
R1 and R2 are independently selected from the group consisting of halogen, halo-C₁₋₆-alk(en/yn)yl, C₁₋₆-alk(en/yn)yl and cyano;
R3 is selected from the group consisting of C₁₋₁₀-alk(en/yn)yl, C₃₋₈-cycloalk(en)yl-C₁₋₆-alk(en/yn)yl, Ar—C₁₋₆-alk(en/yn)yl and Ar; and
R4, R5, R6 and R7 are independently selected from the group consisting of hydrogen and Ar;

In an embodiment of the present invention, the following definitions are applied for formula 5:
The term heteroatom refers to a nitrogen, oxygen or sulphur atom.
Halogen means fluoro, chloro, bromo or iodo.
The expression C₁₋₆-alk(en/yn)yl means a C₁₋₆-alkyl, C₂₋₆-alkenyl or a C₂₋₆-alkynyl group. The term C₁₋₆-alkyl refers to a branched or un-branched alkyl group having from one to six carbon atoms inclusive, including but not limited to methyl, ethyl, 1-propyl, 2-propyl, 1-butyl, 2-butyl, 2-methyl-2-propyl and 2-methyl-1-propyl. Similarly, C₂₋₆-alkenyl and C₂₋₆-alkynyl, respectively, designate such groups having from two to six carbon atoms, including one double bond and one triple bond respectively, including but not limited to ethenyl, propenyl, butenyl, ethynyl, propynyl and butynyl.
The expression C₁₋₁₀-alk(en/yn)yl means a C₁₋₁₀-alkyl, C₂₋₁₀-alkenyl or a C₂₋₁₀-alkynyl group. The term C₁₋₁₀-alkyl refers to a branched or un-branched alkyl group having from one to six carbon atoms inclusive, including but not limited to methyl, ethyl, 1-propyl, 2-propyl, 1-butyl, 2-butyl, 1-pentyl, 1-hexyl, 1-heptyl, 1-octyl, 1-nonyl, 1-decyl, 2-methyl-2-propyl and 2-methyl-1-propyl. Similarly, C₂₋₁₀-alkenyl and C₂₋₁₀-alkynyl, respectively, designate such groups having from two to six carbon atoms, including one double bond and one triple bond respectively, including but not limited to ethenyl, propenyl, butenyl, pentenyl, hexenyl, heptenyl, octenyl, nonenyl, decenyl, ethynyl, propynyl, butynyl, pentynyl, hexynyl, heptynyl, octynyl, nonynyl, and decynyl.
The expression C₃₋₈-cycloalk(en)yl means a C₃₋₈-cycloalkyl- or cycloalkenyl group.
The term C₃₋₈-cycloalkyl designates a monocyclic or bicyclic carbocycle having three to eight C-atoms, including but not limited to cyclopropyl, cyclopentyl, cyclohexyl, cycloheptyl, cyclooctyl, [1.1.1]bicyclopentyl, bicyclo[2.2.1]heptyl, [2.2.2]bicyclooctyl and [3.3.0]bicyclooctyl, etc. The term C₃₋₈-cycloalkenyl designates a monocyclic or bicyclic carbocycle having three to eight C-atoms and including one double bond.
The term halo-C₁₋₆-alk(en/yn)yl designates C₁₋₆-alk(en/yn)yl being substituted with one or more halogen atoms, including but not limited to trifluoromethyl.
Similarly, halo-C₃₋₈-cycloalk(en)yl designates C₃₋₈-cycloalk(en)yl being substituted with one or more halogen atoms.

In the expression halo-C₃₋₈-cycloalk(en)yl-C₁₋₆-alk(en/yn)yl the terms C₁₋₆-alk(en/yn)yl and halo-C₃₋₈-cycloalk(en)yl are as defined above.
When two adjacent substituents together with the aromatic group to which they are attached form a 4-8 membered ring, which optionally contains one, two or three heteroatoms, then a ring system is formed by 4-8 atoms selected from 4-8 carbonatoms and 0-3 heteroatoms selected from N, S, or O, Such two adjacent substituents may together form: —(CH₂)ₐ—CH₂—, —CH═CH—(CH₂)_{b}—, —CH₂—CH═CH—(CH₂)_{c}, —CH═CH—CH═CH—, —(CH₂)ₐ—O—, —(CH₂)_{b}—O—, —CH₂—O—(CH₂)_{c}—O—, —CH₂—O—CH₂—O—CH₂—, —(CH₂)ₐ—S—, —S—(CH₂)_{b}—S—, —CH₂—S—(CH₂)_{c}—S—, —CH₂—S—CH₂—S—CH₂—, —(CH₂)ₐ—NH—, —NH—(CH₂)_{b}—NH—, —CH₂—NH—(CH₂)_{c}—NH—, —CH═CH—NH—, —O—(CH₂)_{b}—NH—, —CH₂—O—(CH₂), —NH— or —O—(CH₂)_{c}—NH—CH₂—, —S—(CH₂)_{b}—NH—, —N═CH—NH—, —N═CH—O— or —N═CH—S— or —N═N—NH—, wherein b is 1, 2 or 3, a is 2, 3 or 4 and c is 1 or 2.
The term Ar refers to optionally substituted aromatic systems of 5-10 carbon atoms, wherein 0, 1, 2, 3 or 4 carbon atoms may be replaced by heteroatoms independently selected from N, S, or O. Examples of such Ar groups are optionally substituted phenyl, optionally substituted naphthyl, optionally substituted pyridine, optionally substituted thiophene, optionally substituted furan, optionally substituted thiazole, optionally substituted quinoline, optionally substituted indole, optionally substituted 2,3-dihydro-benzofuran, optionally substituted pyrimidine, optionally substituted pyrrole and optionally substituted oxazole. Ar may be substituted with one or more substituents independently being hydroxy, halogen, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, C₁₋₆-alk(en/yn)yloxy, C₃₋₈-alk(en/yn)yloxy, acyl, nitro or cyano, —CO—NH—C₁₋₆-alk(en/yn)yl, —CO—N(C₁₋₆-alk(en/yn)yl)₂, —NH₂, —NH—C₁₋₆alk(en/yn)yl, —N(C₁₋₆-alk(en/yn)yl)₂, —S—C₁₋₆-alk(en/yn)yl, —SO₂—C₁₋₆-alk(en/yn)yl, —SO₂N(C₁₋₆-alk(en/yn)yl)₂ and —SO₂NH—C₁₋₆-alk(en/yn)yl; or two adjacent substituents may together with the aromatic group to which they are attached form a 4-8 membered ring, which optionally contains one, two or three heteroatoms.
When Ar is substituted with CO—NH—C₁₋₆-alk(en/yn)yl or CO—N(C₁₋₆-alk(en/yn)yl)₂, then the carbon atom of the CO group is attached to Ar.
When Ar is substituted with NH₂, NH—C₁₋₆-alk(en/yn)yl or N(C₁₋₆-alk(en/yn)yl)₂, then the nitrogen atom is attached to Ar.
When Ar is substituted with —S—C₁₋₆-alk(en/yn)yl, —SO₂—C₁₋₆-alk(en/yn)yl, —SO₂N(C₁₋₆-alk(en/yn)yl)₂ or —SO₂NH—C₁₋₆-alk(en/yn)yl then the sulphur atom is attached to Ar.
The term acyl refers to formyl, C₁₋₆-alk(en/yn)ylcarbonyl, C₃₋₈-cycloalk(en)ylcarbonyl, Ar-carbonyl, Ar—C₁₋₆-alk(en/yn)ylcarbonyl or a C₃₋₈-cycloalk(en)yl-C₁₋₆-alk(en/yn)yl-carbonyl group, wherein C₁₋₆-alk(en/yn)yl, C₃₋₈-cycloalk(en)yl and Ar are as defined above.
The terms C₃₋₈-cycloalk(en)yl-C₁₋₆-alk(en/yn)yl, Ar—C₁₋₆-alk(en/yn)yl, C₁₋₆-alk(en/yn)yloxy and C₃₋₈-cycloalk(en)yloxy; designate such groups in which the C₁₋₆-alk(en/yn)yl, C₃₋₈-cycloalk(en)yl and Ar are as defined above. Similarly, C₃₋₈-cycloalk(en)yl-C₁₋₆-alk(en/yn)yloxy designate such groups in which C₃₋₈-cycloalk(en)yl and C₁₋₆-alk(en/yn)yloxy are as defined above.
The expressions Ar—C₃₋₈-cycloalk(en)yl and Ar—C₃₋₈-cycloalk(en)yl-C₁₋₆-alk(en/yn)yl designate such groups in which the C₁₋₆-alk(en/yn)yl, C₃₋₈-cycloalk(en)yl and Ar are as defined above.

In an embodiment of formula 5 said compound is selected from the group consisting of:
N-(2-Bromo-4-morpholin-4-yl-6-trifluoromethyl-phenyl)-2-(4-fluoro-phenyl)-acetamide;
2-Cyclopentyl-N-(2-bromo-6-trifluoromethyl-4-morpholin-4-yl-phenyl)-acetamide;
N-(2-Bromo-4-morpholin-4-yl-6-trifluoromethyl-phenyl)-3-cyclopentyl-propionamide;
N-(2-Chloro-6-cyano-4-morpholin-1-yl-phenyl)-3-cyclohexyl-propionamide;
2-Cyclopentyl-N-(2,6-Dimethyl-4-thiomorpholin-4-yl-phenyl)-acetamide;
2-Cyclopentyl-N-[2,6-dimethyl-4-(2-phenyl-morpholin-4-yl)-phenyl]-acetamide;
2-Cyclopentyl-N-[2,6-dimethyl-4-(2-phenyl-thiomorpholin-4-yl)-phenyl]-acetamide;
2-Cyclopentyl-N-[2,6-dimethyl-4-(3-pyridin-3-yl-thiomorpholin-4-yl)-phenyl]-acetamide;
2-Cyclopentyl-N-{2,6-dimethyl-4-[2-(4-trifluoromethyl-phenyl)-thiomorpholin-4-yl]-phenyl}-acetamide;
N-{4-[2-(2-Chloro-phenyl)-thiomorpholin-4-yl]-2,6-dimethyl-phenyl}-2-cyclopentyl-acetamide;
2-Bicycolo[2.2.1]hept-2-yl-N-(2,6-dimethyl-4-morpholin-4-ylphenyl)-acetamide;
2-Cyclohexyl-N-(2,6-dimethyl-4-morpholin-4-yl-phenyl)-acetamide;
3-(3,4-Difluoro-phenyl)-N-(2,6-dimethyl-4-morpholin-4-yl-phenyl)-propionamide;
2-Cyclopentyl-N-(2,6-dimethyl-4-morpholin-4-phenyl)-acetamide;
(2,6-Dimethyl-4-morpholin-4-yl-phenyl)-carbamic acid butyl ester;
2-(4-Chloro-phenyl)-N-(2,6-dimethyl-4-morpholin-4-yl-phenyl)-acetamide;
2,3-Dihydro-benzofuran-2-carboxylic acid (2,6-dimethyl-4-morpholin-4-yl-phenyl-amide;
3-Cyclohexyl-N-(2,6-dimethyl-4-morpholin-4-yl-phenyl)-propionamide;
3-Cyclopentyl-N-(2,6-dimethyl-4-morpholin-1-yl-phenyl)-propionamide;
N-(2,6-Dimethyl-4-morpholin-4-yl-phenyl)-2-(4-fluoro-phenyl)-acetamide;
N-(2,6-Dimethyl-4-morpholin-4-yl-phenyl)-2-thiophen-2-yl-acetamide;
N-(2,6-Dimethyl-4-morpholin-4-yl-phenyl)-3,3-dimethyl-butyramide;
Hexanoic acid (2,6-dimethyl-4-morpholin-4-yl-phenyl)-amide;
2-Cycloheptyl-N-(2,6-dimethyl-4-morpholin-4-yl-phenyl)-acetamide;
(2,6-Dimethyl-4-morpholin-4-yl-phenyl)-carbamic acid benzyl ester;
(2,6-Dimethyl-4-morpholin-4-yl-phenyl)-carbamic acid 2-chloro-benzyl ester;
3,5,5-Trimethyl-hexanoic acid (2,6-dimethyl-4-morpholin-4-yl-phenyl)-amide;
Octanoic acid (2,6-dimethyl-4-morpholin-4-yl-phenyl)-amide;
Heptanoic acid (2,6-dimethyl-4-morpholin-4-yl-phenyl)-amide;
N-(2,6-Dimethyl-4-morpholin-4-yl-phenyl)-2-phenyl-acetamide
2-(3,4-Dichloro-phenyl)-N-(2,6-dimethyl-4-morpholin-4-ylphenyl)-acetamide;
2-(4-Allyloxy-3-chloro-phenyl)-N-(2,6-dimethyl-4-morpholin-4-yl-phenyl)-acetamide;
N-(2,6-Dimethyl-4-morpholin-4-yl-phenyl)-2-(3-trifluoromethyl-phenyl)-acetamide;
N-(2,6-Dimethyl-4-morpholin-4-yl-phenyl-(2-naphthalen-2-yl-acetamide;
3-(3-Chloro-phenyl)-N-(2,6-dimethyl-4-morpholin-4-yl-phenyl)-propionamide;
N-(2,6-Dimethyl-4-morpholin-4-yl-phenyl)-2-(3,4-methyl-phenyl)-acetamide;
2-(3-Bromo-phenyl)-N-(2,6-dimethyl-4-morpholin-4-yl-phenyl)-acetamide;
2-(3-Chloro-phenyl)-N-(2,6-dimethyl-4-morpholin-4-yl-phenyl)-acetamide;
N-(2,6-Dimethyl-4-morpholin-4-yl-phenyl)-2-yl-acetamide;
N-(2,6-Dimethyl-4-morpholin-4-yl-phenyl)-2-(3,4-dimethyl-phenyl)-acetamide;
2-(3,4-Difluoro-phenyl)-N-(2,6-dimethyl-4-morpholin-4-p-phenyl)-acetamide;
N-(2,6-Dimethyl-1-morpholin-4-yl-phenyl)-2-(3-fluoro-phenyl)-acetamide;
N-(2-Bromo-4-morpholin-4-yl-6-trifluoromethyl-phenyl)-3-cyclohexyl-propionamide;
N-(2-Bromo-4-morpholin-4-yl-6-trifluoromethyl-phenyl)-2-(3-fluoro-phenyl)-acetamide;
N-(2-Bromo-4-morpholin-4-yl-6-trifluoromethyl-phenyl)-propionamide;
N-(2-Bromo-4-morpholin-4-yl-6-trifluoromethyl-phenyl)-butyramide;
N-(2-Chloro-4-morpholin-4-yl-6-trifluoromethyl-phenyl-2-(3-fluoro-phenyl)-acetamide:
N-(2-Chloro-4-morpholin-4-yl-6-trifluoromethyl-phenyl)-2-cyclopentyl-acetamide;
2-Cyclopentyl-N-{2,6-dimethyl-4-[2-(4-trifluoromethyl-phenyl)-morpholin-4-yl]-phenyl}-acetamide:
N-{4-[2-(2-Chloro-phenyl)-morpholin-4-yl]-2,6-dimethyl-phenyl}-2-cyclopentyl-acetamide;
2-Cyclopentyl-N-{4-[2-(4-fluoro-phenyl)-morpholin-4-yl]-2,6-dimethyl-phenyl}-acetamide;
2-(2-Chloro-phenyl)-N-(2,6-dimethyl-4-morpholin-4-yl-phenyl)-acetamide;
Pentanoic acid (2,6-dimethyl-4-morpholin-4-yl-phenyl)-amide;
4-Methyl-pentanoic acid (2,6-dimethyl-4-morpholin-4-yl-phenyl)-amide;
2-Cyclopent-2-enyl-N-(2,6-dimethyl-4-morpholin-4-yl-phenyl)-acetamide;
5-Methyl-hexanoic acid (2,6-dimethyl-4-morpholin-4-yl-phenyl)-amide;
3-Methyl-pentanoic acid (2,6-dimethyl-4-morpholin-1-yl-phenyl)-amide;
Hex-5-enoic acid (2,6-dimethyl-4-morpholin-4-yl-phenyl)-amide;
3-Ethyl-pentanoic acid (2,6-dimethyl-4-morpholin-4-yl-phenyl)-amide;
2-Cyclopentyl-N-(4-morpholin-4-yl-2-pyridin-3-yl-6-trifluoromethyl-phenyl)-acetamide;
2-Cyclopentyl-N-(5-morpholin-4-yl-3-trifluoromethyl-biphenyl-2-yl)-acetamide:
2-Cyclopentyl-N-(4′-fluoro-5-morpholin-4-yl-3-trifluoromethyl-biphenyl-2-yl)-acetamide;
2-Cyclopentyl-N-(4′-methyl-5-morpholin-4-yl-3-trifluoromethyl-biphenyl-2-yl)-acetamide;
2-Cyclopentyl-N-(3′-methyl-5-morpholin-4-yl-3-trifluoromethyl-biphenyl-2-yl)-acetamide;
2-Cyclopentyl-N-(3′,4′-difluoro-5-morpholin-4-yl-3-trifluoromethyl-biphenyl-2-yl)-acetamide:
2-(4-Fluoro-phenyl)-N-(4-morpholin-4-yl-2-pyridin-3-yl-6-trifluoromethyl-phenyl)-acetamide;
2-Cyclopentyl-N-(2,6-diethyl-4-morpholin-4-yl-phenyl)-acetamide;
2-Cyclopentyl-N-(2,6-diisopropyl-4-morpholin-4-yl-phenyl)-acetamide;
2-Cyclopentyl-N-(2,6-difluoro-4-morpholin-4-yl-phenyl)-acetamide;
Hexanoic acid (2,6-difluoro-4-morpholin-4-yl-phenyl)-amide;
N-(2,6-Difluoro-4-morpholin-4-yl-phenyl)-3,3-dimethyl-butyramide;
N-(2,6-Difluoro-4-morpholin-4-yl-phenyl)-2-(3-fluoro-phenyl)-acetamide;
2-Cyclopent-2-enyl-N-(2,6-difluoro-4-morpholin-4-yl-phenyl)-acetamide;
2-Bicyclo[2.2.1]hept-2-yl-N-(2,6-difluoro-4-morpholin-4-yl-phenyl)-acetamide;
2-Bicyclo[2.2.1]hept-2-yl-N-(2-methyl-4-morpholin-4-yl-6-trifluoromethyl-phenyl)-acetamide;
5-Methyl-pentanoic acid (2-methyl-4-morpholin-4-yl-6-trifluoromethyl-phenyl)-amide;
5-Methyl-hexanoic acid (2-methyl-4-morpholin-4-yl-6-trifluoromethyl-phenyl)-amide;
2-Cyclopent-2-enyl-N-(2-methyl-4-morpholin-4-yl-6-trifluoromethyl-phenyl)-acetamide;
2-Cyclopentyl-N-(2-methyl-4-morpholin-4-yl-6-trifluoromethyl-phenyl)-acetamide;
Hexanoic acid (2-methyl-4-morpholin-4-yl-6-trifluoromethyl-phenyl)-amide;
3,3-Dimethyl-N-(2-methyl-4-morpholin-4-yl-6-trifluoromethyl-phenyl)-butyramide;
2-(3,4-Difluoro-phenyl)-N-(2-methyl-4-morpholin-1-yl-6-trifluoromethyl-phenyl)-acetamide;
Hexanoic acid (2-methoxy-6-methyl-4-morpholin-4-yl-phenyl)-amide;
2-Cyclopentyl-N-(2-methoxy-6-methyl-4-morpholin-1-yl-phenyl)-acetamide;
N-(2-Methoxy-6-methyl-4-morpholin-4-yl-phenyl)-3,3-dimethyl-butyramide;
2-(3,4-Difluoro-phenyl)-N-(2-methoxy-6-methyl-4-morpholin-4-yl-phenyl)-acetamide;
2-Cyclopent-2-enyl-N-(2-methoxy-6-methyl-4-morpholin-1-yl-phenyl)-acetamide;
2-(3-Fluoro-phenyl)-N-(2-methoxy-6-methyl-4-morpholin-4-yl-phenyl)-acetamide;
2-Bicyclo[2.2.1]hept-2-yl-N-(2-methoxy-6-methyl-4-morpholin-1-yl-phenyl)-acetamide;
4-Methyl-pentanoic acid (2-methoxy-6-methyl-4-morpholin-4-yl-phenyl)-amide;
5-Methyl-hexanoic acid (2-methoxy-6-methyl-4-morpholin-1-yl-phenyl)-amide;
N-(2-Chloro-6-methyl-4-morpholin-4-yl-phenyl)-2-(3-fluoro-phenyl)-acetamide; and
N-(2-Chloro-6-methyl-4-morpholin-4-yl-phenyl-2-cyclopentyl-acetamide.
   as the free base or a salt thereof.

Compounds according to formula 5 can be prepared as described in WO2005/087754.

An embodiment of the invention relates to a method wherein said compound is a compound according to formula 6 or salt thereof:

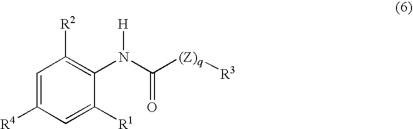

wherein:
Z is O or S; and
q is 0 or 1; and
each of R¹ and R² is independently selected from the group consisting of halogen, cyano, amino, C₁₋₆-alk(en/yn)yl, C₃₋₈-cycloalk(en)yl, C₃₋₈-cycloalk(en)yl-C₁₋₆-alk(en/yn)yl, C₃₋₈-heterocycloalk(en)yl, Aryl, Heteroaryl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy, C₃₋₈-cycloalk(en)yl-C₁₋₆-alk(en/yn)yloxy, C₃₋₈-heterocycloalk(en)yloxy; and
R³ is selected from the group consisting of C₁₋₈-alk(en/yn)yl, C₃₋₈-cycloalk(en)yl, C₃₋₈-cycloalk(en)yl-C₁₋₆-alk(en/yn)yl, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, C₃₋₈-heterocycloalk(en)yl-C₁₋₆-alk(en/yn)yl, C₁₋₆-alk(en/yn)yl-C₃₋₈-heterocycloalk(en)yl-C₁₋₆-alk(en/yn)yl, Heteroaryl-C₁₋₆-alk(en/yn)yl, Heteroaryl-C₃₋₈-cycloalk(en)yl, Heteroaryl-C₃₋₈-cycloalk(en)yl-C₁₋₆-alk(en/yn)yl, amino-C₁₋₆-alk(en/yn)yl, amino-C₃₋₈-cycloalk(en)yl, amino-C₃₋₈-cycloalk(en)yl-C₁₋₆-alk(en/yn)yl, C₁₋₆-alk(en/yn)yloxy-C₁₋₆-alk(en/yn)yl, C₃₋₈-cycloalk(en)yloxy-C₁₋₆-alk(en/yn)yl, C₃₋₈-cycloalk(en)yl-C₁₋₆-alk(en/yn)yloxy-C₁₋₆-alk(en/yn)yl, halo-C₁₋₆-alk(en/yn)yl, halo-C₃₋₈-cycloalk(en)yl and halo-C₃₋₈-cycloalk(en)yl-C₁₋₆-alk(en/yn)yl; and
R⁴ is selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, C₃₋₈-heterocycloalk(en)yl, Aryl, Heteroaryl, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, Aryl-C₃₋₈-heterocyoloalk(en)yl, halo-C₁₋₆-alk(en/yn)yl, halo-C₃₋₈-cycloalk(en)yl, halo-C₃₋₈-cycloalk(en)yl-C₁₋₆-alk(en/yn)yl, halo-C₁₋₆-alk(en/yn)yl-C₃₋₈-heterocycloalk(en)yl-C₁₋₆-alk(en/yn)yl, NR⁵R⁶ and R⁷NH—C₁₋₆-alk(en/yn)yl; wherein R⁵ and R⁶ are independently selected from the group consisting of hydrogen, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, C₁₋₆-alk(en/yn)yl, C₂₋-cycloalk(en)yl, C₃₋₈-cycloalk(en)yl-C₁₋₆-alk(en/yn)yl, Heteroaryl-C₁₋₆-alk(en/yn)yl, Heteroaryl-C₃₋₈-cycloalk(en)yl and Heteroaryl-C₃₋₈-cycloalk(en)yl-C₁₋₆-alk(en/yn)yl with the proviso that R⁵ and R⁶ are not hydrogen at the same time; and R⁷ is selected from the group consisting of C₁₋₆-alk(en/yn)yl, C₃₋₈-cycloalk(en)yl, C₃₋₈-cycloalk(en)yl-C₁₋₆-alk(en/yn)yl, Aryl, halo-C₁₋₆-alk(en/yn)yl, halo-C₃₋₈-cycloalk(en)yl, halo-C₃₋₈-cycloalk(en)yl-C₁₋₆-alk(en/yn)yl, Aryl-C₁₋₆-alk(en/yn)yl, Aryl-C₃₋₈-cycloalk(en)yl and Heteroaryl.

In an embodiment of formula 6
R¹ and R² are independently selected from the group consisting of halogen, amino, C₁₋₆-alk(en/yn)yl, C₃₋₈-heterocycloalk(en)yl, Aryl, Heteroaryl and halo-C₁₋₆-alk(en/yn)yl.
Z is an oxygen atom.
R³ is selected from the group consisting of C₁₋₈-alk(en/yn)yl, C₃₋₈-cycloalk(en)yl, C₃₋₈-cycloalk(en)yl-C₁₋₆-alk(en/yn)yl, Aryl-C₁₋₆-alk(en/yn)yl, Aryl-C₃₋₈-cycloalk(en)yl, Heteroaryl-C₁₋₆-alk(en/yn)yl and amino-C₁₋₆-alk(en/yn)yl.
R⁴ is selected from the group consisting of halogen, C₁₋₆-alk(en/yn)yl, C₃₋₈-heterocycloalk(en)yl, Heteroaryl, Aryl-C₃₋₈-heterocycloalk(en)yl, NR⁵R⁶ and R⁷NH—C₁₋₆-alk(en/yn)yl; wherein
   R⁵ and R⁶ are independently selected from the group consisting of hydrogen, Aryl-C₁₋₆-alk(en/yn)yl, C₁₋₆-alk(en/yn)yl and Heteroaryl-C₁₋₆-alk(en/yn)yl with the proviso that R⁵ and R⁶ are not hydrogen at the same time.
   R⁷ is Aryl.
any Aryl which is mentioned either alone or as a part of a larger substituent is optionally substituted with one or more substituents independently selected from the group consisting of amino, halogen, cyano, C₁₋₆-alk(en/yn)yl, halo-C₁₋₆-alk(en/yn)yl, hydroxy, C₁₋₆-alk(en/yn)yloxy, halo-C₁₋₆-alk(en/yn)yloxy, di-(C₁₋₆-alk(en/yn)yl)amino, C₁₋₆-alk(en/yn)yl-CO—NH— and C₁₋₆-alk(en/yn)yl-sulfonamide; or two adjacent substituents may together with the Aryl group to which they are attached form a 4-8 membered ring, which optionally contains one or two heteroatoms and which is optionally substituted with one or more C₁₋₆-alk(en/yn)yl groups
any Heteroaryl which is mentioned either alone or as a part of a larger substituent is optionally substituted with one or more substituents independently selected from the group consisting of halogen, halo-C₁₋₆-alk(en/yn)yl, C₁₋₆-alk(en/yn)yl, Aryl, C₁₋₆-alk(en/yn)yloxy and C₁₋₆-alk(en/yn)yl-phenoxy.

In an embodiment of the present invention, the following definitions are applied for formula 6:
The term heteroatom refers to a nitrogen, oxygen or sulphur atom.
Halogen means fluoro, chloro, bromo or iodo.
Amino means NH₂.
The expression “C₁₋₆-alk(en/yn)yl” means C₁₋₆-alkyl, C₂₋₆-alkenyl or C₂₋₆-alkynyl.
The term “C₁₋₆-alkyl” refers to a branched or unbranched alkyl group having from one to six carbon atoms inclusive, including but not limited to methyl, ethyl, prop-1-yl, prop-2-yl, 2-methyl-prop-1-yl, 2-methyl-prop-2-yl, 2,2-dimethyl-prop-1-yl, but-1-yl, but-2-yl, 3-methyl-but-1-yl, 3-methyl-but-2-yl, pent-1-yl, pent-2-yl, pent-3-yl, hex-1-yl, hex-2-yl and hex-3-yl. The term “C₂₋₆-alkenyl” designates such groups having from two to six carbon atoms and one double bond, including but not limited to ethenyl, propenyl, and butenyl. The term “C₂₋₆-alkynyl” designates such groups having from two to six carbon atoms and one triple bond, including but not limited to ethynyl, propynyl and butynyl.
The expression “C₁₋₈-alk(en/yn)yl” means C₁₋ₓ-alkyl, C₂₋₈-alkenyl or C₂₋₈-alkynyl. The term “C₁₋₈-alkyl” refers to a branched or unbranched alkyl group having from one to eight carbon atoms inclusive, including but not limited to methyl, ethyl, prop-1-yl, prop-2-yl, 2-methyl-prop-1-yl, 2-methyl-prop-2-yl, 2,2-dimethyl-prop-1-yl, but-1-yl, but-2-yl, 3-methyl-but-1-yl, 3-methyl-but-2-yl, pent-1-yl, pent-2-yl, pent-3-yl, hex-1-yl, hex-2-yl and hex-3-yl, 1-heptyl, 2-heptyl, 3-heptyl and 4-heptyl. The term “C₂₋₈-alkenyl” designates such groups having from two to eight carbon atoms and one double bond, including but not limited to ethenyl, propenyl, and butenyl. The term “C₂₋₈-alkynyl” designates such groups having from two to eight carbon atoms and one triple bond, including but not limited to ethynyl, propynyl and butynyl.
The expression “C₃₋₈-cycloalk(en)yl” means C₃₋₈-cycloalkyl or C₃₋₈-cycloalkenyl. The term “C₃₋₈-cycloalkyl” designates a monocyclic or bicyclic carbocycle having three to eight C-atoms, including but not limited to cyclopropyl, cyclopentyl, cyclohexyl, bicycloheptyl such as 2-bicyclo[2.2.1]heptyl. The term “C₃₋₈-cycloalkenyl” designates a monocyclic or bicyclic carbocycle having three to eight C-atoms and one double bond, including but not limited to cyclopropenyl, cyclopentenyl and cyclohexenyl.
The term “C₃₋₈-heterocycloalk(en)yl” means C₃₋₈-heterocycloalkyl or C₃₋₈-heterocycloalkenyl. The term “C₃₋₈-heterocycloalkyl” designates a monocyclic or bicyclic ring system wherein the ring is formed by 3 to 8 atoms selected from 2-7 carbon atoms and 1 or 2 heteroatoms independently selected from N, S, or O. Examples of C₃₋₈-heterocycloalkyles are pyrrolidine, azepan, morpholine and piperidine. The term “C₃₋₈-heterocycloalkenyl” designates a monocyclic or bicyclic ring system with one double bond, wherein the ring is formed by 3 to 8 atoms selected from 2-7 carbon atoms and 1 or 2 heteroatoms independently selected from N, S, or O.
The term Aryl refers to monocyclic or bicyclic aromatic systems of 5-10 carbon atoms, including but not limited to phenyl and naphthyl. Any Aryl which is mentioned either alone or as a part of a larger substituent is optionally substituted and may thus be substituted with one or more substituents such as with 0, 1, 2, 3 or 4 substituents. Any Aryl which is mentioned either alone or as a part of a larger substituent may thus be substituted with one or more substituents independently selected from the group consisting of amino, halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₃₋₈-heterocycloalk(en)yl, C₁₋₆-alkyl-C₃₋₈-heterocycloalk(en)yl, hydroxy, C₁₋₆-alk(en/yn)yloxy, C₃₋₈-cycloalk(en)yloxy, C₃₋₈-cycloalk(en)yl-C₁₋₆-alk(en/yn)yloxy, halo-C₁₋₆-alk(en/yn)yloxy, halo-C₃₋₈-cycloalk(en)yloxy, halo-C₃₋₈-cycloalk(en)yl-C₁₋₆-alk(en/yn)yloxy, C₁₋₆-alk(en/yn)ylamino, di-(C₁₋₆-alk(en/yn)yl)amino, C₁₋₆-alk(en/yn)yl-CO—NH— and C₁₋₆-alk(en/yn)yl-sulfonamide; or two adjacent substituents may together with the Aryl group to which they are attached form a 4-8 membered ring, which optionally contains one or two heteroatoms and which is optionally substituted with one or more C₁₋₆-alk(en/yn)yl groups. When two adjacent substituents together with the Aryl group to which they are attached form a 4-8 membered ring, which optionally contains one or two heteroatoms, then a ring system is formed by 4-8 atoms selected from 3-8 carbon atoms and 0-2 heteroatoms independently selected from N, S, or O. Such two adjacent substituents may together form: —(CH₂)ₙ—O—, —O—(CH₂)ₘ—O—, —CH₂—O—(CH₂)ₚ—O—, —CH₂—O—CH₂—O—CH₂—, —O—C(CH₃)₂—(CH₂)ₘ—, —(CH₂)ₙ—S—, —S—(CH₂)ₘ—S—, —CH₂—S—(CH₂)ₚ—S— or —CH₂—S—CH₂—S—CH₂—, —S—C(CH₃)₂—(CH₂)ₘ—; wherein m is 1, 2 or 3, n is 2, 3 or 4 and p is 1 or 2.
The term “Heteroaryl” refers to monocyclic or bicyclic heteroaromatic systems of 5-10 atoms selected from 1, 2, 3, 4, 5, 6, 7, 8 or 9 carbon atoms and 1, 2, 3 or 4 heteroatoms independently selected from N, S, or O, including but not limited to pyridine, pyrrole, pyrimidine, quinoline, indole, thiophene, furan, imidazoles such as 3H-imidazol and 1H-imidazol, triazoles such as [1,2,3]triazole and [1,2,4]triazole, tetrazoles such as 2H-tetrazole and oxazole. Any Heteroaryl which is mentioned either alone or as a part of a larger substituent is optionally substituted and may thus be substituted with one or more substituents such as with 0, 1, 2, 3 or 4 substituents. Any Heteroaryl which is mentioned either alone or as a part of a larger substituent may thus be substituted with one or more substituents independently selected from the group consisting of halogen, cyano, amino, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yl, C₃₋₈-cycloalk(en)yl, C₃₋₈-cycloalk(en)yl-C₁₋₆-alk(en/yn)yl, Aryl, Aryl-C₁₋₆-alk(en/yn)yl, C₁₋₆-alk(en/yn)yloxy, C₃₋₈-cycloalk(en)yloxy, C₃₋₈-cycloalk(en)yl-C₁₋₆-alk(en/yn)yloxy, C₁₋₆-alk(en/yn)yl-phenoxy, C₃₋₈-cycloalk(en)yl-phenoxy, C₃₋₈-cycloalk(en)yl-C₁₋₆-alk(en/yn)yl-phenoxy, amino-phenoxy, halo-phenoxy, cyano-phenoxy, halo-C₁₋₆-alk(en/yn)yl-phenoxy, halo-C₃₋₈-cycloalk(en)yl-phenoxy, halo-C₃₋₈-cycloalk(en)yl-C₁₋₆-alk(en/yn)yl-phenoxy, C₃₋₈-heterocycloalk(en)yl-phenoxy, C₁₋₆-alkyl-C₃₋₈-heterocycloalk(en)yl-phenoxy, hydroxy-phenoxy, C₁₋₆-alk(en/yn)yloxy-phenoxy, C₃₋₈-cycloalk(en)yloxy-phenoxy, C₃₋₈-cycloalk(en)yl-C₁₋₆-alk(en/yn)yloxy-phenoxy, halo-C_{—}6-alk(en/yn)yloxy-phenoxy, halo-C₃₋₈-cycloalk(en)yloxy-phenoxy, halo-C₃₋₈-cycloalk(en)yl-C₁₋₆-alk(en/yn)yloxy-phenoxy, C₁₋₆-alk(en/yn)ylamino-phenoxy, di-(C₁₋₆-alk(en/yn)yl)amino-phenoxy, C₁₋₆-alk(en/yn)yl-CO—NH-phenoxy and C₁₋₆-alk(en/yn)yl-sulfonamide-phenoxy.
The term “halo-C₁₋₆-alk(en/yn)yl” designates C₁₋₆-alk(en/yn)yl being substituted with one or more halogen atoms, including but not limited to trifluoromethyl and 3,3,3-trifluoro-1-propyl. Similarly, halo-C₃₋₈-cycloalk(en)yl designates C₃₋₈-cycloalk(en)yl being substituted with one or more halogen atoms and “halo-phenoxy” designates phenoxy being substituted with one or more halogen atoms.
The term “amino-C₁₋₆-alk(en/yn)yl” designates C₁₋₆-alk(en/yn)yl being substituted with one amino group, including but not limited to 1-amino-2-methyl-prop-1-yl and 1-amino-3-methyl-but-1-yl. Similarly, amino-C₃₋₈-cycloalk(en)yl designates C₃₋₈-cycloalk(en)yl being substituted with one amino group and amino-C₃₋₈-cycloalk(en)yl-C₁₋₆-alk(en/yn)yl designates C₃₋₈-cycloalk(en)yl-C₁₋₆-alk(en/yn)yl being wherein C₃₋₈-cycloalk(en)yl is substituted with one amino group.
In the expressions C₃₋₈-cycloalk(en)yl-C₁₋₆-alk(en/yn)yl, C₃₋₈-heterocycloalk(en)yl-C₁₋₆-alk(en/yn)yl, C₁₋₆-alk(en/yn)yl-C₃₋₈-heterocycloalk(en)yl-C₁₋₆-alk(en/yn)yl, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, Aryl-C₃₋₈-heterocycloalk(en)yl, Heteroaryl-C₁₋₆-alk(en/yn)yl, Heteroaryl-C₃₋₈-cycloalk(en)yl, Heteroaryl-C₃₋₈-cycloalk(en)yl-C₁₋₆-alk(en/yn)yl, halo-C₃₋₈-cycloalk(en)yl-C₁₋₆-alk(en/yn)yl, halo-C₁₋₆-alk(en/yn)yl-C₃₋₈-heterocycloalk(en)yl-C₁₋₆-alk(en/yn)yl, C₁₋₆-alk(en/yn)yloxy, C₃₋₈-cycloalk(en)yloxy, C₃₋₈-cycloalk(en)yl-C₁₋₆-alk(en/yn)yloxy, C₃₋₈-heterocycloalk(en)yloxy, C₁₋₆-alk(en/yn)yloxy-C₁₋₆-alk(en/yn)yl, C₃₋₈-cycloalk(en)yloxy-C₁₋₆-alk(en/yn)yl, C₃₋₈-cycloalk(en)yl-C₁₋₆-alk(en/yn)yloxy-C₁₋₆-alk(en/yn)yl, halo-C₁₋₆-alk(en/yn)yloxy, halo-C₃₋₈-cycloalk(en)yloxy, halo-C₃₋₈-cycloalk(en)yl-C₁₋₆-alk(en/yn)yloxy, amino-C₁₋₆-alk(en/yn)yl, amino-C₃₋₈-cycloalk(en)yl, amino-C₃₋₈-cycloalk(en)yl-C₁₋₆-alk(en/yn)yl, R⁷NH—C₁₋₆-alk(en/yn)yl, C₁₋₆-alk(en/yn)ylamino, di-(C₁₋₆-alk(en/yn)yl)amino, C₁₋₆-alk(en/yn)yl-CO—NH—, C₁₋₆-alk(en/yn)yl-sulfonamide C₁₋₆-alk(en/yn)yl-phenoxy, C₃₋₈-cycloalk(en)yl-phenoxy, C₃₋₈-cycloalk(en)yl-C₁₋₆alk(en/yn)yl-phenoxy, halo-phenoxy, halo-C₁₋₆-alk(en/yn)yl-phenoxy, halo-C₃₋₈-cycloalk(en)yl-phenoxy, halo-C₃₋₈-cycloalk(en)yl-C₁₋₆-alk(en/yn)yl-phenoxy, C₃₋₈-heterocycloalk(en)yl-phenoxy, C₁₋₆-alkyl-C₃₋₈-heterocycloalk(en)yl-phenoxy, C₁₋₆-alk(en/yn)yloxy-phenoxy, C₃₋₈-cycloalk(en)yloxy-phenoxy, C₃₋₈-cycloalk(en)yl-C₁₋₆-alk(en/yn)yloxy-phenoxy, halo-C₁₋₆-alk(en/yn)yloxy-phenoxy, halo-C₃₋₈-cycloalk(en)yloxy-phenoxy, halo-C₃₋₈-cycloalk(en)yl-C₁₋₆-alk(en/yn)yloxy-phenoxy, C₁₋₆-alk(en/yn)ylamino-phenoxy, di-(C₁₋₆-alk(en/yn)yl)amino-phenoxy, C₁₋₆-alk(en/yn)yl-CO—NH-phenoxy and C₁₋₆-alk(en/yn)yl-sulfonamide-phenoxy
the terms “C₁₋₆-alk(en/yn)yl”, “C₃₋₈-cycloalk(en)yl”, “C₃₋₈-heterocycloalk(en)yl”, “Aryl”, “Heteroaryl”, “halo-C₁₋₆-alk(en/yn)yl”, “halo-C₃₋₈-cycloalk(en)yl”, “halo-phenoxy”, “amino-C₁₋₆-alk(en/yn)yl”, “amino-C₃₋₈-cycloalk(en)yl” and “amino-C₃₋₈-cycloalk(en)yl-C₁₋₆-alk(en/yn)yl” are as defined above.
Any C₁₋₆-alk(en/yn)yl which is mentioned either alone or as a part of a larger substituent independently contains 1,2,3, 4, 5 or 6 carbon atoms.
Any C₁₋₈-alk(en/yn)yl which is mentioned either alone or as a part of a larger substituent independently contains 1, 2, 3, 4, 5, 6, 7 or 8 carbon atoms.
Any C₃₋₈-cycloalk(en)yl which is mentioned either alone or as a part of a larger substituent independently contains 3, 4, 5, 6, 7 or 8 carbon atoms.
Any C₃₋₈-heterocycloalk(en)yl which is mentioned either alone or as a part of a larger substituent independently contains 2, 3, 4, 5, 6 or 7 carbon atoms and 1 or 2 heteroatoms.
Any Aryl which is mentioned either alone or as a part of a larger substituent independently contains 5, 6, 7, 8, 9 or 10 carbon atoms.
Any Heteroaryl which is mentioned either alone or as a part of a larger substituent independently contains 5, 6, 7, 8, 9 or 10 atoms selected from 1, 2, 3, 4, 5, 6, 7, 8 or 9 carbon atoms and 1, 2, 3 or 4 heteroatoms.

In an embodiment of formula 6 said compound is selected from the group consisting of:
Hexanoic acid (4-bromo-2,6-dimethyl-phenyl)-amide,
N-(4-Bromo-2,6-dimethyl-phenyl)-2-(4-fluoro-phenyl)-acetamide,
N-(2-Bromo-4,6-dimethyl-phenyl)-2-(4-fluoro-phenyl)-acetamide,
N-(2-Bromo-4,6-dimethyl-phenyl)-3,3-dimethyl-butyramide,
N-(2-Bromo-4,6-dimethyl-phenyl)-2-cyclopentyl-acetamide,
N-(2-Bromo-4,6-dichloro-phenyl)-3,3-dimethyl-butyramide,
N-(2-Bromo-4,6-dichloro-phenyl)-2-(4-fluoro-phenyl)-acetamide,
N-(2-Bromo-4,6-dichloro-phenyl)-2-cyclopentyl-acetamide,
Heptanoic acid (4-bromo-2,6-dimethyl-phenyl)-amide,
Cyclohexanecarboxylic acid (4-bromo-2,6-dimethyl-phenyl)-amide,
N-(4-Bromo-2,6-dimethyl-phenyl)-2-thiophen-2-yl-acetamide,
2-Phenyl-cyclopropanecarboxylic acid (4-bromo-2,6-dimethyl-phenyl)-amide,
N-(4-Bromo-2,6-dimethyl-phenyl)-2-(4-chloro-phenyl)-acetamide,
Pentanoic acid (4-bromo-2,6-dimethyl-phenyl)-amide,
Octanoic acid (4-bromo-2,6-dimethyl-phenyl)-amide,
N-(4-Bromo-2,6-dimethyl-phenyl)-2-cyclopentyl-acetamide,
2-Bicyclo[2.2.1]hept-2-yl-N-(2,4-difluoro-6-morpholin-4-yl-phenyl)-acetamide,
(S)-2-Amino-N-{2,6-dimethyl-4-[methyl-(4-trifluoromethyl-benzyl)-amino]-phenyl}-3-methyl-butyramide,
(S)-2-Amino-4-methyl-pentanoic acid {2,6-dimethyl-4-[methyl-(4-trifluoromethyl-benzyl)-amino]-phenyl}-amide,
(4-Bromo-2,6-dimethyl-phenyl)-carbamic acid ethyl ester,
(4-Bromo-2,6-dimethyl-phenyl)-carbamic acid propyl ester,
N-(2-Amino-4-bromo-6-methyl-phenyl)-3,3-dimethyl-butyramide,
2-Cyclopentyl-N-{2,6-dimethyl-4-[2-(4-trifluoromethyl-phenyl)-pyrrolidin-1-yl]-phenyl}-acetamide,
N-(4-Azepan-1-yl-2,6-dimethyl-phenyl)-2-cyclopentyl-acetamide,
2-Cyclopentyl-N-(2,6-dimethyl-4-pyrrol-1-yl-phenyl)-acetamide,
N-(3′-Amino-3,5-dimethyl-biphenyl-2-yl)-2-(4-fluoro-phenyl)-acetamide,
N-(4′-Dimethylamino-3,5-dimethyl-biphenyl-2-yl)-2-(4-fluoro-phenyl)-acetamide,
N-(2,4-Dimethyl-6-quinolin-3-yl-phenyl)-2-(4-fluoro-phenyl)-acetamide,
2-(4-Fluoro-phenyl)-N-(4′-hydroxy-3′-methoxy-3,5-dimethyl-biphenyl-2-yl)-acetamide,
2-(4-Fluoro-phenyl)-N-(3′-hydroxy-3,5-dimethyl-biphenyl-2-yl)-acetamide,
2-(4-Fluoro-phenyl)-N-(2′-methanesulfonylamino-3,5-dimethyl-biphenyl-2-yl)-acetamide,
N-(4′-Isopropyl-3,5-dimethyl-biphenyl-2-yl)-3,3-dimethyl-butyramide,
2-Cyclopentyl-N-(3,5-dimethyl-biphenyl-2-yl)-acetamide,
N-(4′-Fluoro-3,5-dimethyl-biphenyl-2-yl)-2-(4-fluoro-phenyl)-acetamide,
N-(3,5-Dimethyl-3′,5′-bis-trifluoromethyl-biphenyl-2-yl)-2-(4-fluoro-phenyl)-acetamide,
N-(3′-Acetylamino-3,5-dimethyl-biphenyl-2-yl)-2-(4-fluoro-phenyl)-acetamide,
2-(4-Fluoro-phenyl)-N-(2′-methoxy-3,5-dimethyl-biphenyl-2-yl)-acetamide,
N-(3,5-Dimethyl-4′-vinyl-biphenyl-2-yl)-2-(4-fluoro-phenyl)-acetamide,
N-(3′-Cyano-3,5-dimethyl-biphenyl-2-yl)-2-(4-fluoro-phenyl)-acetamide,
N-(3,5-Dimethyl-3′-trifluoromethoxy-biphenyl-2-yl)-2-(4-fluoro-phenyl)-acetamide,
N-[2-(2,3-Dihydro-benzo[1,4]dioxin-6-yl)-4,6-dimethyl-phenyl]-2-(4-fluoro-phenyl)-acetamide,
N-[2,4-Dimethyl-6-(2,2,5-trimethyl-2,3-dihydro-benzofuran-7-yl)-phenyl]-2-(4-fluoro-phenyl)-acetamide,
N-[2,6-Dimethyl-4-(4-trifluoromethyl-benzylamino)-phenyl]-acetamide,
N-{2,6-Dimethyl-4-[methyl-(4-trifluoromethyl-benzyl)-amino]-phenyl}-acetamide,
{4-[(5-Chloro-thiophen-2-ylmethyl)-amino]-2,6-dimethyl-phenyl}-carbamic acid propyl ester,
[4-(4-Fluoro-benzylamino)-2,6-dimethyl-phenyl]-carbamic acid propyl ester,
[2,6-Dimethyl-4-(4-trifluoromethyl-benzylamino)-phenyl]-carbamic acid propyl ester,
[4-(3-Fluoro-4-trifluoromethyl-benzylamino)-2,6-dimethyl-phenyl]-carbamic acid propyl ester,
{2,6-Dimethyl-4-[(4-methyl-2-phenyl-pyrimidin-5-ylmethyl)-amino]-phenyl}-carbamic acid propyl ester,
{2,6-Dimethyl-4-[(6-p-tolyloxy-pyridin-3-ylmethyl)-amino]-phenyl}-carbamic acid propyl ester,
{4-[(6-Methoxy-pyridin-3-ylmethyl)-amino]-2,6-dimethyl-phenyl}-carbamic acid propyl ester,
{4-[(3-Fluoro-4-trifluoromethyl-benzyl)-methyl-amino]-2,6-dimethyl-phenyl}-carbamic acid propyl ester,
2-Cyclopentyl-N-[2,6-dimethyl-4-(4-trifluoromethyl-benzylamino)-phenyl]-acetamide,
2-Cyclopentyl-N-{2,6-dimethyl-4-[methyl-(4-trifluoromethyl-benzyl)-amino]-phenyl}-acetamide,
2-Cyclopentyl-N-{2,6-dimethyl-4-[(6-trifluoromethyl-pyridin-3-ylmethyl)-amino]-phenyl}-acetamide,
N-{2,6-Dimethyl-4-[(6-trifluoromethyl-pyridin-3-ylmethyl)-amino]phenyl}-3,3-dimethyl-butyramide,
N-{2-Bromo-4-[(5-chloro-thiophen-2-ylmethyl)-amino]-6-trifluoromethyl-phenyl}-3-cyclohexyl-propionamide,
{4-[(3-Fluoro-phenylamino)-methyl]-2,6-dimethyl-phenyl}-carbamic acid ethyl ester,
{2,6-Dimethyl-4-[(4-trifluoromethyl-phenylamino)-methyl]-phenyl}-carbamic acid ethyl ester,
2-Cyclopentyl-N-{4-[(3-fluoro-phenylamino)-methyl]-2,6-dimethyl-phenyl}-acetamide,
N-{4-[(3-Chloro-phenylamino)-methyl]-2,6-dimethyl-phenyl}-2-cyclopentyl-acetamide,
2-Cyclopentyl-N-{4-[(3-methoxy-phenylamino)-methyl]-2,6-dimethyl-phenyl}-acetamide,
N-{4-[(4-Chloro-phenylamino)-methyl]-2,6-dimethyl-phenyl}-2-cyclopentyl-acetamide,
2-Cyclopentyl-N-{4-[(3,4-difluoro-phenylamino)-methyl]-2,6-dimethyl-phenyl}-acetamide,
2-Cyclopentyl-N-{2,6-dimethyl-4-[(4-trifluoromethyl-phenylamino)-methyl]-phenyl}-acetamide,
2-Cyclopentyl-N-[2,6-dimethyl-4-(p-tolylamino-methyl)-phenyl]-acetamide,
2-Cyclopentyl-N-{2,6-dimethyl-4-[(3-trifluoromethyl-phenylamino)-methyl]-phenyl}-acetamide,
2-Cyclopentyl-N-{4-[(3,5-difluoro-phenylamino)-methyl]-2,6-dimethyl-phenyl}-acetamide,
{4-[(4-Fluoro-phenylamino)-methyl]-2,6-dimethyl-phenyl}-carbamic acid propyl ester,
{4-[(4-Chloro-phenylamino)-methyl]-2,6-dimethyl-phenyl}-carbamic acid propyl ester,
{2,6-Dimethyl-4-[(4-trifluoromethyl-phenylamino)-methyl]-phenyl}-carbamic acid propyl ester,
{4-[(3,5-Difluoro-phenylamino)-methyl]-2,6-dim ethyl-phenyl}-carbamic acid propyl ester,
{4-[(3-Fluoro-phenylamino)-methyl]-2,6-dimethyl-phenyl}-carbamic acid propyl ester,
N-(4-Bromo-2-methyl-6-morpholin-4-yl-phenyl)-3,3-dimethyl-butyramide,
{4-[(4-Methoxyphenylamino)-methyl]-2,6-dimethylphenyl}-carbamic acid propyl ester,
(R)-2-Amino-4-methylpentanoic acid [2,6-dimethyl-4-(4-trifluoromethylbenzylamino)-phenyl]-amide,
Pentanoic acid {4-[(4-chlorophenylamino)-methyl]-2,6-dimethylphenyl}-amide,
2-(4-Chlorophenyl)-N-{4-[(4-chlorophenylamino)-methyl]-2,6-dimethyl phenyl}-acetamide,
{2,6-Dimethyl-4-[(4-trifluoromethylphenylamino)-methyl]-phenyl}-carbamic acid 2-methoxyethyl ester,
N-{4-[(5-Chloro-pyridin-2-ylamino)-methyl]-2,6-dimethylphenyl}-2-cyclopentylacetamide,
2-Cyclopentyl-N-{4-[(2,6-dichloro-pyridin-4-ylamino)-methyl]-2,6-dimethylphenyl}-acetamide,
N-{2-Chloro-6-methyl-4-[(6-trifluoromethyl-pyridin-3-ylmethyl)-amino]-phenyl}-2-(3-fluoro-phenyl)-acetamide,
N-[2-Chloro-6-trifluoromethyl-4-(4-trifluoromethylbenzylamino)-phenyl]-2-cyclopentylacetamide,
[2-Amino-6-methyl-4-(4-trifluoromethylbenzylamino)-phenyl]-carbamic acid ethyl ester,
3,3-Dimethyl-N-[2-methyl-6-morpholin-4-yl-4-(4-trifluoromethylbenzylamino)-phenyl]-butyramide,
2-Cyclopentyl-N-{2,6-dichloro-4-[(4-fluoro-phenylamino)-methyl]-phenyl}-acetamide,
2-Cyclopentyl-N-{2,6-dichloro-4-[(5-trifluoromethylpyridin-2-ylamino)-methyl]-phenyl}-acetamide;
   and pharmaceutically acceptable salts thereof.

Compounds according to formula 6 can be prepared as described in WO2006/029623.

An embodiment of the invention relates to a method wherein said compound is a compound according to formula 7:

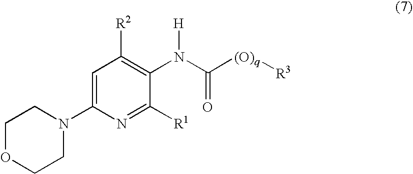

wherein:
q is 0 or 1;
each of R¹ and R² is independently selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy; and
R³ is selected from the group consisting of C₁₋₈-alk(en/yn)yl, C₃₋₈-cycloalk(en)yl, C₃₋₈-cycloalk(en)yl-C₁₋₆-alk(en/yn)yl, optionally substituted Aryl-C₁₋₆-alk(en/yn)yl, optionally substituted Aryl-C₃₋₈-cycloalk(en)yl, optionally substituted Aryl-C₃₋₈-cycloalk(en)yl-C₁₋₆-alk(en/yn)yl, C₁₋₆-alk(en/yn)yl-C₃₋₈-heterocycloalk(en)yl-C₁₋₆-alk(en/yn)yl, C₃₋₈-heterocycloalk(en)yl-C₁₋₆-alk(en/yn)yl, C₁₋₆-alk(en/yn)yl-C₃₋₈-heterocycloalk(en)yl-C₁₋₆-alk(en/yn)yl, Heteroaryl-C₁₋₆-alk(en/yn)yl, Heteroaryl-C₃₋₈-cycloalk(en)yl, Heteroaryl-C₃₋₈-cycloalk(en)yl-C₁₋₆-alk(en/yn)yl, NR⁴R⁵—C₁₋₆-alk(en/yn)yl, NR⁴R⁵—C₃₋₈-cycloalk(en)yl, NR⁴R⁵—C₃₋₈-cycloalk(en)yl-C₁₋₆-alk(en/yn)yl, C₁₋₆-alk(en/yn)yloxy-C₁₋₆-alk(en/yn)yl, C₃₋₈-cycloalk(en)yloxy-C₁₋₆-alk(en/yn)yl, C₃₋₈-cycloalk(en)yl-C₁₋₆-alk(en/yn)yloxy-C₁₋₆-alk(en/yn)yl, halo-C₁₋₆-alk(en/yn)yl, halo-C₃₋₈-cycloalk(en)yl and halo-C₃₋₈-cycloalk(en)yl-C₁₋₆-alk(en/yn)yl; wherein
   each of R⁴ and R⁵ is independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl;
      as the free base or a salt thereof.

In an embodiment of formula 7:
each of R¹ and R² is independently selected from the group consisting of C₁₋₆-alk(en/yn)yl, C₃₋₈-cycloalk(en)yl, C₃₋₈-cycloalk(en)yl-C₁₋₆-alk(en/yn)yl, C₁₋₆-alk(en/yn)yloxy and halogen.
R³ is selected from the group consisting of C₁₋₈-alk(en/yn)yl, C₃₋₈-cycloalk(en)yl-C₁₋₆-alk(en/yn)yl, optionally substituted Aryl-C₁₋₆-alk(en/yn)yl, optionally substituted Aryl-C₃₋₈-cycloalk(en)yl and Heteroaryl-C₁₋₆-alk(en/yn)yl.
optionally substituted Aryl may be substituted with one or more substituent indepentyl selected from the group consisting of halogen, C₁₋₆-alk(en/yn)yl, halo-C₁₋₆-alk(en/yn)yl and C₁₋₆-alk(en/yn)yloxy.

In an embodiment of the present invention, the following definitions are applied for formula 7:
The term “heteroatom” refers to a nitrogen, oxygen or sulphur atom.
“Halogen” means fluoro, chloro, bromo or iodo. “Halo” means halogen.
“Cyano” designates

C≡N
which is attached to the remainder of the molecule via the carbon atom.
The expression “C₁₋₆-alk(en/yn)yl” means C₁₋₆-alkyl, C₂₋₆-alkenyl or C₂₋₆-alkynyl. The term “C₁₋₆-alkyl” refers to a branched or unbranched alkyl group having from one to six carbon atoms, including but not limited to methyl, ethyl, prop-1-yl, prop-2-yl, 2-methyl-prop-1-yl, 2-methyl-prop-2-yl, 2,2-dimethyl-prop-1-yl, but-1-yl, but-2-yl, 3-methyl-but-1-yl, 3-methyl-but-2-yl, pent-1-yl, pent-2-yl, pent-3-yl, hex-1-yl, hex-2-yl and hex-3-yl.
The term “C₂₋₆-alkenyl” refers to a branched or unbranched alkenyl group having from two to six carbon atoms and one double bond, including but not limited to ethenyl, propenyl, and butenyl.
The term “C₂₋₆-alkynyl” refers to a branched or unbranched alkynyl group having from two to six carbon atoms and one triple bond, including but not limited to ethynyl, propynyl and butynyl.
The expression “C₁₋₈-alk(en/yn)yl” means C₁₋₈alkyl, C₂₋₈-alkenyl or C₂₋₈-alkynyl. The term “C₁₋₈-alkyl” refers to a branched or unbranched alkyl group having from one to eight carbon atoms, including but not limited to methyl, ethyl, prop-1-yl, prop-2-yl, 2-methyl-prop-1-yl, 2-methyl-prop-2-yl, 2,2-dimethyl-prop-1-yl, but-1-yl, but-2-yl, 3-methyl-but-1-yl, 3-methyl-but-2-yl, pent-1-yl, pent-2-yl, pent-3-yl, hex-1-yl, hex-2-yl, hex-3-yl, 2-methyl-4,4-dimethyl-pent-1-yl and hept-1-yl.
The term “C₂₋₈-alkenyl” refers to a branched or unbranched alkenyl group having from two to eight carbon atoms and one double bond, including but not limited to ethenyl, propenyl, and butenyl.
The term “C₂₋₈-alkynyl” refers to a branched or unbranched alkynyl group having from two to eight carbon atoms and one triple bond, including but not limited to ethynyl, propynyl and butynyl.
The expression “C₃₋₈-cycloalk(en)yl” means C₃₋₈-cycloalkyl or C₃₋₈-cycloalkenyl The term “C₃₋₈-cycloalkyl” designates a monocyclic or bicyclic carbocycle having three to eight carbon atoms, including but not limited to cyclopropyl, cyclopentyl, cyclohexyl, bicycloheptyl such as 2-bicyclo[2.2.1]heptyl.
The term “C₃₋₈-cycloalkenyl” designates a monocyclic or bicyclic carbocycle having three to eight carbon atoms and one double bond, including but not limited to cyclopentenyl and cyclohexenyl.
The term “C₃₋₈-heterocycloalk(en)yl” means C₃₋₈-heterocycloalkyl or C₃₋₈-heterocycloalkenyl.
The term “C₃₋₈-heterocycloalkyl” designates a monocyclic or bicyclic ring system wherein the ring is formed by 3 to 8 atoms selected from 2-7 carbon atoms and 1 or 2 heteroatoms independently selected from nitrogen, oxygen and sulphur atoms. Examples of C₃₋₈-heterocycloalkyls are pyrrolidine, azepan, morpholine, piperidine, piperazine and tetrahydrofuran.
The term “C₃₋₈-heterocycloalkenyl” designates a monocyclic or bicyclic ring system with one double bond, wherein the ring is formed by 3 to 8 atoms selected from 2-7 carbon atoms and 1 or 2 heteroatoms independently selected from nitrogen, oxygen and sulphur atoms. Examples of C₃₋₈-heterocycloalkenyls are dihydropyrrole, dihydrofuran and dihydrothiophene.
When C₃₋₈-heterocycloalk(en)yl comprises nitrogen then C₃₋₈-heterocycloalk(en)yl is attached to the remainder of the molecule via a carbon atom or nitrogen atom of the heterocyclic ring.
When C₃₋₈-heterocycloalk(en)yl does not comprise nitrogen then C₃₋₈-heterocycloalk(en)yl is attached to the remainder of the molecule via a carbon atom of the heterocyclic ring.
The term “halo-C₁₋₆-alk(en/yn)yl” designates C₁₋₆-alk(en/yn)yl being substituted with halogen, including but not limited to trifluoromethyl.
Similarly, “halo-C₃₋₈-cycloalk(en)yl” designates C₃₋₈-cycloalk(en)yl being substituted with halogen, including but not limited to chlorocyclopropane and chlorocyclohexane.
Similarly, “halo-C₃₋₈-cycloalk(en)yl-C₁₋₆-alk(en/yn)yl” designates halo-C₃₋₈-cycloalk(en)yl being attached to the remainder of the molecule via C₁₋₆-alk(en/yn)yl.
The term “C₁₋₆-alk(en/yn)yloxy” designates C₁₋₆-alk(en/yn)yl being attached to the remainder of the molecule via an oxygen atom. Similarly, “C₃₋₈-cycloalk(en)yloxy” designates C₃₋₈-cycloalk(en)yl being attached to the remainder of the molecule via an oxygen atom.
In the expressions “C₃₋₈-cycloalk(en)yl-C₁₋₆-alk(en/yn)yl”, “Aryl-C₁₋₆-alk(en/yn)yl”, “Aryl-C₃₋₈-cycloalk(en)yl”, “Aryl-C₃₋₈-cycloalk(en)yl-C₁₋₆-alk(en/yn)yl”, “C₃₋₈-heterocycloalk(en)yl-C₁₋₆-alk(en/yn)yl”, “C₁₋₆-alk(en/yn)yl-C₃₋₈-heterocycloalk(en)yl-C₁₋₆-alk(en/yn)yl”, “Heteroaryl-C₁₋₆-alk(en/yn)yl”, “Heteroaryl-C₃₋₈-cycloalk(en)yl”, “Heteroaryl-C₃₋₈-cycloalk(en)yl-C₁₋₆-alk(en/yn)yl”, “NR⁴R⁵—C₁₋₆-alk(en/yn)yl”, “NR⁴R⁵—C₃₋₈-cycloalk(en)yl”, “NR⁴R⁵—C₃₋₈-cycloalk(en)yl-C₁₋₆-alk(en/yn)yl”, “C₃₋₈-cycloalk(en)yl —C₁₋₆-alk(en/yn)yloxy”, “C₁₋₆-alk(en/yn)yloxy-C₁₋₆-alk(en/yn)yl”, “C₃₋₈-cycloalk(en)yloxy-C₁₋₆-alk(en/yn)yl” and “C₃₋₈-cycloalk(en)yl-C₁₋₆-alk(en/yn)yloxy-C₁₋₆-alk(en/yn)yl” the terms “C₁₋₆-alk(en/yn)yl”, “C₃₋₈-cycloalk(en)yl”, “Aryl”, “C₃₋₈-heterocycloalk(en)yl”, “Heteroaryl”, “C₁₋₆-alk(en/yn)yloxy” and “C₃₋₈-cycloalk(en)yloxy” are as defined above.
The term “Heteroaryl” refers to monocyclic or bicyclic heteroaromatic systems being selected from the group consisting of pyridine, thiophene, furan, pyrrole, pyrazole, triazole, tetrazole, oxazole, imidazole, thiazole, benzofuran, benzothiophene and indole.
The term Aryl designates monocyclic or bicyclic aromatic systems being selected from the group consisting of phenyl and naphthyl.
The term “optionally substituted Aryl-C₁₋₆-alk(en/yn)yl” designates Aryl-C₁₋₆-alk(en/yn)yl wherein the Aryl moiety is optionally substituted, such as with 1,2 or 3 substituents independently selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-Cl_{—}6-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy.
Similarly, “optionally substituted Aryl-C₃₋₈-cycloalk(en)yl” designates Aryl-C₃₋₈-cycloalk(en)yl wherein the Aryl moiety is optionally substituted, such as with 1, 2 or 3 substituents independently selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy.
Similarly, “optionally substituted Aryl-C₃₋₈-cycloalk(en)yl-C₁₋₆-alk(en/yn)yl” designates Aryl-C₃₋₈-cycloalk(en)yl-C₁₋₆-alk(en/yn)yl wherein the Aryl moiety is optionally substituted, such as with 1, 2 or 3 substituents independently selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy.

In an embodiment of formula 7 said compound is selected from the group consisting of:
(2,4-Dimethyl-6-morpholin-4-yl-pyridin-3-yl)-carbamic acid benzyl ester;
(2,4-Dimethyl-6-morpholin-4-yl-pyridin-3-yl)-carbamic acid 2-chloro-benzyl ester;
2-(4-Chloro-phenyl)-(2,4-dimethyl-6-morpholin-4-yl-pyridin-3-yl)-acetamide;
2-Phenyl-cyclopropanecarboxylic acid (2,4-dimethyl-6-morpholin-4-yl-pyridin-3-yl)-amide;
N-(2,4-Dimethyl-6-morpholin-4-yl-pyridin-3-yl)-2-thiophen-2-yl-acetamide;
3-Cyclohexyl-N-(2,4-dimethyl-6-morpholin-4-yl-pyridin-3-yl)-propionamide;
(2,4-Dimethyl-6-morpholin-4-yl-pyridin-3-yl)-carbamic acid isobutyl ester;
3-(3-Chloro-phenyl)-N-(2,4-dimethyl-6-morpholin-4-yl-pyridin-3-yl)-propionamide;
N-(2,4-Dimethyl-6-morpholin-4-yl-pyridin-3-yl)-2-(3,5-dimethyl-phenyl)-acetamide;
N-(2,4-Dimethyl-6-morpholin-4-yl-pyridin-3-yl)-3-p-tolyl-propionamide;
2-(3-Chloro-phenyl)-N-(2,4-dimethyl-6-morpholin-4-yl-pyridin-3-yl)-acetamide;
2-(3,4-Dichloro-phenyl)-N-(2,4-dimethyl-6-morpholin-4-yl-pyridin-3-yl)-acetamide;
N-(2,4-Dimethyl-6-morpholin-4-yl-pyridin-3-yl)-2-thiophen-3-yl-acetamide;
N-(2,4-Dimethyl-6-morpholin-4-yl-pyridin-3-yl)-2-p-tolyl-acetamide;
2-(3-Bromo-phenyl)-N-(2,4-dimethyl-6-morpholin-4-yl-pyridin-3-yl)-acetamide;
N-(2,4-Dimethyl-6-morpholin-4-yl-pyridin-3-yl)-2-(3-trifluoromethyl-phenyl)-acetamide;
N-(2,4-Dimethyl-6-morpholin-4-yl-pyridin-3-yl)-2-phenyl-acetamide;
3,5,5-Trimethyl-hexanoic acid (2,4-dimethyl-6-morpholin-4-yl-pyridin-3-yl)-amide;
Octanoic acid (2,4-dimethyl-6-morpholin-4-yl-pyridin-3-yl)-amide;
N-(2,4-Dimethyl-6-morpholin-4-yl-pyridin-3-yl)-2-naphthalen-2-yl-acetamide;
Heptanoic acid (2,4-dimethyl-6-morpholin-4-yl-pyridin-3-yl)-amide;
N-(2,4-Dimethyl-6-morpholin-4-yl-pyridin-3-yl)-2-(3,4-dimethyl-phenyl)-acetamide;
2-Cyclohex-1-enyl-N-(2,4-dimethyl-6-morpholin-4-yl-pyridin-3-yl)-acetamide;
N-(2,4-Dimethyl-6-morpholin-4-yl-pyridin-3-yl)-2-(4-methoxy-3-methyl-phenyl)-acetamide;
N-(2,4-Dimethyl-6-morpholin-4-yl-pyridin-3-yl)-2-(4-methoxy-phenyl)-acetamide;
N-(2,4-Dimethyl-6-morpholin-4-yl-pyridin-3-yl)-3-(4-methoxy-phenyl)-propionamide;
N-(2,4-Dimethyl-6-morpholin-4-yl-pyridin-3-yl)-2-m-tolyl-acetamide;
N-(2,4-Dimethyl-6-morpholin-4-yl-pyridin-3-yl)-2-(4-fluoro-phenyl)-acetamide;
N-(2,4-Dimethyl-6-morpholin-4-yl-pyridin-3-yl)-3,3-dimethyl-butyramide;
N-(2,4-Dimethyl-6-morpholin-4-yl-pyridin-3-yl)-2-(3-fluoro-phenyl)-acetamide;
2-Bicyclo[2.2.1]hept-2-yl-N-(2,4-dimethyl-6-morpholin-4-yl-pyridin-3-yl)-acetamide;
2-(3,4-Difluoro-phenyl)-N-(2,4-dimethyl-6-morpholin-4-yl-pyridin-3-yl)-acetamide;
4-Methyl-pentanoic acid (2,4-dimethyl-6-morpholin-4-yl-pyridin-3-yl)-amide;
2-Cyclopent-2-enyl-N-(2,4-dimethyl-6-morpholin-4-yl-pyridin-3-yl)-acetamide;
2-Cyclohexyl-N-(2,4-dimethyl-6-morpholin-4-yl-pyridin-3-yl)-acetamide;
5-Methyl-hexanoic acid (2,4-dimethyl-6-morpholin-4-yl-pyridin-3-yl)-amide;
2-Cyclopentyl-N-(2,4-dimethyl-6-morpholin-4-yl-pyridin-3-yl)-acetamide;
3-Cyclopentyl-N-(2,4-dimethyl-6-morpholin-4-yl-pyridin-3-yl)-propionamide; and
Hexanoic acid (2,4-dimethyl-6-morpholin-4-yl-pyridin-3-yl)-amide
N-(4-Chloro-2-methoxy-6-morpholin-4-yl-pyridin-3-yl)-2-cyclopentylacetamide
N-(2-Chloro-4-methoxy-6-morpholin-4-yl-pyridin-3-yl)-2-cyclopentylacetamide
N-(2-Chloro-4-methoxy-6-morpholin-4-yl-pyridin-3-yl)-3,3-dimethylbutyramide
N-(4-Chloro-2-methoxy-6-morpholin-4-yl-pyridin-3-yl)-3,3-dimethylbutyramide
N-(4-Chloro-2-methoxy-6-morpholin-4-yl-pyridin-3-yl)-propionamide;
   and salts thereof.

Compounds according to formula 7 can be prepared as described in WO2006/092143.

An embodiment of the invention relates to a method wherein said compound is a compound according to formula 8:

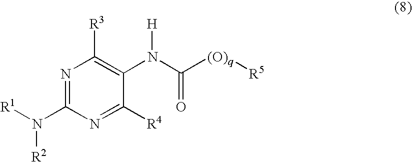

wherein:
q is 0 or 1;
R¹ and R² are independently selected from the group consisting of hydrogen and optionally substituted aryl-C₁₋₆-alk(en/yn)yl, provided that R¹ and R² are not both hydrogen, or R¹ and R² together with the nitrogen to which they are attached form a 5 to 7 membered ring optionally containing a further heteroatom;
R³ and R⁴ are independently selected from hydrogen, halogen, cyano, amino, C₁₋₆-alk(en/yn)yl, C₃₋₈-cycloalk(en)yl, halo-C₁₋₆-alk(en/yn)yl, halo-C₃₋₈-cycloalk(en)yl, C₁₋₆-alk(en/yn)yloxy, C₃₋₈-cycloalk(en)yloxy, C₃₋₈-cycloalk(en)yl-C₁₋₆-alk(en/yn)yloxy, halo-C₁₋₆-alk(en/yn)yloxy, halo-C₃₋₈-cycloalk(en)yloxy and halo-C₃₋₈-cycloalk(en)yl-C₁₋₆-alk(en/yn)yloxy, provided that R³ and R⁴ are not both hydrogen;
R⁵ is selected from the group consisting of C₁₋₁₀-alk(en/yn)yl, C₃₋₈-cycloalk(en)yl-C₁₋₆-alk(en/yn)yl, optionally substituted aryl-C₁₋₆-alk(en/yn)yl and optionally substituted aryl;
   as the free base or a salt thereof.

In an embodiment of formula 8
R¹ and R² are independently selected from hydrogen and optionally substituted aryl-C₁₋₆-alk(en/yn)yl, provided that R¹ and R² are not both hydrogen; or
R¹ and R² together with the nitrogen to which they are attached form a 5 to 7 membered ring optionally containing a further hetero atom; wherein
   said further hetero atom is oxygen.
   said ring is a 6 membered ring, wherein said ring is a morpholine ring
R³ and R⁴ are independently selected from amino and C₁₋₆-alk(en/yn)yl, preferably methyl.
R⁵ is selected from the group consisting of C₁₋₁₀-alk(en/yn)yl, C₃₋₈-cycloalk(en)yl-C₁₋₆-alk(en/yn)yl, optionally substituted aryl-C₁₋₆-alk(en/yn)yl and optionally substituted aryl.

In an embodiment of the present invention, the following definitions are applied for formula 8:
The term “heteroatom” refers to a nitrogen, oxygen or sulphur atom.
“Halogen” means fluoro, chloro, bromo or iodo. “Halo” means halogen.
“Cyano” designates

C≡N
which is attached to the remainder of the molecule via the carbon atom.
“Amino” designates NH₂, which is attached to the remainder of the molecule via the nitrogen atom.
The expression “C₁₋₆-alk(en/yn)yl” means C₁₋₆-alkyl, C₂₋₆-alkenyl or C₂₋₆-alkynyl.
The term “C₁₋₆-alkyl” refers to a branched or unbranched alkyl group having from one to six carbon atoms, including but not limited to methyl, ethyl, prop-1-yl, prop-2-yl, 2-methyl-prop-1-yl, 2-methyl-prop-2-yl, 2,2-dimethyl-prop-1-yl, but-1-yl, but-2-yl, 3-methyl-but-1-yl, 3-methyl-but-2-yl, pent-1-yl, pent-2-yl, pent-3-yl, hex-1-yl, hex-2-yl and hex-3-yl.
The term “C₂₋₆-alkenyl” refers to a branched or unbranched alkenyl group having from two to six carbon atoms and one double bond, including but not limited to ethenyl, propenyl and butenyl.
The term “C₂₋₆-alkynyl” refers to a branched or unbranched alkynyl group having from two to six carbon atoms and one triple bond, including but not limited to ethynyl, propynyl and butynyl.
The expression “C₁₋₁₀-alk(en/yn)yl” means C₁₋₁₀-alkyl, C₂₋₁₀-alkenyl or C₂₋₁₀-alkynyl.
The term “C₁₋₁₀-alkyl” refers to a branched or unbranched alkyl group having from one to ten carbon atoms, including but not limited to methyl, ethyl, prop-1-yl, prop-2-yl, 2-methyl-prop-1-yl, 2-methyl-prop-2-yl, 2,2-dimethyl-prop-1-yl, but-1-yl, but-2-yl, 3-methyl-but-1-yl, 3-methyl-but-2-yl, pent-1-yl, pent-2-yl, pent-3-yl, hex-1-yl, hex-2-yl, hex-3-yl, 2-methyl-4,4-dimethyl-pent-1-yl and hept-1-yl.
The term “C₂₋₁₀-alkenyl” refers to a branched or unbranched alkenyl group having from two to ten carbon atoms and one double bond, including but not limited to ethenyl, propenyl and butenyl.
The term “C₂₋₁₀-alkynyl” refers to a branched or unbranched alkynyl group having from two to ten carbon atoms and one triple bond, including but not limited to ethynyl, propynyl and butynyl.
The expression “C₃₋₈-cycloalk(en)yl” means C₃₋₈-cycloalkyl or C₃₋₈-cycloalkenyl.
The term “C₃₋₈-cycloalkyl” designates a monocyclic or bicyclic carbocycle having three to eight carbon atoms, including but not limited to cyclopropyl, cyclopentyl, cyclohexyl, bicycloheptyl such as 2-bicyclo[2.2.1]heptyl.
The term “C₃₋₈-cycloalkenyl” designates a monocyclic or bicyclic carbocycle having three to eight carbon atoms and one double bond, including but not limited to cyclopentenyl and cyclohexenyl.
The term “halo-C₁₋₆-alk(en/yn)yl” designates C₁₋₆-alk(en/yn)yl being substituted with halogen, including but not limited to trifluoromethyl.
The term “halo-C₁₋₆-alk(en/yn)yloxy” designates C₁₋₆-alk(en/yn)yloxy being substituted with halogen, including but not limited to trifluoromethyloxy.
Similarly, “halo-C₃₋₈-cycloalk(en)yl” designates C₃₋₈-cycloalk(en)yl being substituted with halogen, including but not limited to chlorocyclopropane and chlorocyclohexane.
Similarly, “halo-C₃₋₈-cycloalk(en)yloxy” designates C₃₋₈-cycloalk(en)yloxy being substituted with halogen, including but not limited to chlorocyclopropyloxy and chlorocyclohexyloxy.
Similarly, “halo-C₃₋₈-cycloalk(en)yl-C₁₋₆-alk(en/yn)yloxy” designates halo-C₃₋₈-cycloalk(en)yl being attached to the remainder of the molecule via C₁₋₆-alk(en/yn)yloxy.
The term “C₁₋₆-alk(en/yn)yloxy” designates C₁₋₆-alk(en/yn)yl being attached to the remainder of the molecule via an oxygen atom.
Similarly, “C₃₋₈-cycloalk(en)yloxy” designates C₃₋₈-cycloalk(en)yl being attached to the remainder of the molecule via an oxygen atom.
The term “aryl” designates monocyclic or bicyclic aromatic systems being selected from the group consisting of phenyl, naphthyl, thiophen, furan, benzothiophen and benzofuran.
The term “optionally substituted aryl-C₁₋₆-alk(en/yn)yl” designates aryl-C₁₋₆-alk(en/yn)yl wherein the aryl moiety is optionally substituted, such as with 1, 2 or 3 substituents independently selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy. Similarly, “optionally substituted aryl” designates aryl wherein the aryl is optionally substituted, such as with 1, 2 or 3 substituents independently selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy.
In the expressions “C₃₋₈-cycloalk(en)yl-C₁₋₆-alk(en/yn)yl”, “aryl-C₁₋₆-alk(en/yn)yl” and “C₃₋₈-cycloalk(en)yl-C₁₋₆-alk(en/yn)yloxy”, the terms “C₁₋₆-alk(en/yn)yl”, “C₃₋₈-cycloalk(en)yl”, “aryl” and “C₁₋₆-alk(en/yn)yloxy” are as defined above.

In an embodiment of formula 8 said compound is selected from the group consisting of:
N-[4-Amino-6-methyl-2-(4-trifluoromethylbenzylamino)-pyrimidin-5-yl]-2-cyclopentylacetamide,
N-[4-Amino-6-methyl-2-(4-trifluoromethylbenzylamino)-pyrimidin-5-yl]-3,3-dimethylbutyramide,
N-[4-Amino-6-methyl-2-(4-trifluoromethylbenzylamino)-pyrimidin-5-yl]-2-(4-fluorophenyl)-acetamide,
Hexanoic acid [4-amino-6-methyl-2-(4-trifluoromethylbenzylamino)-pyrimidin-5-yl]-amide,
N-[4-Amino-6-methyl-2-(4-trifluoromethylbenzylamino)-pyrimidin-5-yl]-2-(3-chlorophenyl)-acetamide,
2-Cyclopentyl-N-(4,6-dimethyl-2-morpholin-4-yl-pyrimidin-5-yl)-acetamide,
N-(4,6-Dimethyl-2-morpholin-4-yl-pyrimidin-5-yl)-3,3-dimethylbutyramide,
N-(4,6-Dimethyl-2-morpholin-4-ylpyrimidin-5-yl)-2-(4-fluorophenyl)-acetamide,
2-(3,4-Difluorophenyl)-N-(4,6-dimethyl-2-morpholin-4-ylpyrimidin-5-yl)-acetamide,
N-(4,6-Dimethyl-2-morpholin-4-ylpyrimidin-5-yl)-2-(3-fluorophenyl)-acetamide and
Hexanoic acid (4,6-dimethyl-2-morpholin-4-ylpyrimidin-5-yl)-amide;
   and salts thereof.

Compounds according to formula 8 can be prepared as described in WO2007/065449.

An embodiment of the invention relates to a method wherein said compound is the compound of formula 9:

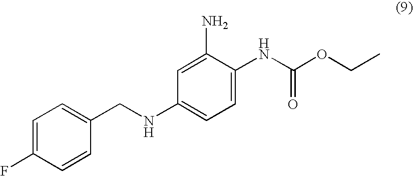

or a pharmaceutically acceptable salt thereof.

The compound according to formula 9 can be prepared as described in EP554543.

An embodiment of the invention relates to a method wherein said compound is wherein said compound is the compound of formula 10:

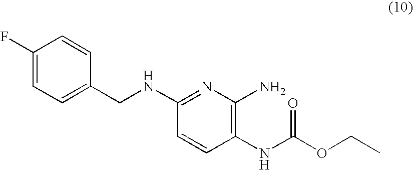

or a pharmaceutically acceptable salt thereof.

The compound according to formula 10 can be prepared as described in Chemiker-Zeitung (1981), 105(7-8), 217-19 and Arzneimittel-Forschung (1993), 43(6), 627-31.

In an embodiment, the invention relates to a method wherein the compound is selected from the group consisting of:
N-(2-amino-4-(4-fluorobenzylamino)-phenyl) carbamic acid ethyl ester;
2-Cyclopentyl-N-(2,6-dimethyl-4-morpholin-4-yl-phenyl)-acetamide;
N-(2,6-Dimethyl-4-morpholin-4-yl-phenyl)-3,3-dimethyl-butyramide;
N-(4,6-Dimethyl-2-morpholin-4-yl-pyrimidin-5-yl)-2-(4-fluoro-phenyl)-acetamide;
Hexanoic acid (2,6-difluoro-4-morpholin-4-yl-phenyl)-amide;
2-Cyclopentyl-N-(4,6-dimethyl-2-morpholin-4-yl-pyrimidin-5-yl)-acetamide;
N-(2-Bromo-4-morpholin-4-yl-6-trifluoromethyl-phenyl)-propionamide;
N-(2,4-Dimethyl-6-morpholin-4-yl-pyridin-3-yl)-3,3-dimethyl-butyramide;
[2-Amino-4-(2,4,6-trimethyl-benzylamino)-phenyl]-carbamic acid ethyl ester; and
2-Cyclopentyl-N-(2-methoxy-6-methyl-4-morpholin-4-yl-phenyl)-acetamide;
   and salts thereof.

The salts of the invention are preferably pharmaceutically acceptable salts. Such salts include pharmaceutical acceptable acid addition salts, pharmaceutically acceptable metal salts, ammonium and alkylated ammonium salts.

The pharmaceutically acceptable salts of the invention are preferably acid addition salts. The acid addition salts of the invention are preferably pharmaceutically acceptable salts of the compounds of the invention formed with non-toxic acids. Acid addition salts include salts of inorganic acids as well as organic acids. Representative examples of suitable inorganic acids include hydrochloric, hydrobromic, hydroiodic, sulfuric, sulfamic, phosphoric and nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, ethanesulfonic, tartaric, ascorbic, pamoic, gluconic, citraconic, aspartic, stearic, palmitic, EDTA, glycolic, p-aminobenzoic, glutamic, bis-methylenesalicylic, methanesulfonic, ethanedisulfonic, itaconic, benzenesulfonic, p-toluenesulfonic acids, theophylline acetic acids, as well as the 8-halotheophyllines, for example 8-bromotheophylline and the like. Further examples of pharmaceutical acceptable inorganic or organic acid addition salts include the pharmaceutically acceptable salts listed in J. Pharm. Sci. 1977, 66, 2, which is incorporated herein by reference.

Examples of metal salts include lithium, sodium, potassium, magnesium salts and the like.

Examples of ammonium and alkylated ammonium salts include ammonium, methyl-, dimethyl-, trimethyl-, ethyl-, hydroxyethyl-, diethyl-, n-butyl-, sec-butyl-, tert-butyl-, tetramethylammonium salts and the like.

Also intended as pharmaceutically acceptable acid addition salts are the hydrates, which the present compounds are able to form.

The compounds of the present invention may have one or more asymmetric centres and it is intended that any optical isomers, as separated, pure or partially purified optical isomers or racemic mixtures thereof are included within the scope of the invention.

Furthermore, when a double bond or a fully or partially saturated ring system is present in the molecule geometric isomers may be formed. It is intended that any geometric isomers, as separated, pure or partially purified geometric isomers or mixtures thereof are included within the scope of the invention. Likewise, molecules having a bond with restricted rotation may form geometric isomers. These are also intended to be included within the scope of the present invention.

Furthermore, some of the compounds of the present invention may exist in different tautomeric forms and it is intended that any tautomeric forms that the compounds are able to form are included within the scope of the present invention.

The compounds of this invention may exist in unsolvated as well as in solvated forms with solvents such as water, ethanol and the like. In general, the solvated forms are considered equivalent to the unsolvated forms for the purposes of this invention. Racemic forms can be resolved into the optical antipodes by known methods, for example, by separation of diastereomeric salts thereof with an optically active acid, and liberating the optically active amine compound by treatment with a base. Another method for resolving racemates into the optical antipodes is based upon chromatography on an optically active matrix. Racemic compounds of the present invention can also be resolved into their optical antipodes, e.g. by fractional crystallization of d- or l- (tartrates, mandelates or camphorsulphonate) salts. The compounds of the present invention may also be resolved by the formation of diastereomeric derivatives.

Additional methods for the resolution of optical isomers, known to those skilled in the art, may be used. Such methods include those discussed by J. Jaques, A. Collet and S. Wilen in “Enantiomers, Racemates, and Resolutions”, John Wiley and Sons, New York (1981).

Optically active compounds can also be prepared from optically active starting materials.

The invention also encompasses prodrugs of the present compounds, which on administration undergo chemical conversion by metabolic processes before becoming pharmacologically active substances.

An aspect of the invention relates to a method for reducing symptoms of or for treating one or more disorders wherein the dopaminergic system is disrupted, said method comprising administering to a host in need thereof an effective amount of a compound able to increase the ion flow through KCNQ potassium channels wherein said compound is given as the only compound having an antipsychotic potential or together with one or more other compounds having an antipsychotic potential. In an embodiment of the invention said compound is given as the only compound having an antipsychotic potential. In an embodiment of the invention said compound is given together with one other compound having an antipsychotic potential. In an embodiment of the invention said compound is given as the only compound having an antipsychotic potential. In an embodiment of the invention said compound is given together with two or more other compounds having an antipsychotic potential. In an embodiment said other compound having an antipsychotic potential is a known antipsychotic compound. In an embodiment of the invention, an appropriate other compound having antipsychotic potential is Sertindole.

An aspect of the invention relates to a method for reducing symptoms of or for treating one or more disorders wherein the dopaminergic system is disrupted, said method comprising administering to a host in need thereof an effective amount of a compound able to increase the ion flow through KCNQ potassium channels wherein said compound is used for the preparation of a pharmaceutical composition.

The present invention also relates to a pharmaceutical composition. The compounds of the invention or salts thereof may be administered alone or in combination with pharmaceutically acceptable carriers or diluents, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the disorder or disease to be treated and the active ingredient chosen.

The pharmaceutical compositions formed by combining the compound of the invention and the pharmaceutical acceptable carriers are then readily administered in a variety of dosage forms suitable for the disclosed routes of administration. The formulations may conveniently be presented in unit dosage form by methods known in the art of pharmacy.

The compounds of this invention are generally utilized as the free substance or as a pharmaceutically acceptable salt thereof. One example is an acid addition salt of a compound having the utility of a free base. When a compound of the invention contains a free base such salts are prepared in a conventional manner by treating a solution or suspension of a free base of the invention with a chemical equivalent of a pharmaceutically acceptable acid. Representative examples are mentioned above.

Pharmaceutical compositions for oral administration may be solid or liquid. Solid dosage forms for oral administration include e.g. capsules, tablets, dragees, pills, lozenges, powders, granules and tablette e.g. placed in a hard gelatine capsule in powder or pellet form or e.g. in the form of a troche or lozenge. Where appropriate, pharmaceutical compositions for oral administration may be prepared with coatings such as enteric coatings or they can be formulated so as to provide controlled release of the active ingredient such as sustained or prolonged release according to methods well known in the art. Liquid dosage forms for oral administration include e.g. solutions, emulsions, suspensions, syrups and elixirs.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid, lower alkyl ethers of cellulose, corn starch, potato starch, gums and the like. Examples of liquid carriers are syrup, peanut oil, olive oil, phospho lipids, fatty acids, fatty acid amines, polyoxyethylene and water.

The carrier or diluent may include any sustained release material known in the art, such as glyceryl monostearate or glyceryl distearate, alone or mixed with a wax.

Any adjuvants or additives usually used for such purposes such as colourings, flavourings, preservatives etc. may be used provided that they are compatible with the active ingredients.

The amount of solid carrier may vary but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

Tablets may be prepared by mixing the active ingredient with ordinary adjuvants or diluents and subsequently compressing the mixture in a conventional tabletting machine.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Depot injectable formulations are also contemplated as being within the scope of the present invention.

For parenteral administration, solutions of the compound of the invention in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Solutions for injections may be prepared by dissolving the active ingredient and possible additives in a part of the solvent for injection, preferably sterile water, adjusting the solution to the desired volume, sterilising the solution and filling it in suitable ampoules or vials. Any suitable additive conventionally used in the art may be added, such as tonicity agents, preservatives, antioxidants, etc.

Other suitable administration forms include suppositories, sprays, ointments, cremes, gels, inhalants, dermal patches, implants, etc.

In an embodiment, the invention relates to a method wherein said compound is administered in an amount of more than 1 mg/day, such as more than 2.5 mg/day, such as about 5 mg/day, about 10 mg/day, about 50 mg/day, about 100 mg/day or about 250 mg/day.

In yet another embodiment, the invention relates to a method wherein said compound is administered in an amount of more than 5 mg/day, more than 10 mg/day or more than 50 mg/day.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the disorder or disease treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.01 to about 1000 mg, such as about 0.01 to 100 mg, preferably from about 0.05 to about 500 mg, and more preferred from about 0.5 mg to about 200 mg.

In yet another embodiment, the invention relates to a method wherein said amount is administered once daily or more than once daily.

For parenteral routes such as intravenous, intrathecal, intramuscular and similar administration, typically doses are in the order of about half the dose employed for oral administration.

Typical examples of recipes for the formulation of the invention are as follows:

1) Tablets containing 5.0 mg of a compound of the invention calculated as the free base:

| | |
|---|---|
| Compound of the invention | 5.0 mg |
| Lactose | 60 mg |
| Maize starch | 30 mg |
| Hydroxypropylcellulose | 2.4 mg |
| Microcrystalline cellulose | 19.2 mg |
| Croscarmellose Sodium Type A | 2.4 mg |
| Magnesium stearate | 0.84 mg |

2) Tablets containing 0.5 mg of a compound of the invention calculated as the free base:

| | |
|---|---|
| Compound of the invention | 0.5 mg |
| Lactose | 46.9 mg |
| Maize starch | 23.5 mg |
| Povidone | 1.8 mg |
| Microcrystalline cellulose | 14.4 mg |
| Croscarmellose Sodium Type A | 1.8 mg |
| Magnesium stearate | 0.63 mg |

3) Syrup containing per millilitre:

| | | |
|---|---|---|
| Compound of the invention | 25 | mg |
| Sorbitol | 500 | mg |
| Hydroxypropylcellulose | 15 | mg |
| Glycerol | 50 | mg |
| Methyl-paraben | 1 | mg |
| Propyl-paraben | 0.1 | mg |
| Ethanol | 0.005 | mL |
| Flavour | 0.05 | mg |
| Saccharin sodium | 0.5 | mg |
| Water | ad 1 | mL |

4) Solution for injection containing per millilitre:

| | | |
|---|---|---|
| Compound of the invention | 0.5 | mg |
| Sorbitol | 5.1 | mg |
| Acetic Acid | 0.05 | mg |
| Saccharin sodium | 0.5 | mg |
| Water | ad 1 | mL |

The present invention covers any combination of the mentioned embodiments as well as any combination of the mentioned embodiments together with any aspect of the invention.

All non-patent references, patents, and patent applications cited and discussed in this specification are incorporated herein by reference in their entirety and to the same extent as if each was individually incorporated by reference.

### EXPERIMENTAL

### Example 1

### Antipsychotic Potential, Part A

Reports have suggested that inhibition of the number of spontaneously active dopaminergic neurones in the ventral tegmental area (VTA), i.e. the mesolimbic system, accounts for the therapeutic potential of an antipsychotic compound (Chiodo and Bunney 1983, J. Neurosci., 5, 2539-2544). In the mesolimbic system, all known antipsychotics initially increase the firing rate of dopaminergic neurons (Tung et al., 1991, J. Neural Transm. Gen Sect., 84(1-2), 53-64). After chronic administration, such antipsychotics eventually (after 3-4 weeks of treatment) decrease the firing rate to below pre-treatment levels (Skarsfeldt 1992, Synapse, 10, 25-33; White and Wang 1983, Science, 221, 1054-1057). This inhibitory effect on dopaminergic neurons is believed to be of therapeutic significance to the antipsychotic effect of antipsychotics (Grace and Bunney 1986, J. Pharmacol. Exp. Ther. 238, 1092-1100). By inference, a compound that causes an acute decrease in the spontaneous firing rate of mesolimbic dopaminergic neurones is anticipated to possess a fast-onset antipsychotic potential, and to have the potential to shorten the time to onset of therapeutic activity of known antipsychotics. The presence of KCNQ subunits on dopamine (DA) neurons in the VTA in rodents is documented (Saganich et al. 2001, J. Neurosci. 21(13)4609-4624; Cooper et al. 2001, J. Neurosci., 21(24)9529-9540, Hansen et al. 2006, JPET 318 (3)1006-1019). In vitro recordings from rat brain slice preparations show that retigabine displays an inhibitory effect on basal spike activity in DA neurons in the VTA (Hansen et al. 2006, JPET 318 (3)1006-1019). The following experiment shows that this effect translates to an in vivo setting where spontaneous DA cell firing in the VTA of anaesthetized rats is inhibited by retigabine.

Subjects. Male Wistar rats (Harlan, The Netherlands) weighing 270-340 g are used. The animals are housed under a 12-hr light/dark cycle under controlled conditions for regular in-door temperature (21±2° C.) and humidity (55±5%) with food and tap water available ad libitum.

Experimental procedure. The rats are anaesthetised with an intraperitoneal injection of chloral hydrate (400 mg/kg). A femoral vein catheter is then inserted for supplementary anaesthetic injections (100 mg/kg) and drug administration. Animals are then mounted in a stereotaxic frame, the skull is exposed, and a hole (0.5×0.5 cm) is drilled above the ventral tegmental area. Extracellular single-cell recordings are performed using electrodes pulled from glass capillaries and filled with 2% Pontamine Sky Blue in 2 M NaCl. The tip of the electrode is broken under microscopic control, yielding an impedance of 2.0-8.0 MΩ at 135 Hz. The electrode is then lowered into the brain, using a hydraulic microdrive, aimed at the following coordinates: 5.5-5.0 mm posterior to Bregma; 0.5-0.9 mm lateral to the midline. Extracellular action potentials are amplified, discriminated and monitored on an oscilloscope and an audiomonitor. Discriminated spikes are collected and analysed using Spike 2 software (Cambridge Electronic Design Ltd., Cambridge, UK) on a PC-based system connected to a CED 1401-interface unit (Cambridge Electronic Design Ltd.). Presumed dopaminergic neurons are typically found 7:0-8.5 mm beneath the brain surface and are characterised by (1) a slow and irregular firing pattern (0.5-10 Hz), and (2) triphasic action potentials with a predominant positive component, a negative component followed by a minor positive component, with an overall duration >2.5 milliseconds (ms) (Bunney et al. 1973, J. Pharmacol. Exp. Ther., 185, 560-571.).

Administration of compound. Once a stable basal firing rate is obtained, cumulated doses of N-(2-amino-4-(4-fluorobenzylamino)-phenyl) carbamic acid ethyl ester (see EP554543); dose range 0.3-6.0 mg/kg; volume range 0.15-1.0 ml/kg), 2-cyclopentyl-N-(2,6-dimethyl-4-morpholin-4-yl-phenyl)-acetamide (see WO2005/087754) (dose range 0.03-0.3 mg/kg; volume range 0.1-1.0 ml/kg) or N-(2,6-dimethyl-4-morpholin-4-yl-phenyl)-3,3-dimethyl-butyramide (see WO2005/087754) (dose range 0.03-0.5 mg/kg; volume range 0.12-1.0 ml/kg) are administered i.v., each injection being separated by at least 3 min. These i.v. doses match the s.c. dose range of 0-10 mg/kg.

Statistical analysis. Drug effects are assessed by statistical comparison of the mean firing rate calculated from the 2-3 min period immediately before the first drug administration (baseline) to the mean firing rate calculated from at least 60 s at the maximal drug effect. Data are analysed statistically by a one-way analysis of variance (ANOVA) followed by Student-Newman-Keuls post hoc test. A p-value less than 0.05 is considered significant.

Results. As can be seen in Table 1, below, N-(2-amino-4-(4-fluorobenzylamino)-phenyl) carbamic acid ethyl ester, 2-cyclopentyl-N-(2,6-dimethyl-4-morpholin-4-yl-phenyl)-acetamide, and N-(2,6-dimethyl-4-morpholin-4-yl-phenyl)-3,3-dimethyl-butyramide significantly, and dose-dependently, inhibited the spontaneous DA cell firing in the VTA of anaesthetised rats following acute administration of compound.

TABLE 1
Effects of compounds on spontaneous DA cell firing in the
VTA of anaesthetised rats.

| | N-(2-amino-4- | 2-Cyclopentyl-N- | N-(2,6-Dimethyl-4- |
|---|---|---|---|
| | (4-fluoro- | (2,6-dimethyl-4- | morpholin-4-yl- |
| Cumulated | benzylamino)- | morpholin-4-yl- | phenyl)-3,3- |
| dose | phenyl) carbamic | phenyl)- | dimethyl- |
| (mg/kg) | acid ethyl ester | acetamide | butyramide |
| 0 (Vehicle) | 97.5 ± 1.2 (6) | 97.8 ± 0.7 (8) | 97.5 ± 0.8 (10) |
| 0.03 | — | 95.1 ± 1.9 (4) | 89.8 ± 4.5 (5) |
| 0.1 | — | 88.2 ± 2.8 (5)* | 81.0 ± 4.0 (5)** |
| 0.25 | — | — | 74.1 ± 6.3 (4)*** |
| 0.3 | 95.4 ± 5.0 (4) | 74.6 ± 3.6 (4)*** | — |
| 0.5 | — | — | 68.1 ± 6.0 (4)*** |
| 0.6 | — | 64.1 ± 7.1 (2)*** | — |
| 0.9 | — | 55.8 ± 6.5 (2)*** | — |
| 1.0 | 90.5 ± 4.6 (5) | — | 57.1 ± 7.9 (3)*** |
| 2.0 | 81.1 ± 5.1 (5)* | — | — |
| 4.0 | 67.1 ± 3.7 (4)*** | — | — |
| 6.0 | 60.6 ± 0.7 (2)*** | — | — |

Spontaneous DA cell firing rates expressed as a percentage of baseline firing rate are listed as mean ± standard error of the mean:
n is indicated in brackets;
*p < 0.05,
**p < 0.01,
***p < 0.001 compared to baseline (pre-drug administration activity).

Conclusion. These data support a potential usefulness of compounds that are able, to increase the ion flow through KCNQ potassium channels (such as, but not limited to, compounds according to any one of formulae 1, 2, 3, 4, 5, 6, 7, 8, 9 or 10) in treatment of psychotic symptoms in humans, with a faster onset of therapeutic activity than known antipsychotics.

The data also suggest that adding compounds that are able to increase the ion flow through KCNQ potassium channels (such as, but not limited to, compounds according to any one of formulae 1, 2, 3, 4, 5, 6, 7, 8, 9 or 10) will shorten the time to onset of clinical therapeutic activity of known antipsychotics if used as adjunctive therapy.

### Example 1

### Antipsychotic Potential, Part B

The administration of psychostimulants such as methylphenidate, cocaine and amphetamine to rodents stimulates an increase in locomotor activity that is caused by the activation of mesolimbic DA receptors in the nucleus accumbens. It is believed that inhibition of a psychostimulant-induced increase in locomotor activity is a reliable method for the evaluation of a compounds antipsychotic potential (Ögren et al., European J. Pharmacol. 1984, 102, 459-464).

Subjects. Male Wistar rats (Taconic, Denmark) weighing 170-240 g are used. The animals are housed under a 12-hr light/dark cycle under controlled conditions for regular in-door temperature (21±2° C.) and humidity (55±5%) with food and tap water available ad libitum. Eight rats are used at each dose level and in the parallel control group receiving the vehicle to the test compound plus d-amphetamine and the group receiving vehicle injections only.

Experimental procedure. The experiment is made in normal light conditions in an undisturbed room. The test substance is injected 30 min before s.c. before the injection of a stimulant drug: d-amphetamine sulphate (0.5 mg/kg) or cocaine (20 mg/kg) or methylphenidate (10 mg/kg). Immediately after injection of the stimulant, the rats are placed individually in the test cages that are placed in a U-frame, equipped with 4 infrared light sources and photocells. The light beams cross the cage 4 cm above the cage floor. Recording of a motility count requires interruption of adjacent light beams, thus avoiding counts induced by stationary movements of the rat. Motility (counts) is recorded for a period of 2 hours. The mean motility induced by vehicle (saline) treatment in the absence of stimulant drug is used as baseline. The 100 percent effect of stimulant drug is accordingly calculated to be total motility counts minus baseline. The response in groups receiving test compound is thus determined by the total motility counts minus baseline, expressed in percent of the similar result recorded in the parallel stimulant drug control group. The percent responses are converted to percent inhibition from which ED50 values are calculated by means of log-probit analyses. In a parallel set of data, the potential sedative properties (exemplified by basal locomotor activity inhibition) of the test compounds are evaluated using essentially the same procedure with the exception of not administering the stimulant drug at the initiation of locomotor assessment.

Results. As can be seen in

Table 2, N-(2-amino-4-(4-fluorobenzylamino)-phenyl) carbamic acid ethyl ester, N-(2,6-dimethyl-4-morpholin-4-yl-phenyl)-3,3-dimethyl-butyramide and 2-cyclopentyl-N-(2,6-dimethyl-4-morpholin-4-yl-phenyl)-acetamide all produced an inhibition of the d-amphetamine induced hyperactivity in rats. The potency with which their effects are exerted is stronger than their potency to inhibit basal locomotor activity, that is, the inhibition of amphetamine-induced hyperactivity cannot be explained by sedative properties of the compounds. Rather, their efficacy reflects an antipsychotic potential of the latter three test compounds.

TABLE 2
Effects of compounds on amphetamine-induced hyperactivity in the rat.

| | Amphetamine | Motility |
|---|---|---|
| | antagonism | inhibition |
| | ED50 (mg/kg) ± | ED50 (mg/kg) ± |
| Compound | std. dev. | std. dev. |
| N-(2-amino-4-(4- | 2.3 (1.2) | >8.1 |
| fluorobenzylamino)- | | |
| phenyl) carbamic acid | | |
| ethyl ester | | |
| N-(2,6-Dimethyl-4- | 2.1 (1.5) | 7.6 (4.8) |
| morpholin-4-yl-phenyl)- | | |
| 3,3-dimethyl-butyramide | | |
| 2-Cyclopentyl-N-(2,6- | 2.6 (2.3) | 5.3 (2.6) |
| dimethyl-4-morpholin-4- | | |
| yl-phenyl)-acetamide | | |
| Lithium-chloride | 12 (1.7) | >40 |
| Olanzapine | 0.21 (1.7) | 0.72 (2.4) |

### Conclusion.

Since both the known antimanic treatment lithium (Goldberg 2000, J. Clin. Psychiatry 61 (Suppl. 13), 12-18) and the known antipsychotic compound olanzapine are efficacious in this model, these data indicate that N-(2-amino-4-(4-fluorobenzylamino)-phenyl) carbamic acid ethyl ester, N-(2,6-dimethyl-4-morpholin-4-yl-phenyl)-3,3-dimethyl-butyramide, 2-cyclopentyl-N-(2,6-dimethyl-4-morpholin-4-yl-phenyl)-acetamide and other compounds that are able to increase the ion flow through KCNQ potassium channels (such as, but not limited to, compounds according to any one of formulae 1, 2, 3, 4, 5, 6, 7, 8, 9 or 10) possess an antipsychotic potential with relevance for treatment of patients suffering from schizophrenia and other psychotic states as well as mania in bipolar spectrum disorders.

As these compounds employ a mechanism of action that is different from that employed by known antipsychotics to produce an antipsychotic effect, it is believed that if a compound that is able to increase the ion flow through KCNQ potassium channels (such as, but not limited to, a compound according to any one of formulae 1, 2, 3, 4, 5, 6, 7, 8, 9 or 10) is combined with a known antipsychotic, a smaller amount of both types of compound will be necessary to induce an equivalent antipsychotic effect.

### Example 1

### Antipsychotic Potential, Part C

In the conditioned avoidance response (CAR) model, rats are trained to respond to a stimulus within a fixed time by moving from one compartment to another in order to avoid a mild foot shock. Known antipsychotics selectively suppress the avoidance response within a certain dose-range without suppressing escape behaviour elicited by the delivery of the foot shock. The CAR model is a predictive and reliable animal model that is sensitive to compounds with an antipsychotic potential. Thus, all known antipsychotics inhibit CAR (Wadenberg and Hicks, Neuroscience and Biobehav Rev 23, 851-862, 1999).

Subjects. Male Wistar rats (Taconic, Denmark) weighing 150 g at the beginning of the study are used. The rats are housed in pairs and maintained on a 12 h light/dark cycle (lights on 06:00). The animals are fed once daily (approx. 6 pellets/rat) in order to keep the rats at 80% of their free-feeding weight. Water is available ad libitum. Temperature (21±1° C.) and relative humidity (55±5%) are automatically controlled.

Experimental procedure. Conditioned avoidance testing is conducted using four automated shuttle-boxes (ENV-010M, MED-Associates) each placed in a sound-attenuated chamber. Each box is divided into two compartments by a partition with an opening. The position of the animal and crossings from one compartment to the other are detected by two photocells placed on either side of the dividing wall. Upon presentation of the conditioned stimuli (CS), tone and light, the animals have 10s to cross to the other compartment of the shuttle-box in order to turn the CS off (end the trial) and avoid the appearance of the unconditioned stimulus (UCS). If the rat remains in the same compartment for more than 10s, the UCS is presented as 0.5 mA scrambled foot-shocks until escape is performed or 10s in maximal duration. The following behavioural variables are evaluated: avoidance (response to CS within 10s); escape (reponse to CS+UCS); escape failures (failure to respond); intertrial crosses and locomotor activity. The rats are habituated to the shuttle-box 3 min before each test session. During training each test session consists of 30 trials with intertrial intervals varying randomly between 20s and 30s. Training is carried out until the rats display an avoidance of 80% or more, on 3 consecutive days. A test is preceded by a pre-test the day before giving rise to a baseline value for each animal, thus the animals serve as their own control. Seven to eight rats are used at each dose level. A parallel control group receiving the vehicle of the test compound is also included.

Administration of compound. N-(2-amino-4-(4-fluorobenzylamino)-phenyl) carbamic acid ethyl ester (5 and 10 mg/kg), N-(2,6-Dimethyl-4-morpholin-4-yl-phenyl)-3,3-dimethyl-butyrannide (2.5 and 5 mg/kg) or 2-Cyclopentyl-N-(2,6-dimethyl-4-morpholin-4-yl-phenyl)-acetamide (2.5 and 5 mg/kg) are administered s.c 30 min before the test, in a volume of 5 ml/kg. All compounds are dissolved in a vehicle of 10% 2-hydroxy-propyl-beta-cyclodextrin (isotonic with glucose, pH 5-7).

Statistical analyses. The effects of compounds on avoidance and escape failure behaviours are statistically evaluated by means of two-way repeated measures ANOVA followed by post hoc comparisons (Student-Newman-Keuls Method) when appropriate. P-levels <0.05 are considered statistically significant.

Results. As can be seen in Table 3, N-(2-amino-4-(4-fluorobenzylamino)-phenyl) carbamic acid ethyl ester, 2-cyclopentyl-N-(2,6-dimethyl-4-morpholin-4-yl-phenyl)-acetamide, and N-(2,6-dimethyl-4-morpholin-4-yl-phenyl)-3,3-dimethyl-butyramide all significantly reduce the number of avoidances, indicative of an antipsychotic activity of 2-cyclopentyl-N-(2,6-dimethyl-4-morpholin-4-yl-phenyl)-acetamide and N-(2,6-dimethyl-4-morpholin-4-yl-phenyl)-3,3-dimethyl-butyramide (5 mg/kg) and N-(2-amino-4-(4-fluorobenzylamino)-phenyl) carbamic acid ethyl ester (10 mg/kg). None of the tested doses cause any incidences of escape failures, corresponding to a lack of effect on motor performance (data not shown).

TABLE 3
Effects of compounds on the conditioned avoidance response in rats.

| | % inhibition of |
|---|---|
| | avoidance (Std. dev.) |
| Treatment | Relative to baseline. |
| Vehicle (10% Hpbeta) | −2 (4.2) |
| N-(2-amino-4-(4-fluorobenzylamino)-phenyl) | 1 (6.7) |
| carbamic acid ethyl ester, 5 mg/kg | |
| N-(2-amino-4-(4-fluorobenzylamino)-phenyl) | 59 (37)*** P < 0.001 |
| carbamic acid ethyl ester, 10 mg/kg | |
| 2-Cyclopentyl-N-(2,6-dimethyl-4-morpholin- | 5 (3.5) |
| 4-yl-phenyl)-acetamide, 2.5 mg/kg | |
| 2-Cyclopentyl-N-(2,6-dimethyl-4-morpholin- | 65 (36)*** P < 0.001 |
| 4-yl-phenyl)-acetamide, 5 mg/kg | |
| N-(2,6-Dimethyl-4-morpholin-4-yl-phenyl)- | 1 (14) |
| 3,3-dimethyl-butyramide, 2.5 mg/kg | |
| N-(2,6-Dimethyl-4-morpholin-4-yl-phenyl)- | 71 (26)*** P < 0.001 |
| 3,3-dimethyl-butyramide, 5 mg/kg | |

Conclusion. These data support an antipsychotic potential of N-(2-amino-4-(4-fluorobenzylamino)-phenyl) carbamic acid ethyl ester, N-(2,6-dimethyl-4-morpholin-4-yl-phenyl)-3,3-dimethyl-butyramide, 2-cyclopentyl-N-(2,6-dimethyl-4-morpholin-4-yl-phenyl)-acetamide and other compounds that are able to increase the ion flow through KCNQ potassium channels (such as, but not limited to, compounds according to any one of formulae 1, 2, 3, 4, 5, 6, 7, 8, 9 or 10).

As these compounds employ a mechanism of action that is different from that employed by known antipsychotics, to produce an antipsychotic effect, it is believed that if a compound that is able to increase the ion flow through KCNQ potassium channels (such as, but not limited to, a compound according to any one of formulae 1,2,3, 4, 5, 6, 7, 8, 9 or 10) is combined with a known antipsychotic, a smaller amount of both types of compound will be necessary to induce an equivalent antipsychotic effect.

### Example 1

### Antipsychotic Potential, Part D

Clinical data imply that amphetamine-naïve schizophrenic and bipolar patients display an exaggerated response to a first dose of amphetamine supporting that these patients show a dopaminergic sensitisation (Strakowski et al. 1996, Biol. Psychiatry 40, 872-880, Lieberman et al. 1987, Psychopharmacology, 91, 415-433, Strakowski et al., 2001, CNS Drugs 15, 701-708). This phenomenon is modeled in rodents when repeated intermittent administration of amphetamine leads to a progressive increase in the behavioural response to an amphetamine challenge, a phenomenon known as behavioural sensitisation (Robinson and Berridge, Brain Research Rev. 1993, 18(3):247-91). The mesolimbic dopamine pathway is believed to be the major neural circuit involved in this behavioural sensitisation (Robinson and Becker, Brain Research 1986, 396(2):157-98). Inhibition of the behavioural response to an acute amphetamine challenge in sensitised animals is a model for evaluating the antipsychotic or antimanic potential of compounds.

Subjects. Male NMRI mice (Charles River) weighing approx. 35 g are used. The animals are housed 6 mice pr cage in a 12-hr light/dark cycle under controlled conditions for regular in-door temperature (21±2° C.) and humidity (55±5%) with food and tap water available ad libitum. 12 mice are used pr experimental group.

Experimental procedure. All mice are pre-treated once daily for five days with either d-amphetamine sulphate (2.5 mg/kg s.c.) or saline (10 ml/kg). For the 17 days between the last day of pre-treatment and the test day, the animals are kept in their homecage receiving the care as described above. The experiment is performed under normal light conditions in an undisturbed room. The mice are treated with test substance or vehicle and placed individually in the test cages for 30 min. The mice are then challenged with D-amphetamine sulphate (1.25 mg/kg s.c.) or saline (5 ml/kg) and replaced in the test-cage and data acquisition is begun. 5×8 infrared light sources and photocells interspaced by 4 cm monitor the locomotor activity. The light beams cross the cage 1.8 cm above the bottom of the cage. The recording of a motility count requires interruption of adjacent light beams, thereby avoiding counts induced by stationary movements of the mice.

Administration of compounds. Amphetamine-pretreated mice and vehicle-pretreated mice are s.c. treated with N-(2-amino-4-(4-fluorobenzylamino)-phenyl) carbamic acid ethyl ester (0-10 mg/kg), 2-cyclopentyl-N-(2,6-dimethyl-4-morpholin-4-yl-phenyl)-acetamide (0-5 mg/kg) or N-(2,6-dimethyl-4-morpholin-4-yl-phenyl)-3,3-dimethyl-butyramide (0-5 mg/kg) or vehicle (10% 2-hydroxy-propyl-beta-cyclodextrin, isotonic, pH 5-7, 5 ml/kg) 30 min prior to the data acquisition.

Data analyses. The total counts obtained in the 30 min test are averaged pr animal group and used for calculation of drug effects in the following manner: The average motility induced by an amphetamine challenge in amphetamine-pretreated animals is used as the sensitised response. The average motility induced by a vehicle challenge to vehicle-pretreated animals is used as a baseline motility response. The baseline value is subtracted from the sensitized amphetamine response value and set as 100% i.e. the sensitised response. This calculation is repeated for each dose group and the value for each dose-group is subsequently expressed relative to the 100% value. That is, the response in amphetamine-sensitized groups receiving test compound is thus determined as the sensitised response minus the baseline motility, expressed in percent of the similar result recorded in the sensitized amphetamine response group. The percent responses are converted to percent inhibition and exposed to log-probit analysis thus producing an ED50 for inhibiting the sensitised response. Similarly an ED50 for inhibiting baseline motility is calculated by expressed the motility response in vehicle-pretreated, vehicle-challenged, drug-treated animals relative to the baseline motility response. A therapeutic index value is subsequently calculated by dividing the first ED50 by the second.

Results. As can be seen in Table 4, N-(2-amino-4-(4-fluorobenzylamino)-phenyl) carbamic acid ethyl ester, N-(2,6-dimethyl-4-morpholin-4-yl-phenyl)-3,3-dimethyl-butyramide and 2-cyclopentyl-N-(2,6-dimethyl-4-morpholin-4-yl-phenyl)-acetamide, as well as the antimanic compound lithium chloride, and the antipsychotic olanzapine, all inhibit the hyperactivity induced by an amphetamine challenge in sensitised mice. The potency with which these compounds exert this effect is stronger than the potency with which these compounds inhibit baseline motility. That is, the compounds possess a calming effect, i.e. antipsychotic/antimanic effect, that is separate from their sedative effects (i.e. therapeutic index >1).

TABLE 4
Effects of compounds on a sensitised behavioural response to
amphetamine in mice. Data are reported as ED50 (mg/kg) ± Std. Dev.

| | Inhibition | | |
|---|---|---|---|
| | of the | | |
| | sensitised | Inhibition | Thera- |
| | amphetamine | of baseline | peutic |
| Compound | response. | motility. | index |
| N-(2-amino-4-(4- | 4.4 (1.4) | 7.6 (1.3) | 2 |
| fluorobenzylamino)-phenyl) | | | |
| carbamic acid ethyl ester | | | |
| N-(2,6-Dimethyl-4-morpholin-4- | 1.6 (1.2) | >2.5 | >1 |
| yl-phenyl)-3,3-dimethyl- | | | |
| butyramide | | | |
| 2-Cyclopentyl-N-(2,6-dimethyl-4- | 1.2 (1.3) | 2.2 (1.3) | 2 |
| morpholin-4-yl-phenyl)- | | | |
| acetamide | | | |
| N-(4,6-Dimethyl-2-morpholin-4- | 0.4 | 2.2 | 5 |
| yl-pyrimidin-5-yl)-2-(4-fluoro- | | | |
| henyl)-acetamide | | | |
| Hexanoic acid (2,6-difluoro-4- | 2.4 | >5 | >2 |
| morpholin-4-yl-phenyl)-amide | | | |
| 2-Cyclopentyl-N-(4,6-dimethyl-2- | 0.9 | 2.5 | 3 |
| morpholin-4-yl-pyrimidin-5-yl)- | | | |
| acetamide | | | |
| N-(2-Bromo-4-morpholin-4-yl-6- | 3.1 | 6.0 | 2 |
| trifluoromethyl-phenyl)- | | | |
| propionamide | | | |
| N-(2,4-Dimethyl-6-morpholin-4- | 1.4 | 3.1 | 2 |
| yl-pyridin-3-yl)-3,3-dimethyl- | | | |
| butyramide | | | |
| [2-Amino-4-(2,4,6-trimethyl- | 1.7 | 4.3 | 3 |
| benzylamino)-phenyl]-carbamic | | | |
| acid ethyl ester | | | |
| 2-Cyclopentyl-N-(2-methoxy-6- | 1.7 | 3.0 | 2 |
| methyl-4-morpholin-4-yl-phenyl)- | | | |
| acetamide | | | |
| Lithium-chloride | 34 (7.2) | >>40 | >>1 |
| Olanzapine | 0.11 (1.4) | >0.31 | >3 |

Conclusion. These data suggest that N-(2-amino-4-(4-fluorobenzylamino)-phenyl) carbamic acid ethyl ester, N-(2,6-dimethyl-4-morpholin-4-yl-phenyl)-3,3-dimethyl-butyramide, 2-cyclopentyl-N-(2,6-dimethyl-4-morpholin-4-yl-phenyl)-acetamide and other compounds that are able to increase the ion flow through KCNQ potassium channels (such as, but not limited to, compounds according to any one of formulae 1, 2, 3, 4, 5, 6, 7, 8, 9 or 10) inhibit the sensitised locomotor activity response to amphetamine in amphetamine-pretreated mice. These data thus indicate potential usefulness of such compounds in treating psychotic symptoms in patients suffering from schizophrenia or mania.

As these compounds employ a mechanism of action that is different from that employed by known antipsychotics to produce an antipsychotic effect, it is believed that if a compound that is able to increase the ion flow through KCNQ potassium channels (such as, but not limited to, a compound according to any one of formulae 1, 2, 3, 4, 5, 6, 7, 8, 9 or 10) is combined with a known antipsychotic, a smaller amount of both types of compound will be necessary to induce an equivalent antipsychotic effect.

### Example 1

### Antipsychotic Potential, Part E

Psychostimulant drugs increase locomotor activity via an increase in extracellular DA levels in the nucleus accumbens, which is the terminal area of the mesolimbic DA projections (Guix et al., 1992, Neurosci. Lett., 138(1), 137-140; Moghaddam et al., 1989, Synapse, 4(2), 156-161). It is therefore believed that the assessment of a compounds ability to inhibit a psychostimulant-induced increase in extracellular DA levels in the nucleus accumbens is another reliable method for the evaluation of a compound's potential to treat a condition that results from an underlying hyperdopaminergic state such as mania or schizophrenia. Hence, the following experiments are conducted to investigate the effect of compounds that are able to increase the ion flow through KCNQ potassium channels, such as N-(2-amino-4-(4-fluorobenzylamino)-phenyl) carbamic acid ethyl ester (i.e. retigabine), 2-cyclopentyl-N-(2,6-dimethyl-4-morpholin-4-yl-phenyl)-acetamide and N-(2,6-dimethyl-4-morpholin-4-yl-phenyl)-3,3-dimethyl-butyramide, on baseline and amphetamine-evoked levels of DA in the nucleus accumbens of freely moving rats.

Subjects. Male Sprague-Dawley rats (Charles River), initially weighing 275-300 g, are used. The animals are housed under a 12-hr light/dark cycle under controlled conditions for regular in-door temperature (21±2° C.) and humidity (55±5%) with food and tap water available ad libitum.

Surgery. Animals are anaesthetized with hypnorm/dormicum (2 ml/kg s.c.) and intracerebral guide cannulas (CMA/12) are stereotaxically implanted, positioning the dialysis probe tip in the nucleus accumbens (co-ordinates: 1.7 mm anterior to bregma, −1.2 mm lateral to bregma, 8.0 mm ventral to the dura). Anchor screws and acrylic cement is applied for fixation of the guide cannula. The body temperature of the animals is maintained at 37° C. by means of a rectal probe and a heating plate. The rats are allowed to recover from surgery for 2 days, housed singly in cages.

Experimental procedure. On the day of the experiment, a microdialysis probe (CMA/12, 0.5 mm diameter, 2 mm length) is inserted through the guide cannula of the conscious animal. The probes are connected to a microinjection pump via a dual channel swivel which allowed the animals unrestricted movements. Perfusion of the microdialysis probe with filtered Ringer solution (145 mM NaCl, 3 mM KCl, 1 mM MgCl₂, 1.2 mM CaCl₂) is maintained for the duration of the experiment at a constant flow rate of 1 μL/min. After 180 min of stabilisation, the experiments are initiated. Dialysates are collected every 20 min. After the experiments the rats are sacrificed by decapitation, their brains removed, frozen and sliced for probe placement verification.

Administration of compounds. 2-Cyclopentyl-N-(2,6-dimethyl-4-morpholin-4-yl-phenyl)-acetamide (5 mg/kg), N-(2,6-dimethyl-4-morpholin-4-yl-phenyl)-3,3-dimethyl-butyramide (5 mg/kg), retigabine (8.1 mg/kg) or vehicle (10% 2-hydroxy-propyl-beta-cyclodextrin, isotonic, pH 5-7) is administered subcutaneously in a volume of 2.5 ml/kg. Thirty minutes later, D-amphetamine sulphate (0.5 mg/kg s.c.) is administered.

Analysis of dialysate. The concentration of DA in the dialysates is assessed by means of HPLC with electrochemical detection. The dialysate constituents are separated by reverse phase liquid chromatography (ODS 150×3 mm, 3 μM). Mobile phase consists of 90 mM NaH₂PO₄, 50 mM sodium citrate, 367 mg/l sodium 1-octanesulfonic acid, 50 μM EDTA and 8% acetonitrile (pH 4.0) at a flow rate of 0.5 ml/min. Electrochemical detection of DA is accomplished using a coulometric detector; potential set at E1=−75 mV and E2=300 mV (guard cell at 350 mV) (Coulochem II, ESA). The dialysate levels of DA in the three dialyse samples preceding the administration of compound are averaged and used as baseline level of DA (100%)

Statistical analysis. The dialysate levels of DA in the three dialyse samples preceding the administration of compound are averaged and used as baseline level of DA (100%). Data are analysed using repeated measure analyses of variance followed by post hoc tests (Tukey test), when appropriate. *P<0.05 were considered significant.

Results. As can be seen in Table 5, below, N-(2-amino-4-(4-fluorobenzylamino)-phenyl) carbamic acid ethyl ester (P<0.001), 2-cyclopentyl-N-(2,6-dimethyl-4-morpholin-4-yl-phenyl)-acetamide (P<0.05) and N-(2,6-dimethyl-4-morpholin-4-yl-phenyl)-3,3-dimethyl-butyramide (P=0.002) acutely and significantly dampen the amphetamine-induced increase in extracellular levels of DA in the nucleus accumbens of freely moving rats. None of the latter three compounds significantly affects the basal extracellular DA level in this region (data not shown).

TABLE 5
Effects of compounds on the amphetamine-evoked increase in DA levels
in the nucleus accumbens of freely moving rats. Normalised DA levels in
the nucleus accumbens of freely moving rats are shown.

| | | Amphetamine + | Amphetamine + | Amphetamine + |
|---|---|---|---|---|
| | | 2- | N-(2,6- | N-(2-amino-4- |
| | | Cyclopentyl-N- | Dimethyl-4- | (4- |
| | | (2,6-dimethyl- | morpholin-4-yl- | fluorobenzylamino)- |
| | | 4-morpholin- | phenyl)-3,3- | phenyl) |
| | | 4-yl-phenyl)- | dimethyl- | carbamic acid |
| | Amphetamine + | acetamide | butyramide | ethyl ester |
| | vehicle | (5 mg/kg) | (5 mg/kg) | (8.1 mg/kg) |
| Time (min) | % of baseline | % of baseline | % of baseline | % of baseline |
| −40 | 91 ± 6 | 95 ± 6 | 108 ± 5 | 105 ± 3 |
| −20 | 96 ± 5 | 106 ± 5 | 100 ± 3 | 99 ± 6 |
| 0 | 112 ± 7 | 99 ± 4 | 91 ± 4 | 96 ± 6 |
| 20 | 168 ± 19 | 125 ± 9 | 112 ± 12 | 160 ± 11 |
| 40 | 338 ± 27 | 264 ± 39 | 227 ± 46 | 241 ± 33 |
| 60 | 375 ± 46 | 265 ± 17* | 262 ± 53* | 221 ± 26* |
| 80 | 319 ± 59 | 231 ± 22 | 195 ± 38* | 217 ± 14* |
| 100 | 232 ± 48 | 167 ± 25 | 172 ± 24 | 173 ± 9 |
| 120 | 162 ± 37 | 109 ± 11 | 166 ± 32 | 139 ± 9 |
| 140 | 129 ± 27 | 93 ± 15 | 129 ± 35 | 120 ± 9 |

*P < 0.05 compared to amphetamine-vehicle group, same time.

Conclusion. These data show that N-(2-amino-4-(4-fluorobenzylamino)-phenyl) carbamic acid ethyl ester, 2-cyclopentyl-N-(2,6-dimethyl-4-morpholin-4-yl-phenyl)-acetamide and N-(2,6-dimethyl-4-morpholin-4-yl-phenyl)-3,3-dimethyl-butyramide all dampen amphetamine-provoked increases in DA extracellular levels, which suggests that these compounds, as well as other compounds that are able to increase the ion flow through KCNQ potassium channels (such as, but not limited to, compounds according to any one of formulae 1, 2, 3, 4, 5, 6, 7, 8, 9 or 10) have potential in the treatment of psychotic symptoms, with a faster onset of therapeutic activity than known antipsychotics.

The data also suggest that adding compounds that are able to increase the ion flow through KCNQ potassium channels (such as, but not limited to, compounds according to any one of formulae 1, 2, 3, 4, 5, 6, 7, 8, 9 or 10) would shorten the time to onset of therapeutic activity of known antipsychotics if used as an adjunctive therapy.

### Example 2

### Potential to Treat Substance Abuse and/or Use

Substance abuse and/or use of substances such as nicotine, cannabis, CNS depressants such as ethanol, psychostimulants such as cocaine and amphetamine, and opioids such as heroin and morphine, represents a serious health problem in many areas of the world, and furthermore represents a deleterious comorbidity in several psychiatric disorders. Preclinical studies suggest that all substances with an addictive potential share the ability to increase DA activity in the mesolimbic DA reward system of the brain and that this common mechanism underlies the reinforcing effects of such substances to ultimately promote their abuse and/or use (Di Chiara and Imperato 1988, PNAS 85(14):5274-8).

Examples of tests that enable the study of a compound's potential to treat the use and/or abuse of a substance in a preclinical setting are outlined below.
Positive data with compounds that are able to increase the ion flow through KCNQ potassium channels (such as, but not limited, to compounds according to any one of formulae 1, 2, 3, 4, 5, 6, 7, 8, 9 or 10) from the aforementioned experiments outlined in Example 1 part A, B, D, and E will indicate potential for use of such compounds as effective therapy options in relation to the abuse and/or use of substances such as nicotine, cannabis, CNS depressants such as ethanol, psychostimulants such as cocaine and amphetamine, and opioids such as heroin and morphine.
The self-administration paradigm represents another important behavioural model of validity for such studies (Ciccocioppo et al., 2003, Psychopharmacology; 168(1-2):208-15, Cervo et al., 2003, Neuropsychopharmacology 28:1150-1159). Compounds that are able to increase the ion flow through KCNQ potassium channels (such as, but not limited to, compounds according to any one of formulae 1, 2, 3, 4, 5, 6, 7, 8, 9 or 10) are believed to reduce or abolish the reinforcing value of a substance and to prevent cue-induced reinstatement (relapse) of self-administration of a substance in abstinent animals. This suggests a potential for such a compound to be an effective therapy option in relation to the abuse and/or use of substances such as nicotine, cannabis, CNS depressants such as ethanol, psychostimulants such as cocaine and amphetamine, and opioids such as heroin and morphine, and consequently that such compounds may be of use in the treatment of addiction states that are caused by abuse of such substances.
The conditioned place preference paradigm represents yet another important behavioural model of validity for such studies (Phillips and LePiane 1980, Pharmacol Biochem Behav 12(6):965-8.) Compounds that are able to increase the ion flow through KCNQ potassium channels (such as, but not limited to, compounds according to any one of formulae 1, 2, 3, 4, 5, 6, 7, 8, 9 or 10) are believed to reduce or abolish the reinforcing value of a substance, illustrated by reducing the preference for the compartment which was previously paired with the administration of the rewarding substance. This suggests a potential for such a compound to be an effective therapy option in relation to the abuse and/or use of substances such as nicotine, cannabis, CNS depressants such as ethanol, psychostimulants such as cocaine and amphetamine, and opioids such as heroin and morphine and consequently that such compounds may be of use in the treatment of addiction states that are caused by abuse of such substances.

### Example 3

### Antidepressant Potential

Mood disorders such as depression are life-threatening disorders with a life-time prevalence in the range of 5-20% (Hirschfeld and Cross, 1982, Archives of General psychiatry 39, 35-46). Known treatment options for these devastating conditions are targeting the monoaminergic and catecholaminergic systems, with the exception of electroconvulsive therapy, and about 70% of the patients are considered to respond favourably to these medications. Hence, there is a need for novel treatments with a better antidepressant or mood-elevating efficacy and this is achieved with compounds with a different mechanism of action. The antidepressant potential of compounds that are able to increase the ion flow through KCNQ potassium channels (such as, but not limited to, compounds according to any one of formulae 1, 2, 3, 4, 5, 6, 7, 8, 9 or 10) is studied in preclinical settings, e.g.:
The chronic mild stress paradigm represents one pharmacological model of use for such an evaluation (Willner 1997, Psychopharmacology 134, 319-329). This paradigm has proven to be sensitive to both antidepressants and thus supports the potential for depression treatment.
The forced swim test is another widely used and extensively validated model for the preclinical evaluation of antidepressant activity (Porsolt et al. 1977, Arch. Int. Pharmacodyn. 229, 327-336). In the following example, N-(2-amino-4-(4-fluorobenzylamino)-phenyl) carbamic acid ethyl ester, 2-cyclopentyl-N-(2,6-dimethyl-4-morpholin-4-yl-phenyl)-acetamide and N-(2,6-dimethyl-4-morpholin-4-yl-phenyl)-3,3-dimethyl-butyramide are tested in the mouse forced swim test.

Subjects. Male NMRI mice (Charles River) weighing 23-25 g are used. The mice are kept 8 mice pr cage in a 12-hr light/dark cycle under controlled conditions for regular in-door temperature (21±2° C.) and humidity (55±5%) with food and tap water available ad libitum. 8 mice are used pr experimental group.

Experimental procedure. The mice are placed in 2000 ml beaker containing 1200 ml of tempered water (25° C.) and left to swim for 6 min. The performance of the mice is videorecorded, digitalized and analysed by means of a digital analysis system (Bioobserve). The time spent immobile for the last 3 min. of the test session is quantified for each mouse.

Treatment. 30 min. before the test, mice are treated s.c. with N-(2,6-dimethyl-4-morpholin-4-yl-phenyl)-3,3-dimethyl-butyramide, 2-cyclopentyl-N-(2,6-dimethyl-4-morpholin-4-yl-phenyl)-acetamide or vehicle (10-%-2-OH-propyl-cyclodextrin, 10 ml/kg). In addition as positive control, imipramine-HCl (40 mg/kg) and a saline control (10 ml/kg) is included.

Analyses. The time spent immobile is statistically compared across the experimental groups against the relevant control group by means of one-way analysis of variance. A post-hoc test (Student-Newman-Keuls) is employed when appropriate. P-levels <0.05 were considered significant.

Results. As can be seen from Table 6,2-cyclopentyl-N-(2,6-dimethyl-4-morpholin-4-yl-phenyl)-acetamide and N-(2,6-dimethyl-4-morpholin-4-yl-phenyl)-3,3-dimethyl-butyramide both significantly reduce the time spent immobile during the 3-6 min swim in mice. Their efficacy is comparable to the effect of an antidepressant dose of imipramine-HCl. In contrast, the antipsychotic olanzapine has only a weak effect in this test.

TABLE 6
Effects of compounds on immobility in the mouse forced swim test.

| | 2-Cyclopentyl- | N-(2,6- | | |
|---|---|---|---|---|
| | N-(2,6- | Dimethyl-4- | | |
| | dimethyl-4- | morpholin-4- | | |
| | morpholin-4- | yl-phenyl)-3,3- | | Imipramine- |
| | yl-phenyl)- | dimethyl- | | HCl |
| | acetamide | butyramide | Olanzapine | Immobility |
| Dose: | Immobility in | Immobility in | Immobility in | in |
| mg/kg | % (±S.D.) | % (±S.D.) | % (±S.D.) | % (±S.D.) |
| Vehicle | 100 (6.6) | 100 (6.6) | 100 (6.61) | 100 (7.1) |
| 0.31 | — | — | 96 (14) | — |
| 1.3 | 97.5 (6.3) | 102 (4.6) | 95 (11) | — |
| 2.5 | 97 (6.7) | 96.7 (7.5) | — | — |
| 5.0 | 81.0 (18)* | 82.3 (20)* | — | — |
| 40 | — | — | — | 73.8 (22)* |

Conclusion. These data indicate an antidepressant potential of 2-cyclopentyl-N-(2,6-dimethyl-4-morpholin-4-yl-phenyl)-acetamide and N-(2,6-dimethyl-4-morpholin-4-yl-phenyl)-3,3-dimethyl-butyramide like that of imipramine. Hence, these compounds and other compounds that are able to increase the ion flow through KCNQ potassium channels (such as, but not limited to, compounds according to any one of formulae 1, 2, 3, 4, 5, 6, 7, 8, 9 or 10) are believed to have a potential to treat depression associated with mood disorders and to generally have a mood-elevating potential of benefit to patients suffering from depressed mood.

### Example 4

### Efficacy Against Negative Symptoms in Schizophrenia

The schizophrenic spectrum of symptoms involves a cluster of negative symptoms that embraces anhedonia, social withdrawal, and emotional flattening. Negative symptoms are inadequately treated by known antipsychotics (Duncan et al. 2004, Schizoph. Res, 71(2-3), 239-248; Meltzer et al. 1986, J. Clin. Psychopharmacol., 6(6), 329-338) with the possible exception of amisulpride (Delcker et al.; 1990, Pharmacopsychiatry 23, 125-130) and represents an important unmet need. A positive effect in the chronic mild stress paradigm (Papp and Wieronska 2000, Journal of Psychopharmacology 14(1), 46-52) or in the social interaction test (Sams-Dodd, 1999, Rev Neurosci., 10(1):59-90) support the potential for alleviating negative symptoms in schizophrenic patients of compounds that are able to increase the ion flow through KCNQ potassium channels (such as, but not limited to, compounds according to any one of formulae 1, 2, 3, 4, 5, 6, 7, 8, 9 or 10). Data generated in the aforementioned example 3 do also support such a potential. In support of the latter, olanzapine does not significantly reduce immobility in the forced swim test (data shown above), which is in line with the observation that olanzapine has an inadequate effect on negative symptoms in humans suffering from schizophrenia.

### Example 5

### Relative Efflux Through the KCNQ2 Channel

The assay measures the relative efflux through the KCNQ2 channel, and is carried out according to a method described by Tang et al. (Tang, W. et. al., J. Biomol. Screen. 2001, 6, 325-331) for hERG potassium channels with the modifications described below. An adequate number of CHO cells stably expressing voltage-gated KCNQ2 channels are plated at a density sufficient to yield a mono-confluent layer on the day of the experiment. Cells are seeded on the day before the experiment and loaded with 1 μCi/ml [⁸⁶Rb] over night. On the day of the experiment cells are washed with a HBSS-containing buffer. Cells are pre-incubated with drug for 30 minutes and the ⁸⁶Rb⁺ efflux is stimulated by a submaximal concentration of 15 mM KCl in the continued presence of drug for additional 30 minutes. After a suitable incubation period, the supernatant is removed and counted in a liquid scintillation counter (Tricarb). Cells are lysed with 2 mM NaOH and the amount of ⁸⁶Rb⁺ is counted. The relative efflux is calculated ((CPMₛᵤₚₑᵣ/(CPMₛᵤₚₑᵣ+CPM_{cell}))_{Cmpd}/(CPMₛᵤₚₑᵣ/(CPMₛᵤₚₑᵣ+CPM_{cell}))_{15mM KCl})*100-100. Subsequently, the EC₅₀ value for the drug can be calculated.

### Example 6

### GABA_{A} Receptors, and Noradrenergic α1_{A} Receptors

It is desirable to treat patients with compounds that induce as few unspecific side-effects as possible. When testing compounds that are able to increase the ion flow through KCNQ potassium channels such as compounds among those according to formulae 1, 2, 3, 4, 5, 6, 7 or 8 in a functional GABA_{A} (gamma-amino-buturic-acid receptor subtype A) assay comprising the α1β3γ2S subunits expressed in xenopus oocytes (Ebert et al., 1997, Mol Pharmacol, 52(6):1150-6) and comparing the results with those for retigabine, it is found that retigabine significantly enhances the response evoked by GABA at an EC20 concentration (˜150% of control levels), while the compounds among those listed above have insignificant effects (90%-100% of control levels). Hence, compounds that are able to increase the ion flow through KCNQ potassium channels such as compounds among those according to formulae 1, 2, 3, 4, 5, 6, 7 or 8 appear to lack the modulatory effect on GABA_{A} receptors that is found for retigabine.

Furthermore, testing compounds that are able to increase the ion flow through KCNQ potassium channels such as compounds among those according to formulae 1, 2, 3, 4, 5, 6, 7 or 8 in a 3H-Prazosin binding assay on the noradrenergic α1_{A} receptor expressed in baby hamster kidney cells (Michel et al., 1989, Brit. J. Pharmacol., 98: 883-889) it is found that Retigabine displays low micromolar affinity towards noradrenergic α1_{A} receptors (Ki=2 μM), whereas compounds that are able to increase the ion flow through KCNQ potassium channels such as the compounds among those according to formulae 1, 2, 3, 4, 5, 6, 7 or 8 show no significant inhibition of 3H-prazosin binding at a concentration of 10 μM.

Conclusion. These differences suggest that compounds that are able to increase the ion flow through KCNQ potassium channels such as compounds among those according to formulae 1, 2, 3, 4, 5, 6, 7 or 8 display an in vitro pharmacological profile that may translate into a superior clinical profile of these compounds compared to retigabine, in terms of reduced side-effects (for instance less sedation, and reduced risk of orthostatic hypotension) and consequently superior patient compliance.

### Example 7

### Catalepsy

All known antipsychotics have an undesirable potential to cause extrapyramidal side-effects. The symptoms, such as akinesia, rigidity and tremor, bear some resemblance to Parkinsonism symptoms. The ability to 1) not induce catalepsy symptoms and 2) ameliorate the catalepsy symptoms induced by a known antipsychotic such as for example haloperidol is tested in one or more preclinical models such as for example the catalepsy test (Hyde et al., 1995, Psychopharmacology, 118(2):142-9). With regard to 1), if compounds that are able to increase the ion flow through KCNQ potassium channels (such as, but not limited to, compounds according to any one of formulae 1, 2, 3, 4, 5, 6, 7, 8, 9 or 10) do not induce behaviour indicative of akinesia, rigidity or tremor, it would indicate that these compounds lack a tendency to cause extrapyramidal side-effects in the clinical setting. With regard to 2), if compounds that are able to increase the ion flow through KCNQ potassium channels (such as, but not limited to, compounds according to any one of formulae 1, 2, 3, 4, 5, 6, 7, 8, 9 or 10) dampen the akinesia, rigidity or tremor symptoms that are induced by a known antipsychotic it would indicate that such compounds may be a valuable adjunctive therapy to such a known antipsychotic and furthermore suggest that such compounds have a potential to treat movements disorders such as for example Tourette's syndrome, Huntington's disease and Parkinson's disease.

### Example 8

### Procognitive Potential

Several psychiatric and neurological disorders, such as schizophrenia, bipolar disorder, Alzheimer's disease and dementia, are characterised by a loss of cognitive ability, exemplified by impairments in attention, short or long-term memory or executive functioning. A compound's ability to treat such symptoms may be tested in a preclinical setting. Compounds that are able to increase the ion flow through KCNQ potassium channels (such as, but not limited to, compounds according to any one of formulae 1, 2, 3, 4, 5, 6, 7, 8, 9 or 10) are believed to be effective against attentional, memory or executive functioning deficits in tests such as the 5-choice serial reaction task model (Carli et al., Behay. Brain Res. 1983 September; 9(3): 361-80), the Morris water maze model (Learn. Motiv., 12, 239-260, 1981) and/or the attentional set-shifting model (Rodefer et al., Eur. J. Neurosci. 2005 February; 21(4): 1070-6) or in other models with similar predictive validity. This indicates that these compounds will potentially be useful for treating patients suffering from impaired cognitive abilities, in either monotherapy or adjunctive treatment therapy.

### Example 9

### ADHD Potential

Attention-deficit/hyperactivity disorder (ADHD) is a highly heritable condition that affects a significant number of children and adults worldwide. The disorder is characterized by symptoms of attentional problems and hyperkinesia. Preclinical data imply that the pathophysiology of this disorder involves a hyperfunctioning mesocortical dopamine system (Viggiano et al., 2003, Neurosci. Biobehay. Rev. 27(7): 683-9). The potential of compounds that are able to increase the ion flow through KCNQ potassium channels (such as, but not limited to, compounds according to any one of formulae 1, 2, 3, 4, 5, 6, 7, 8, 9 or 10) to treat ADHD may be tested in a preclinical model such as, for example, the 5-choice serial reaction task model, which is a model for attention (Carli et al., Behav. Brain Res. 1983 September; 9(3): 361-80). Another preclinical model may be used to assess whether compounds that are able to increase the ion flow through KCNQ potassium channels (such as, but not limited to, compounds according to any one of formulae 1, 2, 3, 4, 5, 6, 7, 8, 9 or 10) will reduce impulsive behaviour, e.g. in the form of tolerance to delay of reward (Evenden and Ryan, 1996, Psychopharmacology 128(2): 161-70). Lastly, the results obtained in the aforementioned Example 1 part A and B will also lend themselves to support a potential to treat ADHD symptoms. Positive effects in such models will support a potential to treat ADHD patients in the clinical setting.

Subjects. male Wistar AF rats (28-41 days old) (Centre d′Elevage René Janvier, France) were used. The animals were housed in groups of 3-5 animals per cage and kept under a 12 h/12 h light dark rhythm (start of the dark phase at 8:00 p.m., CET), 20° C. room temperature and 60% humidity. During the whole study, tap water was provided ad libitum. Food was restricted to 5-10 g/day/animal in order to maintain animals at 65-75% of their free feeding weight

Experimental procedure. The experiments were performed in two identical T-maze apparatus constructed from opaque grey plastic tubing and consisting of a starting runway (30 cm long) and two arms (35 cm long) each leading to a rectangular black plastic goal box (18 cm large, 30 cm wide, 10 cm high). Removable grey plastic guillotine doors could be inserted into the vertical clefts, situated at the entry of the starting runway and at each end of the arms. One of the goal-boxes (left or right, depending on the rats) was constantly provided with a large reward, the other with a small reward. Large and small rewards consisted, respectively, of 5 and 1 pellets (20 mg, Technical & Scientific Equipment GmbH, Germany). The pellets were placed in a translucent food-cup before each trial.

First phase: habituation. Animals were first subjected to two 5-min sessions of habituation. The rat was gently introduced in the starting runway, which then was closed with a guillotine door inserted in the cleft (referred to as c₀ in FIG. 1). The animal was allowed to freely explore the apparatus and to eat food rewards placed in the food cups.

Second phase: pretraining. After a door was placed in the cleft c₂ near each goal-box, the rat was introduced in the starting runway. When it entered one of the two arms, a door was inserted behind it in the cleft c₁ near the choice area and the door placed in cleft c₂ was removed. As soon as the animal entered the goal-box, the door was placed again in the cleft c₂. The rat was removed from the goal-box as soon as the pellets were eaten (usually within less than 1 min). The animal was then returned to its home cage for an intertrial interval of 2 to 3 min. Each rat was submitted one to five times/day to five trials sessions in this procedure. Training was initiated when the rat selected the arm giving access to the large reward in more than 14 out of 15 trials.

Third phase: training. Rats underwent one to five times/day five trials training sessions during which a delay was introduced before access to the large reward. After a door was placed in the cleft c₂ near each goal-box, the rat was introduced in the starting runway. When it entered one of the two arms, a second door was inserted behind it in the cleft c₁ near the choice area, so that rat selecting the arm leading to the large reward could be detained in this arm for a period (referred to as the waiting delay) before having access to the reinforcement. Otherwise, if the animal selected the arm leading to the small reward, the door placed in the cleft c₂ was immediately opened, allowing the animal to enter the goal-box. At the beginning of training, the waiting delay was 30 sec. Then, if the percentage of choices of the large and delayed reward remained high (i.e. ≧40%) for more than six sessions, it was increased to 45 sec, and if necessary to 60 sec, 90 sec and finally 120 sec. Drug testing began when the animal selected the large and delayed reward in two trials out of five (or less) in two consecutive sessions and in one trial out of five (or less) in the following session. Animals that did not comply with this criterion within two sessions with the 120 sec waiting delay were discarded from the experiment.

Drug testing was conducted over six consecutive test-sessions of five trials each, during which the access to the large reward was preceded by the waiting delay. Test sessions were: Two control pre-drug sessions: control pre-drug session 1 and control pre-drug session 2; Two drug sessions: drug session 1 and drug session 2; Two control post-drug sessions: control post-drug session 1 and control post-drug session 2. At least 24 h elapsed between drug session 1 and drug session 2, and between drug session 2 and control post-drug session 1. Placebo was administered before each control (pre-drug and post-drug) sessions, and the compound studied (or placebo for Vehicle group) was administered before each drug session.

Treatment. Rats (10-11 per group) were treated with N-[2-amino-4-(4-fluoro-benzylamino)-phenyl]-carbamic acid ethyl ester (compound 1; see EP554543), 2-cyclopentyl-N-(2-methoxy-6-methyl-4-morpholin-4-yl-phenyl)-acetamide (compound 2; see WO2005/087754), N-(2,4-dimethyl-6-morpholin-4-yl-pyridin-3-yl)-3,3-dimethylbutyramide (compound 3; see WO2006/092143) or vehicle (10% HpbetaCyclodextrin) 30 min (s.c.) before the test. Atomoxetine (1 mg/kg) was administered intraperitoneally (i.p.) 30 min before the test and served as a positive control to validate the predictive validity of the test.

Analysis. For each animal, the percentage of choices of the large-but-30-s-delayed reward was calculated as follows:

Control pre-drug sessions: 100×‘number of choices of the large reward on the two control pre-drug sessions’/10.

Drug sessions: 100×‘number of choices of the large reward on the two drug sessions’/10.

Control post-drug sessions: 100×‘number of choices of the large reward on the two control post-drug sessions’/10.

Control sessions (including pre-drug and post-drug sessions): 100×[‘number of choices of the large reward on the two control pre-drug sessions’+‘number of choices of the large reward on the two control post-drug sessions’]/20.

Then, the difference of the percentage of choices between drug sessions and control sessions (ΔDrug-Control) was calculated for each animal, and statistically compared between groups using a one-way Analysis of Variance (ANOVA).

For each group, comparisons of the group means between control pre-drug session, drug sessions and control post-drug sessions were performed by ANOVAs for repeated measures. For groups for which the ANOVA showed a session effect significant at p<0.10, comparisons of the group means between drug sessions and control (pre and post-drug) sessions, drug sessions and control pre-drug sessions, drug sessions and control post-drug sessions, control pre-drug sessions and control post-drug sessions, were performed by the Student's t-test for paired comparisons.

Results. As can be seen from Table 7, compounds 1, 2 and 3 all significantly increased the percentage of choice of the large-but-delayed reward in juvenile Wistar rats, indicative of an ability of these compounds to enhance the ability to wait and hence to reduce impulsivity, in these animals. The effects of these compounds were comparable to that of Atomoxetine, which served to validate the predictive validity of the test with regard to potential to inhibit impulsivity in humans.

TABLE 7
Effects of compounds on the percentage of choices of a
large-but-delayed vs a small-but-immediate reward,
in the rat T-maze procedure.

% Choice of the large
but delayed reward on

| | Control | Drug |
|---|---|---|
| Groups | sessions | sessions |
| Vehicle s.c. | 9.0 ± 2.9 | 12.0 ± 5.8 |
| Atomoxetine 1 mg/kg i.p. | 10.7 ± 2.3 | 40.0 ± 2.2*** |
| Compound 1, 5 mg/kg s.c. | 10.8 ± 2.0 | 26.7 ± 3.3* |
| Compound 2, 1 mg/kg s.c. | 11.7 ± 2.5 | 33.3 ± 3.3*** |
| Compound 3, 2.5 mg/kg s.c. | 8.3 ± 2.8 | 25.0 ± 6.7* |

*P < 0.05,
***P < 0.001: Post hoc test following one-way ANOVA, statistically significant compared to vehicle group.

### Conclusion

In conclusion, the present study showed that compounds 1, 2 and 3 all induced an improvement of waiting ability, i.e. an improvement of impulse control, in juvenile rats. This result suggests that these three compounds and other compounds that are able to increase the ion flow through KCNQ potassium channels (such as, but not limited to, compounds according to any one of formulae 1, 2, 3, 4, 5, 6, 7, 8, 9 or 10) have a beneficial effect in diseases characterized by a deficit of impulse control, such as ADHD.

### Example 10

### Antimanic/Bipolar Potential

Manic or hypomanic episodes are characteristic for bipolar spectrum disorders, which are debilitating psychiatric illnesses which often increase in severity over time (Post et al., 1995, Ann. N Y Acad. Sci., 771, 677-96). In addition to lithium, several anticonvulsant and antipsychotic drugs are used in the acute treatment of mania. In view of the anticonvulsant efficacy of compounds that are able to increase the ion flow through KCNQ potassium channels (such as, but not limited to, compounds according to any one of formulae 1, 2, 3, 4, 5, 6, 7, 8, 9 or 10), it is believed that such compounds also possess antimanic potential. In the preclinical setting, data from the model described in Example 1 part D support antimanic-like activity. This potential is further supported in the amphetamine+chlordiazepoxide induced hyperactivity rodent model of mania (Arban et al., 2005, Behavioral Brain Res. 158, 123-132) by comparing the effect of said compounds with the effect of lithium. Positive effects in such models will indicate potential to treat mania in the clinical setting. Furthermore, positive modulatory effects of early potassium outward currents by carbamazepine, valproate and lamotrigine have been described (Grunze et al., 1998, Neuropsychobiology; 38: 131-138; Olpe et al., 1991, Experientia; 47: 254-257; Walden et al., 1993, Eur. Neuropsychopharmacol; 3: 137-141), and evidence implies that lithium may indirectly act as a KCNQ2 channel opener (Borsotto et al., 2007, Pharmacogenomics, 7(2), 123-32). Positive KCNQ channel modulation could consequently be a common denominator for antimanic compounds.

### Example 11

### Potential to Treat Aggression

Aggression is an umbrella term for types of behaviour that are intended to inflict harm. These types of behaviour evolved as adaptations to deal with competition, but when expressed out of context, they can have destructive consequences. Uncontrolled aggression has several components, such as impaired recognition of social cues and enhanced impulsivity (Nelson and Trainor, 2007, Nat. Rev. Neurosci., 8(7):536-46). As compounds that are able to increase the ion flow through KCNQ potassium channels (such as, but not limited to, compounds according to any one of formulae 1, 2, 3, 4, 5, 6, 7, 8, 9 or 10) hyperpolarise the neuron membrane and thus reduce the likelihood of an action potential discharge, such compounds are believed to serve a general purpose of reducing firing activity of nerve cells in the brain, with the consequence of inhibiting excessive neuronal activity. Consequently, such compounds are believed to effectively treat aggression and impulsivity symptoms in rats (Wilson et al., 2000, Psychol. Rep. 86(3 Pt 1):941-6). Such symptoms are seen, for example, in patients suffering from psychotic, bipolar spectrum disorders or mood disorders.

Subjects. 5 week old male Wistar rats (Harlan, Germany) were used. The animals were housed in groups of four animals per cage and kept under a 12 h/12 h light dark rhythm (start of the dark phase at 2:00 p.m., CET), at 20° C. room temperature and 60% humidity. During the whole study, standard diet and tap water was provided ad libitum.

Experimental procedure. The tetradic encounter (TE) test is conducted according to the protocol described by Wolffgramm (1990, Behaviour 113: 172-186 and 1990, Behaviour 113: 187-204) and Wolffgramm and Heyne (1990, Behaviour, 113, 205-222). The mutual resident intruder (MRI) test is a modified version of the test described by De Boer et al., 1999 J. Pharmacol. Exp. Therap. 288: 1125-1133: Two rats live for one week in a Makrolon type IV cage that is subdivided in length by an opaque PVC wall. Therefore, half of the cage is the home territory of each animal. At test time, the wall is removed from the cage so that the animals can interact with each other for 15 minutes. Both animals are from the same test group. The cage is illuminated by dim red light. The test starts 1 hour after beginning of the dark phase. All animals are marked individually by means of black symbols on their back. Behaviour is registered by video. Each animal is tested once.

Treatment. 30 min. before the test, rats are treated s.c. with N-[2-amino-4-(4-fluoro-benzylamino)-phenyl]-carbamic acid ethyl ester (compound 1), 2-cyclopentyl-N-(2-methoxy-6-methyl-4-morpholin-4-yl-phenyl)-acetamide (compound 2), N-(2,4-dimethyl-6-morpholin-4-yl-pyridin-3-yl)-3,3-dimethyl-butyramide (compound 3) or vehicle (10-%-2-OH-propyl-cyclodextrin, 10 ml/kg). In addition, D-amphetamine is included as a positive control to ensure the predictive validity of the tests.

Analyses. In both the TE test and the MRI test, the portion of time (%) spent in non-social, friendly, defensive, aggressive or agonistic (sum of defensive and aggressive behaviour) behaviour was compared across the experimental groups against the relevant control group by means of Analysis of Variance (ANOVA) (one-factor).

Results. As can be seen from Table 8 and

Table 9, compounds 1, 2 and 3 all significantly reduced agonistic behaviour (aggressive and defensive behaviour) in both test situations. The effect was highly selective since “friendly” interactions were not significantly affected. As expected, d-amphetamine reduced social interest in general and agonistic interactions in particular, and thus validates the predictive validity of these tests.

TABLE 8
Effects of compounds on % of time displaying different types
of social behaviours in the rat Tetradic Encounter test.

| Dose: mg/kg | Non-social | Friendly | Aggressive | Defensive | Agonistic |
|---|---|---|---|---|---|
| Vehicle | 65.3 | 22.9 | 4.2 | 7.6 | 11.8 |

Compound 1

| | | | | | |
|---|---|---|---|---|---|
| 2 | 65.7 | 21.9 | 5.2 | 7.2 | 12.4 |
| 10 | 69.2 | 24.7 | 2.4 | 3.7* | 6.1* |

Compound 2

| | | | | | |
|---|---|---|---|---|---|
| 1 | 69.3 | 18.2* | 5.3 | 7.2 | 12.5 |
| 5 | 72.7** | 26.2 | 0.4** | 0.8*** | 1.2*** |

Compound 3

| | | | | | |
|---|---|---|---|---|---|
| 1 | 65.4 | 20.2 | 5.9 | 8.5 | 14.4 |
| 5 | 79.9*** | 20.0 | 0.1** | 0.0*** | 0.1*** |

Amphetamine

| | | | | | |
|---|---|---|---|---|---|
| 0.5 | 81.4*** | 17.3* | 0.1*** | 1.2*** | 1.4*** |

*P < 0.05, **P < 0.01,*** P < 0.001: Post hoc test following one-way ANOVA, statistically significant compared to vehicle group.

TABLE 9
Effects of compounds on % of time displaying different types
of social behaviours in the Mutual Resident Intruder rat test.

| Dose: mg/kg | Non-social | Friendly | Aggressive | Defensive | Agonistic |
|---|---|---|---|---|---|
| Vehicle | 67.6 | 2.4 | 15.4 | 14.6 | 30 |

Compound 1

| | | | | | |
|---|---|---|---|---|---|
| 2 | 64.2 | 3.4 | 16.1 | 16.3 | 32.4 |
| 10 | 87.3*** | 4.9* | 4.2*** | 3.6*** | 7.8*** |

Compound 2

| | | | | | |
|---|---|---|---|---|---|
| 1 | 63.1 | 4.0 | 15.5 | 17.3 | 32.8*** |
| 5 | 96.6*** | 2.4 | 0.7*** | 0.2*** | 0.9*** |

Compound 3

| | | | | | |
|---|---|---|---|---|---|
| 1 | 72.3 | 3.4 | 12.0 | 12.3 | 24.3 |
| 5 | 97.4*** | 2.3 | 0.2*** | 0.2*** | 0.3*** |

Amphetamine

| | | | | | |
|---|---|---|---|---|---|
| 0.5 | 86.8*** | 5.3 | 3.9*** | 4.0*** | 7.9*** |

*P < 0.05, **P < 0.01,*** P < 0.001: Post hoc test following one-way ANOVA, statistically significant compared to vehicle group.

Conclusion. These data indicate an anti-aggression potential of compounds 1, 2 and 3. Hence, these compounds and other compounds that are able to increase the ion flow through KCNQ potassium channels (such as, but not limited to, compounds according to any one of formulae 1, 2, 3, 4, 5, 6, 7, 8, 9 or 10) are believed to have potential in treating aggression symptoms in patients suffering from psychotic, bipolar spectrum disorders or mood disorders, or other type of psychiatric or neurological disorder that are associated with aggression symptoms.

### Example 12

### Potential to Treat Sexual Motivational Dysfunction

Loss of sexual motivation or libido is a symptom that is comorbid with several psychiatric and neurological disorders, and it is desirable to develop compounds that may effectively normalise sexual behaviour in such patients. The potential of a compound to stimulate sexual arousal can be evaluated preclinically. Non-contact erections are considered a measure of sexual arousal (Sachs, 2000, Neuroscience and Biobehavioral Reviews, 24, 541-560) and a compound's potential to stimulate sexual arousal can thus be evaluated if studying the effect of the compound on the frequency of penile erections in the rat (Succu et al., 2007, Neuropharmacology 52(3): 1034-43). As another example, the compound's effect on pre-copulatory behaviour in experimental animals may be assessed by studying the latency to approach and time spent in close proximity to the sexual target (Lopez et al., 2007, Pharmacol. Biochem. Behay., 87(3): 369-79).

### Example 13

### Potential to Treat Alzheimer's Disease

Evidence supports the hypothesis that accumulation of amyloid peptides (Aβ) contributes to the pathogenesis of Alzheimer's disease (AD). The progressive accumulation of Aβ in brain regions such as the hippocampus has been proposed to contribute to the cognitive decline in AD. Overexpression of Aβ peptide in transgenic mice overexpressing a mutant form of human amyloid precursor protein (APP) (hAPP_{FAD}) was recently shown to be associated with non-convulsive electroencephalographic (EEG) epileptiform activity within the entorhinal-hippocampal circuitry (Palop et al., Neuron, 2007). This circuitry is heavily involved in learning and in the formation of memory (Nakashiba et al., Science, 2008), and thus the Aβ-associated aberrant neuronal epileptiform activity may underly the impairments in learning and memory that are exhibited by such mice (Palop et al., PNAS, 2003). EEG findings in early Alzheimer-type dementia and mild cognitive impairment subjects include increases in theta and delta rhythms (Babiloni et al., Neuroscience, 2007; Signorino et al., Electroencephal. Clin. Neurophysiol., 1995), while epileptiform EEG activity appears not to have been studied. However, epileptiform EEG is strongly predictive of unprovoked seizures, and literature clearly supports that there is an increased risk of unprovoked seizures in individuals with AD compared with others of the same age (Amatniek et al., Epilepsia, 2006; Hauser et al, Neurology, 1986; Romanelli et al., Arch. Neurol. 1990). Thus, the occurrence of hippocampal aberrant epileptiform activity and associated seizures may underly the pathogenesis of AD, and control of aberrant epileptiform EEG activity in subjects at risk of developing AD, or in individuals with early AD, may thus provide a novel disease-modifying concept for the treatment and/or cure of AD.

Opening of KCNQ2-5 channels is known to stabilize the membrane potential and thus represents a powerful method to control neuronal excitability. Compounds that are able to increase the ion flow through KCNQ potassium channels (such as, but not limited to, compounds according to any one of formulae 1, 2, 3, 4, 5, 6, 7, 8, 9 or 10) effectively suppress induced epileptiform EEG activity in the rat entorhinal cortex or hippocampus and exert potent anticonvulsant effects in animal seizure models (see, for example, WO2005/087754). Taken together, such data imply that use of compounds that are able to increase the ion flow through KCNQ potassium channels (such as, but not limited to, compounds according to any one of formulae 1, 2, 3, 4, 5, 6, 7, 8, 9 or 10) represents a novel disease-modifying concept for the treatment and/or cure of AD, acting through the control of aberrant epileptiform EEG activity in subjects at risk of developing AD, or in individuals with early AD.

1. A Method for reducing symptoms of, or for treating, one or more disorders wherein the dopaminergic system is disrupted, said method comprising administering to a host in need thereof an effective amount of a compound able to increase the ion flow through KCNQ potassium channels.

2. The method according to claim 1, wherein said disorder(s) is/are independently selected from the group consisting of schizophrenia, other psychotic states than schizophrenia, mood disorders, attention-deficit/hyperactivity disorder-(ADHD), aggression, movement disorders, substance use and substance abuse.

3. The method according to claim 1, wherein said disorders are schizophrenia and one or more other psychotic states than schizophrenia.

4.-22. (canceled)

23. The method according to claim 1, wherein said disorder(s) is/are one or more mood disorder(s).

24.-48. (canceled)

49. The method according to claim 1, wherein said disorder is attention-deficit/hyperactivity disorder-(ADHD).

50. (canceled)

51. The method according to claim 1, wherein said disorder is aggression.

52. (canceled)

53. The method according to claim 1, wherein said disorder(s) is/are one or more movement disorder(s).

54.-58. (canceled)

59. The method according to claim 1, wherein said disorder(s) is selected from the group consisting of substance use substance abuse and a combination thereof.

60.-80. (canceled)

81. The method according to claim 1, wherein said compound is a compound according to formula 1: 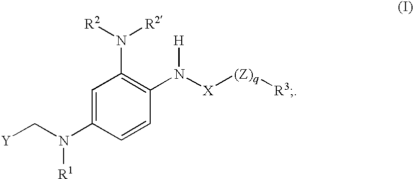
wherein:
R¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl and hydroxy-C₃₋₈-Cycloalk(en)yl;
R² and R^{2′} are independently selected from the group consisting of hydrogen, C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, acyl, hydroxy-C₁₋₆-alk(en/yn)yl and hydroxy-C₃₋₈-cycloalk(en)yl;
R³ is selected from the group consisting of hydrogen, C₁₋₆-alk(en/yn)yl, C₃₋₈-cycloalk(en)yl, aryl, C₃₋₈-cycloalk(en)yl-C₁₋₆-alk(en/yn)yl, aryl-C₃₋₆-alk(en/yn)yl, hydroxy-C₁₋₆-alk(en/yn)yl, aryl-C₃₋₈-cycloalk(en)yl, NR¹⁰R^{10′}—C₁₋₄-alk(en/yn)yl, NR¹⁰R^{10′}—C₃₋₈-Cycloalk(en)yl and hydroxy-C₃₋₈-cycloalk(en)yl; wherein:
R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or
R¹⁰ and R^{10′} together with the nitrogen atom to which they are attached form a 4-8 membered saturated or unsaturated ring that optionally contains 1, 2 or 3 further heteroatoms;
X is CO or SO₂;
Z is O or NR⁴, wherein:
R⁴ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl and hydroxy-C₃₋₈-cycloalk(en)yl; or
R³ and R⁴ together with the nitrogen atom to which they are attached form a 4-8 membered saturated or unsaturated ring that optionally contains 1, 2 or 3 further heteroatoms, and the ring formed by R³ and R⁴ and the nitrogen atom is optionally substituted with one or more substituents independently selected from C₁₋₆-alk(en/yn)yl, aryl and aryl-C₁₋₆-alk(en/yn)yl;
q is 0 or 1; and
Y represents a heteroaryl of formula II or III: 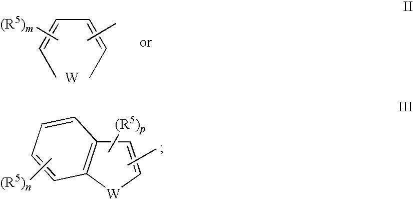
wherein:
W is O or S;
m is 0, 1, 2 or 3;
n is 0, 1, 2, 3 or 4;
p is 0 or 1; and
each R⁵ is independently selected from the group consisting of C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, acyl, halogen, halo-C₁₋₆-alk(en/yn)yl, alk(en/yn)yloxy, —CO—NR⁶R^{6′}, cyano, nitro, —NR⁷R^{4′}, —S—R⁸, —SO₂R⁸, and SO₂OR⁸; wherein:
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and aryl;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, aryl and acyl; and
R⁸ is selected from the group consisting of C₁₋₆-alk(en/yn)yl, C₁₋₆-cycloalk(en)yl, C₃₋₈-cycloalk(en)yl-C₁₋₆-alk(en/yn)yl, aryl and NR⁹R^{9′}; wherein:
R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl;
or a pharmaceutically acceptable salt thereof.

82. (canceled)

83. The method according to claim 81, wherein said compound is selected from the group consisting of:
{2-Amino-4-[(5-chloro-thiophen-2-ylmethyl)-methyl-amino]-phenyl}-carbamic acid ethyl ester;
{2-Amino-4-[(5-chloro-thiophen-2-ylmethyl)-amino]-phenyl}-carbamic acid ethyl ester;
{2-Amino-4-[(5-methyl-thiophen-2-ylmethyl)-methyl-amino]-phenyl}-carbamic acid ethyl ester;
{2-Amino-4-[(5-bromo-thiophen-2-ylmethyl)-amino]-phenyl}-carbamic acid ethyl ester;
{2-Amino-4-[(6-chloro-3-methoxy-benzo[b]thiophen-2-ylmethyl)-amino]-phenyl}-carbamic acid ethyl ester;
{(2-Amino-4-[(benzo[b]thiophen-2-ylmethyl)-amino]-phenyl}-carbamic acid ethyl ester;
{2-Amino-4-[(5-methyl-thiophen-2-ylmethyl)-amino]-phenyl}-carbamic acid ethyl ester;
{2-Amino-4-[(4-bromo-3-methoxy-thiophen-2-ylmethyl)-amino]-phenyl}-carbamic acid ethyl ester;
{2-Amino-4-[(5-phenyl-thiophen-2-ylmethyl)-amino]-phenyl}-carbamic acid ethyl ester;
{2-Amino-4-[(3-chloro-thiophen-2-ylmethyl-amino]-phenyl}-carbamic acid ethyl ester;
(2-Amino-4-{[4-(4-chloro-benzenesulfonyl)-3-methyl-thiophen-2-ylmethyl]-amino}-phenyl)-carbamic acid ethyl ester;
{2-Amino-4-[(3-methyl-thiophen-2-ylmethyl)-amino]-phenyl}-carbamic acid ethyl ester;
{2-Amino-4-[(5-fluoro-benzofuran-3-ylmethyl)-amino]phenyl}-carbamic acid ethyl ester;
{2-Amino-4-[(thiophen-2-ylmethyl)-amino]-phenyl}-carbamic acid ethyl ester;
{2-Amino-4-[(4-bromo-thiophen-2-ylmethyl)-amino]-phenyl}-carbamic acid ethyl ester;
{2-Amino-4-[(5-ethyl-thiophen-2-ylmethyl)-amino]-phenyl}-carbamic acid ethyl ester;
{2-Amino-4-[(thiophen-3-ylmethyl)-amino]-phenyl}-carbamic acid ethyl ester;
{2-Amino-4-[(5-chloro-thiophen-2-ylmethyl)-ethyl-amino]-phenyl}-carbamic acid ethyl ester;
{2-Amino-4-[(benzo[b]thiophen-3-ylmethyl)-amino]-phenyl}-carbamic acid ethyl ester;
{2-Amino-4-[(5-dimethyl-amino-benzo[b]thiophen-3-ylmethyl)-amino]-phenyl}-carbamic acid ethyl ester;
{2-Amino-4-[(5-dimethyl-amino-3-methyl-benzo[b]thiophen-2-ylmethyl)-amino]-phenyl}-carbamic acid ethyl ester;
{2-Amino-4-[(5-fluoro-thiophen-2-ylmethyl)-amino]-phenyl}-carbamic acid ethyl ester;
{2-Amino-4-[(benzo[b]thiophen-2-ylmethyl)-amino]-phenyl}-carbamic acid propyl ester;
{2-Amino-4-[(benzo[b]thiophen-3-ylmethyl)-amino]-phenyl}-carbamic acid propyl ester;
N-{2-Amino-4-[(5-chloro-thiophen-2-ylmethyl)amino]phenyl}-2-(4-fluoro-phenyl)-acetamide;
N-{2-Amino-4-[(5-chloro-thiophen-2-ylmethyl)amino]phenyl}-3,3-dimethyl-butyramide; and
pharmaceutically acceptable salts thereof.

84. The method according to claim 1, wherein said compound is a compound according to formula 2: 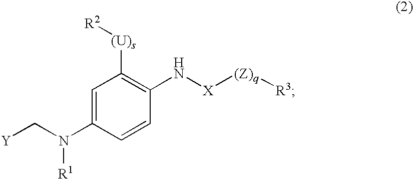
wherein:
s is 0 or 1;
U is O, S, SO₂, SO₂NR¹¹, CO—O or CONR¹¹; wherein:
R¹¹ is selected from the group consisting of hydrogen, C₁₋₆-alk(en/yn)yl, C₃₋₈-cycloalk(en)yl, and C₃₋₈-cycloalk(en)yl-C₁₋₆-alk(en/yn)yl; or
R² and R¹¹ together with the nitrogen atom to which R¹¹ is attached form a 5-8 membered saturated or unsaturated ring that optionally contains 1, 2 or 3 further heteroatoms;
q is 0 or 1;
X is CO or SO₂; with the proviso when X is SO₂, then q is 0;
Z is O or S;
R¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl;
R² is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, NR¹⁰R^{10′}—C₁₋₆-alk(en/yn)yl, NR¹⁰R^{10′}—C₃₋₈-cycloalk(en)yl and NR¹⁰R^{10′}—C₃₋₈-cycloalk(en)yl-C₁₋₆-alk(en/yn)yl; wherein:
R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yl; or
R¹⁰ and R^{10′} together with the nitrogen atom to which they are attached form a 5-8 membered saturated or unsaturated ring that optionally contains 1, 2 or 3 further heteroatoms; and provided that:
when R² is halogen or cyano, then s is 0; and
when s is l and R² is a hydrogen atom or acyl, then U is O or S;
R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, heterocycloalk(en)yl-C₁₋₆-alk(en/yn)yl, Ar, Ar—C₁₋₆-alk(en/yn)yl, Ar—C₃₋₈-cycloalk(en)yl, Ar-heterocycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar—C₁₋₆-alk(en/yn)yl-heterocycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar—C₁₋₆-alk(en/yn)yloxy-C₁₋₆-alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycl oal k(en)yl-C₁₋₆-alk(en/yn)yl oxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl, NR¹²R^{12′}, optionally substituted NR¹²R^{12′}—C₁₋₆-alk(en/yn)yl, optionally substituted NR¹²R^{12′}—C₃₋₈-cycloalk(en)yl, and optionally substituted NR¹²R^{12′}—C₃₋₈-cycloalk(en)yl-C₁₋₆-alk(en/yn)yl; wherein:
R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, Ar-heterocycloalk(en)yl, Ar-oxy-C₁₋₆-alk(en/yn)yl, Ar-oxy-C₃₋₈-cycloalk(en)yl, Ar-oxy-C₃₋₈-cycloalk(en)yl-C₁₋₆-alk(en/yn)yl, Ar-oxy-heterocycloalk(en)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yr)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or
R¹² and R^{12′} together with the nitrogen atom to which they are attached form a 5-8 membered saturated or unsaturated ring that optionally contains 1, 2 or 3 further heteroatoms; provided that:
when R³ is NR¹²R^{12′}, then q is 0; and
Y represents a group of formula XXIV, XXV, XXVI, XXVII, XXVIII, XXXXI or XXXXII: 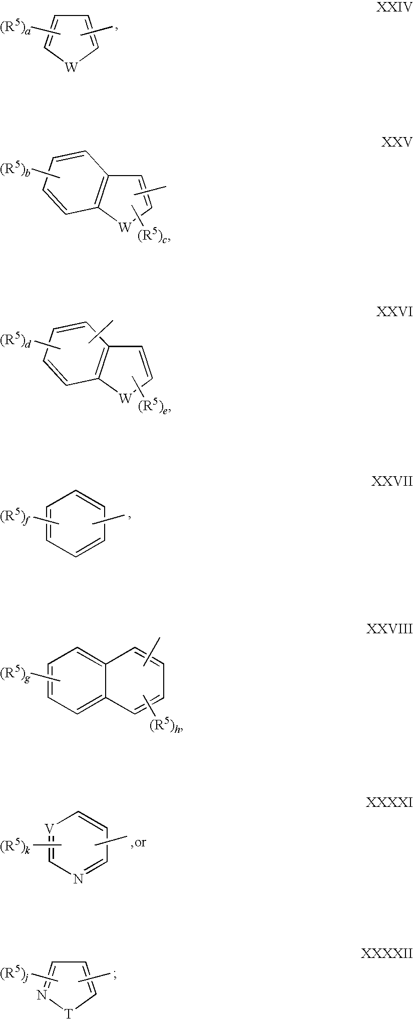
wherein:
the line represents a bond attaching the group represented by Y to the carbon atom;
W is O or S;
V is N, C or CH;
T is N, NH or O;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3;
j is 0, 1 or 2;
k is 0, 1, 2 or 3; and
each R⁵ is independently selected from the group consisting of a C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, Ar-oxy, Ar-oxy-C₁₋₆-alk(en/yn)yl, Ar-oxy-C₃₋₈-cycloalk(en)yl, C₁₋₆-alk(en/yn)yl-heterocycloalk(en)yl, Ar-oxy-C₃₋₈-cycloalk(en)yl-C₁₋₆-alk(en/yn)yl, acyl, C₁₋₆-alk(en/yn)yloxy, C₃₋₈-cycloalk(en)yloxy, C₃₋₈-cycloalk(en)yl-C₁₋₆-alk(en/yn)yloxy, C₁₋₆-alk(en/yn)yloxy-carbonyl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —CO—NR⁶R^{6′}, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, NR⁷R^{7′}, S—R⁸ and SO₂R⁸; or
two adjacent R⁵ together with the aromatic group form a 5-8 membered ring that optionally contains one or two heteroatoms; wherein:
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, heterocycloalk(en)yl-C₁₋₆-alk(en/yn)yl, heterocycloalk(en)yl-C₃₋₈-cycloalk(en)yl, heterocycloalk(en)yl-C₃₋₈-cycloalk(en)yl-C₁₋₆-alk(en/yn)yl, heterocycloalk(en)yl-Ar and acyl; or
R⁷ and R^{7′} together with the nitrogen atom to which they are attached form a 5-8 membered saturated or unsaturated ring that optionally contains 1, 2 or 3 further heteroatoms; and
R⁸ is selected from the group consisting of hydrogen, C₁₋₆-alk(en/yn)yl, C₃₋₈-cycl oal k(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl;
or a pharmaceutically acceptable salt thereof.

85. (canceled)

86. The method according to claim 84, wherein said compound is selected from the group consisting of
{4-[(Benzofuran-2-ylmethyl)-amino]-2-methylphenyl}-carbamic acid propyl ester;
{4-[(5-Chloro-thiophen-2-ylmethyl)-amino]-2-methylphenyl}-carbamic acid ethyl ester;
{4-[(Benzo[b]thiophen-2-ylmethyl)-amino]-2-methylphenyl}-carbamic acid ethyl ester;
{2-Methyl-4-[(5-phenyl-thiophen-2-ylmethyl)-amino]-phenyl}-carbamic acid ethyl ester;
[4-(4-Isopropyl-benzylamino)-2-methylphenyl]-carbamic acid ethyl ester;
[4-(4-Fluoro-benzylamino)-2-methylphenyl]-carbamic acid propyl ester;
(4-{[4-(4-Chloro-benzenesulfonyl)-3-methyl-thiophen-2-ylmethyl]-amino}-2-methylphenyl)-carbamic acid propyl ester;
{4-[(5-Methyl-thiophen-2-ylmethyl)-amino]-2-methylphenyl}-carbamic acid propyl ester;
{4-[(5-Bromo-thiophen-2-ylmethyl)-amino]-2-methylphenyl}-carbamic acid propyl ester;
{4-[(5-Chloro-thiophen-2-ylmethyl)-amino]-2-methylphenyl}-carbamic acid propyl ester;
{4-[(Benzo[b]thiophen-2-ylmethyl)-amino]-2-methylphenyl}-carbamic acid propyl ester;
{2-Methyl-4-[(5-phenyl-thiophen-2-ylmethyl)-amino]-phenyl}-carbamic acid propyl ester;
[4-(4-Isopropyl-benzylamino)-2-methylphenyl]-carbamic acid propyl ester;
{4-[(5-Bromo-thiophen-2-ylmethyl)-amino]-2-chlorophenyl}-carbamic acid ethyl ester;
{4-[(5-Chloro-thiophen-2-ylmethyl)-amino]-2-chlorophenyl}-carbamic acid ethyl ester;
{4-[(Benzo[b]thiophen-2-ylmethyl)-amino]-2-chlorophenyl}-carbamic acid ethyl ester;
[2-Chloro-4-(4-isopropyl-benzylamino)-phenyl]-carbamic acid ethyl ester;
[2-Chloro-4-(4-fluoro-benzylamino)-phenyl]-carbamic acid propyl ester;
2-Chloro-4-{[4-(4-chloro-benzenesulfonyl)-3-methyl-thiophen-2-ylmethyl]-amino)-phenyl}-carbamic acid propyl ester;
{4-[(5-Methyl-thiophen-2-ylmethyl)-amino]-2-chlorophenyl}-carbamic acid propyl ester;
{4-[(5-Bromo-thiophen-2-ylmethyl)-amino]-2-chlorophenyl}-carbamic acid propyl ester;
{2-Chloro-4-[(5-chloro-thiophen-2-ylmethyl)-amino]-phenyl}-carbamic acid propyl ester;
{4-[(Benzo[b]thiophen-2-ylmethyl)-amino]-2-chlorophenyl}-carbamic acid propyl ester;
{4-[(Benzofuran-2-ylmethyl)-amino]-2-chlorophenyl}-carbamic acid propyl ester;
{4-[(5-Chloro-thiophen-2-ylmethyl)-amino]-2-cyanophenyl}-carbamic acid ethyl ester;
{4-[(Benzo[b]thiophen-2-ylmethyl)-amino]-2-methoxyphenyl}-carbamic acid methyl ester;
{4-[(5-Bromo-thiophen-2-ylmethyl)-amino]-2-methoxyphenyl}-carbamic acid isopropyl ester;
{4-[(4-Fluoro-benzyl)-(methyl)amino]-2-methoxyphenyl}-carbamic acid propyl ester;
[4-(Benzo[b]thiophen-2-ylmethyl-(methyl)amino)-2-methoxy-phenyl]-carbamic acid propyl ester;
{4-[(5-Chloro-thiophen-2-ylmethyl)-(methyl)amino]-2-methoxy-phenyl}-carbamic acid propyl ester;
{4-[(5-Bromo-thiophen-2-ylmethyl)-(methyl)amino]-2-methoxy-phenyl}-carbamic acid propyl ester;
{2-Methoxy-4-[methyl-(5-methyl-thiophen-2-ylmethyl)-amino]-phenyl}-carbamic acid propyl ester;
{4-[(4-Fluorobenzyl)-(methyl)-amino]-2-isopropoxyphenyl}-carbamic acid ethyl ester;
[4-(3-Fluorobenzylamino)-2-methoxyphenyl]-carbamic acid ethyl ester;
[4-(4-Isopropylbenzylamino)-2-methoxyphenyl]-carbamic acid ethyl ester;
{2-Methoxy-4-[(3-methylthiophen-2-ylmethyl)-amino]-phenyl}-carbamic acid ethyl ester;
[4-(2,4-Difluorobenzylamino)-2-methoxyphenyl]-carbamic acid ethyl ester;
[2-Cyclopentyloxy-4-(4-methoxybenzylamino)-phenyl]-carbamic acid ethylester;
[2-Cyclopentyloxy-4-(3-fluoro-2-methylbenzylamino)-phenyl]-carbamic acid ethyl ester;
[4-(3-Fluoro-2-methylbenzylamino)-2-phenethyloxyphenyl]-carbamic acid ethyl ester;
[2-Benzyloxy-4-(3-fluoro-2-methylbenzylamino)-phenyl]carbamic acid ethyl ester;
[2-Benzyloxy-4-(4-methylsulfanylbenzylamino)-phenyl]-carbamic acid ethyl ester;
{4-[(Benzo[b]thiophen-3-ylmethyl)-amino]-2-cyclopentyloxyphenyl}-carbamic acid ethyl ester;
[4-(3-Fluoro-2-methylbenzylamino)-2-isopropoxyphenyl]-carbamic acid ethyl ester;
[2-Benzyloxy-4-(3-methoxybenzylamino)-phenyl]-carbamic acid ethyl ester;
{4-[(Benzo[1,3]dioxol-5-ylmethyl)-amino]-2-isopropoxyphenyl}-carbamic acid ethyl ester;
{4-[(5-Bromo-thiophen-2-ylmethyl)-amino]-phenyl}-carbamic acid propyl ester;
{4-[(5-Chloro-thiophen-2-ylmethyl)-amino]-phenyl}-carbamic acid propyl ester;
[2-Cyano-4-(4-isopropylbenzylamino)-phenyl]-carbamic acid ethyl ester;
{4-[(5-Bromo-thiophen-2-ylmethyl)-(methyl)amino]-2-methyl phenyl}-carbamic acid propyl ester;
{4-[(4-Isopropylbenzyl)-(methyl)amino]-2-methyl phenyl}-carbamic acid propyl ester;
{2-Methyl-4-[methyl-(4-trifluoromethyl-benzyl)-amino]-phenyl}-carbamic acid propyl ester;
{2-Methyl-4-[methyl-(4-methylsulfanyl-benzyl)-amino]-phenyl}-carbamic acid propyl ester;
{4-[(4-tert-Butyl-benzyl)-(methyl)amino]-2-chlorophenyl}-carbamic acid ethyl ester;
{2-Chloro-4-[methyl-(4-trifluoromethyl-benzyl)-amino]-phenyl}-carbamic acid ethyl ester;
{2-Chloro-4-[methyl-(4-methylsulfanyl-benzyl)-amino]-phenyl}-carbamic acid ethyl ester;
{4-[(5-Bromo-thiophen-2-ylmethyl)-(methyl)amino]-2-chlorophenyl}-carbamic acid propyl ester;
{2-Chloro-4-[(5-chloro-thiophen-2-ylmethyl)-(methyl)amino]-phenyl}-carbamic acid propyl ester;
{4-[(4-tert-Butyl-benzyl)-(methyl)amino]-2-chlorophenyl}-carbamic acid propyl ester;
{2-Chloro-4-[methyl-(4-trifluoromethyl-benzyl)-amino]-phenyl}-carbamic acid propyl ester;
{4-[(5-Bromo-thiophen-2-ylmethyl)-(methyl)amino]-2-trifluoromethyl-phenyl}-carbamic acid ethyl ester;
{4-[(5-Chloro-thiophen-2-ylmethyl)-(methyl)amino]-2-trifluoromethyl-phenyl}-carbamic acid ethyl ester;
{4-[(4-Isopropyl-benzyl)-(methyl)amino]-2-trifluoromethyl-phenyl}-carbamic acid ethyl ester;
{4-[(4-tert-Butyl-benzyl)-(methyl)amino]-2-trifluoromethyl-phenyl}-carbamic acid ethyl ester;
{4-[Methyl-(4-trifluoromethyl-benzyl)-amino]-2-trifluoromethyl-phenyl}-carbamic acid ethyl ester;
{4-[Methyl-(4-methylsulfanyl-benzyl)-amino]-2-trifluoromethyl-phenyl}-carbamic acid ethyl ester;
{4-[(5-Bromo-thiophen-2-ylmethyl)-(methyl)amino]-2-trifluoromethyl-phenyl}-carbamic acid propyl ester;
{4-[(5-Chloro-thiophen-2-ylmethyl)-(methyl)amino]-2-trifluoromethyl-phenyl}-carbamic acid propyl ester;
{4-[(4-Isopropyl-benzyl)-(methyl)amino]-2-trifluoromethyl-phenyl}-carbamic acid propyl ester;
{4-[(4-tert-Butyl-benzyl)-(methyl)amino]-2-trifluoromethyl-phenyl}-carbamic acid propyl ester;
{4-[Methyl-(4-trifluoromethyl-benzyl)-amino]-2-trifluoromethyl-phenyl}-carbamic acid propyl ester;
{4-[Methyl-(4-methylsulfanyl-benzyl)-amino]-2-trifluoromethyl-phenyl}-carbamic acid propyl ester;
{4-[(5-Bromo-thiophen-2-ylmethyl)-(methyl)amino]-2-cyanophenyl}-carbamic acid propyl ester;
{4-[(4-tert-Butyl-benzyl)-(methyl)amino]-2-cyanophenyl}-carbamic acid propyl ester;
{2-Cyano-4-[methyl-(4-trifluoromethyl-benzyl)-amino]-phenyl}-carbamic acid propyl ester;
{2-Bromo-4-[(5-bromo-thiophen-2-ylmethyl)-(methyl)amino]phenyl}-carbamic acid propyl ester;
{2-Bromo-4-[(5-chloro-thiophen-2-ylmethyl)-(methyl)amino]-phenyl}-carbamic acid propyl ester;
{2-Bromo-4-[(4-isopropylbenzyl)-(methyl)amino]-phenyl}-carbamic acid propyl ester;
{2-Bromo-4-[(4-tert-butyl-benzyl)-(methyl)amino]-phenyl}-carbamic acid propyl ester;
{2-Bromo-4-[methyl-(4-trifluoromethyl-benzyl-amino]-phenyl}-carbamic acid propyl ester;
[2-Iodo-4-(4-isopropyl-benzylamino)-phenyl]-carbamic acid propyl ester;
[4-(4-tert-Butyl-benzylamino)-2-iodophenyl]-carbamic acid propyl ester;
[2-Iodo-4-(4-trifluoromethyl-benzylamino)-phenyl]-carbamic acid propyl ester;
[2-Iodo-4-(4-methylsulfanyl-benzylamino)-phenyl]-carbamic acid propyl ester;
{2-Iodo-4-[4-(4-methylpiperazin-1-yl)-benzylamino]-phenyl}-carbamic acid propyl ester;
{4-[(5-Bromo-thiophen-2-ylmethylyamino]-2-trifluoromethyl-phenyl}-carbamic acid ethyl ester;
{4-[(5-Chloro-thiophen-2-ylmethyl)-amino]-2-trifluoromethyl-phenyl}-carbamic acid ethyl ester;
[4-(4-tert-Butyl-benzylamino)-2-trifluoromethyl-phenyl]-carbamic acid ethyl ester;
[4-(4-Methylsulfanyl-benzylamino)-2-trifluoromethyl-phenyl]-carbamic acid ethyl ester;
{4-[(5-Bromo-thiophen-2-ylmethyl)-amino]-2-trifluoromethyl-phenyl}-carbamic acid propyl ester;
[4-(4-Isopropylbenzylamino)-2-trifluoromethyl-phenyl]-carbamic acid propyl ester;
[4-(4-tert-Butyl-benzylamino)-2-trifluoromethyl-phenyl]-carbamic acid propyl ester;
[2-Trifluoromethyl-4-(4-trifluoromethyl-benzylamino)-phenyl]-carbamic acid propyl ester;
[4-(4-Dimethylamino-benzylamino)-2-trifluoromethyl-phenyl]-carbamic acid propyl ester;
[4-(4-Methylsulfanyl-benzylamino)-2-trifluoromethyl-phenyl]-carbamic acid propyl ester;
{4-[(5-Bromo-thiophen-2-ylmethyl)-amino]-2-cyanophenyl}-carbamic acid propyl ester;
{4-[(5-Chloro-thiophen-2-ylmethyl)-amino]-2-cyanophenyl}-carbamic acid propyl ester;
[2-Cyano-4-(4-trifluoromethyl-benzylamino)-phenyl]-carbamic acid propyl ester;
{2-Bromo-4-[(5-bromo-thiophen-2-ylmethyl)-amino]-phenyl}-carbamic acid propyl ester;
{2-Bromo-4-[(5-chloro-thiophen-2-ylmethyl)-amino]-phenyl}-carbamic acid propyl ester;
[2-Bromo-4-(4-isopropylbenzylamino)-phenyl]-carbamic acid propyl ester;
[2-Bromo-4-(4-tert-butyl-benzylamino)-phenyl]-carbamic acid propyl ester;
[2-Bromo-4-(4-trifluoromethyl-benzylamino)-phenyl]carbamic acid propyl ester;
[2-Bromo-4-(4-methylsulfanyl-benzylamino)-phenyl]-carbamic acid propyl ester;
N-{4-[(5-Bromo-thiophen-2-ylmethyl)-amino]-2-methoxyphenyl}-butyramide;
N-{4-[(5-Chloro-thiophen-2-ylmethyl)-amino]-2-methoxyphenyl}-butyramide;
N-[4-(4-Isopropylbenzylamino)-2-methoxyphenyl]-butyramide;
N-[4-(4-tert-Butyl-benzylamino)-2-methoxyphenyl]-butyramide;
N-[2-Methoxy-4-(4-trifluoromethyl-benzylamino)-phenyl]-butyramide;
{4-[(5-Chloro-thiophen-2-ylmethyl)-amino]-2-furan-2-yl-phenyl}-carbamic acid propyl ester;
[2-Furan-2-yl-4-(4-isopropylbenzylamino)-phenyl]-carbamic acid propyl ester;
[5-(4-Fluorobenzylamino)-biphenyl-2-yl]-carbamic acid propyl ester;
{5-[(5-Chloro-thiophen-2-ylmethyl)-amino]-biphenyl-2-yl}-carbamic acid propyl ester;
[5-(4-Isopropylbenzylamino)-biphenyl-2-yl]-carbamic acid propyl ester;
N-{2-Chloro-4-[(5-chloro-thiophen-2-ylmethyl)-(methyl)amino]-phenyl}-2-phenylacetamide;
N-{2-Chloro-4-[(5-chloro-thiophen-2-ylmethyl)-(methyl)amino]-phenyl}-3,3-dimethylbutyramide;
N-{2-Chloro-4-[(5-chloro-thiophen-2-ylmethyl)-(methyl)amino]-phenyl}-3-phenylpropionamide;
N-{2-Chloro-4-[(5-chloro-thiophen-2-ylmethyl)-(methyl)amino]-phenyl}-butyramide;
Pentanoic acid {2-chloro-4-[(5-chloro-thiophen-2-ylmethyl)-(methyl)amino]-phenyl}-amide;
Cyclopropanecarboxylic acid {2-chloro-4-[(5-chloro-thiophen-2-ylmethyl)-(methyl)amino]-phenyl}-amide;
Cyclobutanecarboxylic acid {2-chloro-4-[(5-chloro-thiophen-2-ylmethyl)-(methyl)amino]-phenyl}-amide;
Cyclopentanecarboxylic acid {2-chloro-4-[(5-chloro-thiophen-2-ylmethyl)-(methyl)amino]-phenyl}-amide;
Cyclohexanecarboxylic acid {2-chloro-4-[(5-chloro-thiophen-2-ylmethyl)-(methyl)amino]-phenyl}-amide;
N-{2-Chloro-4-[(5-chloro-thiophen-2-ylmethyl)-(methyl)amino]-phenyl}-2-thiophen-2-yl-acetamide;
N-{2-Chloro-4-[(5-chloro-thiophen-2-ylmethyl)-(methyl)amino]-phenyl}-2-(3-methoxy-phenyl)-acetamide,
N-{2-Chloro-4-[(5-chloro-thiophen-2-ylmethyl)-(methyl)amino]-phenyl)-2-(4-chloro-phenyl}-acetamide;
N-{2-Chloro-4-[(5-chloro-thiophen-2-ylmethyl)-(methyl)amino]-phenyl}-2-(4-methoxy-phenyl)-acetamide;
N-{2-Chloro-4-[(5-chloro-thiophen-2-ylmethyl)-(methyl)amino]-phenyl)-2-(4-fluoro-phenyl}-acetamide;
N-{2-Chloro-4-[(5-chloro-thiophen-2-ylmethyl)-(methyl)amino]-phenyl}-3-cyclohexylpropionamide;
N-{2-Chloro-4-[(5-chloro-thiophen-2-ylmethyl)-amino]-phenyl}-2,2-dimethylpropionamide;
N-{2-Chloro-4-[(5-chloro-thiophen-2-ylmethyl)-amino]-phenyl}-2-phenoxyacetamide;
N-{2-Chloro-4-[(5-chloro-thiophen-2-ylmethyl)-amino]-phenyl}-2-phenyl acetamide;
N-{2-Chloro-4-[(5-chloro-thiophen-2-ylmethyl)-amino]-phenyl}-3,3-dimethylbutyramide;
N-{2-Chloro-4-[(5-chloro-thiophen-2-ylmethyl)-amino]-phenyl}-butyramide;
Pentanoic acid {2-chloro-4-[(5-chloro-thiophen-2-ylmethyl)-amino]-phenyl}-amide;
Cyclopropanecarboxylic acid {2-chloro-4-[(5-chloro-thiophen-2-ylmethyl)-amino]-phenyl}-amide;
Cyclobutanecarboxylic acid {2-chloro-4-[(5-chloro-thiophen-2-ylmethyl)-amino]-phenyl}-amide;
Cyclopentanecarboxylic acid {2-chloro-4-[(5-chloro-thiophen-2-ylmethyl)-amino]phenyl}-amide;
Cyclohexanecarboxylic acid {2-chloro-4-[(5-chloro-thiophen-2-ylmethyl)-amino]-phenyl}-amide;
N-{2-Chloro-4-[(5-chloro-thiophen-2-ylmethyl)-amino]-phenyl}-2-thiophen-2-yl-acetamide;
N-{2-Chloro-4-[(5-chloro-thiophen-2-ylmethyl)-amino]-phenyl}-2-(3-methoxyphenyl)-acetamide;
N-{2-Chloro-4-[(5-chloro-thiophen-2-ylmethyl)-amino]-phenyl}-2-(4-chlorophenyl)-acetamide;
N-{2-Chloro-4-[(5-chloro-thiophen-2-ylmethyl)-amino]-phenyl}-2-(4-methoxyphenyl)-acetamide;
N-{2-Chloro-4-[(5-chloro-thiophen-2-ylmethyl)-amino]-phenyl}-2-(4-fluorophenyl)-acetamide;
2,3-Dihydro-benzo[1,4]dioxine-6-carboxyl acid {2-chloro-4-[(5-chloro-thiophen-2-ylmethyl)-amino]-phenyl}-amide;
2,3-Dihydro-benzofuran-5-carboxylic acid {2-chloro-4-[(5-chloro-thiophen-2-ylmethyl)-amino]-phenyl}-amide;
N-{2-Chloro-4-[(5-chloro-thiophen-2-ylmethyl)-amino]-phenyl}-3-cyclohexylpropionamide;
N-{4-[(5-Chloro-thiophen-2-ylmethyl)-(methyl)amino]-2-methyl-phenyl}-2,2-dimethylpropionamide;
N-{4-[(5-Chloro-thiophen-2-ylmethyl)-(methyl)amino]-2-methyl-phenyl}-2-phenyl acetamide;
N-{4-[(5-Chloro-thiophen-2-ylmethyl)-(methyl)amino]-2-methyl-phenyl}-3,3-dimethylbutyramide;
N-{4-[(5-Chloro-thiophen-2-ylmethyl)-(methyl)amino]-2-methyl-phenyl}-3-phenylpropionamide;
N-{4-[(5-Chloro-thiophen-2-ylmethyl)-(methyl)amino]-2-methyl-phenyl}-butyramide;
2,2,2-Trichloro-N-{4-[(5-chloro-thiophen-2-ylmethyl)-(methyl)amino]-2-methyl-phenyl}-acetamide;
Cyclopropanecarboxylic acid {4-[(5-chloro-thiophen-2-ylmethyl)-(methyl)amino]-2-methyl-phenyl}-amide;
Cyclobutanecarboxylic acid {4-[(5-chloro-thiophen-2-ylmethyl)-(methyl)amino]-2-methylphenyl}-amide;
Cyclopentanecarboxylic acid {4-[(5-chloro-thiophen-2-ylmethyl)-(methyl)amino]-2-methyl phenyl}-amide;
Cyclohexanecarboxylic acid {4-[(5-chloro-thiophen-2-ylmethyl)-(methyl)amino]-2-methylphenyl}-amide;
N-{4-[(5-Chloro-thiophen-2-ylmethyl)-(methyl)amino]-2-methylphenyl}-2-thiophen-2-yl-acetamide;
N-{4-[(5-Chloro-thiophen-2-ylmethyl)-(methyl)amino]-2-methylphenyl}-2-(3-methoxyphenyl)-acetamide;
N-{4-[(5-Chloro-thiophen-2-ylmethyl)-(methyl)amino]-2-methyl phenyl}-malonamic acid methyl ester;
2-(4-Chlorophenyl)-N-{4-[(5-chloro-thiophen-2-ylmethyl)-(methyl)amino]-2-methylphenyl}-acetamide;
N-{4-[(5-Chloro-thiophen-2-ylmethyl)-(methyl)amino]-2-methyl phenyl}-2-(4-methoxyphenyl)-acetamide;
N-{4-[(5-Chloro-thiophen-2-ylmethyl)-(methyl)amino]-2-methyl phenyl}-2-(4-fluorophenyl)-acetamide;
N-{4-[(5-Chloro-thiophen-2-ylmethyl)-(methyl)amino]-2-methylphenyl}-3-cyclohexylpropionamide;
{2-Chloro-4-[(5-chloro-thiophen-2-ylmethyl)-(methyl)amino]-phenyl}-carbamic acid phenyl ester;
{2-Chloro-4-[(5-chloro-thiophen-2-ylmethyl)-(methyl)amino]-phenyl}-carbamic acid benzyl ester;
{2-Chloro-4-[(5-chloro-thiophen-2-ylmethyl)-(methyl)amino]-phenyl}-carbamic acid isobutyl ester;
{2-Chloro-4-[(5-chloro-thiophen-2-ylmethyl)-(methyl)amino]-phenyl}-carbamic acid butyl ester;
{2-Chloro-4-[(5-chloro-thiophen-2-ylmethyl)-(methyl)amino]-phenyl}-carbamic acid hexyl ester;
{2-Chloro-4-[(5-chloro-thiophen-2-ylmethyl)-(methyl)amino]-phenyl}-carbamic acid 4-nitrobenzyl ester;
{2-Chloro-4-[(5-chloro-thiophen-2-ylmethyl)-(methyl)amino]-phenyl}-carbamic acid but-3-enyl ester;
{2-Chloro-4-[(5-chloro-thiophen-2-ylmethyl)-(methyl)amino]-phenyl}-carbamic acid but-2-ynyl ester;
{2-Chloro-4-[(5-chloro-thiophen-2-ylmethyl)-(methyl)amino]-phenyl}-carbamic acid 2,2-dimethylpropyl ester;
{2-Chloro-4-[(5-chloro-thiophen-2-ylmethyl)-(methyl)amino]-phenyl}-carbamic acid 2-chlorobenzyl ester;
{2-Chloro-4-[(5-chloro-thiophen-2-ylmethyl)-(methyl)amino]-phenyl}-carbamic acid 3-chloropropyl ester;
{2-Chloro-4-[(5-chloro-thiophen-2-ylmethyl)-(methyl)amino]-phenyl}-carbamic acid 2-benzyloxyethyl ester;
3-{2-Chloro-4-[(5-chloro-thiophen-2-ylmethyl)-(methyl)amino]-phenyl}-1-methyl-1-propyl-urea;
1-{2-Chloro-4-[(5-chloro-thiophen-2-ylmethyl)-(methyl)amino]-phenyl}-3-(2-fluorophenyl)-urea;
N-{2-Chloro-4-[(5-chloro-thiophen-2-ylmethyl)-(methyl)amino]-phenyl}-2,2,2-trifluoroacetamide;
N-{2-Chloro-4-[(5-chloro-thiophen-2-ylmethyl)-amino]-phenyl}-2,2,2-trifluoroacetamide;
N-{5-[(5-Chloro-thiophen-2-ylmethyl)-amino]-4′-dimethylamino-biphenyl-2-yl}-2-(4-fluorophenyl)-acetamide;
N-{2-Chloro-4-[(5-chloro-thiophen-2-ylmethyl)-(methyl)amino]-phenyl}-2-(4-chlorophenyl)-acetamide;
[4-(3-Fluoro-4-trifluoromethyl-benzylamino)-2-methylphenyl]-carbamic acid ethyl ester;
2-(4-Fluorophenyl)-N-{2-methyl-4-[(6-p-tolyloxypyridin-3-ylmethyl)-amino]-phenyl}-acetamide;
N-[2-Methyl-4-(4-trifluoromethyl-benzylamino)-phenyl]-butyramide;
2-(4-Fluorophenyl)-N-{2-methyl-4-[6-trifluoromethylpyridin-3-ylmethyl)-amino]-phenyl}-acetamide;
Pentanoic acid {4-[(5-chloro-thiophen-2-ylmethyl)-(methyl)amino]-2-methylphenyl}-amide;
3,3-Dimethyl-N-{2-methyl-4-[(6-p-tolyloxypyridin-3-ylmethyl)-amino]-phenyl}-butyramide;
[2-Methyl-4-(4-trifluoromethyl-benzylamino)-phenyl]-carbamic acid ethyl ester;
N-{2-Chloro-4-[(5-chloro-thiophen-2-ylmethyl)-(methyl)amino]-phenyl}-2-(4-chlorophenyl)-propionamide;
[4-(4-Chloro-benzylamino)-2-methylphenyl]-carbamic acid ethyl ester;
{4-[(6-Methoxy-benzo[b]thiophen-2-ylmethyl)-amino]-2-methylphenyl}-carbamic acid propyl ester;
{4-[(5=Chloro-thiophen-2-ylmethyl)-amino]-2-quinolin-3-yl-phenyl}-carbamic acid ethyl ester;
{4-[(5-Dimethylamino-3-methyl-benzo[b]thiophen-2-ylmethyl)-amino]-2-methylphenyl}-carbamic acid propyl ester;
3,3-Dimethyl-N-{2-methyl-4-[(6-trifluoromethylpyridin-3-ylmethyl)-amino]-phenyl}-butyramide;
N-(4-{[6-(4-Cyanophenoxy)-pyridin-3-ylmethyl]-amino}-2-methylphenyl)-2-(4-fluorophenyl)-acetamide;
{2-Benzyloxy-4-[(4-fluorobenzyl)-(methyl)amino]-phenyl}-thiocarbamic acid S-ethyl ester;
{2-Cyclopentyloxy-4-[(4-fluorobenzyl)-(methyl)amino]-phenyl}-thiocarbamic acid S-ethyl ester;
N-{4-[(6-Chloropyridin-3-ylmethyl)-amino]-2-methylphenyl}-2-(4-fluorophenyl)-acetamide;
{4-[(7-Dimethylamino-benzo[b]thiophen-2-ylmethyl)-amino]-2-methylphenyl}-carbamic acid propyl ester;
1-{2-Cyclopentyloxy-4-[(4-fluorobenzyl)-(methyl)amino]-phenyl}-3-ethyl-urea;
2-Amino-4-methyl-pentanoic acid [2-methyl-4-(4-trifluoromethyl-benzylamino)-phenyl]-amide;
{4-[(6-Methoxy-benzo[b]thiophen-2-ylmethyl)-amino]-2-methylphenyl}-carbamic acid ethyl ester;
2-Amino-4-methyl-pentanoic acid [2-methyl-4-(4-trifluoromethyl-benzylamino)-phenyl]-amide;
2-(4-Fluorophenyl)-N-{2-methyl-4-[(4-methyl-2-phenylpyrimidin-5-ylmethyl)-amino]-phenyl}-acetamide,
3,3-Dimethyl-N-{2-methyl-4-[(2-phenylpyrimidin-5-ylmethyl)-amino]-phenyl}-butyramide;
{4-[(5-Chloro-thiophen-2-ylmethyl)-amino]-2-pyridin-3-yl-phenyl}-carbamic acid ethyl ester;
1-Amino-cyclopropanecarboxylic acid [2-methyl-4-(4-trifluoromethyl-benzylamino)-phenyl]-amide;
{4-[(5-Chloro-thiophen-2-ylmethyl)-amino]-2-pyridin-4-yl-phenyl}-carbamic acid ethyl ester;
N-[2-Methyl-4-(4-trifluoromethyl-benzylamino)-phenyl]-2-piperidin-1-yl-acetamide;
N-(4-{[5-(4-Chlorophenoxy)-1,3-dimethyl-1H-pyrazol-4-ylmethyl]-amino}-2-methylphenyl)-2,2-dimethylpropionamide;
2,2-Dimethyl-N-{2-methyl-4-[(6-phenoxypyridin-3-ylmethyl)-amino]-phenyl}-propionamide;
N-[2-Methyl-4-(4-trifluoromethyl-benzylamino)-phenyl]-2-pyrrolidin-1-yl-acetamide;
[4-[(5-Chloro-thiophen-2-ylmethyl)-amino]-2-(6-methoxypyridin-3-yl)-phenyl]-carbamic acid ethyl ester;
4-[(3-Methyl-4-propoxycarbonylamino-phenyl amino)-methyl]-benzoic acid methyl ester;
N-[2-Methyl-4-(4-trifluoromethyl-benzylamino)-phenyl]-2-morpholin-4-yl-acetamide;
2,2-Dimethyl-N-{2-methyl-4-[(3-methyl-5-phenylisoxazol-4-ylmethyl)-amino]-phenyl}-propionamide;
{4-[(5-Chloro-thiophen-2-ylmethyl-amino]-2-iodophenyl}-carbamic acid ethyl ester;
N-{4-[(5-Chloro-thiophen-2-ylmethyl)-amino]-2-iodophenyl}-2-(4-fluorophenyl)-acetamide;
{4-[(5-Chloro-thiophen-2-ylmethyl)-amino]-2-quinolin-5-yl-phenyl}-carbamic acid ethyl ester; and
pharmaceutically acceptable salts thereof.

87. The method according to claim 1, wherein said compound is a compound according to formula 3: 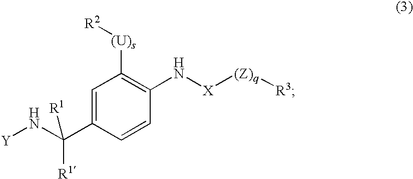
wherein:
U is O, S or NR^{2′};
s is 0 or 1;
X is CO or SO₂;
Z is O, S or NR⁴; wherein R⁴ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl and hydroxy-C₃₋₈-cycloalk(en)yl;
q is 0 or 1;
R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl;
R² is selected from the group consisting of hydrogen, halogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl, halo-C₃₋₈-cycloalk(en)yl and cyano; provided that:
when R² is halogen or cyano, then s is 0; and
when s is 1 and U is NR^{2′}, then R^{2′} is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; or R² and R^{2′} together form a 5-8 membered saturated or unsaturated ring that optionally contains one further heteroatom;
R³ is selected from the group consisting of C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; and
Y represents a group of formula VI, VII, VIII, IX or XXX: 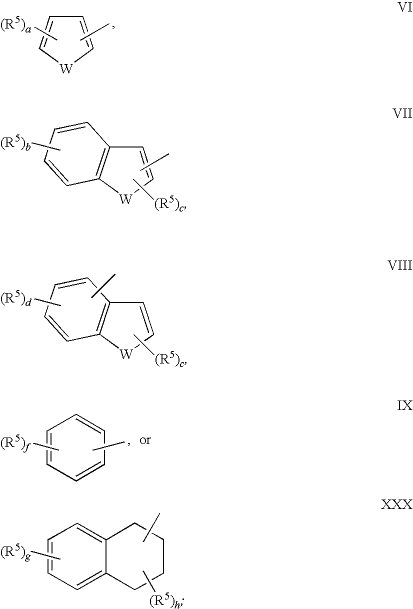
wherein:
the line represents a bond attaching the group represented by Y to the nitrogen atom;
W is O or S;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3; and
each R⁵ is independently selected from the group consisting of a C₁₋₆-alk(en/yn)yl, C₃₋₈-cycloalk(en)yl, Ar, C₃₋₈-cycloalk(en)yl-C₁₋₆-alk(en/yn)yl, Ar—C₁₋₆-alk(en/yn)yl, acyl, C₁₋₆-alk(an/en/yn)yloxy, halogen, halo-C₁₋₆-alk(en/yn)yl, —CO—NR⁶R^{6′}, cyano, nitro, —NR⁷R^{7′}, —S—R⁸ and SO₂OR⁸; or
two adjacent R⁵ substituents together with the aromatic group form a 5-8 membered saturated or unsaturated ring that optionally contains one or two heteroatoms; wherein:
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl; and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; with the provisos that;
when R⁵ is SO₂OR⁸ then R⁸ is not —NR⁹R^{9′}; and
when R⁵ is SO₂R⁸, then R⁸ is not a hydrogen atom;
or a pharmaceutically acceptable salt thereof.

88. (canceled)

89. The method according to claim 87, wherein said compound is selected from the group consisting of:
{2-Amino-4-[(4-tert-butyl phenylamino)-methyl]-phenyl}-carbamic acid ethyl ester;
(2-Amino-4-phenylaminomethyl-phenyl)-carbamic acid ethyl ester;
[2-Amino-4-(aphthalene-2-ylaminomethyl)-phenyl]carbamic acid ethyl ester;
[2-Amino-4-(p-tolylamino-methyl)-phenyl]carbamic acid ethyl ester;
{2-Amino-4-[(4-trifluoromethylphenylamino)-methyl]-phenyl}-carbamic acid ethyl ester;
{2-Amino-4-[(4-chlorophenylamino)-methyl]-phenyl}-carbamic acid ethyl ester;
{2-Amino-4-[(3-fluorophenylamino)-methyl]-phenyl}-carbamic acid ethyl ester;
{2-Amino-4-[(4-fluorophenylamino)-methyl]-phenyl}-carbamic acid ethyl ester;
{2-Amino-4-[(2-fluorophenylamino)-methyl]-phenyl}-carbamic acid ethyl ester;
[2-Amino-4-(biphenyl-4-ylaminomethyl)-phenyl]-carbamic acid ethyl ester;
{2-Amino-4-[(2,4-difluorophenylamino)-methyl]-phenyl}-carbamic acid ethyl ester;
{2-Amino-4-[(4-methoxyphenylamino)-methyl]-phenyl}-carbamic acid ethyl ester;
{2-Amino-4-[(4-cyclohexylphenylamino)-methyl]-phenyl}-carbamic acid ethyl ester;
[2-Amino-4-(indan-5-ylaminomethyl-phenyl]-carbamic acid ethyl ester;
{2-Amino-4-[(4-isopropylphenylamino)-methyl]-phenyl}-carbamic acid ethyl ester;
{2-Amino-4-[(4-butylphenylamino)-methyl]-phenyl}-carbamic acid ethyl ester;
{2-Amino-4-[(4-chloro-3-fluorophenylamino)methyl]phenyl}carbamic acid ethyl ester;
{2-Amino-4-[(2,4-dichlorophenylamino)methyl]phenyl}carbamic acid ethyl ester;
{2-Amino-4-[(2,3-dichlorophenylamino)methyl]phenyl}carbamic acid ethyl ester;
{2-Amino-4-[(3,5-dichlorophenylamino)methyl]phenyl}carbamic acid ethyl ester;
{2-Amino-4-[(3,4-dichlorophenylamino)methyl]phenyl}carbamic acid ethyl ester;
{2-Amino-4-[(3-trifluoromethylphenylamino)methyl]phenyl}carbamic acid ethyl ester;
{2-Amino-4-[(3-fluoro-4-trifluoromethylphenylamino)methyl]phenyl}carbamic acid ethyl ester;
{2-Amino-4-[(3,4-dichlorophenylamino)methyl]phenyl}carbamic acid ethyl ester;
{2-Amino-4-[(4-cyanophenylamino)methyl]phenyl}carbamic acid ethyl ester;
{2-Amino-4-[(4-fluoro-3-trifluoromethylphenylamino)methyl]phenyl}carbamic acid ethyl ester;
{2-Amino-4-[(3-chloro-4-methylphenylamino)methyl]phenyl}carbamic acid ethyl ester;
{2-Amino-4-[(3-chlorophenylamino)methyl]phenyl}carbamic acid ethyl ester;
[2-Amino-4-(m-tolylaminomethyl)phenyl]carbamic acid ethyl ester;
{2-Amino-4-[1-(4-chlorophenylamino)ethyl]phenyl}carbamic acid ethyl ester;
{2-Amino-4-[1-(4-trifluoromethylphenylamino)ethyl]phenyl}carbamic acid ethyl ester;
N-{2-Amino-4-[(3-fluorophenylamino)methyl]phenyl}-2,2-dimethylpropionamide;
{4-[(4-Chlorophenylamino)methyl]phenyl}carbamic acid ethyl ester;
{4-[(4-Trifluoromethylphenylamino)methyl]phenyl}carbamic acid ethyl ester;
{4-[(4-Chlorophenylamino)methyl]phenyl}carbamic acid ethyl ester;
{4-[(4-Fluorophenylamino)methyl]-2-methylphenyl}carbamic acid ethyl ester;
{4-[(4-Chlorophenylamino)methyl]-2-methylphenyl}carbamic acid ethyl ester;
{2-Methyl-4-[(4-trifluoromethylphenylamino)methyl]phenyl}carbamic acid ethyl ester;
{4-[(3,4-Difluorophenylamino)methyl]-2-methylphenyl}carbamic acid ethyl ester;
{4-[(3-Fluorophenylamino)methyl]-2-methylphenyl}carbamic acid ethyl ester;
{2-Chloro-4-[(4-chlorophenylamino)methyl]phenyl}carbamic acid ethyl ester;
{2-Chloro-4-[(4-trifluoromethyl-phenylamino)-methyl]phenyl}-carbamic acid ethyl ester;
{2-Chloro-4-[(4-fluorophenylamino)methyl]phenyl}-carbamic acid ethyl ester;
{2-Chloro-4-[(3-fluorophenylamino)methyl]phenyl}carbamic acid ethyl ester;
{2-Chloro-4-[(3,4-dichlorophenylamino)methyl]phenyl}carbamic acid ethyl ester;
{2-Chloro-4-[(4-chloro-3-fluorophenylamino)methyl]phenyl}carbamic acid ethyl ester;
{4-[(4-Chlorophenylamino)methyl]-2-fluorophenyl}carbamic acid ethyl ester;
{4-[(4-Chloro-3-fluorophenylamino)methyl]-2-fluorophenyl}carbamic acid ethyl ester;
{2-Fluoro-4-[(4-trifluoromethylphenylamino)methyl]phenyl}carbamic acid ethyl ester;
{4′-Dimethylamino-5-[(3-fluorophenylamino)methyl]biphenyl-2-yl}carbamic acid ethyl ester;
{4′-Dimethylamino-5-[(4-trifluoromethylphenylamino)methyl]biphenyl-2-yl}carbamic acid ethyl ester;
{4′-Chloro-5-[(3-fluorophenylamino)methyl]biphenyl-2-yl}carbamic acid ethyl ester;
{4′-Chloro-5-[(4-trifluoromethylphenylamino)methyl]biphenyl-2-yl}carbamic acid ethyl ester;
N-{4-[(4-chlorophenylamino)methyl]phenyl}butyramide;
N-{4-[(3,4-dichlorophenylamino)methyl]phenyl}butyramide;
N-{4-[(4-chloro-3-fluorophenylamino)methyl]phenyl}butyramide;
N-{4-[(4-fluoro-phenylamino)methyl]-2-methyl phenyl}butyramide;
N-{4-[(3-fluorophenylamino)methyl]-2-methylphenyl}butyramide;
N-{4-[(4-chlorophenylamino)methyl]-2-methylphenyl}butyramide;
N-{4-[(3,4-dichlorophenylamino)methyl]-2-methylphenyl}butyramide;
N-{4-[(4-chloro-3-fluorophenylamino)methyl]-2-methyl phenyl}butyramide;
N-{2-chloro-4-[(4-trifluoromethylphenylamino)methyl]phenyl}butyramide;
N-{2-chloro-4-[(4-fluorophenylamino)methyl]phenyl}butyramide;
N-{2-chloro-4-[(3-fluorophenylamino)methyl]phenyl}butyramide;
N-{2-chloro-4-[(4-chlorophenylamino)methyl]phenyl}butyramide;
N-{2-chloro-4-[(3,4-dichlorophenylamino)methyl]phenyl}butyramide;
N-{2-chloro-4-[(4-chloro-3-fluorophenylamino)methyl]phenyl}butyramide;
N-{2-fluoro-4-[(3-fluorophenylamino)methyl]phenyl}butyramide;
N-{4-[(4-chlorophenylamino)methyl]-2-fluorophenyl}butyramide;
N-{2-fluoro-4-[(4-trifluoromethylphenylamino)methyl]phenyl}butyramide;
N-{-4-[(3,4-dichlorophenylamino)methyl]-2-fluorophenyl}butyramide;
N-{4-[(4-chloro-3-fluorophenylamino)methyl]-2-fluorophenyl}butyramide; and
pharmaceutically acceptable salts thereof.

90. The method according to claim 1, wherein said compound is a compound according to formula 4: 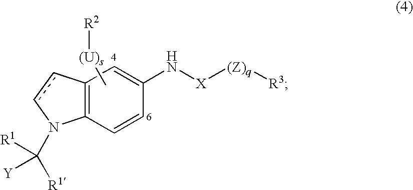
wherein;
the dotted line represents an optional bond;
R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or
R¹ and R^{1′} together with the carbon atom to which they are attached form a 3-8 membered saturated or unsaturated ring that optionally contains 1 or 2 heteroatoms;
s is 0 or 1;
U is O, NR¹¹, S, SO₂, SO₂NR¹¹, CO—O or CO—NR¹¹; wherein:
R¹¹ is selected from the group consisting of hydrogen, C₁₋₆-alk(en/yn)yl, C₃₋₈-cycl oal k(en)yl, and C₃₋₈-cycloalk(en)yl-C₁₋₆-alk(en/yn)yl; or
R² and R^{2′} together with the nitrogen atom to which they are attached form a 4-8 membered saturated or unsaturated ring that optionally contains 1, 2 or 3 further heteroatoms;
R² is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NO₂, NR¹⁰R^{10′—C}₁₋₆-alk(en/yn)yl, NR¹⁰R^{10′}—C₃₋₈-cycloalk(en)yl and NR¹⁰R^{10′}—C₃₋₈-cycloalk(en)yl-C₁₋₆-alk(en/yn)yl; wherein:
R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-Cl_{—}6-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or
R¹⁰ and R^{10′} together with the nitrogen atom to which they are attached form a 4-8 membered saturated or unsaturated ring that optionally contains 1, 2 or 3 further heteroatoms; with the proviso that;
when R² is NO₂, halogen or cyano, then s is 0; and
when R², R² is a hydrogen atom or acyl and s is 1, then U is NR¹¹, O or S; wherein the group —(U)ₛ—R² is linked to position 4 or 6 of the indole or indoline;
q is 0 or 1;
Z is O or S;
X is CO or SO₂; with the proviso that when q is 0, then X is SO₂;
R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, Ar, Ar—C₁₋₆-alk(en/yn)yl, Ar—C₃₋₈-cycloalk(en)yl, Ar-heterocycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar—C₁₋₆-alk(en/yn)yl-heterocycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar—C₁₋₆-alk(en/yn)yloxy-C₁₋₆-alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl, —NR¹²R^{12′}, optionally substituted NR¹²R^{12′}—C₁₋₆-alk(en/yn)yl, optionally substituted NR¹²R^{12′}—C₃₋₈-cycloalk(en)yl, and optionally substituted NR¹²R^{12′}—C₃₋₈-cycloalk(en)yl-C₁₋₆-alk(en/yn)yl; wherein:
R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or
R¹² and R^{12′} together with the nitrogen atom to which they are attached form a 4-8 membered saturated or unsaturated ring that optionally contains 1, 2 or 3 further heteroatoms; with the proviso that when R³ is NR¹²R^{12′}, then q is 0; and
Y represents a group of formula II, III, IV, V, VI, XXX and XXXI: 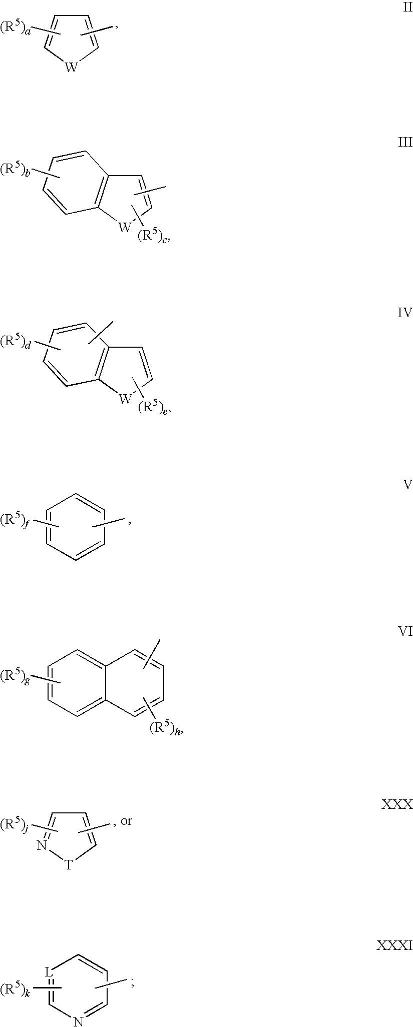
wherein:
the line represents a bond attaching the group represented by Y to the carbon atom;
W is O or S;
T is N, NE or O;
L is N, C or CH;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3;
j is 0, 1, 2 or 3; with the provisos that:
when T is a nitrogen atom, then j is 0, 1, 2 or 3; and
when T is NH or an oxygen atom, then j is 0, 1 or 2;
k is 0, 1, 2, 3 or 4; and
each R⁵ is independently selected from the group consisting of a C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar-thio, Ar-oxy, acyl, C₁₋₆-alk(en/yn)yloxy, C₃₋₈-cycloalk(en)yloxy, C₃₋₈-cycloalk(en)yl-C₁₋₆-alk(en/yn)yloxy, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —CO—NR⁶R^{6′}, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NR⁷R^{7′}, —S—R⁸ and —SO₂R⁸; or
two adjacent R⁵ substituents together with the aromatic group to which they are attached form a 4-8 membered ring that optionally contains one or two heteroatoms; wherein:
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl; and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′};
wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; provided that when R⁸ is —NR⁹R^{9′}, then R⁵ is not —S—R⁸,
or a pharmaceutically acceptable salt thereof.

91. (canceled)

92. The method according to claim 90, wherein said compound is selected from the group consisting of:
N-[4-Chloro-1-(4-trifluoromethylbenzyl)-2,3-dihydro-1H-indol-5-yl]-3,3-dimethylbutyramide;
N-[4-Chloro-1-(5-chlorothiophen-2-ylmethyl)-2,3-dihydro-1H-indol-5-yl]-3,3-dimethylbutyramide;
[1-(4-Fluorobenzyl)-2,3-dihydro-1H-indol-5-yl]-carbamic acid propyl ester;
N-[1-(4-Fluorobenzyl)-2,3-dihydro-1H-indol-5-yl]-C-phenyl-methanesulfonamide;
4-Fluoro-N-[1-(4-fluorobenzyl)-2,3-dihydro-1H-indol-5-yl]-benzamide;
N-[1-(4-Fluorobenzyl)-2,3-dihydro-1H-indol-5-yl]-3,3-dimethylbutyramide;
N-[1-(4-Fluorobenzyl)-2,3-dihydro-1H-indol-5-yl]-2-thiophen-2-ylacetamide;
N-[1-(4-Fluorobenzyl)-2,3-dihydro-1H-indol-5-yl]-2-(4-fluorophenyl)-acetamide;
3-[1-(5-Chlorothiophen-2-ylmethyl)-2,3-dihydro-1H-indol-5-yl]-1,1-diisopropylurea;
Morpholine-4-carboxylic acid [1-(5-chlorothiophen-2-ylmethyl)-2,3-dihydro-1H-indol-5-yl]-amide;
Pyrrolidine-1-carboxylic acid [1-(5-chlorothiophen-2-ylmethyl)-2,3-dihydro-1H-indol-5-yl]-amide;
[1-(5-Chlorothiophen-2-ylmethyl)-2,3-dihydro-1H-indol-5-yl]-carbamic acid 2-benzyloxyethyl ester;
3-[1-(5-Chlorothiophen-2-ylmethyl)-2,3-dihydro-1H-indol-5-yl]-1-methyl-1-propylurea;
[1-(5-Chlorothiophen-2-ylmethyl)-2,3-dihydro-1H-indol-5-yl]-carbamic acid tert-butyl ester;
N-[1-(5-Chlorothiophen-2-ylmethyl)-2,3-dihydro-1H-indol-5-yl]-C-phenyl-methanesulfonamide;
Butane-1-sulfonic acid [1-(5-chlorothiophen-2-ylmethyl)-2,3-dihydro-1H-indol-5-yl]-amide;
N-[1-(5-Chlorothiophen-2-ylmethyl)-2,3-dihydro-1H-indol-5-yl]-4-fluorobenzamide;
N-[1-(5-Chlorothiophen-2-ylmethyl)-2,3-dihydro-1H-indol-5-yl]-2,2-dimethylpropionamide;
N-[1-(5-Chlorothiophen-2-ylmethyl)-2,3-dihydro-1H-indol-5-yl]-2-phenoxyacetamide;
N-[1-(5-Chlorothiophen-2-ylmethyl)-2,3-dihydro-1H-indol-5-yl]-3,3-dimethylbutyramide;
N-[1-(5-Chlorothiophen-2-ylmethyl)-2,3-dihydro-1H-indol-5-yl]-butyramide;
Cyclopentanecarboxylic acid [1-(5-chlorothiophen-2-ylmethyl)-2,3-dihydro-1H-indol-5-yl]-amide;
N-[1-(5-Chlorothiophen-2-ylmethyl)-2,3-dihydro-1H-indol-5-yl]-2-thiophen-2-ylacetamide;
N-[1-(5-Chlorothiophen-2-ylmethyl)-2,3-dihydro-1H-indol-5-yl]-isonicotinamide;
N-[1-(5-Chlorothiophen-2-ylmethyl)-2,3-dihydro-1H-indol-5-yl]-4-dimethylaminobenzamide;
N-[1-(5-Chlorothiophen-2-ylmethyl)-2,3-dihydro-1H-indol-5-yl]-2-(4-fluorophenyl)-acetamide;
N-[1-(5-Chlorothiophen-2-ylmethyl)-2,3-dihydro-1H-indol-5-yl]-6-trifluoromethylnicotinamide;
1-tert-Butyl-3-[1-(5-chlorothiophen-2-ylmethyl)-2,3-dihydro-1H-indol-5-yl]-urea;
1-[1-(5-Chlorothiophen-2-ylmethyl)-2,3-dihydro-1H-indol-5-yl]-3-ethyl urea;
1-Benzyl-3-[1-(5-chlorothiophen-2-ylmethyl)-2,3-dihydro-1H-indol-5-yl]-urea;
1-[1-(5-Chlorothiophen-2-ylmethyl)-2,3-dihydro-1H-indol-5-yl]-3-phenethyl urea;
1-[1-(5-Chlorothiophen-2-ylmethyl)-2,3-dihydro-1H-indol-5-yl]-3-thiophen-2-ylurea;
1-[1-(5-Chlorothiophen-2-ylmethyl)-2,3-dihydro-1H-indol-5-yl]-3-thiophen-3-ylurea;
[1-(5-Chlorothiophen-2-ylmethyl)-2,3-dihydro-1H-indol-5-yl]-carbamic acid propyl ester;
2,2-Dimethyl-N-[6-nitro-1-(4-trifluoromethylbenzyl)-2,3-dihydro-1H-indol-5-yl]-propionamide;
N-[1-(5-Chlorothiophen-2-ylmethyl)-6-nitro-2,3-dihydro-1H-indol-5-yl]-2,2-dimethylpropionamide;
2-(4-Fluorophenyl)-N-[6-nitro-1-(4-trifluoromethylbenzyl)-2,3-dihydro-1H-indol-5-yl]-acetamide;
N-[1-(5-Chlorothiophen-2-ylmethyl)-6-nitro-2,3-dihydro-1H-indol-5-yl]-2-(4-fluorophenyl)-acetamide;
N-[1-(5-Chlorothiophen-2-ylmethyl)-6-nitro-2,3-dihydro-1H-indol-5-yl]-3,3-dimethylbutyramide;
N-[6-Amino-1-(5-chlorothiophen-2-ylmethyl)-2,3-dihydro-1H-indol-5-yl]-3,3-dimethylbutyramide;
N-[6-Amino-1-(4-trifluoromethylbenzyl)-2,3-dihydro-1H-indol-5-yl]-2,2-dimethylpropionamide;
N-[6-Amino-1-(5-chlorothiophen-2-ylmethyl)-2,3-dihydro-1H-indol-5-yl]-2,2-dimethylpropionamide;
N-[6-Amino-1-(4-trifluoromethylbenzyl)-2,3-dihydro-1H-indol-5-yl]-2-(4-fluorophenyl)-acetamide;
N-[6-Amino-1-(4-trifluoromethylbenzyl)-2,3-dihydro-1H-indol-5-yl]-3,3-dimethylbutyramide,
N-[6-Amino-1-(4-fluorobenzyl)-2,3-dihydro-1H-indol-5-yl]-3,3-dimethylbutyramide;
N-[6-Amino-1-(3-fluoro-4-trifluoromethylbenzyl)-2,3-dihydro-1H-indol-5-yl]-3,3-dimethylbutyramide;
N-[1-(5-Chlorothiophen-2-ylmethyl)-1H-indol-5-yl]-3,3-dimethylbutyramide;
N-[6-Bromo-1-(4-trifluoromethylbenzyl)-2,3-dihydro-1H-indol-5-yl]-3,3-dimethylbutyramide;
N-[6-Bromo-1-(5-chlorothiophen-2-ylmethyl)-2,3-dihydro-1H-indol-5-yl]-3,3-dimethylbutyramide;
N-[1-(4-Chlorobenzyl)-2,3-dihydro-1H-indol-5-yl]-3,3-dimethylbutyramide;
3,3-Dimethyl-N-[1-(4-trifluoromethylbenzyl)-2,3-dihydro-1H-indol-5-yl]-butyramide;
N-[1-(4-Isopropylbenzyl)-2,3-dihydro-1H-indol-5-yl]-3,3-dimethylbutyramide;
N-[1-(3-Fluoro-4-trifluoromethylbenzyl)-2,3-dihydro-1H-indol-5-yl]-3,3-dimethylbutyramide;
N-[1-(6-Chlorobenzo[1,3]dioxol-5-ylmethyl)-2,3-dihydro-1H-indol-5-yl]-3,3-dimethylbutyramide,
N-[1-(3,5-Dimethyl-1-phenyl-1H-pyrazol-4-ylmethyl)-2,3-dihydro-1H-indol-5-yl]-3,3-dimethylbutyramide;
N-[1-(2-Chloro-5-trifluoromethylbenzyl)-2,3-dihydro-1H-indol-5-yl]-3,3-dimethylbutyramide;
N-{1-[5-(4-Chlorophenoxy)-1,3-dimethyl-1H-pyrazol-4-ylmethyl]-2,3-dihydro-1H-indol-5-yl}-3,3-dimethylbutyramide;
3,3-Dimethyl-N-[1-(6-p-tolyloxy-pyridin-3-ylmethyl)-2,3-dihydro-1H-indol-5-yl]-butyramide;
N-{1-[6-(4-Chlorophenyl sulfanyl)-pyridin-3-ylmethyl]-2,3-dihydro-1H-indol-5-yl}-3,3-dimethylbutyramide,
N-{1-[6-(4-Cyanophenoxy)-pyridin-3-ylmethyl]-2,3-dihydro-1H-indol-5-yl}-3,3-dimethylbutyramide;
3,3-Dimethyl-N-[1-(6-trifluoromethylpyridin-3-ylmethyl)-2,3-dihydro-1H-indol-5-yl]-butyramide;
3,3-Dimethyl-N-[1-(3-methyl-benzo[b]thiophen-2-ylmethyl)-2,3-dihydro-1H-indol-5-yl]-butyramide;
N-[1-(6-Fluoro-4H-benzo[1,3]dioxin-8-ylmethyl)-2,3-dihydro-1H-indol-5-yl]-3,3-dimethylbutyramide;
3,3-Dimethyl-N-[1-(6-phenoxypyridin-3-ylmethyl)-2,3-dihydro-1H-indol-5-yl]-butyramide;
3,3-Dimethyl-N-[1-(3-methyl-5-phenyl-isoxazol-4-ylmethyl)-2,3-dihydro-1H-indol-5-yl]-butyramide,
N-(1-Benzo[b]thiophen-2-ylmethyl-2,3-dihydro-1H-indol-5-yl)-3,3-dimethylbutyramide;
N-{1-[1-(4-Fluorophenyl)-5-methyl-1H-pyrazol-4-ylmethyl]-2,3-dihydro-1H-indol-5-yl}-3,3-dimethylbutyramide;
3,3-Dimethyl-N-[1-(5-methyl thiophen-2-ylmethyl)-2,3-dihydro-1H-indol-5-yl]-butyramide;
3,3-Dimethyl-N-[1-(4-pyrrol-1-yl-benzyl)-2,3-dihydro-1H-indol-5-yl]-butyramide;
N-[1-(4-Chlorobenzyl)-2,3-dihydro-1H-indol-5-yl]-2-(4-fluorophenyl)-acetamide;
2-(4-Fluorophenyl)-N-[1-(4-trifluoromethylbenzyl)-2,3-dihydro-1H-indol-5-yl]-acetamide;
2-(4-Fluorophenyl)-N-[1-(4-isopropylbenzyl)-2,3-dihydro-1H-indol-5-yl]-acetamide;
2-(4-Fluorophenyl)-N-[1-(3-fluoro-4-trifluoromethylbenzyl)-2,3-dihydro-1H-indol-5-yl]-acetamide;
N-[1-(6-Chlorobenzo[1,3]dioxol-5-ylmethyl)-2,3-dihydro-1H-indol-5-yl]-2-(4-fluorophenyl)-acetamide;
N-[1-(3,5-Dimethyl-1-phenyl-1H-pyrazol-4-ylmethyl)-2,3-dihydro-1H-indol-5-yl]-2-(4-fluorophenyl)-acetamide;
N-[1-(2-Chloro-5-trifluoromethylbenzyl)-2,3-dihydro-1H-indol-5-yl]-2-(4-fluorophenyl)-acetamide;
N-{1-[5-(4-Chlorophenoxy)-1,3-dimethyl-1H-pyrazol-4-ylmethyl]-2,3-dihydro-1H-indol-5-yl}-2-(4-fluorophenyl)-acetamide;
N-{1-[6-(4-Cyanophenoxy)-pyridin-3-ylmethyl]-2,3-dihydro-1H-indol-5-yl}-2-(4-fluorophenyl)-acetamide;
2-(4-Fluorophenyl)-N-[1-(3-methyl-benzo[b]thiophen-2-ylmethyl)-2,3-dihydro-1H-indol-5-yl]-acetamide;
N-[1-(6-Fluoro-4H-benzo[1,3]dioxin-8-ylmethyl)-2,3-dihydro-1H-indol-5-yl]-2-(4-fluorophenyl)-acetamide;
2-(4-Fluorophenyl)-N-[1-(6-phenoxypyridin-3-ylmethyl)-2,3-dihydro-1H-indol-5-yl]-acetamide;
N-(1-Benzo[b]thiophen-2-ylmethyl-2,3-dihydro-1H-indol-5-yl)-2-(4-fluorophenyl)-acetamide;
2-(4-Fluorophenyl)-N-{1-[1-(4-fluorophenyl)-5-methyl-1H-pyrazol-4-ylmethyl]-2,3-dihydro-1H-indol-5-yl}-acetamide;
2-(4-Fluorophenyl)-N-[1-(5-methylthiophen-2-ylmethyl)-2,3-dihydro-1H-indol-5-yl]-acetamide;
2-(4-Fluorophenyl)-N-[1-(4-pyrrol-1-yl-benzyl)-2,3-dihydro-1H-indol-5-yl]-acetamide; and
pharmaceutically acceptable salts thereof.

93. The method according to claim 1, wherein said compound is a compound according to formula 5: 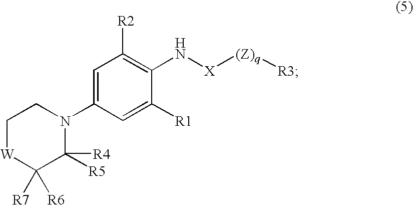
wherein:
q is 0 or 1;
W is O or S;
X is CO;
Z is O;
R1 is selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy;
R2 is selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy, C₃₋₈-cycloalk(en)yl-C₁₋₆-alk(en/yn)yloxy, optionally substituted phenyl and optionally substituted pyridyl; wherein the phenyl and pyridyl are optionally substituted with one or more substituents independently being halogen, C₁₋₆-alk(en/yr)yl, C₃₋₈-cycloalk(en)yl or C₃₋₈-cycloalk(en)yl-C₁₋₆-alk(en/yn)yl;
R3 is selected from the group consisting of C₁₋₁₀-alk(en/yn)yl, C₃₋₈-cycloalk(en)yl, C₃₋₈-cycloalk(en)yl-C₁₋₆-alk(en/yn)yl, Ar—C₁₋₆-alk(en/yn)yl, Ar—C₃₋₈-cycloalk(en)yl, Ar—C₃₋₈-cycloalk(en)yl-C₁₋₆-alk(en/yn)yl and Ar; and
each of R4, R5, R6 and R7 is independently selected from the group consisting of hydrogen and Ar;
as the free base or a salt thereof.

94. (canceled)

95. The method according to claim 93, wherein said compound is selected from the group consisting of:
N-(2-Bromo-4-morpholin-4-yl-6-trifluoromethyl-phenyl)-2-(4-fluoro-phenyl)-acetamide;
2-Cyclopentyl-N-(2-bromo-6-trifluoromethyl-4-morpholin-4-yl-phenyl)-acetamide;
N-(2-Bromo-4-morpholin-4-yl-6-trifluoromethyl-phenyl)-3-cyclopentyl-propionamide;
N-(2-Chloro-6-cyano-4-morpholin-4-yl-phenyl)-3-cyclohexyl-propionamide;
2-Cyclopentyl-N-(2,6-dimethyl-4-thiomorpholin-4-yl-phenyl)-acetamide;
2-Cyclopentyl-N-[2,6-dimethyl-4-(2-phenyl-morpholin-4-yl)-phenyl]-acetamide;
2-Cyclopentyl-N-[2,6-dimethyl-4-(2-phenyl-thiomorpholin-4-yl)-phenyl]-acetamide;
2-Cyclopentyl-N-[2,6-dimethyl-4-(3-pyridin-3-yl-thiomorpholin-4-yl)-phenyl]-acetamide;
2-Cyclopentyl-N-{2,6-dimethyl-4-[2-(4-trifluoromethyl-phenyl)-thiomorpholin-4-yl]-phenyl}-acetamide;
N-{4-[2-(2-Chloro-phenyl)-thiomorpholin-4-yl]-2,6-dimethyl-phenyl}-2-cyclopentyl-acetamide;
2-Bicyclo[2.2.1]hept-2-yl-N-(2,6-dimethyl-4-morpholin-4-yl-phenyl)-acetamide;
2-Cyclohexyl-N-(2,6-dimethyl-4-morpholin-4-yl-phenyl)-acetamide;
3-(3,4-Difluoro-phenyl)-N-(2,6-dimethyl-4-morpholin-4-yl-phenyl)-propionamide;
2-Cyclopentyl-N-(2,6-dimethyl-4-morpholin-4-yl-phenyl)-acetamide;
(2,6-Dimethyl-4-morpholin-4-yl-phenyl)-carbamic acid butyl ester;
2-(4-Chloro-phenyl)-N-(2,6-dimethyl-4-morpholin-4-yl-phenyl)-acetamide;
2,3-Dihydro-benzofuran-2-carboxylic acid (2,6-dimethyl-4-morpholin-4-yl-phenyl)-amide;
3-Cyclohexyl-N-(2,6-dimethyl-4-morpholin-4-yl-phenyl)-propionamide;
3-Cyclopentyl-N-(2,6-dimethyl-4-morpholin-4-yl-phenyl)-propionamide;
N-(2,6-Dimethyl-4-morpholin-4-yl-phenyl)-2-(4-fluoro-phenyl)-acetamide;
N-(2,6-Dimethyl-4-morpholin-4-yl-phenyl)-2-thiophen-2-yl-acetamide;
N-(2,6-Dimethyl-4-morpholin-4-yl-phenyl)-3,3-dimethyl-butyramide;
Hexanoic acid (2,6-dimethyl-4-morpholin-4-yl-phenyl)-amide;
2-Cycloheptyl-N-(2,6-dimethyl-4-morpholin-4-yl-phenyl)-acetamide;
(2,6-Dimethyl-4-morpholin-4-yl-phenyl)-carbamic acid benzyl ester;
(2,6-Dimethyl-4-morpholin-4-yl-phenyl)-carbamic acid 2-chloro-benzyl ester;
3,5,5-Trimethyl-hexanoic acid (2,6-dimethyl-4-morpholin-4-yl-phenyl)-amide;
Octanoic acid (2,6-dimethyl-4-morpholin-4-yl-phenyl)-amide;
Heptanoic acid (2,6-dimethyl-4-morpholin-4-yl-phenyl)-amide;
N-(2,6-Dimethyl-4-morpholin-4-yl-phenyl)-2-phenyl-acetamide;
2-(3,4-Dichloro-phenyl)-N-(2,6-dimethyl-4-morpholin-4-yl-phenyl)-acetamide;
2-(4-Allyloxy-3-chloro-phenyl)-N-(2,6-dimethyl-4-morpholin-4-yl-phenyl)-acetamide;
N-(2,6-Dimethyl-4-morpholin-4-yl-phenyl)-2-(3-trifluoromethyl-phenyl)-acetamide;
N-(2,6-Dimethyl-4-morpholin-4-yl-phenyl)-2-naphthalen-2-yl-acetamide;
3-(3-Chloro-phenyl)-N-(2,6-dimethyl-4-morpholin-4-yl-phenyl)-propionamide;
N-(2,6-Dimethyl-4-morpholin-4-yl-phenyl)-2-(3,4-dimethyl-phenyl)-acetamide;
2-(3-Bromo-phenyl)-N-(2,6-dimethyl-4-morpholin-4-yl-phenyl)-acetamide;
2-(3-Chloro-phenyl)-N-(2,6-dimethyl-4-morpholin-4-yl-phenyl)-acetamide;
N-(2,6-Dimethyl-4-morpholin-4-yl-phenyl)-2-p-tolyl-acetamide;
N-(2,6-Dimethyl-4-morpholin-4-yl-phenyl)-2-m-tolyl-acetamide;
2-(3,4-Difluoro-phenyl)-N-(2,6-dimethyl-4-morpholin-4-yl-phenyl)-acetamide;
N-(2,6-Dimethyl-4-morpholin-4-yl-phenyl)-2-(3-fluoro-phenyl)-acetamide;
N-(2-Bromo-4-morpholin-4-yl-6-trifluoromethyl-phenyl)-3-cyclohexyl-propionamide;
N-(2-Bromo-4-morpholin-4-yl-6-trifluoromethyl-phenyl)-2-(3-fluoro-phenyl)-acetamide;
N-(2-Bromo-4-morpholin-4-yl-6-trifluoromethyl-phenyl)-propionamide;
N-(2-Bromo-4-morpholin-4-yl-6-trifluoromethyl-phenyl)-butyramide;
N-(2-Chloro-4-morpholin-4-yl-6-trifluoromethyl-phenyl)-2-(3-fluoro-phenyl)-acetamide;
N-(2-Chloro-4-morpholin-4-yl-6-trifluoromethyl-phenyl)-2-cyclopentyl-acetamide;
2-Cyclopentyl-N-{2,6-dimethyl-4-[2-(4-trifluoromethyl-phenyl)-morpholin-4-yl]-phenyl}-acetamide;
N-{4-[2-(2-Chloro-phenyl)-morpholin-4-yl]-2,6-dimethyl-phenyl}-2-cyclopentyl-acetamide;
2-Cyclopentyl-N-{4-[2-(4-fluoro-phenyl)-morpholin-4-yl]-2,6-dimethyl-phenyl}-acetamide;
2-(2-Chloro-phenyl)-N-(2,6-dimethyl-4-morpholin-4-yl-phenyl)-acetamide;
Pentanoic acid (2,6-dimethyl-4-morpholin-4-yl-phenyl)-amide;
4-Methyl-pentanoic acid (2,6-dimethyl-4-morpholin-4-yl-phenyl)-amide;
2-Cyclopent-2-enyl-N-(2,6-dimethyl-4-morpholin-4-yl-phenyl)-acetamide;
5-Methyl-hexanoic acid (2,6-dimethyl-4-morpholin-4-yl-phenyl)-amide;
3-Methyl-pentanoic acid (2,6-dimethyl-4-morpholin-4-yl-phenyl)-amide;
Hex-5-enoic acid (2,6-dimethyl-4-morpholin-4-yl-phenyl)-amide;
3-Ethyl-pentanoic acid (2,6-dimethyl-4-morpholin-4-yl-phenyl)-amide;
2-Cyclopentyl-N-(4-morpholin-4-yl-2-pyridin-3-yl-6-trifluoromethyl-phenyl)-acetamide;
2-Cyclopentyl-N-(5-morpholin-4-yl-3-trifluoromethyl-biphenyl-2-yl)-acetamide;
2-Cyclopentyl-N-(4′-fluoro-5-morpholin-4-yl-3-trifluoromethyl-biphenyl-2-yl)-acetamide;
2-Cyclopentyl-N-(4′-methyl-5-morpholin-4-yl-3-trifluoromethyl-biphenyl-2-yl)-acetamide,
2-Cyclopentyl-N-(3′-methyl-5-morpholin-4-yl-3-trifluoromethyl-biphenyl-2-yl)-acetamide;
2-Cyclopentyl-N-(3′,4′-difluoro-5-morpholin-4-yl-3-trifluoromethyl-biphenyl-2-yl)-acetamide;
2-(4-Fluoro-phenyl)-N-(4-morpholin-4-yl-2-pyridin-3-yl-6-trifluoromethyl-phenyl)-acetamide;
2-Cyclopentyl-N-(2,6-diethyl-4-morpholin-4-yl-phenyl)-acetamide;
2-Cyclopentyl-N-(2,6-diisopropyl-4-morpholin-4-yl-phenyl)-acetamide;
2-Cyclopentyl-N-(2,6-difluoro-4-morpholin-4-yl-phenyl)-acetamide;
Hexanoic acid (2,6-difluoro-4-morpholin-4-yl-phenyl)-amide;
N-(2,6-Difluoro-4-morpholin-4-yl-phenyl)-3,3-dimethyl-butyramide;
N-(2,6-Difluoro-4-morpholin-4-yl-phenyl)-2-(3-fluoro-phenyl)-acetamide;
2-Cyclopent-2-enyl-N-(2,6-difluoro-4-morpholin-4-yl-phenyl)-acetamide;
2-Bicyclo[2.2.1]hept-2-yl-N-(2,6-difluoro-4-morpholin-4-yl-phenyl)-acetamide;
2-Bicyclo[2.2.1]hept-2-yl-N-(2-methyl-4-morpholin-4-yl-6-trifluoromethyl-phenyl)-acetamide;
5-Methyl-pentanoic acid (2-methyl-4-morpholin-4-yl-6-trifluoromethyl-phenyl)-amide;
5-Methyl-hexanoic acid (2-methyl-4-morpholin-4-yl-6-trifluoromethyl-phenyl)-amide;
2-Cyclopent-2-enyl-N-(2-methyl-4-morpholin-4-yl-6-trifluoromethyl-phenyl)-acetamide;
2-Cyclopentyl-N-(2-methyl-4-morpholin-4-yl-6-trifluoromethyl-phenyl)-acetamide;
Hexanoic acid (2-methyl-4-morpholin-4-yl-6-trifluoromethyl-phenyl)-amide;
3,3-Dimethyl-N-(2-methyl-4-morpholin-4-yl-6-trifluoromethyl-phenyl)-butyramide;
2-(3,4-Difluoro-phenyl)-N-(2-methyl-4-morpholin-4-yl-6-trifluoromethyl-phenyl)-acetamide;
Hexanoic acid (2-methoxy-6-methyl-4-morpholin-4-yl-phenyl)-amide;
2-Cyclopentyl-N-(2-methoxy-6-methyl-4-morpholin-4-yl-phenyl)-acetamide;
N-(2-Methoxy-6-methyl-4-morpholin-4-yl-phenyl)-3,3-dimethyl-butyramide;
2-(3,4-Difluoro-phenyl)-N-(2-methoxy-6-methyl-4-morpholin-4-yl-phenyl)-acetamide;
2-Cyclopent-2-enyl-N-(2-methoxy-6-methyl-4-morpholin-4-yl-phenyl)-acetamide;
2-(3-Fluoro-phenyl)-N-(2-methoxy-6-methyl-4-morpholin-4-yl-phenyl)-acetamide;
2-Bicyclo[2.2.1]hept-2-yl-N-(2-methoxy-6-methyl-4-morpholin-4-yl-phenyl)-acetamide;
4-Methyl-pentanoic acid (2-methoxy-6-methyl-4-morpholin-4-yl-phenyl)-amide;
5-Methyl-Hexanoic acid (2-methoxy-6-methyl-4-morpholin-4-yl-phenyl)-amide;
N-(2-Chloro-6-methyl-4-morpholin-4-yl-phenyl)-2-(3-fluoro-phenyl)-acetamide; and
N-(2-Chloro-6-methyl-4-morpholin-4-yl-phenyl)-2-cyclopentyl-acetamide;
as the free base or a pharmaceutically acceptable salt thereof.

96. The method according to claim 1, wherein said compound is a compound according to formula 6: 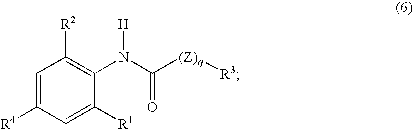
wherein:
Z is O or S;
q is 0 or 1;
each of R¹ and R² is independently selected from the group consisting of halogen, cyano, amino, C₁₋₆-alk(en/yn)yl, C₃₋₈-cycloalk(en)yl, C₃₋₈-cycloalk(en)yl-C₁₋₆-alk(en/yn)yl, C₃₋₈-heterocycloalk(en)yl, Aryl, Heteroaryl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy, C₃₋₈-cycloalk(en)yl-C₁₋₆-alk(en/yn)yloxy, and C₃₋₈-heterocycloalk(en)yloxy;
R³ is selected from the group consisting of C₁₋₈-alk(en/yn)yl, C₃₋₈-cycloalk(en)yl, C₃₋₈-cycloalk(en)yl-C₁₋₆-alk(en/yn)yl, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, C₃₋₈-heterocycloalk(en)yl-C₁₋₆-alk(en/yn)yl, C₁₋₆-alk(en/yn)yl-C₃₋₈-heterocycloalk(en)yl-C₁₋₆-alk(en/yn)yl, Heteroaryl-C alk(en/yn)yl, Heteroaryl-C₃₋₈-cycloalk(en)yl, Heteroaryl-C₃₋₈-cycloalk(en)yl-C₁₋₆-alk(en/yn)yl, amino-C₁₋₆-alk(en/yn)yl, amino-C₃₋₈-cycloalk(en)yl, amino-C₃₋₈-cycloalk(en)yl-C₁₋₆-alk(en/yn)yl, C₁₋₆-alk(en/yn)yloxy-C₁₋₆-alk(en/yn)yl, C₃₋₈-cycloalk(en)yloxy-C₁₋₆-alk(en/yn)yl, C₃₋₈-cycloalk(en)yl-C₁₋₆-alk(en/yn)yloxy-C₁₋₆-alk(en/yn)yl, halo-C₁₋₆-alk(en/yn)yl, halo-C₃₋₈-cycloalk(en)yl and halo-C₃₋₈-cycloalk(en)yl-C₁₋₆-alk(en/yn)yl; and
R⁴ is selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, C₃₋₈-heterocycloalk(en)yl, Aryl, Heteroaryl, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, Aryl-C₃₋₈-heterocycloalk(en)yl, halo-C₁₋₆-alk(en/yn)yl, halo-C₃₋₈-cycloalk(en)yl, halo-C₃₋₈-cycloalk(en)yl-C₁₋₆-alk(en/yn)yl, halo-C₁₋₆-alk(en/yn)yl-C₃₋₈-heterocycloalk(en)yl-C₁₋₆-alk(en/yn)yl, NR⁵R⁶ and R⁷NH—C₁₋₆-alk(en/yn)yl; wherein:
R⁵ and R⁶ are independently selected from the group consisting of hydrogen, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, C₁₋₆-alk(en/yn)yl, C₃₋₈-cycloalk(en)yl, C₃₋₈-cycloalk(en)yl-C₁₋₆-alk(en/yn)yl, Heteroaryl-C₁₋₆-alk(en/yn)yl, Heteroaryl-C₃₋₈-cycloalk(en)yl and Heteroaryl-C₃₋₈-cycloalk(en)yl-C₁₋₆-alk(en/yn)yl, with the proviso that R⁵ and R⁶ are not hydrogen at the same time; and
R⁷ is selected from the group consisting of C₁₋₆-alk(en/yn)yl, C₃₋₈-cycloalk(en)yl, C₃₋₈-cycloalk(en)yl-C₁₋₆-alk(en/yn)yl, Aryl, halo-C₁₋₆-alk(en/yn)yl, halo-C₃₋₈-cycloalk(en)yl, halo-C₃₋₈-cycloalk(en)yl-C₁₋₆-alk(en/yn)yl, Aryl-C₁₋₆-alk(en/yn)yl, Aryl-C₃₋₈-cycloalk(en)yl and Heteroaryl; or
a pharmaceutically acceptable salt thereof.

97. (canceled)

98. The method according to claim 96, wherein said compound is selected from the group consisting of:
Hexanoic acid (4-bromo-2,6-dimethyl-phenyl)-amide;
N-(4-Bromo-2,6-dimethyl-phenyl)-2-(4-fluoro-phenyl)-acetamide;
N-(2-Bromo-4,6-dimethyl-phenyl)-2-(4-fluoro-phenyl)-acetamide;
N-(2-Bromo-4,6-dimethyl-phenyl)-3,3-dimethyl-butyramide;
N-(2-Bromo-4,6-dimethyl-phenyl)-2-cyclopentyl-acetamide;
N-(2-Bromo-4,6-dichloro-phenyl)-3,3-dimethyl-butyramide;
N-(2-Bromo-4,6-dichloro-phenyl)-2-(4-fluoro-phenyl)-acetamide;
N-(2-Bromo-4,6-dichloro-phenyl)-2-cyclopentyl-acetamide;
Heptanoic acid (4-bromo-2,6-dimethyl-phenyl)-amide;
Cyclohexanecarboxylic acid (4-bromo-2,6-dimethyl-phenyl)-amide;
N-(4-Bromo-2,6-dimethyl-phenyl)-2-thiophen-2-yl-acetamide;
2-Phenyl-cyclopropanecarboxylic acid (4-bromo-2,6-dimethyl-phenyl)-amide;
N-(4-Bromo-2,6-dimethyl-phenyl)-2-(4-chloro-phenyl)-acetamide;
Pentanoic acid (4-bromo-2,6-dimethyl-phenyl)-amide;
Octanoic acid (4-bromo-2,6-dimethyl-phenyl)-amide;
N-(4-Bromo-2,6-dimethyl-phenyl)-2-cyclopentyl-acetamide;
2-Bicyclo[2.2.1]kept-2-yl-N-(2,4-difluoro-6-morpholin-4-yl-phenyl)-acetamide;
(S)-2-Amino-N-{2,6-dimethyl-4-[methyl-(4-trifluoromethyl-benzyl)-amino]-phenyl}-3- methyl-butyramide;
(S)-2-Amino-4-methyl-pentanoic acid {2,6-dimethyl-4-[methyl-(4-trifluoromethyl-benzyl)-amino]-phenyl}-amide;
(4-Bromo-2,6-dimethyl-phenyl)-carbamic acid ethyl ester;
(4-Bromo-2,6-dimethyl-phenyl)-carbamic acid propyl ester;
N-(2-Amino-4-bromo-6-methyl-phenyl)-3,3-dimethyl-butyramide;
2-Cyclopentyl-N-{2,6-dimethyl-4-[2-(4-trifluoromethyl-phenyl)-pyrrolidin-1-yl]-phenyl}-acetamide;
N-(4-Azepan-1-yl-2,6-dimethyl-phenyl)-2-cyclopentyl-acetamide;
2-Cyclopentyl-N-(2,6-dimethyl-4-pyrrol-1-yl-phenyl)-acetamide;
N-(3′-Amino-3,5-dimethyl-biphenyl-2-yl)-2-(4-fluoro-phenyl)-acetamide;
N-(4′-Dimethylamino-3,5-dimethyl-biphenyl-2-yl)-2-(4-fluoro-phenyl)-acetamide;
N-(2,4-Dimethyl-6-quinolin-3-yl-phenyl)-2-(4-fluoro-phenyl)-acetamide;
2-(4-Fluoro-phenyl)-N-(4′-hydroxy-3′-methoxy-3,5-dimethyl-biphenyl-2-yl)-acetamide;
2-(4-Fluoro-phenyl)-N-(3′-hydroxy-3,5-dimethyl-biphenyl-2-yl)-acetamide;
2-(4-Fluoro-phenyl)-N-(2′-methanesulfonylamino-3,5-dimethyl-biphenyl-2-yl)-acetamide;
N-(4′-Isopropyl-3,5-dimethyl-biphenyl-2-yl)-3,3-dimethyl-butyramide;
2-Cyclopentyl-N-(3,5-dimethyl-biphenyl-2-yl)-acetamide;
N-(4′-Fluoro-3,5-dimethyl-biphenyl-2-yl)-2-(4-fluoro-phenyl)-acetamide;
N-(3,5-Dimethyl-3′,5′-bis-trifluoromethyl-biphenyl-2-yl)-2-(4-fluoro-phenyl)-acetamide;
N-(3′-Acetylamino-3,5-dimethyl-biphenyl-2-yl)-2-(4-fluoro-phenyl)-acetamide;
2-(4-Fluoro-phenyl)-N-(2′-methoxy-3,5-dimethyl-biphenyl-2-yl)-acetamide;
N-(3,5-Dimethyl-4′-vinyl-biphenyl-2-yl)-2-(4-fluoro-phenyl)-acetamide;
N-(3′-Cyano-3,5-dimethyl-biphenyl-2-yl)-2-(4-fluoro-phenyl)-acetamide;
N-(3,5-Dimethyl-3′-trifluoromethoxy-biphenyl-2-yl)-2-(4-fluoro-phenyl)-acetamide;
N-[2-(2,3-Dihydro-benzo[1,4]dioxin-6-yl)-4,6-dimethyl-phenyl]-2-(4-fluoro-phenyl)-acetamide;
N-[2,4-Dimethyl-6-(2,2,5-trimethyl-2,3-dihydro-benzofuran-7-yl)-phenyl]-2-(4-fluoro-phenyl)-acetamide;
N-[2,6-Dimethyl-4-(4-trifluoromethyl-benzylamino)-phenyl]-acetamide;
N-{2,6-Dimethyl-4-[methyl-(4-trifluoromethyl-benzyl)-amino]-phenyl}-acetamide;
{4-[(5-Chloro-thiophen-2-ylmethyl)-amino]-2,6-dimethyl-phenyl}-carbamic acid propyl ester;
[4-(4-Fluoro-benzylamino)-2,6-dimethyl-phenyl]-carbamic acid propyl ester;
[2,6-Dimethyl-4-(4-trifluoromethyl-benzylamino)-phenyl]-carbamic acid propyl ester;
[4-(3-Fluoro-4-trifluoromethyl-benzylamino)-2,6-dimethyl-phenyl]-carbamic acid propyl ester;
{2,6-Dimethyl-4-[(4-methyl-2-phenyl-pyrimidin-5-ylmethyl)-amino]-phenyl}-carbamic acid propyl ester;
{2,6-Dimethyl-4-[(6-p-tolyloxy-pyridin-3-ylmethyl)-amino]-phenyl}-carbamic acid propyl ester;
{4-[(6-Methoxy-pyridin-3-ylmethyl)-amino]-2,6-dimethyl-phenyl}-carbamic acid propyl ester;
{4-[(3-Fluoro-4-trifluoromethyl-benzyl)-methyl-amino]-2,6-dimethyl-phenyl}-carbamic acid propyl ester;
2-Cyclopentyl-N-[2,6-dimethyl-4-(4-trifluoromethyl-benzylamino)-phenyl]-acetamide
2-Cyclopentyl-N-{2,6-dimethyl-4-[methyl-(4-trifluoromethyl-benzyl)-amino]-phenyl}-acetamide;
2-Cyclopentyl-N-{2,6-dimethyl-4-[(6-trifluoromethyl-pyridin-3-ylmethyl)-amino]-phenyl}-acetamide;
N-{2,6-Dimethyl-4-[(6-trifluoromethyl-pyridin-3-ylmethyl)-amino]-phenyl}-3,3-dimethyl-butyramide;
N-{2-Bromo-4-[(5-chloro-thiophen-2-ylmethyl)-amino]-6-trifluoromethyl-phenyl}-3-cyclohexyl-propionamide;
{4-[(3-Fluoro-phenylamino)-methyl]-2,6-dimethyl-phenyl}-carbamic acid ethyl ester;
{2,6-Dimethyl-4-[(4-trifluoromethyl-phenylamino)-methyl]-phenyl}-carbamic acid ethyl ester;
2-Cyclopentyl-N-{4-[(3-fluoro-phenylamino)-methyl]-2,6-dimethyl-phenyl}-acetamide;
N-{4-[(3-Chloro-phenylamino)-methyl]-2,6-dimethyl-phenyl}-2-cyclopentyl-acetamide;
2-Cyclopentyl-N-{4-[(3-methoxy-phenylamino)-methyl]-2,6-dimethyl-phenyl}-acetamide;
N-{4-[(4-Chloro-phenylamino)-methyl]-2,6-dimethyl-phenyl}-2-cyclopentyl-acetamide;
2-Cyclopentyl-N-{4-[(3,4-difluoro-phenylamino)-methyl]-2,6-dimethyl-phenyl}-acetamide;
2-Cyclopentyl-N-{2,6-dimethyl-4-[(4-trifluoromethyl-phenylamino)-methyl]-phenyl}-acetamide;
2-Cyclopentyl-N-[2,6-dimethyl-4-(p-tolylamino-methyl)-phenyl]-acetamide;
2-Cyclopentyl-N-{2,6-dimethyl-4-[(3-trifluoromethyl-phenylamino)-methyl]-phenyl}-acetamide;
2-Cyclopentyl-N-{-4-[(3,5-difluoro-phenylamino)-methyl]-2,6-dimethyl-phenyl}-acetamide;
{4-[(4-Fluoro-phenylamino)-methyl]-2,6-dimethyl-phenyl}-carbamic acid propyl ester;
{4-[(4-Chloro-phenylamino)-methyl]-2,6-dimethyl-phenyl}-carbamic acid propyl ester;
{2,6-Dimethyl-4-[(4-trifluoromethyl-phenylamino)-methyl]-phenyl}-carbamic acid propyl ester;
{4-[(3,5-Difluoro-phenylamino)-methyl]-2,6-dimethyl-phenyl}-carbamic acid propyl ester;
{4-[(3-Fluoro-phenylamino)-methyl]-2,6-dimethyl-phenyl}-carbamic acid propyl ester;
N-(4-Bromo-2-methyl-6-morpholin-4-yl-phenyl)-3,3-dimethyl-butyramide;
{4-[(4-Methoxyphenylamino)-methyl]-2,6-dimethylphenyl}-carbamic acid propyl ester;
(R)-2-Amino-4-methylpentanoic acid [2,6-dimethyl-4-(4-trifluoromethylbenzylamino)-phenyl]-amide;
Pentanoic acid (4-[(4-chlorophenylamino)-methyl]-2,6-dimethylphenyl)-amide;
2-(4-Chlorophenyl)-N-[4-(4-chlorophenylamino)-methyl]-2,6-dimethyl phenyl)-acetamide;
{2,6-Dimethyl-4-[(4-trifluoromethylphenylamino)-methyl]-phenyl}-carbamic acid 2-methoxyethyl ester;
N-{4-[(5-Chloro-pyridin-2-ylamino)-methyl]-2,6-dimethylphenyl}-2-cyclopentylacetamide;
2-Cyclopentyl-N-{4-[(2,6-dichloro-pyridin-4-ylamino)-methyl]-2,6-dimethylphenyl}-acetamide;
N-{2-Chloro-6-methyl-4-[(6-trifluoromethyl-pyridin-3-ylmethyl)-amino]-phenyl}-2-(3-fluoro-phenyl)-acetamide;
N-[2-Chloro-6-trifluoromethyl-4-(4-trifluoromethylbenzylamino)-phenyl]-2-cyclopentylacetamide;
[2-Amino-6-methyl-4-(4-trifluoromethylbenzylamino)-phenyl]-carbamic acid ethyl ester;
3,3-Dimethyl-N-{2-methyl-6-morpholin-4-yl-4-(4-trifluoromethylbenzylamino)-phenyl}-butyramide;
2-Cyclopentyl-N-{2,6-dichloro-4-[(4-fluoro-phenylamino)-methyl]-phenyl}-acetamide;
2-Cyclopentyl-N-{2,6-dichloro-4-[(5-trifluoromethylpyridin-2-ylamino)-methyl]-phenyl}-acetamide; and
pharmaceutically acceptable salts thereof.

99. The method according to claim 1, wherein said compound is a compound according to formula 7: 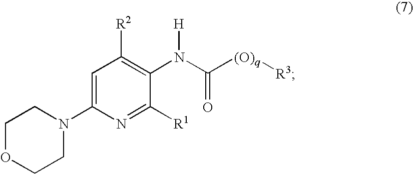
wherein:
q is 0 or 1;
each of R¹ and R² is independently selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy; and
R³ is selected from the group consisting of C₁₋₈-alk(en/yn)yl, C₃₋₈-cycloalk(en)yl, C₃₋₈-cycloalk(en)yl-C₁₋₆-alk(en/yn)yl, optionally substituted Aryl-C₁₋₆-alk(en/yn)yl, optionally substituted Aryl-C₃₋₈-cycloalk(en)yl, optionally substituted Aryl-C₃₋₈-cycloalk(en)yl-C₁₋₆-alk(en/yn)yl, C₁₋₆-alk(en/yn)yl-C₃₋₈-heterocycloalk(en)yl-C₁₋₆-alk(en/yn)yl, C₃₋₈-heterocycloalk(en)yl-C₁₋₆-alk(en/yn)yl, C₁₋₆-alk(en/yn)yl, C₃₋₈-heterocycloalk(en)yl-C₁₋₆-alk(en/yn)yl, Heteroaryl-C₁₋₆-alk(en/yn)yl, Heteroaryl-C₃₋₈-cycloalk(en)yl, Heteroaryl-C₃₋₈-cycloalk(en)yl-C₁₋₆-alk(en/yn)yl, NR⁴R⁵—C₁₋₆-alk(en/yn)yl, NR⁴R⁵—C₃₋₈-cycloalk(en)yl NR⁴R⁵—C₃₋₈-cycloalk(en)yl-C₁₋₆-alk(en/yn)yl, C₁₋₆-alk(en/yn)yloxy-C₁₋₆-alk(en/yn)yl, C₃₋₈-cycloalk(en)yloxy-C₁₋₆-alk(en/yn)yl, C₃₋₈-cycloalk(en)yl-C₁₋₆-alk(en/yn)yloxy-C₁₋₆-alk(en/yn)yl, halo-C₁₋₆-alk(en/yn)yl, halo-C₃₋₈-cycloalk(en)yl and halo-C₃₋₈-cycloalk(en)yl-C₁₋₆-alk(en/yn)yl; wherein each of R⁴ and R⁵ is independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl;
as the free base or a pharmaceutically acceptable salt thereof.

100. (canceled)

101. The method according to claim 99, wherein said compound is selected from the group consisting of:
(2,4-Dimethyl-6-morpholin-4-yl-pyridin-3-yl)-carbamic acid benzyl ester;
(2,4-Dimethyl-6-morpholin-4-yl-pyridin-3-yl)-carbamic acid 2-chloro-benzyl ester;
2-(4-Chloro-phenyl)-(2,4-dimethyl-6-morpholin-4-yl-pyridin-3-yl)-acetamide;
2-Phenyl-cyclopropanecarboxylic acid (2,4-dimethyl-6-morpholin-4-yl-pyridin-3-yl)-amide;
N-(2,4-Dimethyl-6-morpholin-4-yl-pyridin-3-yl)-2-thiophen-2-yl-acetamide;
3-Cyclohexyl-N-(2,4-dimethyl-6-morpholin-4-yl-pyridin-3-yl)-propionamide;
(2,4-Dimethyl-6-morpholin-4-yl-pyridin-3-yl)-carbamic acid isobutyl ester;
3-(3-Chloro-phenyl)-N-(2,4-dimethyl-6-morpholin-4-yl-pyridin-3-yl)-propionamide;
N-(2,4-Dimethyl-6-morpholin-4-yl-pyridin-3-yl)-2-(3,5-dimethyl-phenyl)-acetamide;
N-(2,4-Dimethyl-6-morpholin-4-yl-pyridin-3-yl)-3-p-tolyl-propionamide;
2-(3-Chloro-phenyl)-N-(2,4-dimethyl-6-morpholin-4-yl-pyridin-3-yl)-acetamide;
2-(3,4-Dichloro-phenyl)-N-(2,4-dimethyl-6-morpholin-4-yl-pyridin-3-yl)-acetamide;
N-(2,4-Dimethyl-6-morpholin-4-yl-pyridin-3-yl)-2-thiophen-3-yl-acetamide;
N-(2,4-Dimethyl-6-morpholin-4-yl-pyridin-3-yl)-2-p-tolyl-acetamide;
2-(3-Bromo-phenyl)-N-(2,4-dimethyl-6-morpholin-4-yl-pyridin-3-yl)-acetamide;
N-(2,4-Dimethyl-6-morpholin-4-yl-pyridin-3-yl)-2-(3-trifluoromethyl-phenyl)-acetamide;
N-(2,4-Dimethyl-6-morpholin-4-yl-pyridin-3-yl)-2-phenyl-acetamide;
3,5,5-Trimethyl-hexanoic acid (2,4-dimethyl-6-morpholin-4-yl-pyridin-3-yl)-amide;
Octanoic acid (2,4-dimethyl-6-morpholin-4-yl-pyridin-3-yl)-amide;
N-(2,4-Dimethyl-6-morpholin-4-yl-pyridin-3-yl)-2-naphthalen-2-yl-acetamide;
Heptanoic acid (2,4-dimethyl-6-morpholin-4-yl-pyridin-3-yl)-amide;
N-(2,4-Dimethyl-6-morpholin-4-yl-pyridin-3-yl)-2-(3,4-dimethyl-phenyl)-acetamide;
2-Cyclohex-1-enyl-N-(2,4-dimethyl-6-morpholin-4-yl-pyridin-3-yl)-acetamide;
N-(2,4-Dimethyl-6-morpholin-4-yl-pyridin-3-yl)-2-(4-methoxy-3-methyl-phenyl)-acetamide;
N-(2,4-Dimethyl-6-morpholin-4-yl-pyridin-3-yl)-2-(4-methoxy-phenyl)-acetamide;
N-(2,4-Dimethyl-6-morpholin-4-yl-pyridin-3-yl)-3-(4-methoxy-phenyl)-propionamide;
N-(2,4-Dimethyl-6-morpholin-4-yl-pyridin-3-yl)-2-m-tolyl-acetamide;
N-(2,4-Dimethyl-6-morpholin-4-yl-pyridin-3-yl)-2-(4-fluoro-phenyl)-acetamide;
N-(2,4-Dimethyl-6-morpholin-4-yl-pyridin-3-yl)-3,3-dimethyl-butyramide;
N-(2,4-Dimethyl-6-morpholin-4-yl-pyridin-3-yl)-2-(3-fluoro-phenyl)-acetamide;
2-Bicyclo[2.2.1]kept-2-yl-N-(2,4-dimethyl-6-morpholin-4-yl-pyridin-3-yl)-acetamide;
2-(3,4-Difluoro-phenyl)-N-(2,4-dimethyl-6-morpholin-4-yl-pyridin-3-yl)-acetamide;
4-Methyl-pentanoic acid (2,4-dimethyl-6-morpholin-4-yl-pyridin-3-yl)-amide;
2-Cyclopent-2-enyl-N-(2,4-dimethyl-6-morpholin-4-yl-pyridin-3-yl)-acetamide;
2-Cyclohexyl-N-(2,4-dimethyl-6-morpholin-4-yl-pyridin-3-yl)-acetamide;
5-Methyl-hexanoic acid (2,4-dimethyl-6-morpholin-4-yl-pyridin-3-yl)-amide;
2-Cyclopentyl-N-(2,4-dimethyl-6-morpholin-4-yl-pyridin-3-yl)-acetamide;
3-Cyclopentyl-N-(2,4-dimethyl-6-morpholin-4-yl-pyridin-3-yl)-propionamide;
Hexanoic acid (2,4-dimethyl-6-morpholin-4-yl-pyridin-3-yl)-amide;
N-(4-Chloro-2-methoxy-6-morpholin-4-yl-pyridin-3-yl)-2-cyclopentylacetamide;
N-(2-Chloro-4-methoxy-6-morpholin-4-yl-pyridin-3-yl)-2-cyclopentylacetamide;
N-(2-Chloro-4-methoxy-6-morpholin-4-yl-pyridin-3-yl)-3,3-dimethylbutyramide;
N-(4-Chloro-2-methoxy-6-morpholin-4-yl-pyridin-3-yl)-3,3-dimethylbutyramide;
N-(4-Chloro-2-methoxy-6-morpholin-4-yl-pyridin-3-yl)-propionamide; and
pharmaceutically acceptable salts thereof.

102. The method according to claim 1, wherein said compound is a compound according to formula 8: 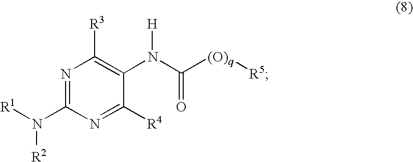
wherein:
q is 0 or 1;
R¹ and R² are independently selected from the group consisting of hydrogen and optionally substituted aryl-C₁₋₆-alk(en/yn)yl, provided that R¹ and R² are not both hydrogen; or
R¹ and R² together with the nitrogen to which they are attached form a 5 to 7 membered ring optionally containing a further heteroatom;
R² and R⁴ are independently selected from hydrogen, halogen, cyano, amino, C₁₋₆-alk(en/yn)yl, C₃₋₈-cycloalk(en)yl, halo-C₁₋₆-alk(en/yn)yl, halo-C₃₋₈-cycloalk(en)yl, C₁₋₆-alk(en/yn)yloxy, C₃₋₈-cycloalk(en)yloxy, C₃₋₈-cycloalk(en)yl-C₁₋₆-alk(en/yn)yloxy, halo-C₁₋₆-alk(en/yn)yloxy, halo-C₃₋₈-cycloalk(en)yloxy and halo-C₃₋₈-cycloalk(en)yl-C₁₋₆-alk(en/yn)yloxy, provided that R³ and R⁴ are not both hydrogen; and
R⁵ is selected from the group consisting of C₁₋₁₀-alk(en/yn)yl, C₃₋₈-cycloalk(en)yl-C₁₋₆-alk(en/yn)yl, optionally substituted aryl-C₁₋₆-alk(en/yn)yl and optionally substituted aryl;
as the free base or a pharmaceutically acceptable salt thereof.

103. (canceled)

104. The method according to claim 102, wherein said compound is selected from the group consisting of:
N-[4-Amino-6-methyl-2-(4-trifluoromethylbenzylamino)-pyrimidin-5-yl]-2-cyclopentylacetamide;
N-[4-Amino-6-methyl-2-(4-trifluoromethylbenzylamino)-pyrimidin-5-yl]-3,3-dimethylbutyramide;
N-[4-Amino-6-methyl-2-(4-trifluoromethylbenzylamino)-pyrimidin-5-yl]-2-(4-fluorophenyl)-acetamide;
Hexanoic acid [4-amino-6-methyl-2-(4-trifluoromethylbenzylamino)-pyrimidin-5-yl]-amide;
N-[4-Amino-6-methyl-2-(4-trifluoromethylbenzylamino)-pyrimidin-5-yl]-2-(3-chlorophenyl)-acetamide;
2-Cyclopentyl-N-(4,6-dimethyl-2-morpholin-4-yl-pyrimidin-5-yl)-acetamide;
N-(4,6-Dimethyl-2-morpholin-4-yl-pyrimidin-5-yl)-3,3-dimethylbutyramide;
N-(4,6-Dimethyl-2-morpholin-4-ylpyrimidin-5-yl)-2-(4-fluorophenyl)-acetamide;
2-(3,4-Difluorophenyl)-N-(4,6-dimethyl-2-morpholin-4-ylpyrimidin-5-yl)-acetamide;
N-(4,6-Dimethyl-2-morpholin-4-ylpyrimidin-5-yl)-2-(3-fluorophenyl)-acetamide; and
Hexanoic acid (4,6-dimethyl-2-morpholin-4-ylpyrimidin-5-yl)-amide;
as the free base or a pharmaceutically acceptable salt thereof.

105. The method according to claim 1
wherein said compound is the compound of formula 9: 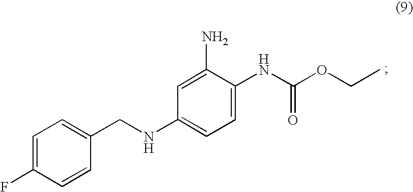
or a pharmaceutically acceptable salt thereof.

106. The method according to claim 1, wherein said compound is the compound of formula 10: 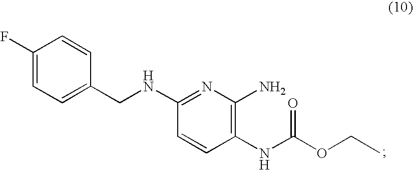
or a pharmaceutically acceptable salt thereof.

107. The method according to claim 1, wherein the compound is selected from the group consisting of:
2-Cyclopentyl-N-(2,6-dimethyl-4-morpholin-4-yl-phenyl)-acetamide;
N-(2,6-Dimethyl-4-morpholin-4-yl-phenyl)-3,3-dimethyl-butyramide;
N-(4,6-Dimethyl-2-morpholin-4-yl-pyrimidin-5-yl)-2-(4-fluoro-phenyl)-acetamide;
Hexanoic acid (2,6-difluoro-4-morpholin-4-yl-phenyl)-amide;
2-Cyclopentyl-N-(4,6-dimethyl-2-morpholin-4-yl-pyrimidin-5-yl)-acetamide;
N-(2-Bromo-4-morpholin-4-yl-6-trifluoromethyl-phenyl)-propionamide;
N-(2,4-Dimethyl-6-morpholin-4-yl-pyridin-3-yl)-3,3-dimethyl-butyramide;
[2-Amino-4-(2,4,6-trimethyl-benzylamino)-phenyl]-carbamic acid ethyl ester;
2-Cyclopentyl-N-(2-methoxy-6-methyl-4-morpholin-4-yl-phenyl)-acetamide; and
pharmaceutically acceptable salts thereof.

108.-112. (canceled)
